(19) United States
(12) Patent Application Publication
(10) Pub. No.: US 2010/0093748 A1
(43) Pub. Date: Apr. 15, 2010

(54) SUBSTITUTED PIPERIDINES HAVING PROTEIN KINASE INHIBITING ACTIVITY

(75) Inventors: Steven John Woodhead, Cambridge (GB); Martyn Frederickson, Cambridge (GB); Christopher Hamlett, Cambridge (GB); Andrew James Woodhead, Cambridge (GB); Marinus Leendert Verdonk, Burwell (GB); Hannah Fiona Sore, Cambridge (GB); David Winter Walker, Cambridge (GB); Peter Blurton, Welwyn Garden City (GB); Ian Collins, Redhill (GB); Kwai Ming Cheung, Staines (GB); John Caldwell, Sutton (GB); Tatiana Faria Da Fonseca McHardy, Sunbury-on-Thames (GB); Richard William Arthur Luke, Cheshire (GB); Zbigniew Stanley Matusiak, Cheshire (GB); Andrew Leach, Cheshire (GB); Jeffrey James Morris, Cheshire (GB)

Correspondence Address: HESLIN ROTHENBERG FARLEY & MESITI PC, 5 COLUMBIA CIRCLE, ALBANY, NY, 12203, US

(73) Assignees: ASTEX THERAPEUTICS LIMITED, Cambridge (GB); THE INSTITUTE OF CANCER RESEARCH: ROYAL CANCER HOS, London (GB); Cancer Research Technology Limited, London (GB)

(21) Appl. No.: 12/520,303

(22) PCT Filed: Dec. 21, 2007
(86) PCT No.: PCT/GB07/50776

§ 371 Date: Sep. 30, 2009

Related U.S. Application Data
(60) Provisional application No. 60/982,633, filed on Oct. 25, 2007.
(60) Provisional application No. 60/871,386, filed on Dec. 21, 2006.

(30) Foreign Application Priority Data
Dec. 21, 2006 (GB) 0625668.9

(51) Int. Cl. A61K 31/497; C07D 487/04; A61K 31/519; C07D 471/04; A61K 31/437

(52) U.S. Cl. 514/252.16; 544/280; 514/265.1; 544/230; 514/262.1; 544/256; 546/113; 514/300

(57) ABSTRACT

The invention provides PKA and PKB kinase-inhibiting compounds of the formula (I):

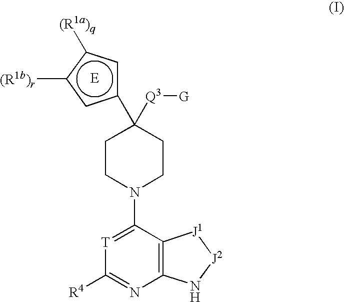

or salts, solvates, tautomers or N-oxides thereof, wherein E is a five membered heteroaryl ring containing 1, 2, 3 or 4 heteroatoms selected from O, N and S provided that no more than 1 heteroatom may be other than N; q and r are each is 0 or 1; provided that q+r is 1 or 2; T is N or a group CR⁵; J¹-J² is N═C(R⁶), (R⁷)C═N, (R⁸)N—C(O), (R⁸)₂C—C(O), N═N or (R⁷)C═C(R⁶); Q³ is a bond or a saturated C₁₋₃ hydrocarbon linker group optionally substituted by fluorine and hydroxy; G is NR²R³, CN or OH; m and n are each 0 or 1, provided that m+n is 1 or 2, and provided also that m or n are each 0 when the adjacent ring member of ring E is S or O; R^{1a} and R^{1b} are the same or different and each is hydrogen or a substituent R¹⁰; or R^{1a} and R^{1b} together with the carbon atoms or heteroatoms to which they are attached form a 5 or 6-membered aryl or heteroaryl ring, wherein the aryl or heteroaryl rings are optionally substituted by one or more substituents R¹⁰; and R², R³, R⁴, R⁵, R⁷, R⁶, R⁸, and R¹⁰ are as defined in the claims.

This invention relates to purine, purinone and deazapurine and deazapurinone compounds or structural isomers thereof that inhibit or modulate the activity of protein kinase B (PKB) and/or protein kinase A (PKA), to the use of the compounds in the treatment or prophylaxis of disease states or conditions mediated by PKB and/or PKA, and to novel compounds having PKB and/or PKA inhibitory or modulating activity. Also provided are pharmaceutical compositions containing the compounds and novel chemical intermediates.

### BACKGROUND OF THE INVENTION

Protein kinases constitute a large family of structurally related enzymes that are responsible for the control of a wide variety of signal transduction processes within the cell (Hardie, G. and Hanks, S. (1995) The Protein Kinase Facts Book. I and II, Academic Press, San Diego, Calif.). The kinases may be categorized into families by the substrates they phosphorylate (e.g., protein-tyrosine, protein-serine/threonine, lipids, etc.). Sequence motifs have been identified that generally correspond to each of these kinase families (e.g., Hanks, S. K., Hunter, T., FASEB J., 9:576-596 (1995); Knighton, et al., Science, 253:407-414 (1991); Hiles, et al., Cell, 70:419-429 (1992); Kunz, et al., Cell, 73:585-596 (1993); Garcia-Bustos, et al., EMBO J., 13:2352-2361 (1994)).

Protein kinases may be characterized by their regulation mechanisms. These mechanisms include, for example, autophosphorylation, transphosphorylation by other kinases, protein-protein interactions, protein-lipid interactions, and protein-polynucleotide interactions. An individual protein kinase may be regulated by more than one mechanism.

Kinases regulate many different cell processes including, but not limited to, proliferation, differentiation, apoptosis, motility, transcription, translation and other signalling processes, by adding phosphate groups to target proteins. These phosphorylation events act as molecular on/off switches that can modulate or regulate the target protein biological function. Phosphorylation of target proteins occurs in response to a variety of extracellular signals (hormones, neurotransmitters, growth and differentiation factors, etc.), cell cycle events, environmental or nutritional stresses, etc. The appropriate protein kinase functions in signalling pathways to activate or inactivate (either directly or indirectly), for example, a metabolic enzyme, regulatory protein, receptor, cytoskeletal protein, ion channel or pump, or transcription factor. Uncontrolled signalling due to defective control of protein phosphorylation has been implicated in a number of diseases, including, for example, inflammation, cancer, allergy/asthma, diseases and conditions of the immune system, diseases and conditions of the central nervous system, and angiogenesis.

Apoptosis or programmed cell death is an important physiological process which removes cells no longer required by an organism. The process is important in early embryonic growth and development allowing the non-necrotic controlled breakdown, removal and recovery of cellular components. The removal of cells by apoptosis is also important in the maintenance of chromosomal and genomic integrity of growing cell populations. There are several known checkpoints in the cell growth cycle at which DNA damage and genomic integrity are carefully monitored. The response to the detection of anomalies at such checkpoints is to arrest the growth of such cells and initiate repair processes. If the damage or anomalies cannot be repaired then apoptosis is initiated by the damaged cell in order to prevent the propagation of faults and errors. Cancerous cells consistently contain numerous mutations, errors or rearrangements in their chromosomal DNA. It is widely believed that this occurs in part because the majority of tumours have a defect in one or more of the processes responsible for initiation of the apoptotic process. Normal control mechanisms cannot kill the cancerous cells and the chromosomal or DNA coding errors continue to be propagated. As a consequence restoring these pro-apoptotic signals or suppressing unregulated survival signals is an attractive means of treating cancer.

The signal transduction pathway containing the enzymes phosphatidylinositol 3-kinase (PI3K), PDK1 and PKB amongst others, has long been known to mediate increased resistance to apoptosis or survival responses in many cells. There is a substantial amount of data to indicate that this pathway is an important survival pathway used by many growth factors to suppress apoptosis. The enzymes of the PI3K family are activated by a range of growth and survival factors e.g. EGF, PDGF and through the generation of polyphosphatidylinositols, initiates the activation of the downstream signalling events including the activity of the kinases PDK1 and protein kinase B (PKB) also known as akt. This is also true in host tissues, e.g. vascular endothelial cells as well as neoplasias. PKB is a protein ser/thr kinase consisting of a kinase domain together with an N-terminal PH domain and C-terminal regulatory domain. The enzyme PKBₐₗₚₕₐ (akt1) itself is phosphorylated on Thr 308 by PDK1 and on Ser 473 by ‘PDK2’ now believed to be constituted from the target of rapamycin (TOR) kinase and its associated protein rictor. Full activation requires phosphorylation at both sites whilst association between PIP3 and the PH domain is required for anchoring of the enzyme to the cytoplasmic face of the lipid membrane providing optimal access to substrates.

At least 10 kinases have been suggested to function as a Ser 473 kinase including mitogen-activated protein (MAP) kinase-activated protein kinase-2 (MK2), integrin-linked kinase (ILK), p38 MAP kinase, protein kinase Calpha (PKCalpha), PKCbeta, the NIMA-related kinase-6 (NEK6), the mammalian target of rapamycin (mTOR), the double-stranded DNA-dependent protein kinase (DNK-PK), and the ataxia telangiectasia mutated (ATM) gene product. Available data suggest that multiple systems may be used in cells to regulate the activation of PKB. Full activation of PKB requires phosphorylation at both sites whilst association between PIP3 and the PH domain is required for anchoring of the enzyme to the cytoplasmic face of the lipid membrane providing optimal access to substrates.

Recently, it has been reported that somatic mutations within the PI3K catalytic subunit, PIK3CA, are common (25-40%) among colorectal, gastric, breast, ovarian cancers, and high-grade brain tumors. PIK3CA mutations are a common event that can occur early in bladder carcinogenesis. In invasive breast carcinomas, PIK3CA alterations are mainly present in lobular and ductal tumours. The PI3K pathway is extensively activated in endometrial carcinomas, and that combination of PIK3CA/PTEN alterations might play an important role in development of these tumors. Tumours activated by mutations of PI3 kinase and loss of PTEN will have sustained activation of PKB and will be as a result disproportionately sensitive to inihibition by PKA/PKB inhibitors.

Activated PKB in turns phosphorylates a range of substrates contributing to the overall survival response. Whilst we cannot be certain that we understand all of the factors responsible for mediating the PKB dependent survival response, some important actions are believed to be phosphorylation and inactivation of the pro-apoptotic factor BAD and caspase 9, phosphorylation of Forkhead transcription factors e.g. FKHR leading to their exclusion from the nucleus, and activation of the NfkappaB pathway by phosphorylation of upstream kinases in the cascade.

In addition to the anti-apoptotic and pro-survival actions of the PKB pathway, the enzyme also plays an important role in promoting cell proliferation. This action is again likely to be mediated via several actions, some of which are thought to be phosphorylation and inactivation of the cyclin dependent kinase inhibitor of p21^{Cip1/WAF1}, and phosphorylation and activation of mTOR, a kinase controlling several aspects of cell size, growth and protein translation.

The phosphatase PTEN which dephosphorylates and inactivates polyphosphatidyl-inositols is a key tumour suppressor protein which normally acts to regulate the PI3K/PKB survival pathway. The significance of the PI3K/PKB pathway in tumourigenesis can be judged from the observation that PTEN is one of the most common targets of mutation in human tumours, with mutations in this phosphatase having been found in ˜50% or more of melanomas (Guldberg et al 1997, Cancer Research 57, 3660-3663) and advanced prostate cancers (Cairns et al 1997 Cancer Research 57, 4997). These observations and others suggest that a wide range of tumour types are dependent on the enhanced PKB activity for growth and survival and would respond therapeutically to appropriate inhibitors of PKB.

There are 3 closely related isoforms of PKB called alpha, beta and gamma (AKT1, 2 and 3), which genetic studies suggest have distinct but overlapping functions. Evidence suggests that they can all independently play a role in cancer. For example PKB beta has been found to be over-expressed or activated in 10-40% of ovarian and pancreatic cancers (Bellacosa et al 1995, Int. J. Cancer 64, 280-285; Cheng et al 1996, PNAS 93, 3636-3641; Yuan et al 2000, Oncogene 19, 2324-2330), PKB alpha is amplified in human gastric, prostate and breast cancer (Staal 1987, PNAS 84, 5034-5037; Sun et al 2001, Am. J. Pathol. 159, 431-437) and increased PKB gamma activity has been observed in steroid independent breast and prostate cell lines (Nakatani et al 1999, J. Biol. Chem. 274, 21528-21532).

The PKB pathway also functions in the growth and survival of normal tissues and may be regulated during normal physiology to control cell and tissue function. Thus disorders associated with undesirable proliferation and survival of normal cells and tissues may also benefit therapeutically from treatment with a PKB inhibitor. Examples of such disorders are disorders of immune cells associated with prolonged expansion and survival of cell population leading to a prolonged or up regulated immune response. For example, T and B lymphocyte response to cognate antigens or growth factors such as interferon gamma activates the PI3K/PKB pathway and is responsible for maintaining the survival of the antigen specific lymphocyte clones during the immune response. Under conditions in which lymphocytes and other immune cells are responding to inappropriate self or foreign antigens, or in which other abnormalities lead to prolonged activation, the PKB pathway contributes an important survival signal preventing the normal mechanisms by which the immune response is terminated via apoptosis of the activated cell population. There is a considerable amount of evidence demonstrating the expansion of lymphocyte populations responding to self antigens in autoimmune conditions such as multiple sclerosis and arthritis. Expansion of lymphocyte populations responding inappropriately to foreign antigens is a feature of another set of conditions such as allergic responses and asthma. In summary inhibition of PKB could provide a beneficial treatment for immune disorders.

Other examples of inappropriate expansion, growth, proliferation, hyperplasia and survival of normal cells in which PKB may play a role include but are not limited to atherosclerosis, cardiac myopathy and glomerulonephritis.

In addition to the role in cell growth and survival, the PKB pathway functions in the control of glucose metabolism by insulin. Available evidence from mice deficient in the alpha and beta isoforms of PKB suggests that this action is mediated by the beta isoform primarily. As a consequence, modulators of PKB activity may also find utility in diseases in which there is a dysfunction of glucose metabolism and energy storage such as diabetes, metabolic disease and obesity.

Cyclic AMP-dependent protein kinase (PKA) is a serine/threonine protein kinase that phosphorylates a wide range of substrates and is involved in the regulation of many cellular processes including cell growth, cell differentiation, ion-channel conductivity, gene transcription and synaptic release of neurotransmitters. In its inactive form, the PKA holoenzyme is a tetramer comprising two regulatory subunits and two catalytic subunits.

PKA acts as a link between G-protein mediated signal transduction events and the cellular processes that they regulate. Binding of a hormone ligand such as glucagon to a transmembrane receptor activates a receptor-coupled G-protein (GTP-binding and hydrolyzing protein). Upon activation, the alpha subunit of the G protein dissociates and binds to and activates adenylate cyclase, which in turn converts ATP to cyclic-AMP (cAMP). The cAMP thus produced then binds to the regulatory subunits of PKA leading to dissociation of the associated catalytic subunits. The catalytic subunits of PKA, which are inactive when associated with the regulatory sub-units, become active upon dissociation and take part in the phosphorylation of other regulatory proteins.

For example, the catalytic sub-unit of PKA phosphorylates the kinase Phosphorylase Kinase which is involved in the phosphorylation of Phosphorylase, the enzyme responsible for breaking down glycogen to release glucose. PKA is also involved in the regulation of glucose levels by phosphorylating and deactivating glycogen synthase. Thus, modulators of PKA activity (which modulators may increase or decrease PKA activity) may be useful in the treatment or management of diseases in which there is a dysfunction of glucose metabolism and energy storage such as diabetes, metabolic disease and obesity.

PKA has also been established as an acute inhibitor of T cell activation. Anndahl et al, have investigated the possible role of PKA type I in HIV-induced T cell dysfunction on the basis that T cells from HIV-infected patients have increased levels of cAMP and are more sensitive to inhibition by cAMP analogues than are normal T cells. From their studies, they concluded that increased activation of PKA type I may contribute to progressive T cell dysfunction in HIV infection and that PKA type I may therefore be a potential target for immunomodulating therapy.-Aandahl, E. M., Aukrust, P., Skålhegg, B. S., Müller, F., Frøland, S. S., Hansson, V., Taskén, K. Protein kinase A type I antagonist restores immune responses of T cells from HIV-infected patients. FASEB J. 12, 855-862 (1998).

It has also been recognised that mutations in the regulatory sub-unit of PKA can lead to hyperactivation in endocrine tissue.

Because of the diversity and importance of PKA as a messenger in cell regulation, abnormal responses of cAMP can lead to a variety of human diseases derived from this, such as irregular cell growth and proliferation (Stratakis, C. A.; Cho-Chung, Y. S.; Protein Kinase A and human diseases. Trends Endrocri. Metab. 2002, 13, 50-52). Over-expression of PKA has been observed in a variety of human cancer cells including those from ovarian, breast and colon patients Inhibition of PKA would therefore be an approach to treatment of cancer (Li, Q.; Zhu, G-D.; Current Topics in Medicinal Chemistry, 2002, 2, 939-971).

For a review of the role of PKA in human disease, see for example, Protein Kinase A and Human Disease, Edited by Constantine A. Stratakis, Annals of the New York Academy of Sciences, Volume 968, 2002, ISBN 1-57331-412-9.

Several classes of compounds have been disclosed as having PKA and PKB inhibitory activity.

WO 99/65909 (Pfizer) discloses a class of pyrrole[2,3-d]pyrimidine compounds having protein tyrosine kinase activity and which are of potential use as immunosuppressant agents.

WO 2004/074287 (AstraZeneca) discloses piperazinyl-pyridyl amides for use in treating autoimmune diseases such as arthritis. The piperazine group in the compounds can be linked to a purine group.

WO02/18348 (F. Hoffman La Roche) discloses a class of amino-quinazoline derivatives as alpha-1 adrenergic antagonists. A method for preparing the amino-quinazoline compounds involves the use of a gem-disubstituted cyclic amine such as piperidine in which one of the gem substituents is an aminomethyl group.

WO03/088908 (Bristol Myers Squibb) discloses N-heteroaryl-4,4-disubstituted piperidines as potassium channel inhibitors.

WO01/07050 (Schering) discloses substituted piperidines as nociceptin receptor ORL-1 agonists for use in treating cough.

US 2003/0139427 (OSI) discloses pyrrolidine- and piperidine-substituted purines and purine analogues having adenosine receptor binding activity.

WO 2004/043380 (Harvard College et al.) discloses technetium and rhenium labelled imaging agents containing disubstituted piperidine metal ion-chelating ligands.

WO 97/38665 (Merck) discloses gem-disubstituted piperidine derivatives having farnesyl transferase inhibitory activity.

EP 1568699 (Eisai) discloses 1,3-dihydroimidazole fused ring compounds having DPPIV-inhibiting activity. The compounds are described as having a range of potential uses including the treatment of cancer.

US 2003/0073708 and US 2003/045536 (both in the name of Castelhano et al), WO 02/057267 (OSI Pharmaceuticals) and WO 99/62518 (Cadus Pharmaceutical Corporation) each disclose a class of 4-aminodeazapurines in which the 4-amino group can form part of a cyclic amine such as azetidine, pyrrolidine and piperidine, The compounds are described as having adenosine receptor antagonist activity.

U.S. Pat. No. 6,162,804 (Merck) discloses a class of benzimidazole and aza-benzimidazole compounds that have tyrosine kinase inhibitor activity.

WO 2005/003128 (Merck) discloses a class of acyl-thiazoly-piperidine compounds as MTP inhibitors and apoprotein B secretin inhibitors. The compounds are said of be useful in the treatment of lipid metabolism disorders and obesity.

WO 2006/071819 (Exelixis) discloses a class of [1H-pyrazolo[3,4-d]pyrimidin-4-yl]piperidine compounds as inhibitors of Akt1, Akt2 and P70S6K. The compounds are stated to be useful for the treatment of immunological, inflammatory and proliferative diseases.

### SUMMARY OF THE INVENTION

The invention provides compounds that have protein kinase B (PKB) and/or protein kinase A (PKA) inhibiting or modulating activity, and which it is envisaged will be useful in preventing or treating disease states or conditions mediated by PKB and/or PKA.

Accordingly, in one aspect, the invention provides a compound of the formula (I):

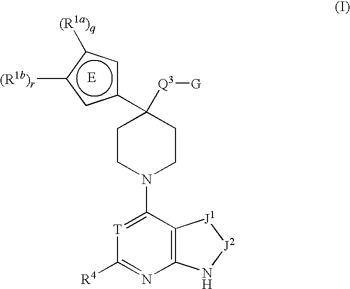

or salts, solvates, tautomers or N-oxides thereof, wherein
the ring E is a five membered heteroaryl ring containing 1, 2, 3 or 4 heteroatoms selected from O, N and S provided that no more than 1 heteroatom may be other than N;
q and r are each 0 or 1;
T is N or a group CR⁵;
J¹-J² represents a group selected from N═C(R⁶), (R⁷)C═N, (R⁸)N—C(O), (R⁸)₂C—C(O), N═N and (R⁷)C═C(R⁶);
Q³ is a bond or a saturated hydrocarbon linker group containing from 1 to 3 carbon atoms; wherein the carbon atoms of the linker group may optionally bear one or more substituents selected from fluorine and hydroxy, provided that the hydroxy group when present is not located at a carbon atom α with respect to the G group;
G is selected from NR²R³, CN and OH;
R^{1a} and R^{1b} are the same or different and each is hydrogen or a substituent R¹⁰; or R^{1a} and R^{1b} together with the carbon atoms or heteroatoms to which they are attached form a 5 or 6-membered aryl or heteroaryl ring, wherein the aryl or heteroaryl rings are optionally substituted by one or more substituents R¹⁰; provided that when R^{1a} and R^{1b} are each hydrogen or R¹⁰, then the heteroaryl ring E is other than a thiophene or furan ring;
R² and R³ are independently selected from hydrogen; C₁₋₄ hydrocarbyl and C₁₋₄ acyl wherein the hydrocarbyl and acyl groups are optionally substituted by one or more substituents selected from fluorine, hydroxy, cyano, amino, methylamino, dimethylamino, methoxy and a monocyclic or bicyclic aryl or heteroaryl group;
or R² and R³ together with the nitrogen atom to which they are attached form a cyclic group selected from an imidazole group and a saturated monocyclic heterocyclic group having 4-7 ring members and optionally containing a second heteroatom ring member selected from O and N;
or one of R² and R³ together with the nitrogen atom to which they are attached and one or more atoms from the group Q³ form a saturated monocyclic heterocyclic group having 4-7 ring members and optionally containing a second heteroatom ring member selected from O and N; and
R⁴, R⁶ and R⁸ are each independently selected from hydrogen, halogen, C₁₋₅ saturated hydrocarbyl, cyano, CONH₂, CF₃ and NH₂;
R⁵ and R⁷ are each independently selected from hydrogen, halogen, C₁₋₅ saturated hydrocarbyl, cyano and CF₃; and
R¹⁰ is selected from halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and C₁₋₈ hydrocarbyl optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{c} is selected from hydrogen and C₁₋₄ hydrocarbyl; and
X¹ is O, S or NR^{c} and X² is ═O, ═S or ═NR^{c}.

The invention also provides:
A compound of the formula (I), (II), (III), (IV), (IVa), (IVb), (V) or any sub-group thereof as defined herein for use in the prophylaxis or treatment of a disease state or condition mediated by protein kinase B and/or protein kinase A.
The use of a compound of formula (I), (II), (III), (IV), (IVa), (IVb), (V) or any sub-group thereof as defined herein for the manufacture of a medicament for the prophylaxis or treatment of a disease state or condition mediated by protein kinase B and/or protein kinase A.
A method for the prophylaxis or treatment of a disease state or condition mediated by protein kinase B and/or protein kinase A, which method comprises administering to a subject in need thereof a compound of the formula (I), (II), (III), (IV), (IVa), (IVb), (V) or any sub-group thereof as defined herein.
A method for treating a disease or condition comprising or arising from abnormal cell growth or abnormally arrested cell death in a mammal, the method comprising administering to the mammal a compound of the formula (I), (II), (III), (IV), (IVa), (IVb), (V) or any sub-group thereof as defined herein in an amount effective to inhibit protein kinase B activity.
A method of inhibiting protein kinase B, which method comprises contacting the kinase with a kinase-inhibiting compound of the formula (I), (II), (III), (IV), (IVa), (IVb), (V) or any sub-group thereof as defined herein.
A method of modulating a cellular process (for example cell division) by inhibiting the activity of a protein kinase B using a compound of the formula (I), (II), (III), (IV), (IVa), (IVb), (V) or any sub-group thereof as defined herein.
A compound of the formula (I), (II), (III), (IV), (IVa), (IVb), (V) or any sub-group or embodiment thereof as defined herein for use in the prophylaxis or treatment of a disease state or condition mediated by protein kinase A.
The use of a compound of formula (I), (II), (III), (IV), (IVa), (IVb), (V) or any sub-group or embodiment thereof as defined herein for the manufacture of a medicament for the prophylaxis or treatment of a disease state or condition mediated by protein kinase A.
A method for the prophylaxis or treatment of a disease state or condition mediated by protein kinase A, which method comprises administering to a subject in need thereof a compound of the formula (I), (II), (III), (IV), (IVa), (IVb), (V) or any sub-group or embodiment thereof as defined herein.
A method for treating a disease or condition comprising or arising from abnormal cell growth or abnormally arrested cell death in a mammal, the method comprising administering to the mammal a compound of the formula (I), (II), (III), (IV), (IVa), (IVb), (V) or any sub-group or embodiment thereof as defined herein in an amount effective to inhibit protein kinase A activity.
A method of inhibiting protein kinase A, which method comprises contacting the kinase with a kinase-inhibiting compound of the formula (I), (II), (III), (IV), (IVa), (IVb), (V) or any sub-group or embodiment thereof as defined herein.
A compound of the formula (I), (II), (III), (IV), (IVa), (IVb), (V) or any sub-group thereof as defined herein for use in modulating a cellular process (for example cell division) by inhibiting the activity of a protein kinase B and/or protein kinase A.
The use of a compound of formula (I), (II), (III), (IV), (IVa), (IVb), (V) or any sub-group thereof as defined herein for the manufacture of a medicament for modulating a cellular process (for example cell division) by inhibiting the activity of a protein kinase B.
A method of modulating a cellular process (for example cell division) by inhibiting the activity of a protein kinase A using a compound of the formula (I), (II), (III), (IV), (IVa), (IVb), (V) or any sub-group or embodiment thereof as defined herein.
The use of a compound of the formula (I), (II), (III), (IV), (IVa), (IVb), (V) or any sub-group thereof as defined herein for the manufacture of a medicament for the prophylaxis or treatment of a disease state or condition arising from abnormal cell growth or abnormally arrested cell death.
A method for treating a disease or condition comprising or arising from abnormal cell growth in a mammal, which method comprises administering to the mammal a compound of the formula (I), (II), (III), (IV), (IVa), (IVb), (V) or any sub-group thereof as defined herein in an amount effective in inhibiting abnormal cell growth or abnormally arrested cell death.
A method for alleviating or reducing the incidence of a disease or condition comprising or arising from abnormal cell growth or abnormally arrested cell death in a mammal, which method comprises administering to the mammal a compound of the formula (I), (II), (III), (IV), (IVa), (IVb), (V) or any sub-group thereof as defined herein in an amount effective in inhibiting abnormal cell growth.
A pharmaceutical composition comprising a novel compound of the formula (I), (II), (III), (IV), (IVa), (IVb), (V) or any sub-group thereof as defined herein and a pharmaceutically acceptable carrier.
A compound of the formula (I), (II), (III), (IV), (IVa), (IVb), (V) or any sub-group thereof as defined herein for use in medicine.
The use of a compound of the formula (I), (II), (III), (IV), (IVa), (IVb), (V) or any sub-group thereof as defined herein for the manufacture of a medicament for the prophylaxis or treatment of any one of the disease states or conditions disclosed herein.
A method for the treatment or prophylaxis of any one of the disease states or conditions disclosed herein, which method comprises administering to a patient (e.g. a patient in need thereof) a compound (e.g. a therapeutically effective amount) of the formula (I), (II), (III), (IV), (IVa), (IVb), (V) or any sub-group thereof as defined herein.
A method for alleviating or reducing the incidence of a disease state or condition disclosed herein, which method comprises administering to a patient (e.g. a patient in need thereof) a compound (e.g. a therapeutically effective amount) of the formula (I), (II), (III), (IV), (IVa), (IVb), (V) or any sub-group thereof as defined herein.
A method for the diagnosis and treatment of a disease state or condition mediated by protein kinase B, which method comprises (i) screening a patient to determine whether a disease or condition from which the patient is or may be suffering is one which would be susceptible to treatment with a compound having activity against protein kinase B; and (ii) where it is indicated that the disease or condition from which the patient is thus susceptible, thereafter administering to the patient a compound of the formula (I), (II), (III), (IV), (IVa), (IVb), (V) or any sub-group thereof as defined herein.
The use of a compound of the formula (I), (II), (III), (IV), (IVa), (IVb), (V) or any sub-group thereof as defined herein for the manufacture of a medicament for the treatment or prophylaxis of a disease state or condition in a patient who has been screened and has been determined as suffering from, or being at risk of suffering from, a disease or condition which would be susceptible to treatment with a compound having activity against protein kinase B.
A method for the diagnosis and treatment of a disease state or condition mediated by protein kinase A, which method comprises (i) screening a patient to determine whether a disease or condition from which the patient is or may be suffering is one which would be susceptible to treatment with a compound having activity against protein kinase A; and (ii) where it is indicated that the disease or condition from which the patient is thus susceptible, thereafter administering to the patient a compound of the formula (I), (II), (III), (IV), (IVa), (IVb), (V) or any sub-group or embodiment thereof as defined herein.
The use of a compound of the formula (I), (II), (III), (IV), (IVa), (IVb), (V) or any sub-group or embodiment thereof as defined herein for the manufacture of a medicament for the treatment or prophylaxis of a disease state or condition in a patient who has been screened and has been determined as suffering from, or being at risk of suffering from, a disease or condition which would be susceptible to treatment with a compound having activity against protein kinase A.
A compound of the formula (I), (II), (III), (IV), (IVa), (IVb), (V) or any sub-group or embodiment thereof as defined herein for use as a modulator (e.g. inhibitor) of protein kinase B and/or protein kinase A.
The use of a compound of the formula (I), (II), (III), (IV), (IVa), (IVb), (V) or any sub-group or embodiment thereof as defined herein for the manufacture of a medicament for modulating (e.g. inhibiting) protein kinase B and/or protein kinase A.
A method of modulating (e g inhibiting) protein kinase B and/or protein kinase A; which method comprises bringing the protein kinase B and/or protein kinase A (e.g. in a cellular environment—for example in vivo) into contact with a compound of the formula (I), (II), (III), (IV), (IVa), (IVb), (V) or any sub-group or embodiment thereof as defined herein.

General Preferences and Definitions

The following general preferences and definitions shall apply to each of the moieties T, G, Q¹, Q², Q³, J¹, J², T and R^{1a} to R¹⁰ and any sub-definition, sub-group or embodiment thereof, unless the context indicates otherwise.

Any references to Formula (I) herein shall be taken also to refer to formulae (II), (III), (IV), (IVa), (IVb), (V) and any other sub-group of compounds within formula (I), or embodiment thereof, unless the context requires otherwise.

As used herein, the term “modulation”, as applied to the activity of a kinase, is intended to define a change in the level of biological activity of the protein kinase. Thus, modulation encompasses physiological changes which effect an increase or decrease in the relevant protein kinase activity. In the latter case, the modulation may be described as “inhibition”. The modulation may arise directly or indirectly, and may be mediated by any mechanism and at any physiological level, including for example at the level of gene expression (including for example transcription, translation and/or post-translational modification), at the level of expression of genes encoding regulatory elements which act directly or indirectly on the levels of kinase activity. Thus, modulation may imply elevated/suppressed expression or over- or under-expression of a kinase, including gene amplification (i.e. multiple gene copies) and/or increased or decreased expression by a transcriptional effect, as well as hyper- (or hypo-)activity and (de)activation of the protein kinase(s) (including (de)activation) by mutation(s). The terms “modulated”, “modulating” and “modulate” are to be interpreted accordingly.

As used herein, the term “mediated”, as used e.g. in conjunction with a kinase as described herein (and applied for example to various physiological processes, diseases, states, conditions, therapies, treatments or interventions) is intended to operate limitatively so that the various processes, diseases, states, conditions, treatments and interventions to which the term is applied are those in which the kinase plays a biological role. In cases where the term is applied to a disease, state or condition, the biological role played by a kinase may be direct or indirect and may be necessary and/or sufficient for the manifestation of the symptoms of the disease, state or condition (or its aetiology or progression). Thus, kinase activity (and in particular aberrant levels of kinase activity, e.g. kinase over-expression) need not necessarily be the proximal cause of the disease, state or condition: rather, it is contemplated that the kinase mediated diseases, states or conditions include those having multifactorial aetiologies and complex progressions in which the kinase in question is only partially involved. In cases where the term is applied to treatment, prophylaxis or intervention, the role played by the kinase may be direct or indirect and may be necessary and/or sufficient for the operation of the treatment, prophylaxis or outcome of the intervention. Thus, a disease state or condition mediated by a kinase includes the development of resistance to any particular cancer drug or treatment.

In this specification, references to “the bicyclic group” shall, unless the context indicates otherwise, be taken to refer to the group:

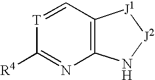

References to “carbocyclic” and “heterocyclic” groups as used herein shall, unless the context indicates otherwise, include both aromatic and non-aromatic ring systems. In general, such groups may be monocyclic or bicyclic and may contain, for example, 3 to 12 ring members, more usually 5 to 10 ring members. Examples of monocyclic groups are groups containing 3, 4, 5, 6, 7, and 8 ring members, more usually 3 to 7, and preferably 5 or 6 ring members. Examples of bicyclic groups are those containing 8, 9, 10, 11 and 12 ring members, and more usually 9 or 10 ring members.

The carbocyclic or heterocyclic groups can be aryl or heteroaryl groups having from 5 to 12 ring members, more usually from 5 to 10 ring members. The term “aryl” as used herein refers to a carbocyclic group having aromatic character and the term “heteroaryl” is used herein to denote a heterocyclic group having aromatic character. The terms “aryl” and “heteroaryl” embrace polycyclic (e.g. bicyclic) ring systems wherein one or more rings are non-aromatic, provided that at least one ring is aromatic. In such polycyclic systems, the group may be attached by the aromatic ring, or by a non-aromatic ring. The aryl or heteroaryl groups can be monocyclic or bicyclic groups and can be unsubstituted or substituted with one or more substituents, for example one or more groups R¹⁰ as defined herein.

The term non-aromatic group embraces unsaturated ring systems without aromatic character, partially saturated and fully saturated carbocyclic and heterocyclic ring systems. The terms “unsaturated” and “partially saturated” refer to rings wherein the ring structure(s) contains atoms sharing more than one valence bond i.e. the ring contains at least one multiple bond e.g. a C═C, C≡C or N═C bond. The term “fully saturated” refers to rings where there are no multiple bonds between ring atoms. Saturated carbocyclic groups include cycloalkyl groups as defined below. Partially saturated carbocyclic groups include cycloalkenyl groups as defined below, for example cyclopentenyl, cycloheptenyl and cyclooctenyl.

Examples of heteroaryl groups are monocyclic and bicyclic groups containing from five to twelve ring members, and more usually from five to ten ring members. The heteroaryl group can be, for example, a five membered or six membered monocyclic ring or a bicyclic structure formed from fused five and six membered rings or two fused six membered rings. Each ring may contain up to about four heteroatoms typically selected from nitrogen, sulphur and oxygen. Typically the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Examples of five membered heteroaryl groups include but are not limited to pyrrole, furan, thiophene, imidazole, furazan, oxazole, oxadiazole, oxatriazole, isoxazole, thiazole, isothiazole, pyrazole, triazole and tetrazole groups.

Examples of six membered heteroaryl groups include but are not limited to pyridine, pyrazine, pyridazine, pyrimidine and triazine.

A bicyclic heteroaryl group may be, for example, a group selected from:
a) a benzene ring fused to a 5- or 6-membered ring containing 1, 2 or 3 ring heteroatoms;
b) a pyridine ring fused to a 5- or 6-membered ring containing 1, 2 or 3 ring heteroatoms;
c) a pyrimidine ring fused to a 5- or 6-membered ring containing 1 or 2 ring heteroatoms;
d) a pyrrole ring fused to a a 5- or 6-membered ring containing 1, 2 or 3 ring heteroatoms;
e) a pyrazole ring fused to a a 5- or 6-membered ring containing 1 or 2 ring heteroatoms;
f) a pyrazine ring fused to a 5- or 6-membered ring containing 1 or 2 ring heteroatoms;
g) an imidazole ring fused to a 5- or 6-membered ring containing 1 or 2 ring heteroatoms;
h) an oxazole ring fused to a 5- or 6-membered ring containing 1 or 2 ring heteroatoms;
i) an isoxazole ring fused to a 5- or 6-membered ring containing 1 or 2 ring heteroatoms;
j) a thiazole ring fused to a 5- or 6-membered ring containing 1 or 2 ring heteroatoms;
k) an isothiazole ring fused to a 5- or 6-membered ring containing 1 or 2 ring heteroatoms;
l) a thiophene ring fused to a 5- or 6-membered ring containing 1, 2 or 3 ring heteroatoms;
m) a furan ring fused to a 5- or 6-membered ring containing 1, 2 or 3 ring heteroatoms;
n) a cyclohexyl ring fused to a 5- or 6-membered ring containing 1, 2 or 3 ring heteroatoms; and
o) a cyclopentyl ring fused to a 5- or 6-membered ring containing 1, 2 or 3 ring heteroatoms.

Particular examples of bicyclic heteroaryl groups containing a six membered ring fused to a five membered ring include but are not limited to benzfuran, benzthiophene, benzimidazole, benzoxazole, benzisoxazole, benzthiazole, benzisothiazole, isobenzofuran, indole, isoindole, indolizine, indoline, isoindoline, purine (e.g., adenine, guanine), indazole, benzodioxole and pyrazolopyridine groups.

Particular examples of bicyclic heteroaryl groups containing two fused six membered rings include but are not limited to quinoline, isoquinoline, chroman, thiochroman, chromene, isochromene, chroman, isochroman, benzodioxan, quinolizine, benzoxazine, benzodiazine, pyridopyridine, quinoxaline, quinazoline, cinnoline, phthalazine, naphthyridine and pteridine groups.

Examples of polycyclic aryl and heteroaryl groups containing an aromatic ring and a non-aromatic ring include tetrahydronaphthalene, tetrahydroisoquinoline, tetrahydroquinoline, dihydrobenzthiene, dihydrobenzfuran, 2,3-dihydro-benzo[1,4]dioxine, benzo[1,3]dioxole, 4,5,6,7-tetrahydrobenzofuran, indoline and indane groups.

Examples of carbocyclic aryl groups include phenyl, naphthyl, indenyl, and tetrahydronaphthyl groups.

Examples of non-aromatic heterocyclic groups include unsubstituted or substituted (by one or more groups R¹⁰) heterocyclic groups having from 3 to 12 ring members, typically 4 to 12 ring members, and more usually from 5 to 10 ring members. Such groups can be monocyclic or bicyclic, for example, and typically have from 1 to 5 heteroatom ring members (more usually 1, 2, 3 or 4 heteroatom ring members) typically selected from nitrogen, oxygen and sulphur.

When sulphur is present, it may, where the nature of the adjacent atoms and groups permits, exist as —S—, —S(O)— or —S(O)₂—.

The heterocylic groups can contain, for example, cyclic ether moieties (e.g. as in tetrahydrofuran and dioxane), cyclic thioether moieties (e.g. as in tetrahydrothiophene and dithiane), cyclic amine moieties (e.g. as in pyrrolidine), cyclic amide moieties (e.g. as in pyrrolidone), cyclic urea moieties (e.g. as in imidazolidin-2-one), cyclic thiourea moieties, cyclic thioamides, cyclic thioesters, cyclic ester moieties (e.g. as in butyrolactone), cyclic sulphones (e.g. as in sulpholane and sulpholene), cyclic sulphoxides, cyclic sulphonamides and combinations thereof (e.g. morpholine and thiomorpholine and its S-oxide and S,S-dioxide).

Examples of monocyclic non-aromatic heterocyclic groups include 5-, 6-and 7-membered monocyclic heterocyclic groups. Particular examples include morpholine, thiomorpholine and its S-oxide and S,S-dioxide (particularly thiomorpholine), piperidine (e.g. 1-piperidinyl, 2-piperidinyl 3-piperidinyl and 4-piperidinyl), N-alkyl piperidines such as N-methyl piperidine, piperidone, pyrrolidine (e.g. 1-pyrrolidinyl, 2-pyrrolidinyl and 3-pyrrolidinyl), pyrrolidone, azetidine, pyran (2H-pyran or 4H-pyran), dihydrothiophene, dihydropyran, dihydrofuran, dihydrothiazole, tetrahydrofuran, tetrahydrothiophene, dioxane, tetrahydropyran (e.g. 4-tetrahydro pyranyl), imidazoline, imidazolidinone, oxazoline, thiazoline, 2-pyrazoline, pyrazolidine, piperazone, piperazine, and N-alkyl piperazines such as N-methyl piperazine, N-ethyl piperazine and N-isopropylpiperazine. In general, preferred non-aromatic heterocyclic groups include piperidine, pyrrolidine, azetidine, morpholine, piperazine and N-alkyl piperazines.

Examples of non-aromatic carbocyclic groups include cycloalkane groups such as cyclohexyl and cyclopentyl, cycloalkenyl groups such as cyclopentenyl, cyclohexenyl, cycloheptenyl and cyclooctenyl, as well as cyclohexadienyl, cyclooctatetraene, tetrahydronaphthenyl and decalinyl.

Preferred non-aromatic carbocyclic groups are monocyclic rings and most preferably saturated monocyclic rings.

Typical examples are three, four, five and six membered saturated carbocyclic rings, e.g. optionally substituted cyclopentyl and cyclohexyl rings.

One sub-set of non-aromatic carbocyclic groups includes unsubstituted or substituted (by one or more groups R¹⁰) monocyclic groups and particularly saturated monocyclic groups, e.g. cycloalkyl groups. Examples of such cycloalkyl groups include cyclopropyl, cyclobutyl, cyclopentyl, cyclohexyl and cycloheptyl; more typically cyclopropyl, cyclobutyl, cyclopentyl and cyclohexyl, particularly cyclohexyl.

Further examples of non-aromatic cyclic groups include bridged ring systems such as bicycloalkanes and azabicycloalkanes although such bridged ring systems are generally less preferred. By “bridged ring systems” is meant ring systems in which two rings share more than two atoms, see for example Advanced Organic Chemistry, by Jerry March, 4^{th} Edition, Wiley Interscience, pages 131-133, 1992. Examples of bridged ring systems include bicyclo[2.2.1]heptane, aza-bicyclo[2.2.1]heptane, bicyclo[2.2.2]octane, aza-bicyclo[2.2.2]octane, bicyclo[3.2.1]octane and aza-bicyclo[3.2.1]octane.

Where reference is made herein to carbocyclic and heterocyclic groups, the carbocyclic or heterocyclic ring can, unless the context indicates otherwise, be unsubstituted or substituted by one or more substituent groups R¹⁰ selected from halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{c} is selected from hydrogen and C₁₋₄ hydrocarbyl; and
X¹ is O, S or NR^{c} and X² is ═O, ═S or ═NR^{c}.

Where the substituent group R¹⁰ comprises or includes a carbocyclic or heterocyclic group, the said carbocyclic or heterocyclic group may be unsubstituted or may itself be substituted with one or more further substituent groups R¹⁰ (or substituent groups R^{10a}, R^{10b} or R^{10c} as defined herein). In one sub-group of compounds of the formula (I), such further substituent groups R¹⁰ (or substituent groups R^{10a}, R^{10b} or R^{10c} as defined herein) may include carbocyclic or heterocyclic groups, which are typically not themselves further substituted. In another sub-group of compounds of the formula (I), the said further substituents do not include carbocyclic or heterocyclic groups but are otherwise selected from the groups listed above in the definition of R¹⁰ (or substituent groups R^{10a}, R^{10b} or R^{10c} as defined herein).

When the or a substituent R¹⁰ (or substituent group R^{10a}, R^{10b} or R^{10c} as defined herein) is a carbocyclic or heterocyclic group, it is preferably an optionally substituted monocyclic carbocyclic or heterocyclic group. Examples of such groups are as set out above. The optional substituents for the optionally substituted monocyclic carbocyclic or heterocyclic group are preferably acyclic substituents selected from R¹⁰, R^{10a}, R^{10b} or R^{10c} as defined herein. In one sub-group of compounds, R¹⁰ is selected from optionally substituted monocyclic aryl and heteroaryl groups of 5 or 6 ring members of which up to 2 are heteroatoms selected from O, N and S; optionally substituted C₃₋₇ cycloalkyl groups; and optionally substituted 4-7 membered monocyclic non-aromatic heterocyclic groups containing 1 or 2 heteroatom ring members selected from O, N and S and oxidised forms thereof; wherein, in each case, the optional substituents for the optionally substituted monocyclic carbocyclic or heterocyclic group are preferably acyclic substituents selected from R¹⁰, R^{10a}, R^{10b} or R^{10c} as defined herein

The substituents R¹⁰ (or substituent groups R^{10a}, R^{10b} or R^{10c} as defined herein) may be selected such that they contain no more than 20 non-hydrogen atoms, for example, no more than 15 non-hydrogen atoms, e.g. no more than 12, or 10, or 9, or 8, or 7, or 6, or 5 non-hydrogen atoms.

One sub-group of substituents R¹⁰ is represented by R^{10a} which consists of substituents selected from halogen, hydroxy, trifluoromethyl, cyano, nitro, carboxy, amino, mono- or di-C₁₋₄ hydrocarbylamino, carbocyclic and heterocyclic groups having from 3 to 7 ring members; a group R^{a}-R^{b} wherein R^{a} is a bond, O, CO, OC(O), NR^{c}C(O), OC(NR^{c}), C(O)O, C(O)NR^{c}, OC(O)O, NR^{c}C(O)O, OC(O)NR^{c}, NR^{c}C(O)NR^{c}, S, SO, SO₂, NR^{c}, SO₂NR^{c} or NR^{c}SO₂; and R^{b} is selected from hydrogen, carbocyclic and heterocyclic groups having from 3 to 7 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 7 ring members and wherein one or more carbon atoms of the C₁₋₈ hydrocarbyl group may optionally be replaced by O, S, SO, SO₂, NR^{c}, OC(O), NR^{c}C(O), OC(NR^{c}), C(O)O, C(O)NR^{c}, OC(O)O, NR^{c}C(O)O, OC(O)NR^{c} or NR^{c}C(O)NR^{c}; R^{c} is selected from hydrogen and C₁₋₄ hydrocarbyl.

Another sub-group of substituents R¹⁰ is represented by R^{10b} which consists of substituents selected from halogen, hydroxy, trifluoromethyl, cyano, amino, mono- or di-C₁₋₄ alkylamino, cyclopropylamino, carbocyclic and heterocyclic groups having from 3 to 7 ring members; a group R^{a}-R^{b} wherein R^{a} is a bond, O, CO, OC(O), NR^{c}C(O), OC(NR^{c}), C(O)O, C(O)NR^{c}, S, SO, SO₂, NR^{c}, SO₂NR^{c} or NR^{c}SO₂; and R^{b} is selected from hydrogen, carbocyclic and heterocyclic groups having from 3 to 7 ring members, and a C₁₋₈ hydrocarbyl group optionally substituted by one or more substituents selected from hydroxy, oxo, halogen, cyano, amino, mono- or di-C₁₋₄ alkylamino, carbocyclic and heterocyclic groups having from 3 to 7 ring members and wherein one or more carbon atoms of the C₁₋₈ hydrocarbyl group may optionally be replaced by O, S, SO, SO₂ or NR^{c}; provided that R^{a} is not a bond when R^{b} is hydrogen; and R^{c} is selected from hydrogen and C₁₋₄ alkyl.

A further sub-group of substituents R¹⁰ is represented by R^{10c} which consists of substituents selected from:

halogen,

hydroxy,

trifluoromethyl,

cyano,

amino, mono- or di-C₁₋₄ alkylamino,

cyclopropylamino,

monocyclic carbocyclic and heterocyclic groups having from 3 to 7 ring members of which 0, 1 or 2 are selected from O, N and S and the remainder are carbon atoms, wherein the monocyclic carbocyclic and heterocyclic groups are optionally substituted by one or more substituents selected from halogen, hydroxy, trifluoromethyl, cyano and methoxy; a group R^{a}-R^{b};

R^{a} is a bond, O, CO, OC(O), NR^{c}C(O), OC(NR^{c}), C(O)O, C(O)NR^{c}, S, SO, SO₂, NR^{c}, SO₂NR^{c} or NR^{c}SO₂;

R^{b} is selected from hydrogen, monocyclic carbocyclic and heterocyclic groups having from 3 to 7 ring members of which 0, 1 or 2 are selected from O, N and S and the remainder are carbon atoms, wherein the monocyclic carbocyclic and heterocyclic groups are optionally substituted by one or more substituents selected from halogen, hydroxy, trifluoromethyl, cyano and methoxy;

and R^{b} is further selected from a C₁₋₈ hydrocarbyl group optionally substituted by one or more substituents selected from hydroxy, oxo, halogen, cyano, amino, mono- or di-C₁₋₄ alkylamino, monocyclic carbocyclic and heterocyclic groups having from 3 to 7 ring members of which 0, 1 or 2 are selected from O, N and S and the remainder are carbon atoms, wherein the monocyclic carbocyclic and heterocyclic groups are optionally substituted by one or more substituents selected from halogen, hydroxy, trifluoromethyl, cyano and methoxy, and wherein one or two carbon atoms of the C₁₋₈ hydrocarbyl group may optionally be replaced by O, S or NR^{c}; provided that R^{a} is not a bond when R^{b} is hydrogen; and

R^{c} is selected from hydrogen and C₁₋₄ alkyl.

Where the carbocyclic and heterocyclic groups have a pair of substituents on adjacent ring atoms, the two substituents may be linked so as to form a cyclic group. For example, an adjacent pair of substituents on adjacent carbon atoms of a ring may be linked via one or more heteroatoms and optionally substituted alkylene groups to form a fused oxa-, dioxa-, aza-, diaza- or oxa-aza-cycloalkyl group. Examples of such linked substituent groups include:

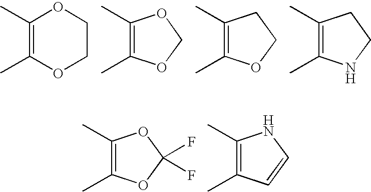

Examples of halogen substituents include fluorine, chlorine, bromine and iodine. Fluorine and chlorine are particularly preferred.

In the definition of the compounds of the formula (I) above and as used hereinafter, the term “hydrocarbyl” is a generic term encompassing aliphatic, alicyclic and aromatic groups having an all-carbon backbone and consisting of carbon and hydrogen atoms, except where otherwise stated.

In certain cases, as defined herein, one or more of the carbon atoms making up the carbon backbone may be replaced by a specified atom or group of atoms. Examples of hydrocarbyl groups include alkyl, cycloalkyl, cycloalkenyl, carbocyclic aryl, alkenyl, alkynyl, cycloalkylalkyl, cycloalkenylalkyl, and carbocyclic aralkyl, aralkenyl and aralkynyl groups. Such groups can be unsubstituted or, where stated, can be substituted by one or more substituents as defined herein. The examples and preferences expressed below apply to each of the hydrocarbyl substituent groups or hydrocarbyl-containing substituent groups referred to in the various definitions of substituents for compounds of the formula (I) and sub-groups thereof as defined herein unless the context indicates otherwise.

Generally by way of example, the hydrocarbyl groups can have up to eight carbon atoms, unless the context requires otherwise. Within the sub-set of hydrocarbyl groups having 1 to 8 carbon atoms, particular examples are C₁₋₆ hydrocarbyl groups, such as C₁₋₄ hydrocarbyl groups (e.g. C₁₋₃ hydrocarbyl groups or C₁₋₂ hydrocarbyl groups), specific examples being any individual value or combination of values selected from C₁, C₂, C₃, C₄, C₅, C₆, C₇ and C₈ hydrocarbyl groups.

The term “saturated hydrocarbyl”, whether used alone or together with a suffix such as “oxy” (e.g. as in “hydrocarbyloxy”), refers to a non-aromatic hydrocarbon group containing no multiple bonds such as C═C and C≡C.

Particular hydrocarbyl groups are saturated hydrocarbyl groups such as alkyl and cycloalkyl groups as defined herein.

The term “alkyl” covers both straight chain and branched chain alkyl groups. Examples of alkyl groups include methyl, ethyl, propyl, isopropyl, n-butyl, isobutyl, tert-butyl, n-pentyl, 2-pentyl, 3-pentyl, 2-methyl butyl, 3-methyl butyl, and n-hexyl and its isomers. Within the sub-set of alkyl groups having 1 to 8 carbon atoms, particular examples are C₁₋₆ alkyl groups, such as C₁₋₄ alkyl groups (e.g. C₁₋₃ alkyl groups or C₁₋₂ alkyl groups).

Examples of cycloalkyl groups are those derived from cyclopropane, cyclobutane, cyclopentane, cyclohexane and cycloheptane. Within the sub-set of cycloalkyl groups the cycloalkyl group will have from 3 to 8 carbon atoms, particular examples being C₃₋₆ cycloalkyl groups.

Examples of alkenyl groups include, but are not limited to, ethenyl (vinyl), 1-propenyl, 2-propenyl(allyl), isopropenyl, butenyl, buta-1,4-dienyl, pentenyl, and hexenyl. Within the sub-set of alkenyl groups the alkenyl group will have 2 to 8 carbon atoms, particular examples being C₂₋₆ alkenyl groups, such as C₂₋₄ alkenyl groups.

Examples of cycloalkenyl groups include, but are not limited to, cyclopropenyl, cyclobutenyl, cyclopentenyl, cyclopentadienyl and cyclohexenyl. Within the sub-set of cycloalkenyl groups the cycloalkenyl groups have from 3 to 8 carbon atoms, and particular examples are C₃₋₆ cycloalkenyl groups.

Examples of alkynyl groups include, but are not limited to, ethynyl and 2-propynyl(propargyl) groups. Within the sub-set of alkynyl groups having 2 to 8 carbon atoms, particular examples are C₂₋₆ alkynyl groups, such as C₂₋₄ alkynyl groups.

Examples of carbocyclic aryl groups include substituted and unsubstituted phenyl, naphthyl, indane and indene groups.

Examples of cycloalkylalkyl, cycloalkenylalkyl, carbocyclic aralkyl, aralkenyl and aralkynyl groups include phenethyl, benzyl, styryl, phenylethynyl, cyclohexylmethyl, cyclopentylmethyl, cyclobutylmethyl, cyclopropylmethyl and cyclopentenylmethyl groups.

The term C₁₋₈ hydrocarbyl as used herein refers to a group consisting of carbon and hydrogen atoms and having 1 to 8 carbon atoms. The term encompasses C₁₋₈ alkyl, C₂₋₈ alkenyl, C₂₋₈ alkynyl, C₃₋₈ cycloalkyl, C₃₋₈ cycloalkenyl, phenyl, benzyl and phenylethyl groups wherein the preferences for and examples of each of the aforesaid groups are as defined above. Within this definition, particular C₁₋₈ hydrocarbyl groups are alkyl groups of 1 to 6 carbon atoms (e.g. up to 5 or up to 4 or up to 3 carbon atoms), cycloalkyl groups of 3 to 7 (more preferably 3 to 6) carbon atoms, phenyl, benzyl and phenylethyl (e.g. 1-phenylethyl or 2-phenylethyl) groups, one subset of C₁₋₈ hydrocarbyl groups consisting of C₁₋₆ alkyl and C₃₋₆ cycloalkyl groups and in particular C₁₋₄ alkyl and C₃₋₆ cycloalkyl groups such as methyl, ethyl, n-propyl, isopropyl, n-butyl, isobutyl, tert-butyl, cyclopropyl and cyclobutyl.

The term C₁₋₅ hydrocarbyl defines a subset of C₁₋₈ hydrocarbyl groups and refers to a group consisting of carbon and hydrogen atoms and having 1 to 5 carbon atoms. The term encompasses C₁₋₅ alkyl, C₂₋₅ alkenyl, C₂₋₅ alkynyl, C₃₋₅ cycloalkyl, and C₃₋₅ cycloalkenyl groups wherein the preferences for and examples of each of the aforesaid groups are as defined above. Within this definition, particular C₁₋₅ hydrocarbyl groups are C₁₋₅ alkyl and C₃₋₅ cycloalkyl groups. Particular examples of C₁₋₅ alkyl and C₃₋₅ cycloalkyl groups are methyl, ethyl, n-propyl, isopropyl, n-butyl, isobutyl, tert-butyl, cyclopropyl and cyclobutyl.

The term C₁₋₄ hydrocarbyl defines a subset of C₁₋₅ hydrocarbyl groups and refers to a group consisting of carbon and hydrogen atoms and having 1 to 4 carbon atoms. The term encompasses C₁₋₄ alkyl, C₂₋₄ alkenyl, C₂₋₄ alkynyl, C₃₋₄ cycloalkyl, and C₃₋₄ cycloalkenyl groups wherein the preferences for and examples of each of the aforesaid groups are as defined above. Within this definition, particular C₁₋₄ hydrocarbyl groups are C₁₋₄ alkyl and C₃₋₄ cycloalkyl groups, such as methyl, ethyl, n-propyl, isopropyl, n-butyl, isobutyl, tert-butyl, cyclopropyl and cyclobutyl.

When present, and where stated, a hydrocarbyl group can be optionally substituted by one or more substituents selected from hydroxy, oxo, alkoxy, carboxy, halogen, cyano, nitro, amino, mono- or di-C₁₋₄ hydrocarbylamino, and monocyclic or bicyclic carbocyclic and heterocyclic groups having from 3 to 12 (typically 3 to 10 and more usually 5 to 10) ring members. Preferred substituents include halogen such as fluorine. Thus, for example, the substituted hydrocarbyl group can be a partially fluorinated or perfluorinated group such as difluoromethyl or trifluoromethyl. In one embodiment preferred substituents include monocyclic carbocyclic and heterocyclic groups having 3-7 ring members.

Where stated, one or more carbon atoms of a hydrocarbyl group may optionally be replaced by O, S, SO, SO₂, NR^{c}, X¹C(X²), C(X²)X¹ or X¹C(X²)X¹ (or a sub-group thereof) wherein X¹ and X² are as hereinbefore defined, provided that at least one carbon atom of the hydrocarbyl group remains. For example, 1, 2, 3 or 4 carbon atoms of the hydrocarbyl group may be replaced by one of the atoms or groups listed, and the replacing atoms or groups may be the same or different. In general, the number of linear or backbone carbon atoms replaced will correspond to the number of linear or backbone atoms in the group replacing them. Examples of groups in which one or more carbon atom of the hydrocarbyl group have been replaced by a replacement atom or group as defined above include ethers and thioethers (C replaced by O or S), amides, esters, thioamides and thioesters (C—C replaced by X¹C(X²) or C(X²)X¹), sulphones and sulphoxides (C replaced by SO or SO₂), amines (C replaced by NR^{c}). Further examples include ureas, carbonates and carbamates (C—C—C replaced by X¹C(X²)X¹).

Where an amino group has two hydrocarbyl substituents, they may, together with the nitrogen atom to which they are attached, and optionally with another heteroatom such as nitrogen, sulphur, or oxygen, link to form a ring structure of 4 to 7 ring members.

The term C₁₋₄ acyl as used herein (whether as a discrete moiety or as part of another group such as an acylamino or acyloxy group) refers to a group containing up to 4 carbon atoms and having the formula:

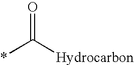

where the asterisk shows the point of attachment to the remainder the molecule and “hydrocarbon” is a hydrocarbon group of 1 to 3 carbon atoms. The hydrocarbon group can be saturated or unsaturated and can be an alkyl, alkenyl or alkynyl group as defined herein or a cyclopropyl ring. In one general embodiment, the hydrocarbon group is an alkyl or cyclopropyl group. In another general embodiment, the hydrocarbon group is an alkyl group.

Particular C₁₋₄ acyl groups are acetyl, propanoyl and isopropanoyl.

The term “aza-cycloalkyl” as used herein refers to a cycloalkyl group in which one of the carbon ring members has been replaced by a nitrogen atom. Thus examples of aza-cycloalkyl groups include piperidine and pyrrolidine. The term “oxa-cycloalkyl” as used herein refers to a cycloalkyl group in which one of the carbon ring members has been replaced by an oxygen atom. Thus examples of oxa-cycloalkyl groups include tetrahydrofuran and tetrahydropyran. In an analogous manner, the terms “diaza-cycloalkyl”, “dioxa-cycloalkyl” and “aza-oxa-cycloalkyl” refer respectively to cycloalkyl groups in which two carbon ring members have been replaced by two nitrogen atoms, or by two oxygen atoms, or by one nitrogen atom and one oxygen atom.

The definition “R^{a}-R^{b}” as used herein, either with regard to substituents present on a carbocyclic or heterocyclic moiety, or with regard to other substituents present at other locations on the compounds of the formula (I), includes inter alia compounds wherein R^{a} is selected from a bond, O, CO, OC(O), SC(O), NR^{c}C(O), OC(S), SC(S), NR^{c}C(S), OC(NR^{c}), SC(NR^{c}), NR^{c}C(NR^{c}), C(O)O, C(O)S, C(O)NR^{c}, C(S)O, C(S)S, C(S)NR^{c}, C(NR^{c})O, C(NR^{c})S, C(NR^{c})NR^{c}, OC(O)O, SC(O)O, NR^{c}C(O)O, OC(S)O, SC(S)O, NR^{c}C(S)O, OC(NR^{c})O, SC(NR^{c})O, NR^{c}C(NR^{c})O, OC(O)S, SC(O)S, NR^{c}C(O)S, OC(S)S, SC(S)S, NR^{c}C(S)S, OC(NR^{c})S, SC(NR^{c})S, NR^{c}C(NR^{c})S, OC(O)NR^{c}, SC(O)NR^{c}, NR^{c}C(O)NR^{c}, OC(S)NR^{c}, SC(S)NR^{c}, NR^{c}C(S)NR^{c}, OC(NR^{c})NR^{c}, SC(NR^{c})NR^{c}, NR^{c}C(NR^{c}NR^{c}, S, SO, SO₂, NR^{c}, SO₂NR^{c} and NR^{c}SO₂ wherein R^{c} is as hereinbefore defined.

The moiety R^{b} can be hydrogen or it can be a group selected from carbocyclic and heterocyclic groups having from 3 to 12 ring members (typically 3 to 10 and more usually from 5 to 10), and a C₁₋₈ hydrocarbyl group optionally substituted as hereinbefore defined. Examples of hydrocarbyl, carbocyclic and heterocyclic groups are as set out above.

When R^{a} is O and R^{b} is a C₁₋₈ hydrocarbyl group, R^{a} and R^{b} together form a hydrocarbyloxy group. Preferred hydrocarbyloxy groups include saturated hydrocarbyloxy such as alkoxy (e.g. C₁₋₆ alkoxy, more usually C₁₋₄ alkoxy such as ethoxy and methoxy, particularly methoxy), cycloalkoxy (e.g. C₃₋₆ cycloalkoxy such as cyclopropyloxy, cyclobutyloxy, cyclopentyloxy and cyclohexyloxy) and cycloalkyalkoxy (e.g. C₃₋₆ cycloalkyl-C₁₋₂ alkoxy such as cyclopropylmethoxy).

The hydrocarbyloxy groups can be substituted by various substituents as defined herein. For example, the alkoxy groups can be substituted by halogen (e.g. as in difluoromethoxy and trifluoromethoxy), hydroxy (e.g. as in hydroxyethoxy), C₁₋₂ alkoxy (e.g. as in methoxyethoxy), hydroxy-C₁₋₂ alkyl (as in hydroxyethoxyethoxy) or a cyclic group (e.g. a cycloalkyl group or non-aromatic heterocyclic group as hereinbefore defined). Examples of alkoxy groups bearing a non-aromatic heterocyclic group as a substituent are those in which the heterocyclic group is a saturated cyclic amine such as morpholine, piperidine, pyrrolidine, piperazine, C₁₋₄-alkyl-piperazines, C₃₋₇-cycloalkyl-piperazines, tetrahydropyran or tetrahydrofuran and the alkoxy group is a C₁₋₄ alkoxy group, more typically a C₁₋₃ alkoxy group such as methoxy, ethoxy or n-propoxy.

Alkoxy groups may be substituted by, for example, a monocyclic group such as pyrrolidine, piperidine, morpholine and piperazine and N-substituted derivatives thereof such as N-benzyl, N—C₁₋₄ acyl and N—C₁₋₄ alkoxycarbonyl. Particular examples include pyrrolidinoethoxy, piperidinoethoxy and piperazinoethoxy.

When R^{a} is a bond and R^{b} is a C₁₋₈ hydrocarbyl group, examples of hydrocarbyl groups R^{a}-R^{b} are as hereinbefore defined. The hydrocarbyl groups may be saturated groups such as cycloalkyl and alkyl and particular examples of such groups include methyl, ethyl and cyclopropyl. The hydrocarbyl (e.g. alkyl) groups can be substituted by various groups and atoms as defined herein. Examples of substituted alkyl groups include alkyl groups substituted by one or more halogen atoms such as fluorine and chlorine (particular examples including bromoethyl, chloroethyl, difluoromethyl, 2,2,2-trifluoroethyl and perfluoroalkyl groups such as trifluoromethyl), or hydroxy (e.g. hydroxymethyl and hydroxyethyl), C₁₋₈ acyloxy (e.g. acetoxymethyl and benzyloxymethyl), amino and mono- and dialkylamino (e.g. aminoethyl, methylaminoethyl, dimethylaminomethyl, dimethylaminoethyl and tert-butylaminomethyl), alkoxy (e.g. C₁₋₂ alkoxy such as methoxy—as in methoxyethyl), and cyclic groups such as cycloalkyl groups, aryl groups, heteroaryl groups and non-aromatic heterocyclic groups as hereinbefore defined).

Particular examples of alkyl groups substituted by a cyclic group are those wherein the cyclic group is a saturated cyclic amine such as morpholine, piperidine, pyrrolidine, piperazine, C₁₋₄-alkyl-piperazines, C₃₋₇-cycloalkyl-piperazines, tetrahydropyran or tetrahydrofuran and the alkyl group is a C₁₋₄ alkyl group, more typically a C₁₋₃ alkyl group such as methyl, ethyl or n-propyl. Specific examples of alkyl groups substituted by a cyclic group include pyrrolidinomethyl, pyrrolidinopropyl, morpholinomethyl, morpholinoethyl, morpholinopropyl, piperidinylmethyl, piperazinomethyl and N-substituted forms thereof as defined herein.

Particular examples of alkyl groups substituted by aryl groups and heteroaryl groups include benzyl, phenethyl and pyridylmethyl groups.

When R^{a} is SO₂NR^{c}, R^{b} can be, for example, hydrogen or an optionally substituted C₁₋₈ hydrocarbyl group, or a carbocyclic or heterocyclic group. Examples of R^{a}-R^{b} where R^{a} is SO₂NR^{c} include aminosulphonyl, C₁₋₄ alkylaminosulphonyl and di-C₁₋₄ alkylaminosulphonyl groups, and sulphonamides formed from a cyclic amino group such as piperidine, morpholine, pyrrolidine, or an optionally N-substituted piperazine such as N-methyl piperazine.

Examples of groups R^{a}-R^{b} where R^{a} is SO₂ include alkylsulphonyl, heteroarylsulphonyl and arylsulphonyl groups, particularly monocyclic aryl and heteroaryl sulphonyl groups. Particular examples include methylsulphonyl, phenylsulphonyl and toluenesulphonyl.

When R^{a} is NR^{c}, R^{b} can be, for example, hydrogen or an optionally substituted C₁₋₈ hydrocarbyl group, or a carbocyclic or heterocyclic group. Examples of R^{a}-R^{b} where R^{a} is NR^{c} include amino, C₁₋₄ alkylamino (e.g. methylamino, ethylamino, propylamino, isopropylamino, tert-butylamino), di-C₁₋₄ alkylamino (e g dimethylamino and diethylamino) and cycloalkylamino (e.g. cyclopropylamino, cyclopentylamino and cyclohexylamino).

In one general embodiment, the compound of formula (I) is other than a compound in which E is a thiazole ring having a carbonyl group attached thereto (see WO 2005/003128).

### Specific Embodiments of and Preferences for T, G, E, Q³, J¹, J² and R¹ to R¹⁰

T

In formula (I), T can be nitrogen or a group CR⁵ and J¹-J² can represent a group selected from N═C(R⁶), N═N, (R⁷)C═N, (R⁸)N—C(O), (R⁸)₂C—C(O) and (R⁷)C═C(R⁶). Thus the bicyclic group can take the form of, for example:
a purine (T is N, J¹-J² is N═C(R⁶));
a 3H-imidazo[4,5-b]pyridine (T is CR⁵, J¹-J² is N═C(R⁶));
a 7H-pyrrolo[2,3-d]pyrimidine (T is N, J¹-J² is (R⁷)C═C(R⁶));
a 1H-pyrrolo[2,3-b]pyridine (T is CR⁵, J¹-J² is (R⁷)C═C(R⁶));
a 5,7-dihydro-pyrrolo[2,3-d]pyrimidin-6-one (T is N, J¹-J² is (R⁸)₂C—C(O));
a 3H-[1,2,3]triazolo[4,5-d]pyrimidine (T is N, J¹-J² is N═N);
a 3H-[1,2,3]triazolo[4,5-b]pyridine (T is CR⁵, J¹-J² is N═N);
a 7,9-dihydro-purin-8-one (T is N, J¹-J² is (R⁸)N—C(O));
a 1H-pyrazolo[3,4-d]pyrimidine (T is N, J¹-J² is (R⁷)C═N); or
a pyrazolo[3,4-b]pyridine (T is CR⁵, J¹-J² is (R⁷)C═N).

R⁴

R⁴ is selected from hydrogen, halogen, C₁₋₅ saturated hydrocarbyl, cyano, CONH₂, CF₃ and NH₂.

More typically, R⁴ is selected from hydrogen; halogen; C₁₋₅ saturated hydrocarbyl; cyano; CF₃ and NH₂.

Preferably, R⁴ is selected from hydrogen, NH₂, chlorine, fluorine and methyl, and more preferably R⁴ is hydrogen.

R⁵

R⁵ is selected from hydrogen; halogen; C₁₋₅ saturated hydrocarbyl; CN; and CF₃. More typically, R⁵ is selected from hydrogen, chlorine, fluorine and methyl, and more preferably R⁵ is hydrogen.

R⁶

R⁶ is selected from hydrogen, halogen, C₁₋₅ saturated hydrocarbyl, cyano, CONH₂, CF₃ and NH₂.

More typically, R⁶ is selected from hydrogen; halogen; C₁₋₅ saturated hydrocarbyl; CN; and CF₃.

Preferably, R⁶ is selected from hydrogen, chlorine, fluorine and methyl, and more preferably R⁶ is hydrogen.

R⁷

R⁷ is selected from hydrogen; halogen; C₁₋₅ saturated hydrocarbyl; CN; and CF₃. More typically, R⁷ is selected from hydrogen, chlorine, fluorine and methyl, and more preferably R⁷ is hydrogen.

In one embodiment, J¹-J² is a group (R⁷)C═C(R⁶) wherein R⁶ is selected from hydrogen, chlorine, fluorine and methyl; and R⁷ is selected from hydrogen, chlorine, fluorine and methyl.

In another embodiment, J¹-J² is a group (R⁷)C═C(R⁶) wherein R⁶ is selected from hydrogen, chlorine, fluorine and methyl; and R⁷ is selected from hydrogen, bromine, chlorine, fluorine and methyl.

When J¹-J² is a group (R⁷)C═C(R⁶), it is preferred that at least one of R⁶ and R⁷ is hydrogen. In one particular subset of compounds, both R⁶ and R⁷ are hydrogen. In another particular subset of compounds, R^{6 is} hydrogen and R⁷ is selected from hydrogen, bromine, chlorine, fluorine and methyl (and more preferably R⁷ is selected from hydrogen, bromine, chlorine and methyl).

In another embodiment, J¹-J² is a group N═C(R⁶). Within this embodiment, R⁶ is typically selected from hydrogen; halogen; C₁₋₅ saturated hydrocarbyl; CN; and CF₃. Preferably, R⁶ is selected from hydrogen, chlorine, fluorine and methyl. More preferably, R⁶ is hydrogen.

In a further embodiment, J¹-J² is a group (R⁷)C═N. Within this embodiment, R⁷ is typically selected from hydrogen, chlorine, fluorine and methyl. Preferably, R⁷ is hydrogen.

R⁸

R⁸ is selected from hydrogen, halogen, C₁₋₅ saturated hydrocarbyl, cyano, CONH₂, CF₃ and NH₂.

More typically, R⁸ is selected from hydrogen; halogen; C₁₋₅ saturated hydrocarbyl; CN; and CF₃.

In one embodiment, when attached to a nitrogen atom, R⁸ is selected from hydrogen and C₁₋₅ saturated hydrocarbyl (e.g. alkyl) and more typically is selected from hydrogen, methyl and ethyl; and preferably is hydrogen.

In another embodiment, when attached to a carbon atom, R⁸ is selected from hydrogen, chlorine, fluorine, methyl, and ethyl; and preferably is hydrogen.

In another embodiment of the invention, J¹-J² is a group (R⁸)N—C(O). Within this embodiment, R⁸ is typically selected from hydrogen and C₁₋₅ saturated hydrocarbyl (e.g. alkyl). More typically, R⁸ is selected from hydrogen, methyl and ethyl; and preferably is hydrogen.

In a further embodiment, J¹-J² is a group (R⁸)₂C—C(O). Within this embodiment, R⁸ is typically selected from hydrogen, chlorine, fluorine, methyl, and ethyl; and preferably is hydrogen.

Q¹

Q³ is a bond or a saturated hydrocarbon linker group containing from 1 to 3 carbon atoms; wherein the carbon atoms of the linker group may optionally bear one or more substituents selected from fluorine and hydroxy, provided that the hydroxy group when present is not located at a carbon atom α with respect to the G group.

In one embodiment of the invention, no fluorine atoms are present in the linker group Q³.

In another embodiment of the invention, no hydroxy groups are present in the linker group Q³.

In one group of compounds of the formula (I) neither hydroxy groups nor fluorine atoms are present in the linker group Q³.

In another group of compounds of the invention, the linker group Q³ can have a branched configuration at the carbon atom attached to G, when present. For example, the carbon atom attached to G can be attached to a pair of gem-dimethyl groups.

Preferably Q³ is a bond or a group (CH₂)ₐ wherein a is 1, 2 or 3, more particularly 1 or 2, and preferably 1.

Most preferably, Q³ is a bond or a group (CH₂)ₐ wherein a is 1.

In one particularly preferred group of compounds, Q³ is a bond.

In another particularly preferred group of compounds, Q³ is CH₂.

G

The moiety G is selected from NR²R³, CN and OH.

In one embodiment, G is NR²R³.

Within the sub-group of compounds in which G is NR²R³, R² and R³ can be independently selected from hydrogen; C₁₋₄ hydrocarbyl and C₁₋₄ acyl wherein the hydrocarbyl and acyl groups are optionally substituted by one or more substituents selected from fluorine, hydroxy, cyano, amino, methylamino, dimethylamino, methoxy and a monocyclic or bicyclic aryl or heteroaryl group;

Within this group of compounds are the compounds wherein R² and R³ are independently selected from hydrogen; C₁₋₄ hydrocarbyl and C₁₋₄ acyl wherein the hydrocarbyl and acyl groups are each optionally substituted by a monocyclic or bicyclic aryl or heteroaryl group.

Also within this group of compounds is the sub-group of compounds of the invention wherein R² and R³ are independently selected from hydrogen, C₁₋₄ hydrocarbyl and C₁₋₄ acyl.

In each of the foregoing groups and sub-groups of compounds, the hydrocarbyl group forming part of NR²R³ typically is an alkyl group, more usually a C₁, C₂ or C₃ alkyl group, for example a methyl group.

In a particular sub-group of compounds, R² and R³ are independently selected from hydrogen and methyl and hence NR²R³ can be an amino, methylamino or dimethylamino group, and more preferably an amino or methylamino group.

In one embodiment, NR²R³ is an amino group. In another particular embodiment, NR²R³ is a methylamino group.

In another group of compounds, R² and R³ together with the nitrogen atom to which they are attached form a saturated monocyclic heterocyclic group having 4-7 ring members and optionally containing a second heteroatom ring member selected from O and N.

In a further group of compounds, one of R² and R³ together with the nitrogen atom to which they are attached and one or more atoms from the linker group Q³ form a saturated monocyclic heterocyclic group having 4-7 ring members and optionally containing a second heteroatom ring member selected from O and N.

In each case, the saturated monocyclic ring can be an azacycloalkyl group such as an azetidine, pyrrolidine, piperidine or azepane ring, and such rings are typically unsubstituted.

Alternatively, the saturated monocyclic ring can contain an additional heteroatom selected from O and N, and examples of such groups include morpholine and piperazine. Where an additional N atom is present in the ring, this can form part of an NH group or an N-C₁₋₄alkyl group such as an N-methyl, N-ethyl, N-propyl or N-isopropyl group.

Particular groups wherein NR²R³ forms a cyclic moiety are azetidine, pyrrolidine, piperidine (e.g. 2-piperidine or 4-piperidine—preferably 4-piperidine), azepine, piperazine, N—C₁₋₄ alkylpiperazine (e.g. N-methylpiperazine), morpholine and thiomorpholine and S-oxides and S,S-dioxides thereof. Preferred monocyclic heterocyclic rings are pyrrolidine, piperidine (e.g. 4-piperidine), piperazine, N-methylpiperazine and morpholine.

Preferred moieties Q³-G are the groups NH₂, NHMe, CH₂NH₂ or CH₂NHMe.

In one embodiment, Q³-G is NH₂ or NHMe.

In another embodiment, Q³-G is CH₂NH₂ or CH₂NHMe.

E

The ring E is a five membered heteroaryl ring containing 1, 2, 3 or 4 heteroatoms selected from O, N and S provided that no more than 1 heteroatom may be other than nitrogen. As stated in the definitions of R^{1a} and R^{1b}, when R^{1a} and R^{1b} are each hydrogen or R¹⁰, then the heteroaryl ring E is other than a thiophene or furan ring. Thus the definition of E excludes inter alia thiophene and furan rings that are not fused to another ring. However, the definition includes groups E wherein thiophene or furan rings are fused to another ring such as a benzene or pyridine ring, and hence the combination of E, R^{1a} and R^{1b} covers bicyclic groups such as benzothiophene and benzofuran.

The heteroaryl ring can be any of the five-membered heteroaryl rings described above in the General Preferences and Definitions section provided that they meet the requirements as to the number and type of the heteroatoms.

Preferably, there are 1, 2 or 3 heteroatoms.

Examples of heteroaryl rings include imidazole, oxazole, thiazole, thiadiazole, oxadiazole, pyrazole, furazan, pyrrole, furan (when fused to another ring) and thiophene (when fused to another ring).

Particular examples of heteroaryl rings, with the positions of attachment of R^{1a} and R^{1b} shown, and the point of attachment to the piperidine ring indicated by means of an asterisk, are set out in Table 1.

TABLE 1

| | | |
|---|---|---|
| 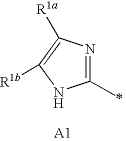 | 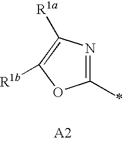 | 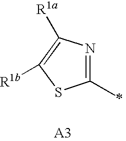 |
| 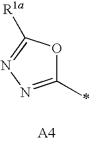 | 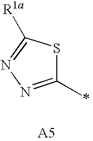 | 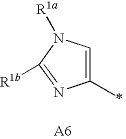 |
| 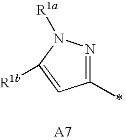 | | |

Preferred heteroaryl rings are rings A1 and A2.

A particularly preferred heteroaryl ring is A1.

q & r

The integers q and r are each 0 or 1.

The values for q and r will depend on the nature of the adjacent ring atom in the ring E. If the relevant ring atom is oxygen or sulphur, then no group R^{1a} (or R^{1b}) will be present, i.e. the integer q or r is 0. If the relevant ring atom is nitrogen, then the integer q or r can be 0 or 1 depending upon whether or not the nitrogen atom forms a formal double bond with a neighbouring ring atom. If the relevant ring atom is carbon, then the integer q or r will be 1.

In one preferred group of compounds, the sum of q+r is 1 or 2.

R^{1a} and R^{1b}

R^{1a} and R^{1b} are the same or different and each is hydrogen or a substituent R¹⁰; or R^{1a} and R^{1b} together with the carbon atoms or heteroatoms to which they are attached form a 5 or 6-membered aryl or heteroaryl ring, wherein the aryl or heteroaryl rings are optionally substituted by one or more substituents R¹⁰; provided that when R^{1a} and R^{1b} are each hydrogen or R¹⁰, then the heteroaryl ring E is other than a thiophene or furan ring.

In one embodiment, R^{1a} and R^{1b} together with the carbon atoms to which they are attached form a five or six-membered aryl or heteroaryl ring, thereby giving a bicyclic fused ring structure. The said five or six-membered aryl or heteroaryl rings are optionally substituted by one or more substituents R¹⁰.

The aryl or heteroaryl ring may be any of the five or six membered aryl and heteroaryl rings defined above in the General Preferences and Definitions section.

In one embodiment, the aryl and heteroaryl rings are six-membered rings.

The aryl and heteroaryl rings are preferably selected from benzene and pyridine rings.

Examples of the moiety:

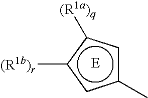

in which R^{1a} and R^{1b} combine with the ring E to form a bicyclic fused ring structure are shown in Table 2.

TABLE 2

| | |
|---|---|
| 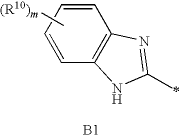 | 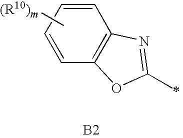 |
| 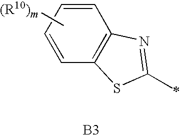 | 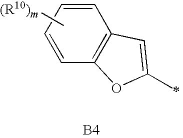 |
| 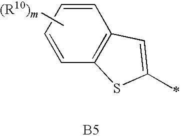 | 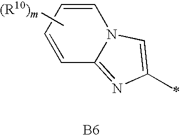 |
| 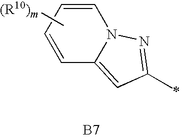 | 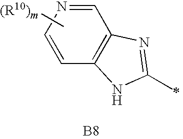 |
| 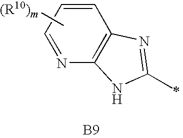 | |

One sub-group of bicyclic fused ring structures consists of structures B1 to B8.

One group of preferred bicyclic fused ring structures consists of structures B1, B2, B8 and B9.

Another group of preferred bicyclic fused ring structures consists of B1, B2 and B8.

Particularly preferred fused ring structures are B1 and B2.

A more particular fused ring structure is B1.

The five and six-membered aryl and heteroaryl rings making up the bicyclic fused ring structure can be unsubstituted or substituted by one or more substituents R¹⁰ as defined herein. For example, there can be 0, 1, 2 or 3 substituents on the aryl or heteroaryl ring, more usually 0, 1 or 2, for example 0 substituents or 1 substituent or 2 substituents.

Examples of substituents are the substituents selected from R^{10a}, R^{10b} and R^{10c} as defined herein.

Particular substituents are selected from a group R^{10d} consisting of:
hydroxy;
halogen (e.g. fluorine and chlorine);
cyano;
amino;
mono-C₁₋₄-alkylamino or di-C₁₋₄-alkylamino wherein the C₁₋₄-alkyl moieties of the mono-C₁₋₄-alkylamino and di-C₁₋₄-alkylamino groups are optionally substituted by hydroxy (other than α-hydroxy), C₁₋₂ alkoxy, amino, mono-C₁₋₂-alkylamino, di-C₁₋₂-alkylamino, C₁₋₂ acylamino;
optionally substituted C₁₋₄ alkyl (e.g. methyl);
optionally substituted C₁₋₄ alkoxy (e.g. methoxy) groups;
optionally substituted C₁₋₄ alkylthio; wherein the optional substituents for the C₁₋₄ alkyl, C₁₋₄ alkoxy and C₁₋₄ alkylthio groups are selected from halogen (e.g. fluorine), hydroxy, C₁₋₂ acyloxy, C₁₋₂ alkoxy (e.g. methoxy), amino, mono-C₁₋₄-alkylamino, di-C₁₋₄-alkylamino and C₁₋₄ acylamino;
a group R^{cyc};
a group O—R^{cyc};
a group (O)ᵣ—(CH₂)ₜ—R^{cyc}, wherein r is 0 or 1; t is 0, 1 or 2;

R^{cyc} is:
a 5-6 membered aryl or heteroaryl ring optionally substituted by one or more substituents selected from C₁₋₄ alkyl, C₁₋₄ alkoxy, halogen, hydroxy, C₁₋₄ acyloxy, cyano, trifluoromethyl, trifluoromethoxy, difluoromethoxy and a group (CH₂)ₓR¹⁷ where x is 0, 1 or 2 (preferably 0 or 1) and R¹⁷ is amino, mono-C₁₋₄-alkylamino, di-C₁₋₄-alkylamino, C₁₋₄ acylamino, aminosulphonyl, mono-C₁₋₄-alkylaminosulphonyl, di-C₁₋₄-alkylaminosulphonyl or C₁₋₄-alkylsulphonyl; or
a 3-7 membered non-aromatic carbocyclic or heterocyclic ring optionally substituted by C₁₋₄ alkyl, C₁₋₄ alkoxy, halogen, hydroxy, C₁₋₄ acyloxy, cyano, trifluoromethyl, trifluoromethoxy, difluoromethoxy, amino, mono-C₁₋₄-alkylamino, di-C₁₋₄-alkylamino; C₁₋₄ acylamino or C₁₋₄ alkoxycarbonyl.

Within group R^{10d}, a sub-set of particular substituents is selected from a group R^{10e} consisting of:
hydroxy;
halogen (e.g. fluorine and chlorine);
cyano;
amino;
mono-C₁₋₄-alkylamino or di-C₁₋₄-alkylamino wherein the C₁₋₄-alkyl moieties of the mono-C₁₋₄-alkylamino and di-C₁₋₄-alkylamino groups are optionally substituted by hydroxy (other than α-hydroxy), C₁₋₂ alkoxy, amino, mono-C₁₋₂-alkylamino, di-C₁₋₂-alkylamino, C₁₋₂ acylamino;
optionally substituted C₁₋₄ alkyl (e.g. methyl);
optionally substituted C₁₋₄ alkoxy (e.g. methoxy) groups;
optionally substituted C₁₋₄ alkylthio; wherein the optional substituents for the C₁₋₄ alkyl, C₁₋₄ alkoxy and C₁₋₄ alkylthio groups are selected from halogen (e.g. fluorine), hydroxy, C₁₋₂ acyloxy, C₁₋₂ alkoxy (e.g. methoxy), amino, mono-C₁₋₄-alkylamino, di-C₁₋₄-alkylamino and C₁₋₄ acylamino;
a group R^{cyc};
a group O—R^{cyc};
a group (O)ᵣ—(CH₂)ₜ—R^{cyc′}, wherein r is 0 or 1; t is 0, 1 or 2;

R^{cyc} is:
a 5-6 membered aryl or heteroaryl ring optionally substituted by C₁₋₄ alkyl, C₁₋₄ alkoxy, halogen, hydroxy, C₁₋₄ acyloxy, cyano, trifluoromethyl, trifluoromethoxy, difluoromethoxy, amino, mono-C₁₋₄-alkylamino, di-C₁₋₄-alkylamino, C₁₋₄ acylamino, aminosulphonyl, mono-C₁₋₄-alkylaminosulphonyl, di-C₁₋₄-alkylaminosulphonyl or C₁₋₄-alkylsulphonyl; or
a 3-7 membered non-aromatic carbocyclic or heterocyclic ring optionally substituted by C₁₋₄ alkyl, C₁₋₄ alkoxy, halogen, hydroxy, C₁₋₄ acyloxy, cyano, trifluoromethyl, trifluoromethoxy, difluoromethoxy, amino, mono-C₁₋₄-alkylamino, di-C₁₋₄-alkylamino; C₁₋₄ acylamino or C₁₋₄ alkoxycarbonyl.

Within group R^{10e}, a further sub-set of particular substituents is selected from a group R^{10f} consisting of:
hydroxy;
halogen (e.g. fluorine and chlorine);
cyano;
amino;
mono-C₁₋₄-alkylamino or di-C₁₋₄-alkylamino wherein the C₁₋₄-alkyl moieties of the mono-C₁₋₄-alkylamino and di-C₁₋₄-alkylamino groups are optionally substituted by hydroxy (other than α-hydroxy), C₁₋₂ alkoxy, amino, mono-C₁₋₂-alkylamino, di-C₁₋₂-alkylamino, C₁₋₂ acylamino;
optionally substituted C₁₋₄ alkyl (e.g. methyl);
optionally substituted C₁₋₄ alkoxy (e.g. methoxy) groups; wherein the optional substituents for the C₁₋₄ alkyl and C₁₋₄ alkoxy groups are selected from halogen (e.g. fluorine), hydroxy, C₁₋₂ acyloxy, C₁₋₂ alkoxy (e.g. methoxy), amino, mono-C₁₋₄-alkylamino, di-C₁₋₄-alkylamino and C₁₋₄ acylamino; and
a group (CH₂)ₜ—R^{cyc″}, wherein t is 0, 1 or 2 and R^{cyc″} is:
   a 5-6 membered aryl or heteroaryl ring optionally substituted by C₁₋₄ alkyl, C₁₋₄ alkoxy, halogen, hydroxy, C₁₋₄ acyloxy, cyano, trifluoromethyl, trifluoromethoxy, difluoromethoxy, amino, mono-C₁₋₄-alkylamino, di-C₁₋₄-alkylamino or C₁₋₄ acylamino;
   a 3-7 membered non-aromatic carbocyclic or heterocyclic ring optionally substituted by C₁₋₄ alkyl, C₁₋₄ alkoxy, halogen, hydroxy, C₁₋₄ acyloxy, cyano, trifluoromethyl, trifluoromethoxy, difluoromethoxy, amino, mono-C₁₋₄-alkylamino, di-C₁₋₄-alkylamino; C₁₋₄ acylamino or C₁₋₄ alkoxycarbonyl.

One sub-set of substituents consists of phenyl, pyridyl, thienyl, furanyl, imidazoyl, pyrazolyl, methylpyrazolyl, pyrrolidinyl, piperidinyl, piperazinyl, N-methylpiperazinyl, morpholinyl, hydroxy, halogen (e.g. fluorine and chlorine); C₁₋₄ alkyl (e.g. methyl) and C₁₋₄ alkoxy (e.g. methoxy) groups wherein the C₁₋₄ alkyl and C₁₋₄ alkoxy groups are each optionally substituted by fluorine and methoxy.

Another subset of substituents consists of:
phenyl [optionally substituted by one or two substituents selected from C₁₋₄ alkoxy (e.g. methoxy), C₁₋₄ alkylsulphonyl (e.g. methylsulphonyl), C₁₋₄ alkylsulphonylmethyl (e.g. methylsulphonylmethyl), C₁₋₄ alkylaminosulphonyl, C₁₋₄ alkylaminosulphonylmethyl, aminosulphonyl, aminosulphonylmethyl, chlorine, bromine and fluorine]
pyridyl;
thienyl;
furanyl;
imidazoyl;
pyrazolyl or methylpyrazolyl;
pyrrolidinyl;
piperidinyl;
piperazinyl or N-methylpiperazinyl;
morpholinyl;
hydroxyl;
halogen (e.g. fluorine and chlorine);
C₁₋₄ alkyl (e.g. methyl) and C₁₋₄ alkoxy (e.g. methoxy) groups [wherein the C₁₋₄ alkyl and C₁₋₄ alkoxy groups are each optionally substituted by fluorine (i.e. one or more fluorine atoms), methoxy, hydroxyl, amino, C₁₋₄ alkylsulphonyl, (e.g. methylsulphonyl), C₁₋₄ alkylsulphonylmethyl (e.g. methylsulphonylmethyl), C₁₋₄ alkylaminosulphonyl, C₁₋₄ alkylaminosulphonylmethyl, aminosulphonyl or aminosulphonylmethyl].

In another embodiment, R^{1a} and R^{1b} are the same or different and each is hydrogen or a substituent R¹⁰.

Within this embodiment, one sub-group consists of compounds wherein one of R^{1a} and R^{1b} is selected from hydrogen, methyl, chlorine or trifluoromethyl and the other of R^{1a} and R^{1b} is a 5- or 6-membered aryl or heteroaryl ring optionally substituted by one or more substituents R¹¹; where R¹¹ is selected from halogen, hydroxy, trifluoromethyl, cyano, nitro, carboxy, amino, mono- or di-C₁₋₄ hydrocarbylamino, monocyclic carbocyclic and heterocyclic groups having from 3 to 7 ring members; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, monocyclic carbocyclic and heterocyclic groups having from 3 to 7 ring members, and C₁₋₈ hydrocarbyl optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
wherein in each case the monocyclic carbocyclic and heterocyclic groups having from 3 to 7 ring members are unsubstituted or substituted by one or more further substituents R¹¹ provided that such further substituents are not cyclic;
R^{c} is selected from hydrogen and C₁₋₄ hydrocarbyl; and
X¹ is O, S or NR^{c} and X² is ═O, ═S or ═NR^{c}.

Particular examples of the moiety:

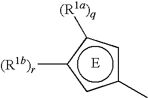

are shown in Table 3.

TABLE 3

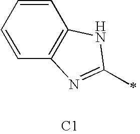

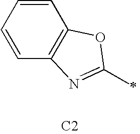

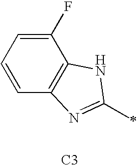

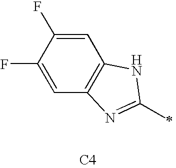

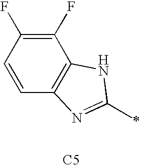

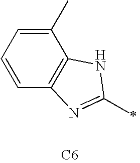

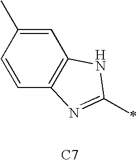

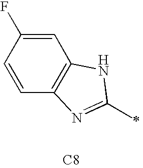

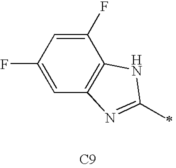

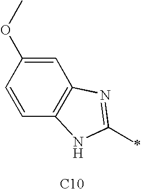

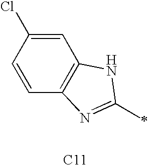

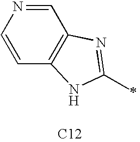

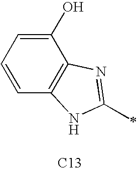

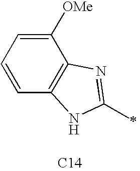

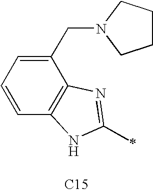

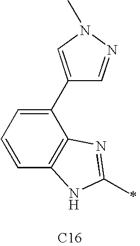

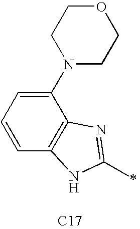

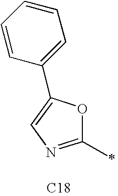

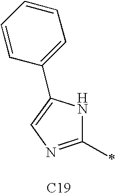

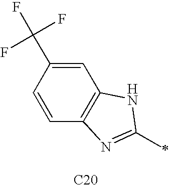

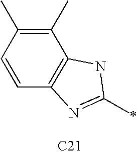

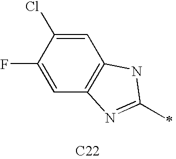

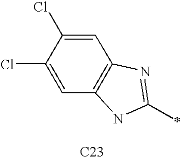

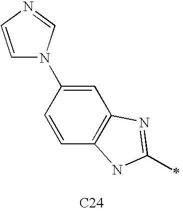

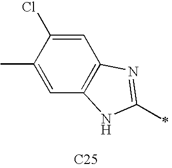

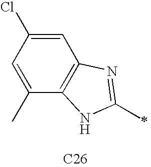

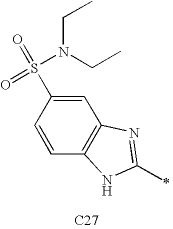

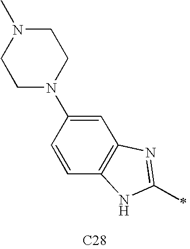

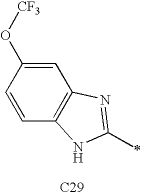

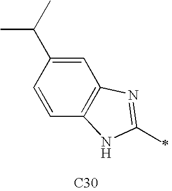

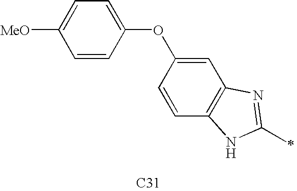

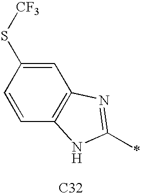

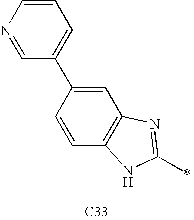

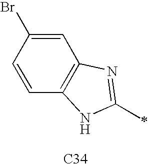

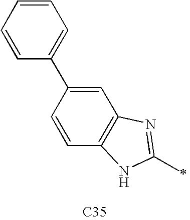

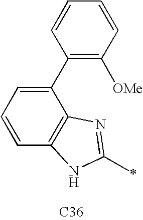

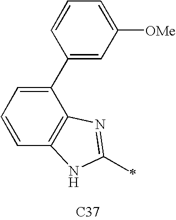

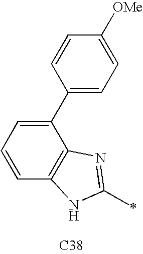

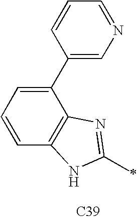

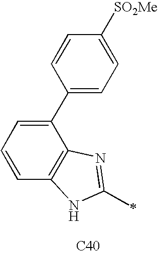

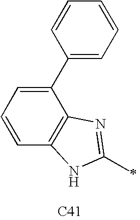

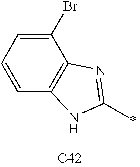

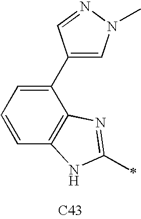

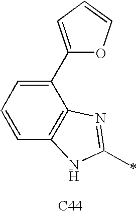

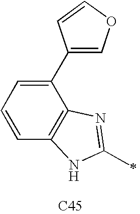

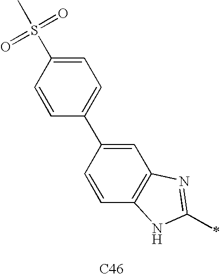

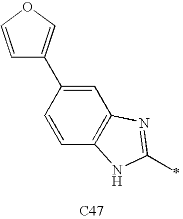

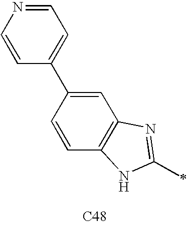

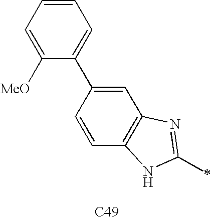

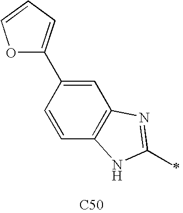

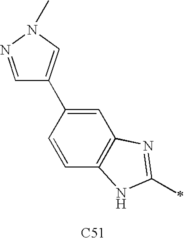

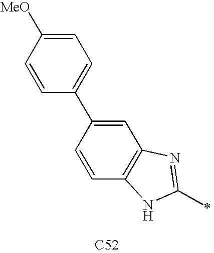

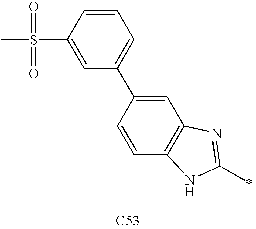

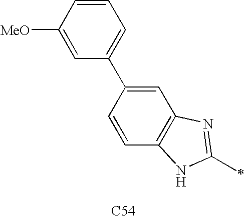

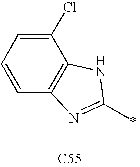

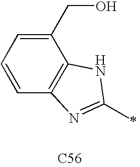

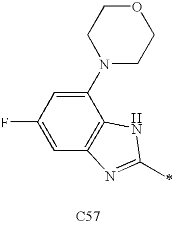

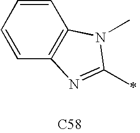

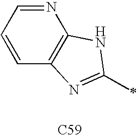

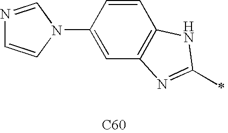

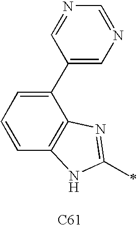

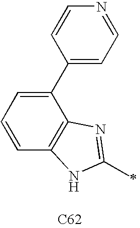

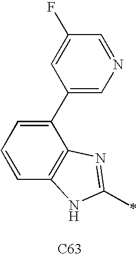

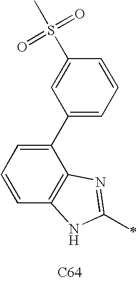

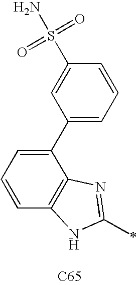

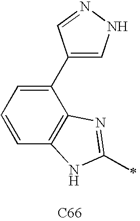

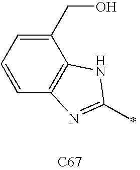

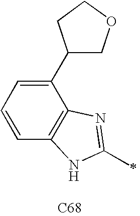

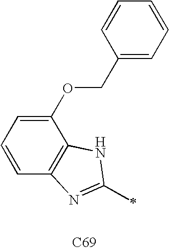

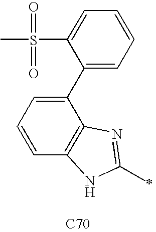

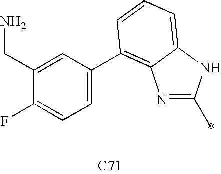

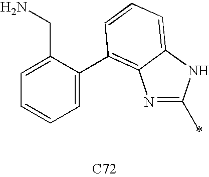

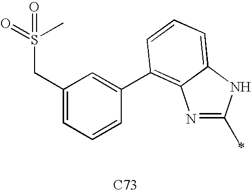

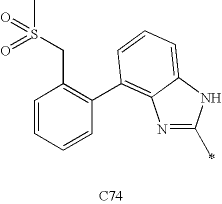

It will be appreciated that each of the benzimidazole structures shown in the table can exist in two tautomeric forms. Although only one tautomer is shown, a reference to a particular structure is intended to refer to both tautomers, unless the context indicates otherwise.

One preferred set of groups within Table 3 consists of groups C1 to C19.

Another set of of preferred groups in Table 3 consists of C1, C7, C9, C10, C11 and C14.

Particular and Preferred Sub-Groups of the Formula (I)

One sub-group of compounds of the formula (I) has the general formula (II):

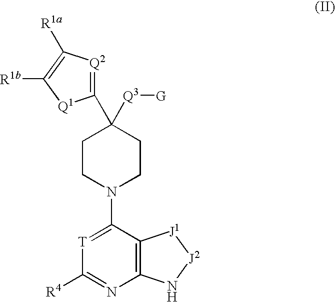

or salts, solvates, tautomers or N-oxides thereof, wherein
T is N or a group CR⁵;
J¹-J² represents a group selected from N═C(R⁶), (R⁷)C═N, (R⁸)N—C(O), (R⁸)₂C—C(O), N═N and (R⁷)C═C(R⁶);
Q¹ is NH, S or O;
Q² is N or CH;
Q³ is a bond or a saturated hydrocarbon linker group containing from 1 to 3 carbon atoms; wherein the carbon atoms of the linker group may optionally bear one or more substituents selected from fluorine and hydroxy, provided that the hydroxy group when present is not located at a carbon atom α with respect to the G group;
G is selected from NR²R³, CN and OH;
R^{1a} and R^{1b} are the same or different and each is hydrogen or a substituent R¹⁰; or R^{1a} and R^{1b} together with the carbon atoms to which they are attached form a 6-membered aryl or heteroaryl ring, wherein the aryl or heteroaryl rings are optionally substituted by one or more substituents R¹⁰; provided that when Q¹ is S or O and R^{1a} and R^{1b} are each hydrogen or R¹⁰, then Q² is N;
R² and R³ are independently selected from hydrogen; C₁₋₄ hydrocarbyl and C₁₋₄ acyl wherein the hydrocarbyl and acyl groups are optionally substituted by one or more substituents selected from fluorine, hydroxy, cyano, amino, methylamino, dimethylamino, methoxy and a monocyclic or bicyclic aryl or heteroaryl group;
or R² and R³ together with the nitrogen atom to which they are attached form a cyclic group selected from an imidazole group and a saturated monocyclic heterocyclic group having 4-7 ring members and optionally containing a second heteroatom ring member selected from O and N;
or one of R² and R³ together with the nitrogen atom to which they are attached and one or more atoms from the group Q³ form a saturated monocyclic heterocyclic group having 4-7 ring members and optionally containing a second heteroatom ring member selected from O and N; and
R⁴, R⁶ and R⁸ are each independently selected from hydrogen, halogen, C₁₋₅ saturated hydrocarbyl, cyano, CONH₂, CF₃ and NH₂;
R⁵ and R⁷ are each independently selected from hydrogen, halogen, C₁₋₅ saturated hydrocarbyl, cyano and CF₃; and
R¹⁰ is selected from halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and C₁₋₈ hydrocarbyl optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{c} is selected from hydrogen and C₁₋₄ hydrocarbyl; and
X¹ is O, S or NR^{c} and X² is ═O, ═S or ═NR^{c}.

In formula (II), particular and preferred values for T, R^{1a}, R^{1b}, R⁴, R⁵, R⁶, R⁷, R⁸, Q³, G, J¹ and J² are as defined above in relation to Formula (I).

In formula (II), Q¹ is NH, S or O and Q² is N or CH.

Preferably Q¹ is NH or O.

Preferably Q² is N.

In one embodiment, Q¹ is NH, S or O and Q² is N.

In a further embodiment, Q¹ is NH or O and Q² is N.

In another embodiment, Q¹ is NH and Q² is N.

In another embodiment, Q¹ is O and Q² is N.

One sub-group of compounds of the formula (II) has the general formula (III):

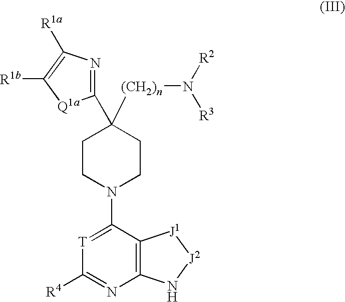

or salts, solvates, tautomers or N-oxides thereof, wherein Q^{1a} is NH or O; n is 0, 1 or 2; and R^{1a}, R^{1b}, R², R³, R⁴, T, J¹ and J² are as defined herein in respect of formulae (I) and (II) and sub-groups, examples and preferences thereof.

In one embodiment, Q^{1a} is NH.

In another embodiment, Q^{1a} is O.

Within formula (III), one sub-group of compounds can be represented by the formula (IV):

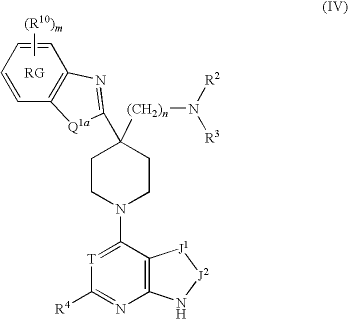

or salts, solvates, tautomers or N-oxides thereof, wherein Q^{1a} is NH or O; the ring RG is a benzene or pyridine ring, m is 0, 1 or 2; n is 0, 1 or 2; and R², R³, R⁴, R¹⁰, T, J¹ and J² are as defined herein in respect of formula (I) and sub-groups, examples and preferences thereof.

In one embodiment, Q^{1a} is NH.

Within this embodiment, in one group of compounds (i) RG is a benzene ring and in another group of compounds (ii) RG is a pyridine ring.

In another embodiment, Q^{1a} is O.

Within formula (IV), one group of preferred compounds is represented by the formula (IV⁰).

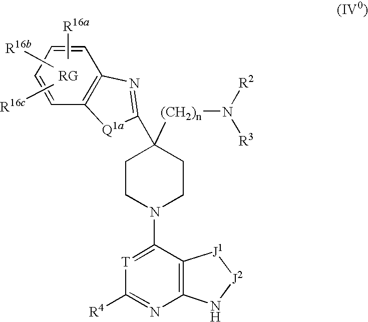

or salts, solvates, tautomers or N-oxides thereof, wherein n, T, Q^{1a}, J¹-J², R², R³ and R⁴ are as defined herein in respect of formula (I) and sub-groups, examples and preferences thereof;

R^{16a} is selected from:
hydrogen;
hydroxy;
halogen (e.g. fluorine, chlorine and bromine);
cyano;
amino;
mono-C₁₋₄-alkylamino or di-C₁₋₄-alkylamino wherein the C₁₋₄-alkyl moieties of the mono-C₁₋₄-alkylamino and di-C₁₋₄-alkylamino groups are optionally substituted by hydroxy (other than α-hydroxy), C₁₋₂ alkoxy, amino, mono-C₁₋₂-alkylamino, di-C₁₋₂-alkylamino, C₁₋₂ acylamino;
optionally substituted C₁₋₄ alkyl (e.g. methyl);
optionally substituted C₁₋₄ alkoxy (e.g. methoxy) groups;
optionally substituted C₁₋₄ alkylthio; wherein the optional substituents for the C₁₋₄ alkyl, C₁₋₄ alkoxy and C₁₋₄ alkylthio groups are selected from halogen (e.g. fluorine), hydroxy, C₁₋₂ acyloxy, C₁₋₂ alkoxy (e.g. methoxy), amino, mono-C₁₋₄-alkylamino, di-C₁₋₄-alkylamino and C₁₋₄ acylamino;
a group R^{cyc′};
a group O—R^{cyc′};
a group (O)ᵣ—(CH₂)ₜ—R^{cyc′}, wherein r is 0 or 1; t is 0, 1 or 2;

R^{cyc′} is:
a 5-6 membered aryl or heteroaryl ring optionally substituted by C₁₋₄ alkyl, C₁₋₄ alkoxy, halogen, hydroxy, C₁₋₄ acyloxy, cyano, trifluoromethyl, trifluoromethoxy, difluoromethoxy, amino, mono-C₁₋₄-alkylamino, di-C₁₋₄-alkylamino, C₁₋₄ acylamino, aminosulphonyl, mono-C₁₋₄-alkylaminosulphonyl, di-C₁₋₄-alkylaminosulphonyl or C₁₋₄-alkylsulphonyl; or
a 3-7 membered non-aromatic carbocyclic or heterocyclic ring optionally substituted by C₁₋₄ alkyl, C₁₋₄ alkoxy, halogen, hydroxy, C₁₋₄ acyloxy, cyano, trifluoromethyl, trifluoromethoxy, difluoromethoxy, amino, mono-C₁₋₄-alkylamino, di-C₁₋₄-alkylamino; C₁₋₄ acylamino or C₁₋₄ alkoxycarbonyl;

R^{16b} is selected from:
hydrogen;
hydroxy;
halogen (e.g. fluorine and chlorine);
cyano;
trifluoromethyl;
trifluoromethoxy;
difluoromethoxy;
amino;
C₁₋₄ alkylamino;
di-C₁₋₄ alkylamino;
C₁₋₄ alkyl; and
C₁₋₄ alkoxy; and

R^{16c} is selected from:
hydrogen;
fluorine;
chlorine; and
methyl.

In one general embodiment, R^{16a} can be selected from each of the moieties listed in the above definition of R^{16a} provided that when R^{16a} is a group (O)ᵣ—(CH₂)ₜ—R^{cyc′}, then r is 0.

In another general embodiment, R^{16a} can be selected from each of the moieties listed in the above definition of R^{16a} provided that when R^{16a} is a group (O)ᵣ—(CH₂)ₜ—R^{cyc′}, then r is 1.

In formula (IV⁰), typically at least one of R^{16a}, R^{16b} and R^{16c} is hydrogen.

In one subset of compounds, or salts, solvates, tautomers or N-oxides thereof:

R^{16a} is selected from:
hydrogen;
hydroxy;
fluorine;
chlorine;
bromine;
cyano;
amino;
mono-C₁₋₄-alkylamino or di-C₁₋₄-alkylamino wherein the C₁₋₄-alkyl moieties of the mono-C₁₋₄-alkylamino and di-C₁₋₄-alkylamino groups are optionally substituted by hydroxy (other than α-hydroxy), C₁₋₂ alkoxy, amino, mono-C₁₋₂-alkylamino, di-C₁₋₂-alkylamino, C₁₋₂ acylamino;
optionally substituted C₁₋₄ alkyl (e.g. methyl);
optionally substituted C₁₋₄ alkoxy (e.g. methoxy) groups;
optionally substituted C₁₋₄ alkylthio; wherein the optional substituents for the C₁₋₄ alkyl, C₁₋₄ alkoxy and C₁₋₄ alkylthio groups are selected from fluorine, methoxy, amino, mono-C₁₋₂-alkylamino, di-C₁₋₂-alkylamino and C₁₋₂ acylamino;
a group R^{cyc};
a group O—R^{cyc}; and
a group (CH₂)ₜ—R^{cyc′}, wherein t is 0, 1 or 2 and R^{cyc′} is:
   optionally substituted phenyl or an optionally substituted 5-6 membered heteroaryl ring containing 1 or 2 heteroatom ring members selected from O, N and S (and more preferably O and N), wherein the optional substituents for the phenyl group and heteroaryl ring are selected from C₁₋₄ alkyl, C₁₋₄ alkoxy, fluorine, chlorine, bromine, hydroxy, C₁₋₂ acyloxy, cyano, trifluoromethyl, trifluoromethoxy, difluoromethoxy, amino, mono-C₁₋₂-alkylamino, di-C₁₋₂-alkylamino, C₁₋₂ acylamino, aminosulphonyl, mono-C₁₋₂-alkylaminosulphonyl, di-C₁₋₂-alkylaminosulphonyl or C₁₋₂-alkylsulphonyl;
   a 4-6 membered non-aromatic carbocyclic or heterocyclic ring containing up to 2 heteroatom ring members selected from O, N and N and being optionally substituted by C₁₋₄ alkyl, C₁₋₄ alkoxy, halogen, hydroxy, C₁₋₄ acyloxy, cyano, trifluoromethyl, trifluoromethoxy, difluoromethoxy, amino, mono-C₁₋₄-alkylamino, di-C₁₋₄-alkylamino; C₁₋₄ acylamino or C₁₋₄ alkoxycarbonyl;

R^{16b} is selected from:
hydrogen;
hydroxy;
fluorine;
chlorine;
C₁₋₄ alkyl; and
C₁₋₄ alkoxy; and

R^{16c} is selected from:
hydrogen;
fluorine;
chlorine; and
methyl.

When R^{16a} is a group R^{cyc′}, it can be for example a non-aromatic group such as azetidine, pyrrolidine, piperidine, piperazine, N-methylpiperazine, morpholine, thiomorpholine and the S-oxide and S,S-dioxides of thiomorpholine, or it can be a monocyclic aromatic group such as phenyl, pyridyl, thienyl, furanyl, imidazolyl, pyrazolyl, each of which monocyclic aromativ groups is optionally substituted by methoxy, methyl, methylsulphonyl, fluorine, chlorine, trifluoromethyl or trifluoromethoxy.

When R^{16a} is a group R^{cyc′}, R^{16b} is preferably selected from hydrogen, fluorine, chlorine, methoxy or methyl and R^{16c} is preferably hydrogen. More preferably, R^{16b} is hydrogen.

Another sub-set of compounds within formula (IV⁰) can be represented by the formula (IV⁰⁰):

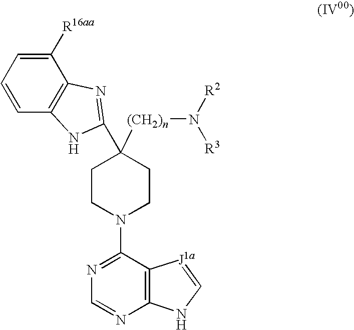

or a salt, solvate, tautomer or N-oxide thereof, wherein n, R² and R³ are as defined herein; J^{1a} is CH or N; and R^{16aa} is a monocyclic aromatic group selected from phenyl, pyridyl, pyrimidinyl, thienyl, furanyl, imidazolyl and pyrazolyl, each of which monocyclic aromatic groups is optionally substituted by methoxy, methyl, aminosulphonyl, methylsulphonyl, fluorine, chlorine, trifluoromethyl or trifluoromethoxy.

One particular subset of monocyclic aromatic groups R^{16aa} consists of phenyl, pyridyl, thienyl, furanyl, imidazolyl and pyrazolyl, each of which monocyclic aromatic groups is optionally substituted by methoxy, methyl, methylsulphonyl, fluorine, chlorine, trifluoromethyl or trifluoromethoxy.

Preferably, R^{16aa} is an optionally substituted monocyclic aromatic group selected from phenyl, furanyl and pyrazolyl. In this sub-set of compounds, J^{1a} is preferably CH.

When R^{16a} is other than a group R^{cyc′}, O—R^{cyc′} or (CH₂)ₜ—R^{cyc′} (or is other than a group R^{cyc″}, O—R^{cyc″} or (O)ᵣ—(CH₂)ₜ—R^{cyc″}) particular combinations of R^{16a}, R^{16b} and R^{16c} are those in which R^{16c} is hydrogen, R^{16a} is selected from hydrogen, fluorine, chlorine, bromine, C₁₋₃ alkyl, hydroxy, methoxy, trifluoromethyl, di-C₁₋₂ alkylaminosulphonyl, trifluoromethoxy, trifluoromethylthio; and R^{16b} is selected from hydrogen, fluorine and methyl.

One subset of compounds wherein R^{16a} is other than a group R^{cyc′}, O—R^{cyc′} or (CH₂)ₜ—R^{cyc′} (or is other than a group R^{cyc′}, O—R^{cyc″} or (O)ᵣ—(CH₂)ₜ—R^{cyc″}) is represented by formula (IV⁰⁰⁰):

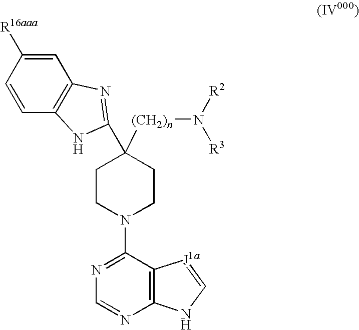

or a salt, solvate, tautomer or N-oxide thereof, wherein n, R² and R³ are as defined herein; J^{1a} is CH or N; and R^{16aaa} is selected from hydrogen, fluorine, chlorine, bromine, C₁₋₃ alkyl, hydroxy, methoxy, trifluoromethyl, di-C₁₋₂ alkylaminosulphonyl, trifluoromethoxy and trifluoromethylthio. Preferably, R^{16aaa} is selected from hydrogen, methyl, fluorine, chlorine and bromine

In each of the foregoing embodiments and sub-sets of compounds within formula (IV⁰), Q^{1a} is NH or O. In one embodiment, Q^{1a} is NH. In another embodiment, Q^{1a} is O.

Within formula (IV), another group of preferred compounds is represented by the formula (IVa):

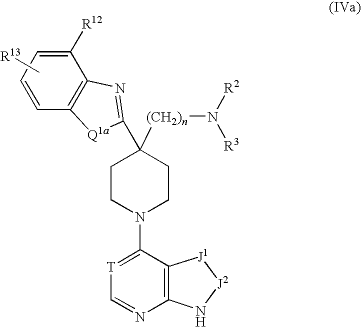

or salts, solvates, tautomers or N-oxides thereof, wherein n, T, Q^{1a}, J¹-J², R² and R³ are as defined herein in respect of formula (I) and sub-groups, examples and preferences thereof;

R¹² is selected from:
hydrogen;
hydroxy;
halogen (e.g. fluorine and chlorine);
cyano;
amino;
mono-C₁₋₄-alkylamino or di-C₁₋₄-alkylamino wherein the C₁₋₄-alkyl moieties of the mono-C₁₋₄-alkylamino and di-C₁₋₄-alkylamino groups are optionally substituted by hydroxy (other than α-hydroxy), C₁₋₂ alkoxy, amino, mono-C₁₋₂-alkylamino, di-C₁₋₂-alkylamino, C₁₋₂ acylamino;
optionally substituted C₁₋₄ alkyl (e.g. methyl);
optionally substituted C₁₋₄ alkoxy (e.g. methoxy) groups; wherein the optional substituents for the C₁₋₄ alkyl and C₁₋₄ alkoxy groups are selected from halogen (e.g. fluorine), hydroxy, C₁₋₂ acyloxy, C₁₋₂ alkoxy (e.g. methoxy), amino, mono-C₁₋₄-alkylamino, di-C₁₋₄-alkylamino and C₁₋₄ acylamino; and
a group (CH₂)ₜ—R^{cyc″}, wherein t is 0, 1 or 2 and R^{cyc″} is:
   a 5-6 membered aryl or heteroaryl ring optionally substituted by C₁₋₄ alkyl, C₁₋₄ alkoxy, halogen, hydroxy, C₁₋₄ acyloxy, cyano, trifluoromethyl, trifluoromethoxy, difluoromethoxy, amino, mono-C₁₋₄-alkylamino, di-C₁₋₄-alkylamino or C₁₋₄ acylamino;
   a 3-7 membered non-aromatic carbocyclic or heterocyclic ring optionally substituted by C₁₋₄ alkyl, C₁₋₄ alkoxy, halogen, hydroxy, C₁₋₄ acyloxy, cyano, trifluoromethyl, trifluoromethoxy, difluoromethoxy, amino, mono-C₁₋₄-alkylamino, di-C₁₋₄-alkylamino; C₁₋₄ acylamino or C₁₋₄ alkoxycarbonyl;
      and R¹³ is selected from:
hydrogen;
hydroxy;
halogen (e.g. fluorine and chlorine);
cyano;
trifluoromethyl;
trifluoromethoxy;
difluoromethoxy;
amino;
methylamino;
dimethylamino;
methyl; and
methoxy.

Within each of formulae (III), (IV), (IV⁰) and (IVa), the moiety NR²R³ is preferably amino or methylamino and most preferably is amino.

One preferred sub-group of compounds within formula (IVa) can be represented by the formula (IVb):

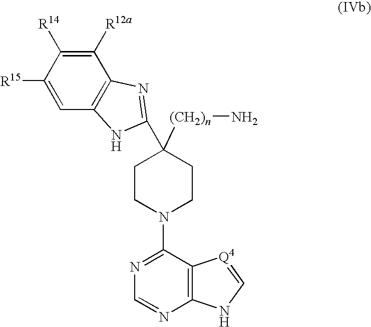

or salts, solvates, tautomers or N-oxides thereof, wherein Q⁴ is N or CH; n is 0 or 1; R^{12a} is selected from hydrogen, fluorine and chlorine; R¹⁴ is selected from hydrogen, fluorine, chlorine, methyl and methoxy; and R¹⁵ is selected from hydrogen and fluorine; provided that at least one of R^{12a}, R¹⁴ and R¹⁵ is hydrogen.

Within formula (IVb), particular sub-groups of compounds are compounds wherein:

(i) R¹⁴ is hydrogen, chlorine, methyl or methoxy; R¹⁵ is hydrogen and R^{12a} is hydrogen;

(ii) R¹⁴ is hydrogen; R¹⁵ is hydrogen and R^{12a} is methoxy; and

(iii) R¹⁴ is hydrogen; R¹⁵ is fluorine and R^{12a} is fluorine.

Another sub-group of compounds within formula (III) can be represented by the formula (V):

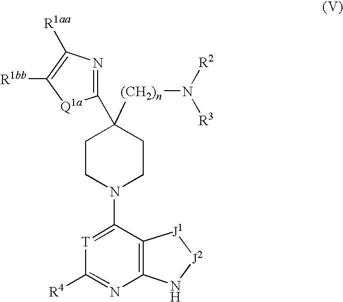

wherein R^{1aa} is hydrogen or a substituent R¹⁰, R^{1bb} is hydrogen or a substituent R¹⁰, Q^{1a} is NH or O; n is 0, 1 or 2; and R², R³, R⁴, R¹⁰, T, J¹ and J² are as defined herein in respect of formula (I) and sub-groups, examples and preferences thereof.

In one sub-group of compounds within formula (V), one of R^{1aa} and R^{1bb} is hydrogen, methyl, chlorine, cyano, fluorine or trifluoromethyl and the other of R^{1aa} and R^{1bb} is a five or six-membered aryl or heteroaryl group optionally substituted by one or two substituents R¹⁰ as defined herein. Within this sub-group, preferably one of R^{1aa} and R^{1bb} is hydrogen or methyl and the other of R^{1aa} and R^{1bb} is an optionally substituted phenyl group (e.g. an unsubstituted phenyl group).

Within each of formulae (II), (III), (IV), (IV⁰), (IVa) and (V), T is preferably nitrogen and R⁴ is preferably hydrogen. Q^{1a} can be NH or O. In one embodiment, Q^{1a} is NH. In another embodiment, Q^{1a} is O.

Within each of formulae (II), (III), (IV), (IV⁰), (IVa) and (V), J¹-J² is preferably selected from N═CH, HC═N, HN—C(O), H₂C—C(O), HC═CH, (Cl)C═CH, (Br)C═CH and (CH₃)C═CH. More preferably, J¹-J² is selected from N═CH and HC═CH.

In one subset of compounds within each of formulae (II), (III), (IV), (IV⁰), (IVa) and (V), T is nitrogen, R⁴ is hydrogen and J¹-J² is N═CH.

In another subset of compounds within each of formulae (II), (III), (IV), (IV⁰), (IVa) and (V), T is nitrogen, R⁴ is hydrogen and J¹-J² is HC═CH.

For the avoidance of doubt, it is to be understood that each general and specific preference, embodiment and example of each R group may be combined with each general and specific preference, embodiment and example of each other R group and/or J¹-J² and/or ring E and/or T and/or Q¹ and/or Q² and/or Q³ and that all such combinations are embraced by this application.

The various functional groups and substituents making up the compounds of the formula (I) are typically chosen such that the molecular weight of the compound of the formula (I) does not exceed 1000. More usually, the molecular weight of the compound will be less than 750, for example less than 700, or less than 650, or less than 600, or less than 550. More preferably, the molecular weight is less than 525 and, for example, is 500 or less.

Particular compounds of the invention are as illustrated in the examples below.

Salts, Solvates, Tautomers, Isomers, N-Oxides, Esters, Prodrugs and Isotopes

In this section, as in all other sections of this application, unless the context indicates otherwise, references to formula (I) include references to formulae (II), (III), (IV), (IVa), (IVb) and (V) and all other sub-groups, preferences and examples thereof as defined herein.

Unless otherwise specified, a reference to a particular compound also includes ionic, salt, solvate, and protected forms thereof, for example, as discussed below.

Many compounds of the formula (I) can exist in the form of salts, for example acid addition salts or, in certain cases salts of organic and inorganic bases such as carboxylate, sulphonate and phosphate salts. All such salts are within the scope of this invention, and references to compounds of the formula (I) include the salt forms of the compounds. As in the preceding sections of this application, all references to formula (I) should be taken to refer also to formulae (II), (III), (IV), (IVa), (IVb) and (V) and sub-groups thereof unless the context indicates otherwise.

Salt forms may be selected and prepared according to methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002. For example, acid addition salts may be prepared by dissolving the free base in an organic solvent in which a given salt form is insoluble or poorly soluble and then adding the required acid in an appropriate solvent so that the salt precipitates out of solution.

Acid addition salts may be formed with a wide variety of acids, both inorganic and organic. Examples of acid addition salts include salts formed with an acid selected from the group consisting of acetic, 2,2-dichloroacetic, adipic, alginic, ascorbic (e.g. L-ascorbic), L-aspartic, benzenesulphonic, benzoic, 4-acetamidobenzoic, butanoic, (+) camphoric, camphor-sulphonic, (+)-(1S)-camphor-10-sulphonic, capric, caproic, caprylic, cinnamic, citric, cyclamic, dodecylsulphuric, ethane-1,2-disulphonic, ethanesulphonic, 2-hydroxyethanesulphonic, formic, fumaric, galactaric, gentisic, glucoheptonic, D-gluconic, glucuronic (e.g. D-glucuronic), glutamic (e.g. L-glutamic), α-oxoglutaric, glycolic, hippuric, hydrobromic, hydrochloric, hydriodic, isethionic, lactic (e.g. (+)-L-lactic and (±)-DL-lactic), lactobionic, maleic, malic, (−)-L-malic, malonic, (±)-DL-mandelic, methanesulphonic, naphthalenesulphonic (e.g. naphthalene-2-sulphonic), naphthalene-1,5-disulphonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulphuric, tannic, (+)-L-tartaric, thiocyanic, toluenesulphonic (e.g. p-toluenesulphonic), undecylenic and valeric acids, as well as acylated amino acids and cation exchange resins.

One particular group of acid addition salts includes salts formed with hydrochloric, hydriodic, phosphoric, nitric, sulphuric, citric, lactic, succinic, maleic, malic, isethionic, fumaric, benzenesulphonic, toluenesulphonic, methanesulphonic, ethanesulphonic, naphthalenesulphonic, valeric, acetic, propanoic, butanoic, malonic, glucuronic and lactobionic acids. Within this group of salts, a sub-set of salts consists of salts formed with hydrochloric acid or acetic acid.

Another group of acid addition salts includes salts formed from acetic, adipic, ascorbic, aspartic, citric, DL-Lactic, fumaric, gluconic, glucuronic, hippuric, hydrochloric, glutamic, DL-malic, methanesulphonic, sebacic, stearic, succinic and tartaric acids.

The compounds of the invention may exist as mono- or di-salts depending upon the pKa of the acid from which the salt is formed. In stronger acids, the basic pyrazole nitrogen, as well as the nitrogen atom in the group NR²R³, may take part in salt formation. For example, where the acid has a pKa of less than about 3 (e.g. an acid such as hydrochloric acid, sulphuric acid or trifluoroacetic acid), the compounds of the invention will typically form salts with 2 molar equivalents of the acid.

For example, if the compound is anionic, or has a functional group which may be anionic (e.g., —COOH may be —COO⁻), then a salt may be formed with a suitable cation. Examples of suitable inorganic cations include, but are not limited to, alkali metal ions such as Na⁻ and K⁺, alkaline earth cations such as Ca²⁺ and Mg²⁺, and other cations such as Al³⁺.

Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Where the compounds of the formula (I) contain an amine function, these may form quaternary ammonium salts, for example by reaction with an alkylating agent according to methods well known to the skilled person. Such quaternary ammonium compounds are within the scope of formula (I).

The salt forms of the compounds of the invention are typically pharmaceutically acceptable salts, and examples of pharmaceutically acceptable salts are discussed in Berge et al., 1977, “Pharmaceutically Acceptable Salts,” J. Pharm. Sci., Vol. 66, pp. 1-19. However, salts that are not pharmaceutically acceptable may also be prepared as intermediate forms which may then be converted into pharmaceutically acceptable salts. Such non-pharmaceutically acceptable salts forms, which may be useful, for example, in the purification or separation of the compounds of the invention, also form part of the invention.

Compounds of the formula (I) containing an amine function may also form N-oxides. A reference herein to a compound of the formula (I) that contains an amine function also includes the N-oxide.

Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle.

N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (MCPBA), for example, in an inert solvent such as dichloromethane.

Compounds of the formula (I) may exist in a number of different geometric isomeric, and tautomeric forms and references to compounds of the formula (I) include all such forms. For the avoidance of doubt, where a compound can exist in one of several geometric isomeric or tautomeric forms and only one is specifically described or shown, all others are nevertheless embraced by formula (I).

For example, when J¹-J² is N═CR⁶, the tautomeric forms A and B are possible for the bicyclic group.

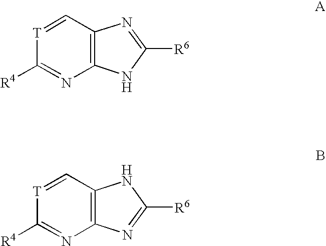

When J¹-J² is N═N, the tautomeric forms C and D are possible for the bicyclic group.

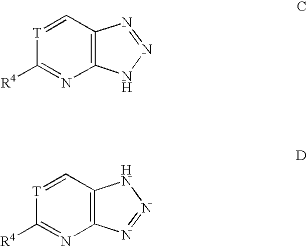

When J¹-J² is HN—CO, the tautomeric forms E, F and G are possible for the bicyclic group.

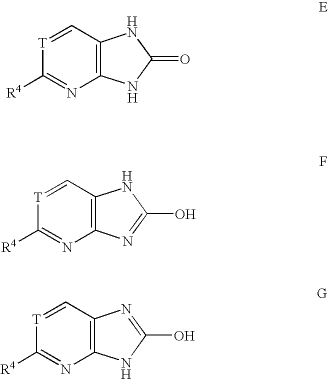

All such tautomers are embraced by formula (I).

Other examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

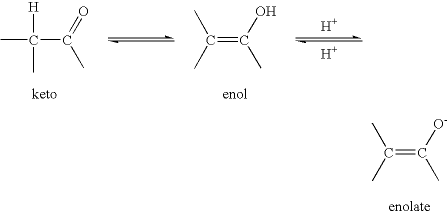

Where compounds of the formula (I) contain one or more chiral centres, and can exist in the form of two or more optical isomers, references to compounds of the formula (I) include all optical isomeric forms thereof (e.g. enantiomers, epimers and diastereoisomers), either as individual optical isomers, or mixtures (e.g. racemic or scalemic mixtures) or two or more optical isomers, unless the context requires otherwise.

The optical isomers may be characterised and identified by their optical activity (i.e. as + and − isomers, or d and l isomers) or they may be characterised in terms of their absolute stereochemistry using the “R and S” nomenclature developed by Cahn, Ingold and Prelog, see Advanced Organic Chemistry by Jerry March, 4^{th} Edition, John Wiley & Sons, New York, 1992, pages 109-114, and see also Cahn, Ingold & Prelog, Angew. Chem. Int. Ed. Engl., 1966, 5, 385-415.

Optical isomers can be separated by a number of techniques including chiral chromatography (chromatography on a chiral support) and such techniques are well known to the person skilled in the art.

As an alternative to chiral chromatography, optical isomers can be separated by forming diastereoisomeric salts with chiral acids such as (+)-tartaric acid, (−)-pyroglutamic acid, (−)-di-toluloyl-L-tartaric acid, (+)-mandelic acid, (−)-malic acid, and (−)-camphorsulphonic, separating the diastereoisomers by preferential crystallisation, and then dissociating the salts to give the individual enantiomer of the free base.

Where compounds of the formula (I) exist as two or more optical isomeric forms, one enantiomer in a pair of enantiomers may exhibit advantages over the other enantiomer, for example, in terms of biological activity. Thus, in certain circumstances, it may be desirable to use as a therapeutic agent only one of a pair of enantiomers, or only one of a plurality of diastereoisomers. Accordingly, the invention provides compositions containing a compound of the formula (I) having one or more chiral centres, wherein at least 55% (e.g. at least 60%, 65%, 70%, 75%, 80%, 85%, 90% or 95%) of the compound of the formula (I) is present as a single optical isomer (e.g. enantiomer or diastereoisomer). In one general embodiment, 99% or more (e.g. substantially all) of the total amount of the compound of the formula (I) may be present as a single optical isomer (e.g. enantiomer or diastereoisomer).

The compounds of the invention include compounds with one or more isotopic substitutions, and a reference to a particular element includes within its scope all isotopes of the element. For example, a reference to hydrogen includes within its scope ¹H, ²H (D), and ³H (T). Similarly, references to carbon and oxygen include within their scope respectively ¹²C, ¹³C and ¹⁴C and ¹⁶O and ¹⁸O.

The isotopes may be radioactive or non-radioactive. In one embodiment of the invention, the compounds contain no radioactive isotopes. Such compounds are preferred for therapeutic use. In another embodiment, however, the compound may contain one or more radioisotopes. Compounds containing such radioisotopes may be useful in a diagnostic context.

Esters such as carboxylic acid esters and acyloxy esters of the compounds of formula (I) bearing a carboxylic acid group or a hydroxyl group are also embraced by Formula (I). In one embodiment of the invention, formula (I) includes within its scope esters of compounds of the formula (I) bearing a carboxylic acid group or a hydroxyl group. In another embodiment of the invention, formula (I) does not include within its scope esters of compounds of the formula (I) bearing a carboxylic acid group or a hydroxyl group. Examples of esters are compounds containing the group —C(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. Particular examples of ester groups include, but are not limited to, —C(═O)OCH₃, —C(═O)OCH₂CH₃, —C(═O)OC(CH₃)₃, and —C(═O)OPh. Examples of acyloxy (reverse ester) groups are represented by —OC(═O)R, wherein R is an acyloxy substituent, for example, a C₁₋₇ alkyl group, a C₃₋₂₀ heterocyclyl group, or a C₅₋₂₀ aryl group, preferably a C₁₋₇ alkyl group. Particular examples of acyloxy groups include, but are not limited to, —OC(═O)CH₃ (acetoxy), —OC(═O)CH₂CH₃, —OC(═O)C(CH₃)₃, —OC(═O)Ph, and —OC(═O)CH₂Ph.

Also encompassed by formula (I) are any polymorphic forms of the compounds, solvates (e.g. hydrates), complexes (e.g. inclusion complexes or clathrates with compounds such as cyclodextrins, or complexes with metals) of the compounds, and pro-drugs of the compounds. By “prodrugs” is meant for example any compound that is converted in vivo into a biologically active compound of the formula (I).

For example, some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required.

Examples of such metabolically labile esters include those of the formula —C(═O)OR

wherein R is:

C₁₋₇alkyl

(e.g., -Me, -Et, -nPr, -iPr, -nBu, -sBu, -iBu, -tBu);

C₁₋₇aminoalkyl

(e.g., aminoethyl; 2-(N,N-diethylamino)ethyl; 2-(4-morpholino)ethyl); and

acyloxy-C₁₋₇alkyl

(e.g., acyloxymethyl;

acyloxyethyl;

pivaloyloxymethyl;

acetoxymethyl;

1-acetoxyethyl;

1-(1-methoxy-1-methyl)ethyl-carbonxyloxyethyl;

1-(benzoyloxy)ethyl; isopropoxy-carbonyloxymethyl;

1-isopropoxy-carbonyloxyethyl; cyclohexyl-carbonyloxymethyl;

1-cyclohexyl-carbonyloxyethyl;

cyclohexyloxy-carbonyloxymethyl;

1-cyclohexyloxy-carbonyloxyethyl;

(4-tetrahydropyranyloxy)carbonyloxymethyl;

1-(4-tetrahydropyranyloxy)carbonyloxyethyl;

(4-tetrahydropyranyl)carbonyloxymethyl; and

1-(4-tetrahydropyranyl)carbonyloxyethyl).

Also, some prodrugs are activated enzymatically to yield the active compound, or a compound which, upon further chemical reaction, yields the active compound (for example, as in Antibody-directed Enzyme Prodrug Therapy (ADEPT), Gene-directed Enzyme Prodrug Therapy (GDEPT), Polymer-directed Enzyme Prodrug Therapy (PDEPT), Ligand-directed Enzyme Prodrug Therapy (LIDEPT), etc.). For example, the prodrug may be a sugar derivative or other glycoside conjugate, or may be an amino acid ester derivative.

Methods for the Preparation of Compounds of the Formula (I)

In this section, references to compounds of the formula (I) include (II), (III), (IV) and each of the sub-groups thereof as defined herein unless the context requires otherwise.

In a further aspect, the invention provides a process for the preparation of a compound of the formula (I) as defined herein.

Compounds of the formula (I) can be prepared by reaction of a compound of the formula (X) with a compound of the formula (XI) where (X) and (XI) may be suitably protected and wherein T, J¹, J², Q³, G, E, R¹ to R⁵ are as hereinbefore defined, and Hal is a halogen, typically chlorine or fluorine.

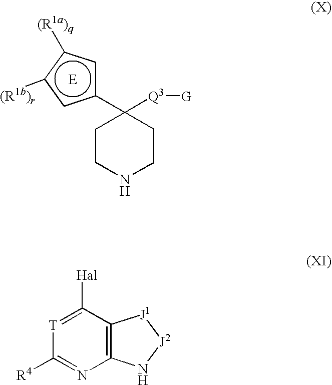

The reaction is typically carried out in a polar solvent such as an alcohol (e.g. ethanol, propanol or n-butanol) or N-methylpyrrolidin-2-one at an elevated temperature, for example a temperature in the region from 80° C. to 200° C., optionally in the presence of a non-interfering amine such as triethylamine. The reaction may be carried out in a sealed tube, particularly where the desired reaction temperature exceeds the boiling point of the solvent. Examples of sealed tubes include the “Reacti-Vial” tubes available from Pierce Chemical, Rockford, Ill., USA. When T is N, the reaction is typically carried out at a temperature in the range from about 100° C. to 130° C. but, when T is CH, higher temperatures may be required, for example up to about 160° C., and hence higher boiling solvents such as N-methylpyrrolidin-2-one or dimethylformamide may be used. In general, an excess of the nucleophilic amine will be used and/or an additional non-reacting base such as triethylamine will be included in the reaction mixture. Heating of the reaction mixture may be accomplished by normal means or by the use of a microwave heater.

In order to prepare compounds of the formula (I) wherein T is CH, the hydrogen atom of the group CH may be replaced by an activating group in order to facilitate nucleophilic displacement of the chlorine atom by the amine (XVII). The activating group is typically one which can be removed subsequent to the nucleophilic displacement reaction. One such activating group is an ester group such as ethoxycarbonyl or methoxycarbonyl which can be removed by hydrolysis and decarboxylation. Hydrolysis of the ethoxycarbonyl or methoxycarbonyl group to the carboxylic acid is typically carried out using an aqueous alkali such as sodium hydroxide, and the decarboxylation step is typically conducted by heating to an elevated temperature (e.g. 150° C. to 190° C.).

Compounds of the formula (XI) are commercially available or can be prepared according to methods well known to the skilled person.

Commercially available compounds of the formula (XI) include 6-chloro-9H-purine, 2-amino-6-chloropurine, 2-methylthio-6-chloropurine, 4-chloropyrrolo[2,3-d]pyrimidine, 4-chloro-1h-pyrazolo[3,4-d]pyrimidine, 6-chloro-2-methoxy-7-deazapurine, 6-chloro-7-deazaguanine, 4-chloro-1h-pyrazolo[3,4-d]pyrimidin-6-ylamine, 7-chloro-3h-[1,2,3]triazolo[4,5-d]pyrimidine, 4-fluoro-7-azaindole, 4-chloro-7-azaindole, 3-bromo-4-chloro-1H-pyrazolo[3,4-d]pyrimidine, 6-bromo-4-chloro-7h-pyrrolo[2,3-d]pyrimidine and 6-chloro-2-(trifluoromethyl)-9h-purine.

Compounds of the formula (XI) where T is N and J¹-J² is (Br)C═CH or (Cl)C═CH can be prepared from the corresponding compound wherein J¹-J² is HC═CH by reaction with N-bromosuccinimide (NBS) or N-clorosuccinimide (NCS) respectively. The reaction is typically carried out in a non-protic solvent such as dichloromethane and preferably under nitrogen. Compounds wherein J¹-J² is (Br)C═CH can be converted to the corresponding compound wherein J¹-J² is (R⁷)C═CH where R⁷ is an alkyl group such as methyl by lithiation with an alkyl lithium compound followed by reaction with an alkyl halide such as methyl iodide.

Compounds of the formula (XI) where T is N and J¹-J² is CH═N can be prepared from the corresponding hydroxy compound by reaction with a chlorinating agent such as POCl₃. Compounds of the formula (XI) where J¹-J² is HN—C(O) can be prepared by the reaction of an ortho-diamino compound of the formula (XII) with carbonyl di-imidazole in the presence of a non-interfering base such as triethylamine.

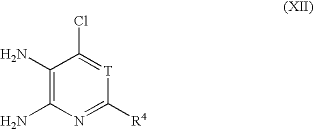

Compounds of the formula (XI) where T is CR⁵ and J¹-J² is (R⁷)H═CH(R⁶) can be prepared from the corresponding N-oxide of the formula (XIII) by reaction with phosphorus oxychloride at an elevated temperature, for example the reflux temperature of POCl₃.

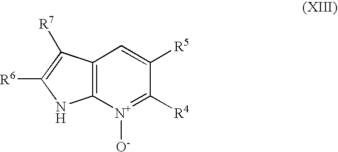

The compound of formula (XI) wherein T is N and J¹-J² is CH═CH can be made by the process shown in Scheme 1 below.

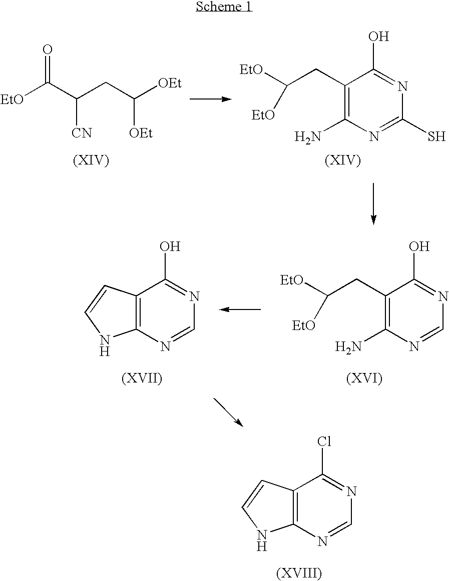

In Scheme 1, the nitrile (XIV) is reacted with thiourea in ethanol in the presence of sodium ethoxide with heating to 80-90° C. followed by the addition of ammonium chloride to give the pyrimidine thiol (XV). The pyrimidine thiol (XV) is then treated with Raney nickel to remove the thiol group and give the amino(hydroxy)pyrimidine (XVI) which is cyclised to the 7H-pyrrolo[2,3-d]pyrimidin-4-ol (XVII) using the method of J. Davoll, J. Chem. Soc., 1960, pp. 131-138. The 7H-pyrrolo[2,3-d]pyrimidin-4-ol (XVII) is then converted to the corresponding chlorine compound (XVIII) by reaction with POCl₃.

Compounds of the formula (X) wherein R^{1a} and R^{1b} together with the carbon atoms to which they are attached form a 6-membered aryl or heteroaryl ring, Q¹ is NH or O and Q² is N, can be prepared by the sequence of reactions shown in Scheme 2.

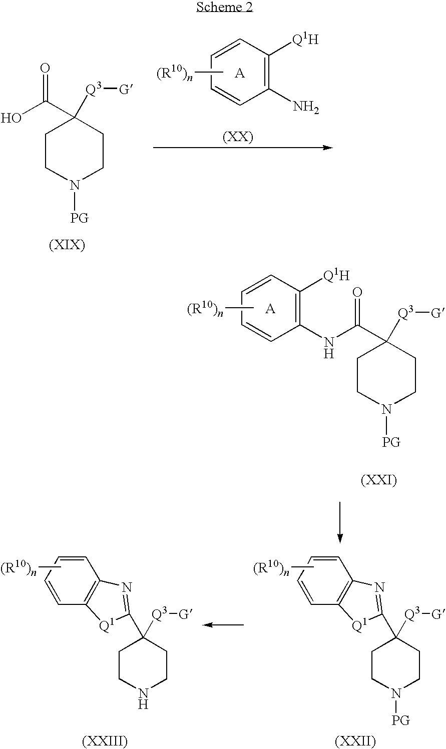

In Scheme 2, the piperidine carboxylic acid (XIX), wherein G′ is a protected form of the group G, is reacted with an aromatic amine of the formula (XX), wherein A is an aryl or heteroaryl ring and Q¹ is NH or O, to form the intermediate amide (XXI). The amide-forming reaction is preferably carried out in the presence of a reagent of the type commonly used in the formation of peptide linkages. Examples of such reagents include 1,3-dicyclohexylcarbodiimide (DCC) (Sheehan et al, J. Amer. Chem Soc. 1955, 77, 1067), 1-ethyl-3-(3′-dimethylaminopropyl)-carbodiimide (referred to herein either as EDC or EDAC) (Sheehan et al, J. Org. Chem., 1961, 26, 2525), uronium-based coupling agents such as O-(7-azabenzotriazol-1-yl)-N,N,N′,N′-tetramethyluronium hexafluorophosphate (HATU) and phosphonium-based coupling agents such as 1-benzo-triazolyloxytris-(pyrrolidino)phosphonium hexafluorophosphate (PyBOP) (Castro et al, Tetrahedron Letters, 1990, 31, 205). Carbodiimide-based coupling agents are advantageously used in combination with 1-hydroxy-7-azabenzotriazole (HOAt) (L. A. Carpino, J. Amer. Chem. Soc., 1993, 115, 4397) or 1-hydroxybenzotriazole (HOBt) (Konig et al, Chem. Ber., 103, 708, 2024-2034). Preferred coupling reagents include EDC (EDAC) and DCC in combination with HOAt or HOBt.

The coupling reaction is typically carried out in a non-aqueous, non-protic solvent such as acetonitrile, dioxan, dimethylsulphoxide, dichloromethane, dimethylformamide or N-methylpyrrolidinone, or in an aqueous solvent optionally together with one or more miscible co-solvents. The reaction can be carried out at room temperature or, where the reactants are less reactive (for example in the case of anilines) at an appropriately elevated temperature, for example a temperature up to about 100° C., e.g. 50-80° C. The reaction may optionally be carried out in the presence of a non-interfering base, for example a tertiary amine such as triethylamine or N,N-diisopropylethylamine

As an alternative, a reactive derivative of the carboxylic acid, e.g. an anhydride or acid chloride, may be used. Reaction with a reactive derivative such an anhydride is typically accomplished by stirring the amine and anhydride at room temperature in the presence of a base such as pyridine.

The amide (XXI) is subjected to acid-catalysed or acid-induced cyclisation to give the bicyclic compound (XXII). Cyclisation can be achieved, for example, by dissolving the amide (XXI) in acetic acid and then heating to a temperature of up to about 100° C., more preferably up to about 80° C., for a suitably prolonged period, for example in excess of 10 hours, e.g. about 16 hours. As an alternative to acetic acid induced cyclisation, the amide can be heated in a non-aqueous solvent such as toluene in the presence of para-toluene sulphonic acid.

The bicyclic compound (XXII) is then deprotected by standard methods to give compound (XXIII). Where the protecting groups are boc groups, deprotection can be accomplished using an acid such as a strong organic acid (e.g. trifluoroacetic acid) or a mineral acid (e.g. hydrochloric acid) in dioxane or ether. The deprotection reaction is typically carried out at room temperature.

An alternative route to compounds of the formula (XXIII), wherein Q³-G is a group CH₂—NH₂ and Q¹ is O, S or NH is illustrated in Scheme 3.

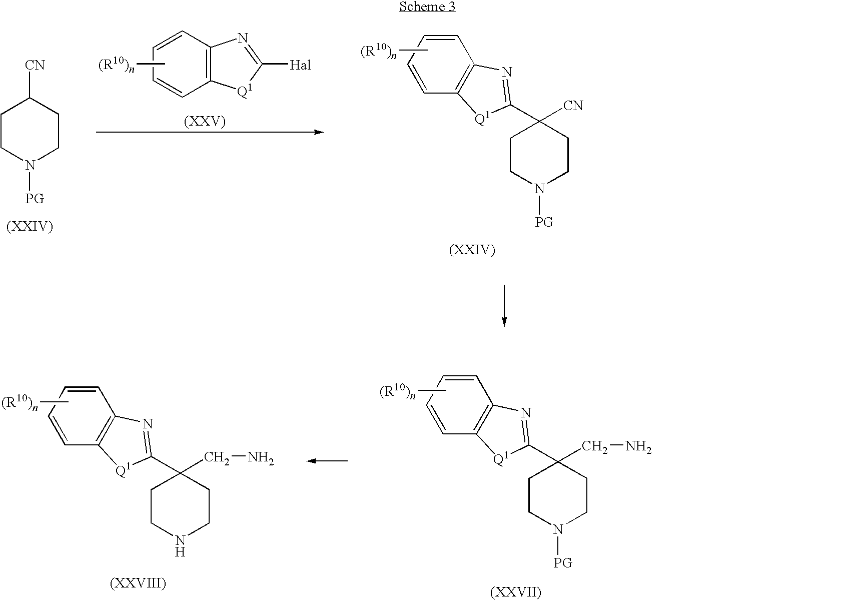

In Scheme 3, the protected cyanopiperidine (XXIV) is reacted with the halo-compound (XXV), wherein Hal is fluorine, chlorine or bromine, in the presence of a strong base such as sodium hexamethyldisilazide (NaHMDS) to give the gem-disubstituted piperidine compound (XXVI). The reaction is typically carried out in a polar aprotic solvent such as THF, see for example the article in J. Org. Chem., 2005, 70 (24), 10186-10189.

When Q¹ is NH, it is typically protected during the reaction with the base and the halo-compound. One suitable protecting group is the dimethylsulphamoyl group which can be introduced by reacting the compound of the formula (XXV) with dimethylsuphamoyl chloride in the presence of a non-interfering base such as DABCO.

The nitrile group in the gem-disubstituted piperidine compound (XXVI) is reduced to an aminomethyl group by hydrogenation in the presence of Raney nickel in an organic solvent (e.g. N,N-dimethylformamide, ethanol and/or tetrahydrofuran), optionally with added base (e.g. aqueous sodium hydroxide solution or methanolic ammonia). The resulting aminomethyl compound (XXVII) is then deprotected in standard fashion, for example using TFA when the protecting group is a boc group.

Compounds of the formula (X) wherein the ring E is an imidazole group, R^{1a} is hydrogen and R^{1b} is an aryl, heteroaryl or alkyl group can be prepared by the reaction sequence shown in Scheme 4.

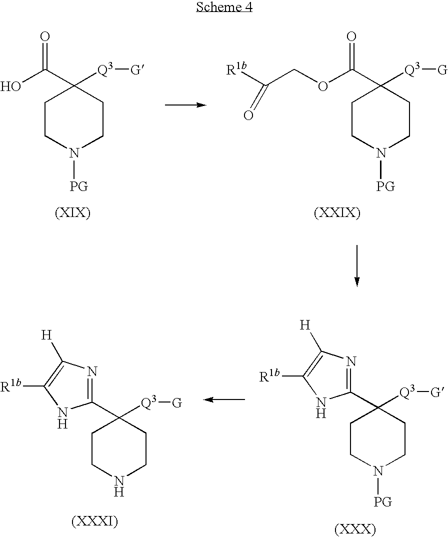

In Scheme 4, the protected piperidine carboxylic acid (XIX) is converted to a metal carboxylate salt by reaction with caesium carbonate in an anhydrous alcohol such as methanol, and the carboxylate salt is then reacted with an α-bromoketone R^{1b}C(O)CH₂Br to give the ester (XXIX). The ester (XXIX) is cyclised to the imidazole compound (XXX) by heating with ammonium acetate in a high boiling solvent such as xylene, e.g. to a temperature in the range 130-140° C. The protecting groups can then be removed from the piperidine nitrogen and the group G by standard methods to give the deprotected imidazolyl piperidine compound (XXXI).

Compounds of the formula (X) wherein the ring E is an oxazole group, R^{1a} is hydrogen and R^{1b} is an aryl, heteroaryl or alkyl group can be prepared by the reaction sequence shown in Scheme 5.

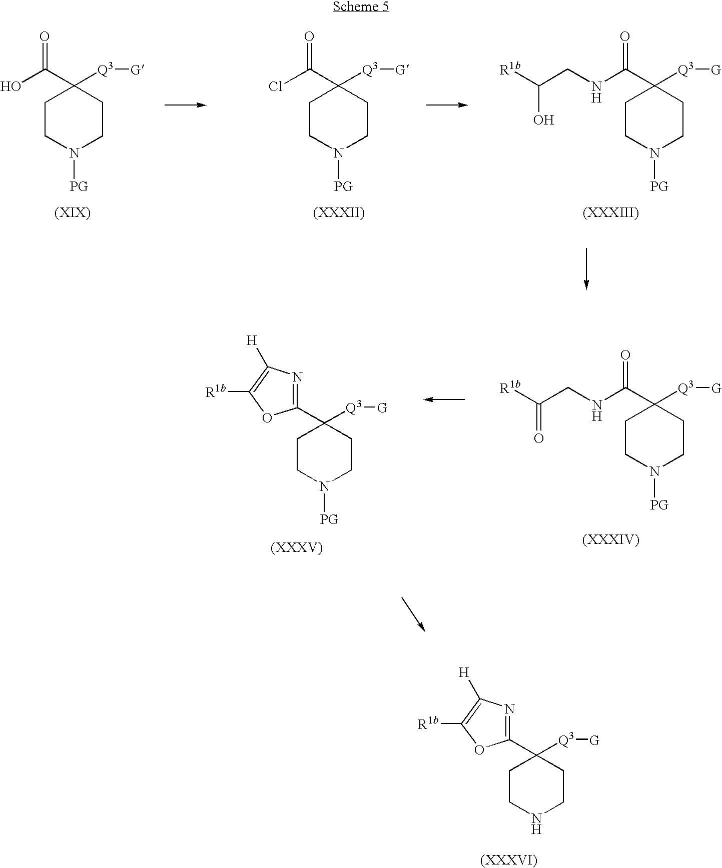

In Scheme 5, the protected piperidine (XIX), in which PG is, for example, a base-sensitive protecting group such as an Fmoc group, is converted to the corresponding acid chloride (XXXII) by reaction with thionyl chloride. The acid chloride (XXXII) is reacted with a compound of the formula R^{1b}—CH(OH)—CH₂—NH₂ in an anhydrous organic solvent such as dichloromethane in the presence of a non-interfering base such as triethylamine to give the hydroxy-amide compound (XXXIII). The hydroxy-amide compound (XXXIII) is oxidised to the corresponding ketone (XXXIV) using Corey's reagent (pyridinium chlorochromate) in dichloromethane and the ketone is then cyclised to the oxazole (XXXV) by heating to a temperature in excess of 100° C. in the presence of phosphorus oxychloride. The fmoc protecting group is then removed by treatment with base (e.g. piperidine followed by 1,8-diazabicyclo[5.4.0]undec-7-ene) to give the deprotected piperidine (XXXVI).

Another synthetic route to intermediate compounds of the formula (X) is illustrated in Scheme 6.

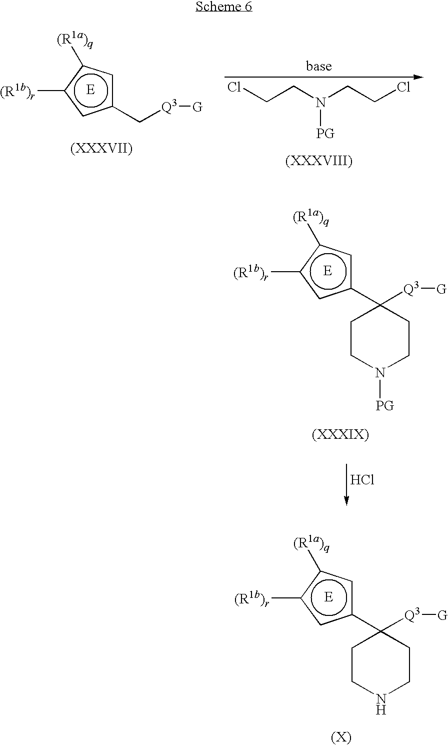

The starting material for the route shown in Scheme 6 is a compound of the formula (XXXVII) wherein the nature of the group Q³-G and the ring E is such that an anion can be formed at the methylene group lying between Q³-G and the ring E. Thus, for example, in the group Q³-G, Q³ can be a bond and G can be CN. Alternatively, or additionally, the arrangement of heteroatoms in the ring E can be such as to help facilitate formation of or stabilise an anion at the methylene group.

The compound of formula (XXXVII) is treated with a strong base in a polar aprotic solvent such as DMF and then reacted with the protected nitrogen mustard compound (XXXVIII) to give the protected piperidine intermediate (XXXIX). The protecting group can then be removed in conventional fashion to give the compound (X). For example, if the protecting group is a boc group, it can be removed by treatment with an acid such as hydrochloric acid.

The protected nitrogen mustard compound (XXXVIII) can be prepared according to the method described in J. Chem. Soc., Perkin Trans 1, 2000, p 3444-3450.

Compounds of the formula (I) wherein the ring E together with R^{1a} and R^{1b} form a benzoimidazole ring or aza-anaoogue thereof, can be prepared by the reaction of a compound of the formula (XXXX):

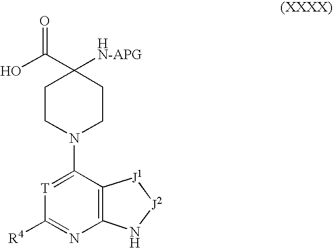

wherein APG is an amine protecting group such as a Boc group and T, R⁴, J¹ and J² are as defined herein, with a compound of the formula (XX) (see Scheme 2 above) wherein A is a benzene or pyridine ring and Q¹ is NH under amide forming and cyclisation conditions. The amide forming step can be brought about using a reagent of the type conventionally used to form amide linkages (see Scheme 2 above). One such reagent is HATU. The reaction is typically carried out in the presence of a non-interfering base such as a tertiary amine, e.g. N,N-diisopropylethylamine Cyclisation of the amide intermediate (not shown) may subsequently be carried out by heating in the presence of acid such as hydrochloric acid, which may also serve to remove the amine protecting group APG to give the desired product.

Once formed, a compound of the formula (I) can be converted into a different compound of the formula (I) by any of a wide varierty of well known and standard methods. Examples of interconversions include the reduction of compounds of the formula (I) in which G is a nitrile group to the corresponding amine Compounds in which NR²R³ is an NH₂ group can be converted to the corresponding alkylamine by reductive alkylation, or by formation of the N-Boc derivative and reaction with an alkylating agent such as methyl iodide in the presence of a base. Alternatively, the amine can be converted to a cyclic group by methods well known to the skilled person.

Compounds of the formula (IV) wherein Q^{1a} is NH and R¹⁰ is an aryl or heteroaryl group can be prepared from the corresponding compounds of formula (IV) wherein R¹⁰ is a halogen atom (particularly bromine) by reaction with an aryl or heteroaryl boronate or boronic acid under Suzuki coupling conditions. Thus, the coupling reaction is carried out in the presence of a palladium catalyst such as bis(tri-t-butylphosphine)palladium (0) or tetrakis(triphenylphosphine)palladium(0) in the presence of a base (e.g. a carbonate such as potassium carbonate). The reaction may be carried out in an aqueous solvent system, for example aqueous ethanol, and the reaction mixture is typically subjected to heating, for example to a temperature in excess of 100° C.

Many boronic acids and boronates suitable for use in preparing compounds of the invention are commercially available, for example from Boron Molecular Limited of Noble Park, Australia, or from Combi-Blocks Inc, of San Diego, USA. Where the boronates are not commercially available, they can be prepared by methods known in the art, for example as described in the review article by N. Miyaura and A. Suzuki, Chem. Rev. 1995, 95, 2457. Thus, boronates can be prepared by reacting the corresponding bromo-compound with an alkyl lithium such as butyl lithium and then reacting with a borate ester. The resulting boronate ester derivative can, if desired, be hydrolysed to give the corresponding boronic acid.

Further examples of functional group interconversions and reagents and conditions for carrying out such conversions can be found in, for example, Advanced Organic Chemistry, by Jerry March, 4^{th} edition, 119, Wiley Interscience, New York, Fiesers' Reagents for Organic Synthesis, Volumes 1-17, John Wiley, edited by Mary Fieser (ISBN: 0-471-58283-2), and Organic Syntheses, Volumes 1-8, John Wiley, edited by Jeremiah P. Freeman (ISBN: 0-471-31192-8).

Protecting Groups

In many of the reactions described above, it may be necessary to protect one or more groups to prevent reaction from taking place at an undesirable location on the molecule. Examples of protecting groups, and methods of protecting and deprotecting functional groups, can be found in Protective Groups in Organic Synthesis (T. Green and P. Wuts; 3rd Edition; John Wiley and Sons, 1999).

A hydroxy group may be protected, for example, as an ether (—OR) or an ester (—OC(═O)R), for example, as: a t-butyl ether; a benzyl, benzhydryl (diphenylmethyl), or trityl(triphenylmethyl)ether; a trimethylsilyl or t-butyldimethylsilyl ether; or an acetyl ester (—OC(═O)CH₃, —OAc). An aldehyde or ketone group may be protected, for example, as an acetal (R—CH(OR)₂) or ketal (R₂C(OR)₂), respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid. An amine group may be protected, for example, as an amide (—NRCO—R) or a urethane (—NRCO—OR), for example, as: a methyl amide (—NHCO—CH₃); a benzyloxy amide (—NHCO—OCH₂C₆H₅, —NH-Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH-Fmoc), as a 6-nitroveratryloxy amide (—NH-Nvoc), as a 2-trimethylsilylethyloxy amide (—NH-Teoc), as a 2,2,2-trichloroethyloxy amide (—NH-Troc), as an allyloxy amide (—NH-Alloc), or as a 2-(phenylsulphonyl)ethyloxy amide (—NH-Psec). Other protecting groups for amines, such as cyclic amines and heterocyclic N—H groups, include toluenesulphonyl(tosyl) and methanesulphonyl(mesyl) groups and benzyl groups such as a para-methoxybenzyl (PMB) group. A carboxylic acid group may be protected as an ester for example, as: an C₁₋₇ alkyl ester (e.g., a methyl ester; a t-butyl ester); a C₁₋₇ haloalkyl ester (e.g., a C₁₋₇ trihaloalkyl ester); a triC₁₋₇ alkylsilyl-C₁₋₇alkyl ester; or a C₅₋₂₀ aryl-C₁₋₇ alkyl ester (e.g., a benzyl ester; a nitrobenzyl ester); or as an amide, for example, as a methyl amide. A thiol group may be protected, for example, as a thioether (—SR), for example, as: a benzyl thioether; an acetamidomethyl ether (—S—CH₂NHC(═O)CH₃).

### Isolation and Purification of the Compounds of the Invention

The compounds of the invention can be isolated and purified according to standard techniques well known to the person skilled in the art. One technique of particular usefulness in purifying the compounds is preparative liquid chromatography using mass spectrometry as a means of detecting the purified compounds emerging from the chromatography column.

Preparative LC-MS is a standard and effective method used for the purification of small organic molecules such as the compounds described herein. The methods for the liquid chromatography (LC) and mass spectrometry (MS) can be varied to provide better separation of the crude materials and improved detection of the samples by MS.

Optimisation of the preparative gradient LC method will involve varying columns, volatile eluents and modifiers, and gradients. Methods are well known in the art for optimising preparative LC-MS methods and then using them to purify compounds. Such methods are described in Rosentreter U, Huber U.; Optimal fraction collecting in preparative LC/MS; J Comb Chem.; 2004; 6(2), 159-64 and Leister W, Strauss K, Wisnoski D, Zhao Z, Lindsley C., Development of a custom high-throughput preparative liquid chromatography/mass spectrometer platform for the preparative purification and analytical analysis of compound libraries; J Comb Chem.; 2003; 5(3); 322-9.

Chemical Intermediates

Many of the chemical intermediates described above are novel per se and such novel intermediates form a further aspect of the invention.

Pharmaceutical Formulations

While it is possible for the active compound to be administered alone, it is preferable to present it as a pharmaceutical composition (e.g. formulation) comprising at least one active compound of the invention together with one or more pharmaceutically acceptable carriers, adjuvants, excipients, diluents, fillers, buffers, stabilisers, preservatives, lubricants, or other materials well known to those skilled in the art and optionally other therapeutic or prophylactic agents.

Thus, the present invention further provides pharmaceutical compositions, as defined above, and methods of making a pharmaceutical composition comprising admixing at least one active compound, as defined above, together with one or more pharmaceutically acceptable carriers, excipients, buffers, adjuvants, stabilizers, or other materials, as described herein.

The term “pharmaceutically acceptable” as used herein pertains to compounds, materials, compositions, and/or dosage forms which are, within the scope of sound medical judgment, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Accordingly, in a further aspect, the invention provides compounds of the formula (I) and sub-groups thereof as defined herein in the form of pharmaceutical compositions.

The pharmaceutical compositions can be in any form suitable for oral, parenteral, topical, intranasal, ophthalmic, otic, rectal, intra-vaginal, or transdermal administration. Where the compositions are intended for parenteral administration, they can be formulated for intravenous, intramuscular, intraperitoneal, subcutaneous administration or for direct delivery into a target organ or tissue by injection, infusion or other means of delivery.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use.

Extemporaneous injection solutions and suspensions may be prepared from sterile powders, granules and tablets.

In one preferred embodiment of the invention, the pharmaceutical composition is in a form suitable for i.v. administration, for example by injection or infusion.

In another preferred embodiment, the pharmaceutical composition is in a form suitable for sub-cutaneous (s.c.) administration.

Pharmaceutical dosage forms suitable for oral administration include tablets, capsules, caplets, pills, lozenges, syrups, solutions, powders, granules, elixirs and suspensions, sublingual tablets, wafers or patches and buccal patches.

Pharmaceutical compositions containing compounds of the formula (I) can be formulated in accordance with known techniques, see for example, Remington's Pharmaceutical Sciences, Mack Publishing Company, Easton, Pa., USA.

Thus, tablet compositions can contain a unit dosage of active compound together with an inert diluent or carrier such as a sugar or sugar alcohol, e.g. lactose, sucrose, sorbitol or mannitol; and/or a non-sugar derived diluent such as sodium carbonate, calcium phosphate, calcium carbonate, or a cellulose or derivative thereof such as methyl cellulose, ethyl cellulose, hydroxypropyl methyl cellulose, and starches such as corn starch. Tablets may also contain such standard ingredients as binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures. Such excipients are well known and do not need to be discussed in detail here.

Capsule formulations may be of the hard gelatin or soft gelatin variety and can contain the active component in solid, semi-solid, or liquid form. Gelatin capsules can be formed from animal gelatin or synthetic or plant derived equivalents thereof.

The solid dosage forms (e.g. tablets, capsules etc.) can be coated or un-coated, but typically have a coating, for example a protective film coating (e.g. a wax or varnish) or a release controlling coating. The coating (e.g. a Eudragit™ type polymer) can be designed to release the active component at a desired location within the gastro-intestinal tract. Thus, the coating can be selected so as to degrade under certain pH conditions within the gastrointestinal tract, thereby selectively release the compound in the stomach or in the ileum or duodenum.

Instead of, or in addition to, a coating, the drug can be presented in a solid matrix comprising a release controlling agent, for example a release delaying agent which may be adapted to selectively release the compound under conditions of varying acidity or alkalinity in the gastrointestinal tract. Alternatively, the matrix material or release retarding coating can take the form of an erodible polymer (e.g. a maleic anhydride polymer) which is substantially continuously eroded as the dosage form passes through the gastrointestinal tract. As a further alternative, the active compound can be formulated in a delivery system that provides osmotic control of the release of the compound. Osmotic release and other delayed release or sustained release formulations may be prepared in accordance with methods well known to those skilled in the art.

Compositions for topical use include ointments, creams, sprays, patches, gels, liquid drops and inserts (for example intraocular inserts). Such compositions can be formulated in accordance with known methods.

Compositions for parenteral administration are typically presented as sterile aqueous or oily solutions or fine suspensions, or may be provided in finely divided sterile powder form for making up extemporaneously with sterile water for injection.

Examples of formulations for rectal or intra-vaginal administration include pessaries and suppositories which may be, for example, formed from a shaped moldable or waxy material containing the active compound.

Compositions for administration by inhalation may take the form of inhalable powder compositions or liquid or powder sprays, and can be administrated in standard form using powder inhaler devices or aerosol dispensing devices. Such devices are well known. For administration by inhalation, the powdered formulations typically comprise the active compound together with an inert solid powdered diluent such as lactose.

The compounds of the inventions will generally be presented in unit dosage form and, as such, will typically contain sufficient compound to provide a desired level of biological activity. For example, a formulation intended for oral administration may contain from 0.1 milligrams to 2 grams of active ingredient, more usually from 10 milligrams to 1 gram, for example, 50 milligrams to 500 milligrams.

The active compound will be administered to a patient in need thereof (for example a human or animal patient) in an amount sufficient to achieve the desired therapeutic effect.

Protein Kinase Inhibitory Activity

The activity of the compounds of the invention as inhibitors of protein kinase A and protein kinase B can be measured using the assays set forth in the examples below and the level of activity exhibited by a given compound can be defined in terms of the IC50 value. Preferred compounds of the present invention are compounds having an IC₅₀ value of less than 1 μM, more preferably less than 0.1 μM, against protein kinase B.

Some of the compounds of the formula (I) are selective inhibitors of PKB relative to PKA, i.e. the IC₅₀ values against PKB are from 5 to 10 times lower, and more preferably greater than 10 times lower, than the IC₅₀ values against PKA.

Therapeutic Uses

Prevention or Treatment of Proliferative Disorders

The compounds of the formula (I) are inhibitors of protein kinase A and protein kinase B. As such, they are expected to be useful in providing a means of preventing the growth of or inducing apoptosis of neoplasias. It is therefore anticipated that the compounds will prove useful in treating or preventing proliferative disorders such as cancers. In particular tumours with deletions or inactivating mutations in PTEN or loss of PTEN expression or rearrangements in the (T-cell lytmphocyte) TCL-1 gene may be particularly sensitive to PKB inhibitors. Tumours which have other abnormalities leading to an upregulated PKB pathway signal may also be particularly sensitive to inhibitors of PKB. Examples of such abnormalities include but are not limited to overexpression of one or more PI3K subunits, over-expression of one or more PKB isoforms, or mutations in PI3K, PDK1, or PKB which lead to an increase in the basal activity of the enzyme in question, or upregulation or overexpression or mutational activation of a growth factor receptor such as a growth factor selected from the epidermal growth factor receptor (EGFR), fibroblast growth factor receptor (FGFR), platelet derived growth factor receptor (PDGFR), insulin-like growth factor 1 receptor (IGF-1R) and vascular endothelial growth factor receptor (VEGFR) families.

It is also envisaged that the compounds of the invention will be useful in treating other conditions which result from disorders in proliferation or survival such as viral infections, and neurodegenerative diseases for example. PKB plays an important role in maintaining the survival of immune cells during an immune response and therefore PKB inhibitors could be particularly beneficial in immune disorders including autoimmune conditions.

Therefore, PKB inhibitors could be useful in the treatment of diseases in which there is a disorder of proliferation, apoptosis or differentiation.

PKB inhibitors may also be useful in diseases resulting from insulin resistance and insensitivity, and the disruption of glucose, energy and fat storage such as metabolic disease and obesity.

Examples of cancers which may be inhibited include, but are not limited to, a carcinoma, for example a carcinoma of the bladder, breast, colon (e.g. colorectal carcinomas such as colon adenocarcinoma and colon adenoma), kidney, epidermal, liver, lung, for example adenocarcinoma, small cell lung cancer and non-small cell lung carcinomas, oesophagus, gall bladder, ovary, pancreas e.g. exocrine pancreatic carcinoma, stomach, cervix, endometrium, thyroid, prostate, or skin, for example squamous cell carcinoma; a hematopoctic malignancy for example acute myeloid leukaemia, acute promyclocytic leukaemia, acute lymphoblastic leukaemia, chronic myeloid leukaemia, chronic lymphocytic leukaemia and other B-cell lymphoproliferative diseases, myelodysplastic syndrome, T-cell lymphoproliferative diseases including those derived from Natural Killer cells, Non-Hodgkin's lymphoma and Hodgkin's disease; Bortezomib sensitive and refractory multiple myeloma; hematopoetic diseases of abnormal cell proliferation whether pre malignant or stable such as myeloproliferative diseases including polycythemia vera, essential thrombocythemia and primary myelofibrosis; hairy cell lymphoma, or Burkett's lymphoma; a hematopoietic tumour of myeloid lineage, for example acute and chronic myelogenous leukaemias, myelodysplastic syndrome, or promyelocytic leukaemia; thyroid follicular cancer; a tumour of mesenchymal origin, for example fibrosarcoma or habdomyosarcoma; a tumour of the central or peripheral nervous system, for example astrocytoma, neuroblastoma, glioma or schwannoma; melanoma; seminoma; teratocarcinoma; osteosarcoma; xenoderoma pigmentosum; keratoctanthoma; thyroid follicular cancer; or Kaposi's sarcoma.

One subset of cancers which may be inhibited includes, but is not limited to, a carcinoma, for example a carcinoma of the bladder, breast, colon (e.g. colorectal carcinomas such as colon adenocarcinoma and colon adenoma), kidney, epidermal, liver, lung, for example adenocarcinoma, small cell lung cancer and non-small cell lung carcinomas, oesophagus, gall bladder, ovary, pancreas e.g. exocrine pancreatic carcinoma, stomach, cervix, endometrium, thyroid, prostate, or skin, for example squamous cell carcinoma; a hematopoietic tumour of lymphoid lineage, for example leukaemia, acute lymphocytic leukaemia, B-cell lymphoma, T-cell lymphoma, Hodgkin's lymphoma, non-Hodgkin's lymphoma, hairy cell lymphoma, or Burkett's lymphoma; a hematopoietic tumour of myeloid lineage, for example acute and chronic myelogenous leukaemias, myelodysplastic syndrome, or promyelocytic leukaemia; thyroid follicular cancer; a tumour of mesenchymal origin, for example fibrosarcoma or habdomyosarcoma; a tumour of the central or peripheral nervous system, for example astrocytoma, neuroblastoma, glioma or schwannoma; melanoma; seminoma; teratocarcinoma; osteosarcoma; xenoderoma pigmentosum; keratoctanthoma; thyroid follicular cancer; or Kaposi's sarcoma.

Thus, in the pharmaceutical compositions, uses or methods of this invention for treating a disease or condition comprising abnormal cell growth, the disease or condition comprising abnormal cell growth in one embodiment is a cancer.

Particular subsets of cancers include breast cancer, ovarian cancer, colon cancer, prostate cancer, oesophageal cancer, squamous cancer and non-small cell lung carcinomas.

A further subset of cancers includes breast cancer, ovarian cancer, prostate cancer, endometrial cancer and glioma.

It is also possible that some protein kinase B inhibitors can be used in combination with other anticancer agents. For example, it may be beneficial to combine of an inhibitor that induces apoptosis with another agent which acts via a different mechanism to regulate cell growth thus treating two of the characteristic features of cancer development. Examples of such combinations are set out below.

Immune Disorders

Immune disorders for which PKA and PKB inhibitors may be beneficial include but are not limited to autoimmune conditions and chronic inflammatory diseases, for example systemic lupus erythematosus, autoimmune mediated glomerulonephritis, rheumatoid arthritis, psoriasis, inflammatory bowel disease, and autoimmune diabetes mellitus, Eczema hypersensitivity reactions, asthma, COPD, rhinitis, and upper respiratory tract disease.

Other Therapeutic Uses

PKB plays a role in apoptosis, proliferation, differentiation and therefore PKB inhibitors could also be useful in the treatment of the following diseases other than cancer and those associated with immune dysfunction; viral infections, for example herpes virus, pox virus, Epstein-Barr virus, Sindbis virus, adenovirus, HIV, HPV, HCV and HCMV; prevention of AIDS development in HIV-infected individuals; cardiovascular diseases for example cardiac hypertrophy, restenosis, atherosclerosis; neurodegenerative disorders, for example Alzheimer's disease, AIDS-related dementia, Parkinson's disease, amyotropic lateral sclerosis, retinitis pigmentosa, spinal muscular atropy and cerebellar degeneration; glomerulonephritis; myelodysplastic syndromes, ischemic injury associated myocardial infarctions, stroke and reperfusion injury, degenerative diseases of the musculoskeletal system, for example, osteoporosis and arthritis, aspirin-sensitive rhinosinusitis, cystic fibrosis, multiple sclerosis, kidney diseases.

### Advantages of Compounds of the Invention

Compounds of the formula (I) and sub-groups thereof as defined herein will have advantages over prior art compounds.

In particular, the compounds of formulae (II), (II), (III), (IV), (IVa), (IVb) and (V) have advantages over prior art compounds.

Potentially the compounds of the invention have physiochemical properties suitable for oral exposure.

Compounds of the formula (I) should exhibit improved oral bioavailability relative to prior art compounds. Oral bioavailability can be defined as the ratio (F) of the plasma exposure of a compound when dosed by the oral route to the plasma exposure of the compound when dosed by the intravenous (i.v.) route, expressed as a percentage.

Compounds having an oral bioavailability (F value) of greater than 30%, more preferably greater than 40%, are particularly advantageous in that they may be administered orally rather than, or as well as, by parenteral administration.

Furthermore, it is envisaged that compounds of the invention are both more potent and more selective in their activities against different kinases, and demonstrate enhanced selectivity for and potency against PKB in particular.

It is also envisaged that compounds of the invention are advantageous over prior art compounds in that they have different susceptibilities to P450 enzymes and and in that they exhibit improvements with regard to drug metabolism and pharmacokinetic properties.

Furthermore, it is considered that compounds of the invention should exhibit reduced dosage requirements.

Compounds of the invention are advantageous in that they have improved thermodynamic solubilities, thereby leading potentially to an improved dose: solubility ratio and reduced development risk.

It is further envisaged that compounds of the invention also demonstrate improved cell activity in proliferation and clonogenic assays thereby indicating improved anti-cancer activity.

Compounds of the invention are potentially less toxic than prior art compounds.

hERG

In the late 1990s a number of drugs, approved by the US FDA, had to be withdrawn from sale in the US when it was discovered they were implicated in deaths caused by heart malfunction. It was subsequently found that a side effect of these drugs was the development of arrhythmias caused by the blocking of hERG channels in heart cells. The hERG channel is one of a family of potassium ion channels the first member of which was identified in the late 1980s in a mutant Drosophila melanogaster fruitfly (see Jan, L. Y. and Jan, Y. N. (1990). A Superfamily of Ion Channels. Nature, 345(6277):672). The biophysical properties of the hERG potassium ion channel are described in Sanguinetti, M. C., Jiang, C., Curran, M. E., and Keating, M. T. (1995). A Mechanistic Link Between an Inherited and an Acquired Cardiac Arrhythmia: HERG encodes the Ikr potassium channel. Cell, 81:299-307, and Trudeau, M. C., Warmke, J. W., Ganetzky, B., and Robertson, G. A. (1995). HERG, a Human Inward Rectifier in the Voltage-Gated Potassium Channel Family. Science, 269:92-95.

The elimination of hERG blocking activity remains an important consideration in the development of any new drug.

Compounds of formulae (I), (II), (III), (IV), (IVa), (IVb) and (V) in particular have reduced, negligible or no hERG ion channel blocking activity.

Methods of Treatment

It is envisaged that the compounds of the formula (I) will useful in the prophylaxis or treatment of a range of disease states or conditions mediated by protein kinase A and/or protein kinase B. Examples of such disease states and conditions are set out above.

Compounds of the formula (I) are generally administered to a subject in need of such administration, for example a human or animal patient, preferably a human.

The compounds will typically be administered in amounts that are therapeutically or prophylactically useful and which generally are non-toxic. However, in certain situations (for example in the case of life threatening diseases), the benefits of administering a compound of the formula (I) may outweigh the disadvantages of any toxic effects or side effects, in which case it may be considered desirable to administer compounds in amounts that are associated with a degree of toxicity.

The compounds may be administered over a prolonged term to maintain beneficial therapeutic effects or may be administered for a short period only. Alternatively they may be administered in a pulsatile manner.

A typical daily dose of the compound of formula (I) can be in the range from 100 picograms to 100 milligrams per kilogram of body weight, more typically 5 nanograms to 25 milligrams per kilogram of bodyweight, and more usually 10 nanograms to 15 milligrams per kilogram (e.g. 10 nanograms to 10 milligrams, and more typically 1 microgram per kilogram to 20 milligrams per kilogram, for example 1 microgram to 10 milligrams per kilogram) per kilogram of bodyweight although higher or lower doses may be administered where required. The compound of the formula (I) can be administered on a daily basis or on a repeat basis every 2, or 3, or 4, or 5, or 6, or 7, or 10 or 14, or 21, or 28 days for example.

The compounds of the invention may be administered orally in a range of doses, for example 1 to 1500 mg, 2 to 800 mg, or 5 to 500 mg, e.g. 2 to 200 mg or 10 to 1000 mg, particular examples of doses including 10, 20, 50 and 80 mg. The compound may be administered once or more than once each day. The compound can be administered continuously (i.e. taken every day without a break for the duration of the treatment regimen). Alternatively, the compound can be administered intermittently, i.e. taken continuously for a given period such as a week, then discontinued for a period such as a week and then taken continuously for another period such as a week and so on throughout the duration of the treatment regimen. Examples of treatment regimens involving intermittent administration include regimens wherein administration is in cycles of one week on, one week off; or two weeks on, one week off; or three weeks on, one week off; or two weeks on, two weeks off; or four weeks on two weeks off; or one week on three weeks off—for one or more cycles, e.g. 2, 3, 4, 5, 6, 7, 8, 9 or 10 or more cycles.

In one particular dosing schedule, a patient will be given an infusion of a compound of the formula (I) for periods of one hour daily for up to ten days in particular up to five days for one week, and the treatment repeated at a desired interval such as two to four weeks, in particular every three weeks.

More particularly, a patient may be given an infusion of a compound of the formula (I) for periods of one hour daily for 5 days and the treatment repeated every three weeks.

In another particular dosing schedule, a patient is given an infusion over 30 minutes to 1 hour followed by maintenance infusions of variable duration, for example 1 to 5 hours, e.g. 3 hours.

In a further particular dosing schedule, a patient is given a continuous infusion for a period of 12 hours to 5 days, an in particular a continuous infusion of 24 hours to 72 hours.

Ultimately, however, the quantity of compound administered and the type of composition used will be commensurate with the nature of the disease or physiological condition being treated and will be at the discretion of the physician.

The compounds of the formula (I) can be administered as the sole therapeutic agent or they can be administered in combination therapy with one of more other compounds for treatment of a particular disease state, for example a neoplastic disease such as a cancer as hereinbefore defined. In one embodiment, examples of other therapeutic agents or treatments that may be administered together (whether concurrently or at different time intervals) with the compounds of the formula (I) include but are not limited to:
Topoisomerase I inhibitors
Antimetabolites
Tubulin targeting agents
DNA binder and topo II inhibitors
Alkylating Agents
Monoclonal Antibodies.
Anti-Hormones
Signal Transduction Inhibitors
Proteasome Inhibitors
DNA methyl transferases
Cytokines and retinoids
Chromatin targeted therapies
Radiotherapy, and,
Other therapeutic or prophylactic agents; for example agents that reduce or alleviate some of the side effects associated with chemotherapy. Particular examples of such agents include anti-emetic agents and agents that prevent or decrease the duration of chemotherapy-associated neutropenia and prevent complications that arise from reduced levels of red blood cells or white blood cells, for example erythropoietin (EPO), granulocyte macrophage-colony stimulating factor (GM-CSF), and granulocyte-colony stimulating factor (G-CSF). Also included are agents that inhibit bone resorption such as bisphosphonate agents e.g. zoledronate, pamidronate and ibandronate, agents that suppress inflammatory responses (such as dexamethazone, prednisone, and prednisolone) and agents used to reduce blood levels of growth hormone and IGF-I in acromegaly patients such as synthetic forms of the brain hormone somatostatin, which includes octreotide acetate which is a long-acting octapeptide with pharmacologic properties mimicking those of the natural hormone somatostatin. Further included are agents such as leucovorin, which is used as an antidote to drugs that decrease levels of folic acid, or folinic acid it self and agents such as megestrol acetate which can be used for the treatment of side-effects including oedema and thromoembolic episodes.

Thus, as described above, the anti-cancer treatment defined hereinbefore may be applied as a sole therapy or may involve, in addition to the compound of the invention, radiotherapy or chemotherapy. The anti-cancer treatment may also involve conventional surgery.

In another embodiment, the chemotherapy may include one or more of the following categories of anti-tumour agents:

(i) other antiproliferative/antineoplastic drugs and combinations thereof, as used in medical oncology, such as alkylating agents (for example cis-platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5-fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);

(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;

(iii) anti-invasion agents [for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341), N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661) and bosutinib (SKI-606), and metalloproteinase inhibitors like marimastat, inhibitors of urokinase plasminogen activator receptor function or antibodies to Heparanase];

(iv) inhibitors of growth factor function and cell signalling: for example such inhibitors include growth factor antibodies and growth factor receptor antibodies (for example the anti-erbB2 antibody trastuzumab [Herceptin™], the anti-EGFR antibody panitumumab, the anti-erbB1 antibody cetuximab [Erbitux, C225] and any growth factor or growth factor receptor antibodies disclosed by Stern et al. Critical reviews in oncology/haematology, 2005, Vol. 54, pp 11-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib); inhibitors of the hepatocyte growth factor family; inhibitors of the insulin growth factor family; inhibitors of the platelet-derived growth factor family such as imatinib and/or nilotinib (AMN107); inhibitors of serine/threonine kinases (for example Ras/Raf signalling inhibitors such as farnesyl transferase inhibitors, for example sorafenib (BAY 43-9006), tipifarnib (R115777) and lonafarnib (SCH66336)), inhibitors of cell signalling through MEK (for example AZD6244) and/or AKT kinases, c-kit inhibitors, ab1 kinase inhibitors, PI3 kinase inhibitors, Plt3 kinase inhibitors, CSF-1R kinase inhibitors, IGF receptor (insulin-like growth factor) kinase inhibitors; aurora kinase inhibitors (for example AZD1152, PH739358, VX-680, MLN8054, R763, MP235, MP529, VX-528 AND AX39459) and cyclin dependent kinase inhibitors such as CDK2 and/or CDK4 inhibitors;

(v) antiangiogenic agents such as those which inhibit the effects of vascular endothelial growth factor, [for example the anti-vascular endothelial cell growth factor antibody bevacizumab (Avastin™) and for example, a VEGF receptor tyrosine kinase inhibitor such as vandetanib (ZD6474), vatalanib (PTK787), sunitinib (SU11248), axitinib (AG-013736), pazopanib (GW 786034) and 4-(4-fluoro-2-methylindol-5-yloxy)-6-methoxy-7-(3-pyrrolidin-1-ylpropoxy)quinazoline (AZD2171; Example 240 within WO 00/47212), compounds such as those disclosed in International Patent Applications WO97/22596, WO 97/30035, WO 97/32856 and WO 98/13354 and compounds that work by other mechanisms (for example linomide, inhibitors of integrin αvβ3 function and angiostatin)];

(vi) vascular damaging agents such as Combretastatin A4 and compounds disclosed in International Patent Applications WO 99/02166, WO 00/40529, WO 00/41669, WO 01/92224, WO 02/04434 and WO 02/08213;

(vii) an endothelin receptor antagonist, for example zibotentan (ZD4054) or atrasentan;

(viii) antisense therapies, for example those which are directed to the targets listed above, such as ISIS 2503, an anti-ras antisense;

(ix) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and

(x) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

Each of the compounds present in the combinations of the invention may be given in individually varying dose schedules and via different routes.

Where the compound of the formula (I) is administered in combination therapy with one, two, three, four or more other therapeutic agents (preferably one or two, more preferably one), the compounds can be administered simultaneously or sequentially. When administered sequentially, they can be administered at closely spaced intervals (for example over a period of 5-10 minutes) or at longer intervals (for example 1, 2, 3, 4 or more hours apart, or even longer periods apart where required), the precise dosage regimen being commensurate with the properties of the therapeutic agent(s).

The compounds of the invention may also be administered in conjunction with non-chemotherapeutic treatments such as radiotherapy, photodynamic therapy, gene therapy; surgery and controlled diets.

For use in combination therapy with another chemotherapeutic agent, the compound of the formula (I) and one, two, three, four or more other therapeutic agents can be, for example, formulated together in a dosage form containing two, three, four or more therapeutic agents. In an alternative, the individual therapeutic agents may be formulated separately and presented together in the form of a kit, optionally with instructions for their use.

A person skilled in the art would know through their common general knowledge the dosing regimes and combination therapies to use.

Methods of Diagnosis

Prior to administration of a compound of the formula (I), a patient may be screened to determine whether a disease or condition from which the patient is or may be suffering is one which would be susceptible to treatment with a compound having activity against protein kinase A and/or protein kinase B.

For example, a biological sample taken from a patient may be analysed to determine whether a condition or disease, such as cancer, that the patient is or may be suffering from is one which is characterised by a genetic abnormality or abnormal protein expression which leads to up-regulation of PKA and/or PKB or to sensitisation of a pathway to normal PKA and/or PKB activity, or to upregulation of a signal transduction component upstream of PKA and/or PKB such as, in the case of PKB, P13K, GF receptor and PDK 1 & 2.

Alternatively, a biological sample taken from a patient may be analysed for loss of a negative regulator or suppressor of the PKB pathway such as PTEN. In the present context, the term “loss” embraces the deletion of a gene encoding the regulator or suppressor, the truncation of the gene (for example by mutation), the truncation of the transcribed product of the gene, or the inactivation of the transcribed product (e.g. by point mutation) or sequestration by another gene product.

The term up-regulation includes elevated expression or over-expression, including gene amplification (i.e. multiple gene copies) and increased expression by a transcriptional effect, and hyperactivity and activation, including activation by mutations. Thus, the patient may be subjected to a diagnostic test to detect a marker characteristic of up-regulation of PKA and/or PKB. The term diagnosis includes screening. By marker we include genetic markers including, for example, the measurement of DNA composition to identify mutations of PKA and/or PKB. The term marker also includes markers which are characteristic of up regulation of PKA and/or PKB and/or other factors which lead to an upregulation of the relevant pathways, including enzyme activity, enzyme levels, enzyme state (e.g. phosphorylated or not) and mRNA levels of the aforementioned proteins.

The above diagnostic tests and screens are typically conducted on a biological sample selected from tumour biopsy samples, blood samples (isolation and enrichment of shed tumour cells), stool biopsies, sputum, chromosome analysis, pleural fluid, peritoneal fluid, bone marrow or urine.

Identification of an individual carrying a mutation in PKA and/or PKB or a rearrangement of TCL-1 or loss of PTEN expression may mean that the patient would be particularly suitable for treatment with a PKA and/or PKB inhibitor. Tumours may preferentially be screened for presence of a PKA and/or PKB variant prior to treatment. The screening process will typically involve direct sequencing, oligonucleotide microarray analysis, or a mutant specific antibody.

Methods of identification and analysis of mutations and up-regulation of proteins are known to a person skilled in the art. Screening methods could include, but are not limited to, standard methods such as reverse-transcriptase polymerase chain reaction (RT-PCR) or in-situ hybridisation.

In screening by RT-PCR, the level of mRNA in the tumour is assessed by creating a cDNA copy of the mRNA followed by amplification of the cDNA by PCR. Methods of PCR amplification, the selection of primers, and conditions for amplification, are known to a person skilled in the art. Nucleic acid manipulations and PCR are carried out by standard methods, as described for example in Ausubel, F. M. et al., eds. Current Protocols in Molecular Biology, 2004, John Wiley & Sons Inc., or Innis, M. A. et-al., eds. PCR Protocols: a guide to methods and applications, 1990, Academic Press, San Diego. Reactions and manipulations involving nucleic acid techniques are also described in Sambrook et al., 2001, 3^{rd} Ed, Molecular Cloning: A Laboratory Manual, Cold Spring Harbor Laboratory Press. Alternatively a commercially available kit for RT-PCR (for example Roche Molecular Biochemicals) may be used, or methodology as set forth in U.S. Pat. Nos. 4,666,828; 4,683,202; 4,801,531; 5,192,659, 5,272,057, 5,882,864, and 6,218,529 and incorporated herein by reference.

An example of an in-situ hybridisation technique for assessing mRNA expression would be fluorescence in-situ hybridisation (FISH) (see Angerer, 1987 Meth. Enzymol., 152: 649).

Generally, in situ hybridization comprises the following major steps: (1) fixation of tissue to be analyzed; (2) prehybridization treatment of the sample to increase accessibility of target nucleic acid, and to reduce nonspecific binding; (3) hybridization of the mixture of nucleic acids to the nucleic acid in the biological structure or tissue; (4) post-hybridization washes to remove nucleic acid fragments not bound in the hybridization, and (5) detection of the hybridized nucleic acid fragments. The probes used in such applications are typically labeled, for example, with radioisotopes or fluorescent reporters. Preferred probes are sufficiently long, for example, from about 50, 100, or 200 nucleotides to about 1000 or more nucleotides, to enable specific hybridization with the target nucleic acid(s) under stringent conditions. Standard methods for carrying out FISH are described in Ausubel, F. M. et al., eds. Current Protocols in Molecular Biology, 2004, John Wiley & Sons Inc and Fluorescence In Situ Hybridization: Technical Overview by John M. S. Bartlett in Molecular Diagnosis of Cancer, Methods and Protocols, 2nd ed.; ISBN: 1-59259-760-2; March 2004, pps. 077-088; Series: Methods in Molecular Medicine.

Alternatively, the protein products expressed from the mRNAs may be assayed by immunohistochemistry of tumour samples, solid phase immunoassay with microtitre plates, Western blotting, 2-dimensional SDS-polyacrylamide gel electrophoresis, ELISA, flow cytometry and other methods known in the art for detection of specific proteins. Detection methods would include the use of site specific antibodies. The skilled person will recognize that all such well-known techniques for detection of upregulation of PKB, or detection of PKB variants could be applicable in the present case.

Therefore all of these techniques could also be used to identify tumours particularly suitable for treatment with PKA and/or PKB inhibitors.

For example, as stated above, PKB beta has been found to be upregulated in 10-40% of ovarian and pancreatic cancers (Bellacosa et al 1995, Int. J. Cancer 64, 280-285; Cheng et al 1996, PNAS 93, 3636-3641; Yuan et al 2000, Oncogene 19, 2324-2330). Therefore it is envisaged that PKB inhibitors, and in particular inhibitors of PKB beta, may be used to treat ovarian and pancreatic cancers.

PKB alpha is amplified in human gastric, prostate and breast cancer (Staal 1987, PNAS 84, 5034-5037; Sun et al 2001, Am. J. Pathol. 159, 431-437). Therefore it is envisaged that PKB inhibitors, and in particular inhibitors of PKB alpha, may be used to treat human gastric, prostate and breast cancer.

Increased PKB gamma activity has been observed in steroid independent breast and prostate cell lines (Nakatani et al 1999, J. Biol. Chem. 274, 21528-21532). Therefore it is envisaged that PKB inhibitors, and in particular inhibitors of PKB gamma, may be used to treat steroid independent breast and prostate cancers.

Experimental

The invention will now be illustrated, but not limited, by reference to the specific embodiments described in the following procedures and examples.

The starting materials for each of the procedures described below are commercially available unless otherwise specified.

Proton magnetic resonance (¹H NMR) spectra were recorded on a Bruker AV400 instrument operating at 400.13 MHz, in DMSO-d6 or MeOH-d4 (as indicated) at 27° C., unless otherwise stated and are reported as follows: chemical shift δ/ppm (number of protons, multiplicity where s=singlet, d=doublet, t=triplet, q=quartet, m=multiplet, br=broad). The residual protic solvent MeOH (δ_{H}=3.31 ppm) was used as the internal reference.

Mass spectra were recorded using a Waters Platform LCMS system using a Micromass platform LC mass spectrum detector.

Where chlorine is present, the mass quoted for the compound is for ³⁵Cl. The operating conditions used are described below.

General Procedure GP1—Preparation of Diaminobenzenes

Diaminobenzenes can be obtained commercially or prepared from nitroanilines using the reduction method set out below.

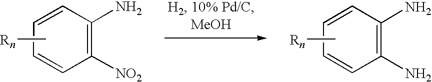

A mixture of nitroarene (5.0 mmol) and 10% palladium on carbon (200 mg) in methanol (10 ml) was stirred for 16 hours under an atmosphere of hydrogen. The catalyst was removed by filtration and the solvent removed in vacuo to afford the desired 1,2-diaminobenzene which was used without further purification.

The following compounds were prepared from the stated precursors according to the method described above:

| | | Chemical | | | |
|---|---|---|---|---|---|
| Compound | Structure | Name | Precursor | N.M.R. Data | M.S. |
| A1-1 | 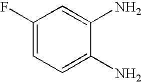 | 1,2-Diamino-4- fluorobenzene | 4-Fluoro-2- nitroaniline | ¹H NMR (DMSO-d₆) 6.44(1H, dd), 6.31 (1H, dd), 6.12(1H, td), 4.73(2H, br s), 4.23 (2H, br s) | MS: [M + H]⁺ 127 |

Preparation 1

### Synthesis of 5-fluoro-3-morpholin-4-yl-benzene-1,2-diamine

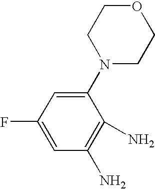

### 1A. 5-Fluoro-3-morpholin-4-yl-2-nitro-phenylamine

A mixture of 2-nitro-3,5-difluoroaniline (1 g; 5.75 mmol), morpholine (600 μl; 1.2 equiv) and triethylamine (960 μl; 1.2 equiv) in THF (20 ml) was heated at 50 C overnight then evaporated. The residue was partitioned between EtOAc and brine. The ethyl acetate layer was separated, dried (Na₂SO₄), filtered and evaporated to give 1.34 g of 5-fluoro-3-morpholin-4-yl-2-nitro-phenylamine as an orange solid. ¹H NMR (DMSO-d₆) 6.41 (2H, d), 6.27 (1H, dd), 6.16 (1H, dd), 3.63 (4H, t), 2.89 (4H, t). MS: [M+H]⁺ 242.

### 1B. 5-fluoro-3-morpholin-4-yl-benzene-1,2-diamine

A solution of 5-fluoro-3-morpholin-4-yl-2-nitro-phenylamine (1.23 g) in methanol (25 ml) was treated with 10% palladium on carbon then hydrogenated at room temperature and pressure overnight. The catalyst was removed by filtration through Celite and the filtrate evaporated to give 1.0 g of the title compound as a brown solid. ¹H NMR (DMSO-d₆) 6.18 (1H, dd), 6.12 (1H, dd), 4.80 (2H, s), 4.00 (2H, s), 3.74 (4H, t), 2.73 (4H, t). MS: [M+H]⁺ 212.

General Procedure GP2—Amide Coupling & Cyclisation

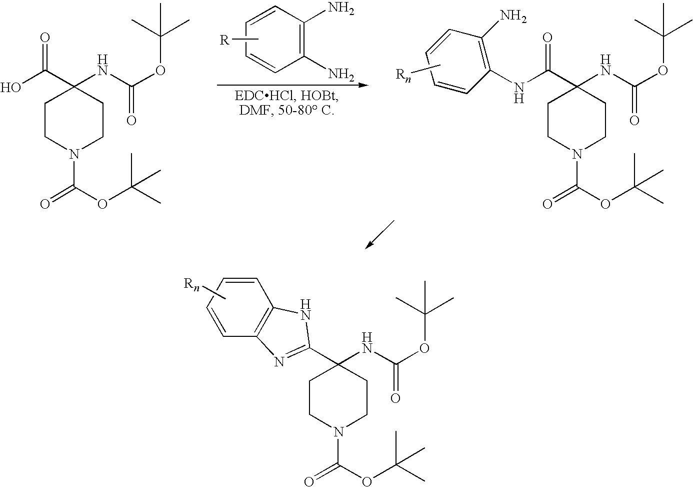

GP2A. Amide Formation

A mixture of 4-tert-butoxycarbonylamino-piperidine-1,4-dicarboxylic acid mono-tert-butyl ester (1 equivalent) (obtainable from Pharmacore Inc., High Point, N.C., USA), 1,2-diaminobenzene derivative (1.1 equivalents), N-ethyl-N′-(3-dimethylaminopropyl)carbodiimide hydrochloride (1-3 equivalents) and 1-hydroxybenzotriazole (1-2 equivalents) in N,N-dimethylformamide (1-10 volumes) was stirred at 50-80° C. for 16 hours. Upon cooling the solvent was removed in vacuo and the residue partitioned between ethyl acetate and water. The organic layer was separated and washed twice with saturated sodium bicarbonate, twice with 2M hydrochloric acid and finally with water. The organic layer was separated and the solvent removed in vacuo to afford the crude amide intermediate. This was used in either the crude state or purified on silica eluting with ethyl acetate/petrol mixtures.

GP2B. Cyclisation Using acetic acid

The amide product of step GP2A was dissolved in acetic acid (5 ml) and stirred at 80° C. for 16 hours. Upon cooling the solvent was removed in vacuo, the residue partitioned between ethyl acetate and water and the organic layer washed with saturated sodium bicarbonate and water. The organic layer was separated, the solvent removed in vacuo and the residue subjected to column chromatography on silica. Elution with 30-40% ethyl acetate in petroleum ether afforded the pure benzoimidazole products.

GP2C. Cyclisation Using p-toluenesulphonic acid

To a mixture of the amide product (1 equivalent) of step GP2A in toluene was added toluene-4-sulphonic acid (0.045 equivalents). The mixture was heated at reflux using a Dean-Stark water trap for 12-18 hours. Upon cooling the reaction mixture was diluted with ethyl acetate and the organic was washed with water followed by aqueous 1M NaOH. The organic was separated, dried (MgSO₄) and the solvent was removed in vacuo to afford the crude product. The product was then purified by flash column chromatography on silica gel, typically using ethyl acetate/petroleum ether as eluent.

By following General Procedure GP2 and either the acetic acid-induced cyclisation step (GP2B) or the p-toluene sulphonic acid-induced cyclisation step (GP2C), the boc-protected intermediate compounds A2-1 to A2-13 were prepared.

| | | | | GP2 | |
|---|---|---|---|---|---|
| Compound | Structure | Chemical Name | Precursor | B/C | M.S. |
| A2-1 | 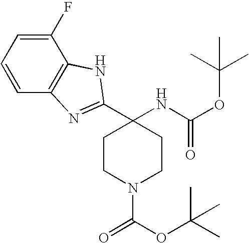 | 4-tert- Butoxycarbonyl- amino-4-(7-fluoro- 1H-benzoimidazol-2- yl)-piperidine-1- carboxylic acid tert- butyl ester | 1,2-diamino-3- fluorobenzene | GP2B | [M + H]⁺ 435 |
| A2-2 | 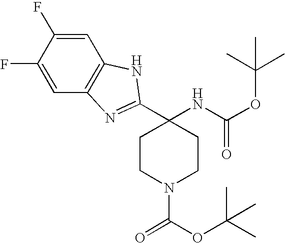 | 4-tert- Butoxycarbonyl- amino-4-(5,6- difluoro-1H- benzoimidazol-2-yl)- piperidine-1- carboxylic acid tert- butyl ester | 1,2-Diamino- 4,5-difluoro- benzene | GP2B | [M + H]⁺ 453 |
| A2-3 | 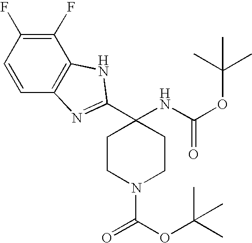 | 4-tert- Butoxycarbonyl- amino-4-(6,7- difluoro-1H- benzoimidazol-2-yl)- piperidine-1- carboxylic acid tert- butyl ester | 1,2-diamino- 3,4-difluoro- benzene | GP2B | [M + H]⁺ 453 |
| A2-4 | 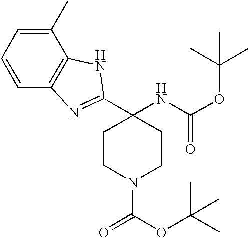 | 4-tert- Butoxycarbonyl- amino-4-(7-methyl- 1H-benzoimidazol-2- yl)-piperidine-1- carboxylic acid tert- butyl ester | 2,3-diamino- toluene | GP2B | [M + H]⁺ 431 |
| A2-5 | 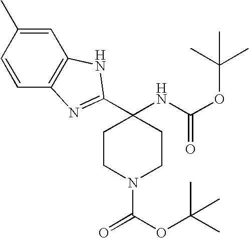 | 4-tert- Butoxycarbonyl- amino-4-(6-methyl- 1H-benzoimidazol-2- yl)-piperidine-1- carboxylic acid tert- butyl ester | 3,4-diamino- toluene | GP2B | [M + H]⁺ 431 |
| A2-6 | 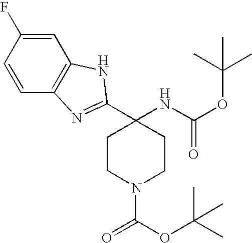 | 4-tert- Butoxycarbonyl- amino-4-(6-fluoro- 1H-benzimidazol-2- yl)-piperidine-1- carboxylic acid tert- butyl ester | 1,2-Diamino- 4- fluorobenzene | GP2B | [M + H]⁺ 435 |
| A2-7 | 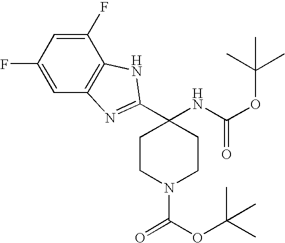 | 4-tert- Butoxycarbonylamino- 4-(5,7-difluoro-1H- benzoimidazol-2-yl)- piperidine-1- carboxylic acid tert- butyl ester | 1,2-Diamino- 3,5-difluoro- benzene | GP2B | [M + H]⁺ 453 |
| A2-8 | 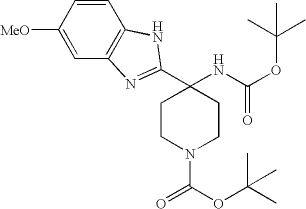 | 4-tert- Butoxycarbonyl- amino-4-(5-methoxy- 1H-benzoimidazol-2- yl)-piperidine-1- carboxylic acid tert- butyl ester | 1,2-Diamino- 4-methoxy- benzene | GP2B | [M + H]⁺ 447 |
| A2-9 | 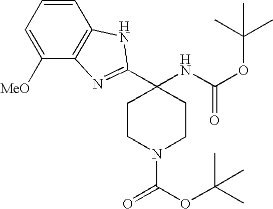 | 4-tert-Butoxy- carbonylamino-4-(4- methoxy-1H- benzoimidazol-2-yl)- piperidine-1- carboxylic acid tert- butyl ester | 1,2-Diamino- 3-methoxy- benzene | GP2B | [M + H]⁺ 447 |
| A2-10 | 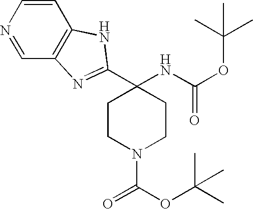 | 4-tert-Butoxy- carbonylamino-4- (1H-imidazo[4,5- c]yridine-2-yl)- piperidine-1- carboxylic acid tert- butyl ester | 3,4-Diamino- pyridine | GP2B | Not run |
| A2-11 | 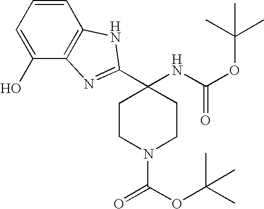 | 4-tert-Butoxy- carbonylamino-4-(4- hydroxy-1H- benzoimidazol-2-yl)- piperidine-1- carboxylic acid tert- butyl ester | 1,2-Diamino- 3-hydroxy- benzene | GP2B | [M + H]⁺ 433 |
| A2-12 | 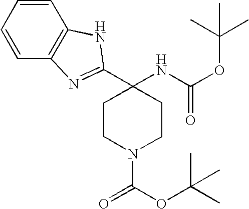 | 4-tert-Butoxy- carbonylamino-4- (1H-benzoimidazol- 2-yl)-piperidine-1- carboxylic acid tert- butyl ester | 1,2-Diamino- benzene | GP2C | [M + H]⁺ 417 |
| A2-13 | 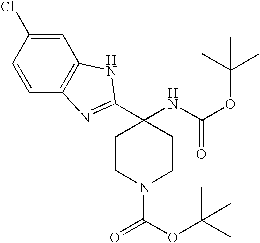 | 4-tert-Butoxy- carbonylamino-4-(6- chloro-1H- benzoimidazol-2-yl)- piperidine-1- carboxylic acid tert- butyl ester | 1,2-Diamino- 4-chloro- benzene | GP2C | [M + H]⁺ 451 |
| A2-14 | 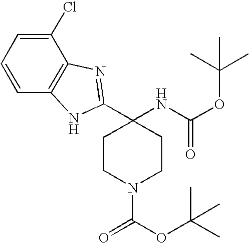 | 4-tert-Butoxy- carbonylamino-4-(4- chloro-1H- benzoimidazol-2-yl)- piperidine-1- carboxylic acid tert- butyl ester | 1,2-diamino-3- chlorobenzene (prepared as in WO05002552 A2) | GP2B | [M + H]⁺ 435 |
| A2-15 | 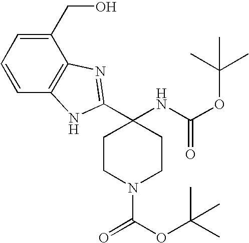 | 4-tert-Butoxy- carbonyl-amino-4-(4- hydroxymethyl-1H- benzoimidazol-2-yl)- piperidine-1- carboxylic acid tert- butyl ester | 2,3- diaminobenzyl alcohol (prepared as in WO05002552 A2) | GP2B | [M + H]⁺ 453 |
| A2-16 | 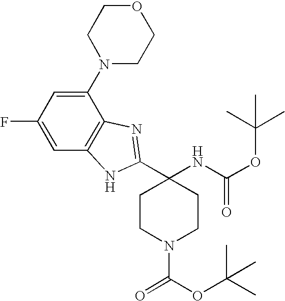 | 4-tert-Butoxy- carbonyl-amino-4-(6- fluoro-4-morpholin- 4-yl-1H-benzo- imidazol-2-yl)- piperidine-1- carboxylic acid tert- butyl ester | 5-fluoro-3- morpholin-4- yl-benzene- 1,2-diamine (new synthesis) | GP2B | [M + H]⁺ 453 |

Compounds A2-1 to A2-13 are believed to be novel compounds and, as such, represent a further aspect of the invention.

General Procedure GP3—Removal of the BOC Protecting Group

To the protected piperidine product of General Procedure GP2, optionally dissolved in a suitable organic solvent (typically dichloromethane or methanol), was added a strong organic (e.g. trifluoroacetic acid) or inorganic (e.g. hydrochloric acid in 1,4-dioxane or ether) acid. The mixture was stirred at room temperature for between 10 minutes and 18 hours to furnish the crude amine as a salt. If necessary, purification could be achieved via silica column chromatography using a mixture of dichloromethane, methanol, acetic acid and H₂0 or dichloromethane, methanol and ammonia, and/or via ion exchange chromatography and/or by preparative HPLC.

By following General Procedure GP3, the substituted piperidine intermediates A3-1 to A3-16 were formed.

| Compound | Structure | Chemical Name | Precursor | N.M.R. Data | M.S. |
|---|---|---|---|---|---|
| A3-1 | 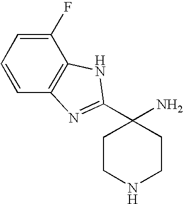 | 4-(7-Fluoro-1H- benzoimidazol- 2-yl)-piperidin- 4-ylamine | 4-tert- Butoxycarbonyl amino-4-(7- fluoro-1H- benzoimidazol- 2-yl)- piperidine-1- carboxylic acid tert-butyl ester | ¹H NMR (DMSO-d₆) 9.25(4H, br s), 9.12(1H, br s), 7.48(1H, d), 7.30(1H, m), 7.11(1H, dd), 3.50(2H, dm), 2.98(2H, m), 2.92(2H, dm), 2.41(2H, tm) | [M + H]⁺ 235 |
| A3-2 | 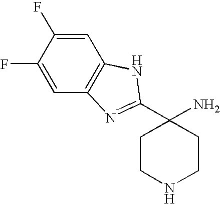 | 4-(5,6-Difluoro- 1H- benzoimidazol- 2-yl)-piperidin- 4-ylamine | 4-tert- Butoxycarbonyl amino-4-(5,6- difluoro-1H- benzoimidazol- 2-yl)- piperidine-1- carboxylic acid tert-butyl ester | ¹H NMR (aDMSO-d₆) 9.18(4H, br s), 9.04(1H, br s), 7.77(2H, dd), 3.46(2H, dm), 2.96(2H, m), 2.86(2H, dm), 2.37(2H, tm) | [M + H]⁺ 253 |
| A3-3 | 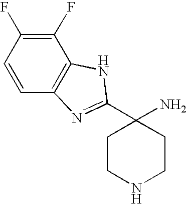 | 4-(6,7-Difluoro- 1H- benzoimidazol- 2-yl)-piperidin- 4-ylamine | 4-tert- Butoxycarbonyl amino-4-(6,7- difluoro-1H- benzoimidazol- 2-yl)- piperidine-1- carboxylic acid tert-butyl ester | ¹H NMR (DMSO-d₆) 9.25(4H, br s), 9.14(1H, br s), 7.46(1H, dd), 7.38(1H, m), 3.48(2H, dm), 3.00(2H, m), 2.90(2H, dm), 2.39(2H, tm) | [M + H]⁺ 253 |
| A3-4 | 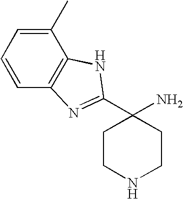 | 4-(7-Methyl-1H- benzoimidazol- 2-yl)-piperidin- 4-ylamine | 4-tert- Butoxycarbonyl amino-4-(7- methyl-1H- benzoimidazol- 2-yl)- piperidine-1- carboxylic acid tert-butyl ester | ¹H NMR (MeOH-d₄) 7.60(1H, d), 7.38(1H, t), 7.28(1H, d), 3.65(2H, dm), 3.22(4H, m), 2.70(3H, s), 2.57(2H, tm) | [M + H]⁺ 231 |
| A3-5 | 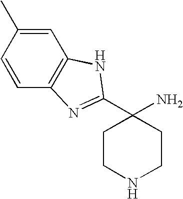 | 4-(6-Methyl-1H- benzoimidazol- 2-yl)-piperidin- 4-ylamine | 4-tert- Butoxycarbonyl amino-4-(6- methyl-1H- benzoimidazol- 2-yl)- piperidine-1- carboxylic acid tert-butyl ester | ¹H NMR (DMSO-d₆) 9.25(5H, br s), 7.58(1H, d), 7.48(1H, d), 7.14(1H, dd), 3.48(2H, dm), 2.98(2H, m), 2.90(2H, dm), 2.48(3H, s), 2.44(2H, tm) | [M + H]⁺ 231 |
| A3-6 | 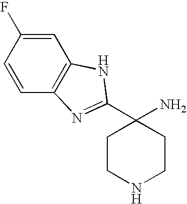 | 4-(6-Fluoro-1H- benzoimidazol- 2-yl)-piperidin- 4-ylamine | 4-tert- Butoxycarbonyl amino-4-(6- fluoro-1H- benzoimidazol- 2-yl)- piperidine-1- carboxylic acid tert-butyl ester | ¹H NMR (DMSO-d₆) 9.20(4H, br s), 9.10(1H, br s), 7.68(1H, dd), 7.48(1H, dd), 7.17(1H, td), 3.46(2H, dm), 2.96(2H, m), 2.86(2H, dm), 2.37(2H, tm) | [M + H]⁺ 235 |
| A3-7 | 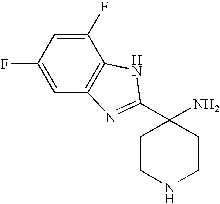 | 4-(5,7-Difluoro- 1H- benzoimidazol- 2-yl)-piperidin- 4-ylamine | 4-tert- Butoxycarbonyl amino-4-(5,7- difluoro-1H- benzoimidazol- 2-yl)- piperidine-1- carboxylic acid tert-butyl ester | ¹H NMR (DMSO-d₆) 9.22(4H, br s), 9.10(1H, br s), 7.34(1H, d), 7.20(1H, td), 3.48(2H, dm), 2.99(2H, m), 2.88(2H, dm), 2.41(2H, tm) | [M + H]⁺ 253 |
| A3-8 | 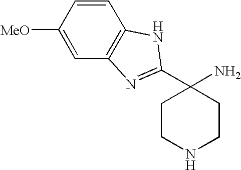 | 4-(5-Methoxy- 1H- benzoimidazol- 2-yl)-piperidin- 4-ylamine | 4-tert- Butoxycarbonyl amino-4-(5- methoxy-1H- benzoimidazol- 2-yl)- piperidine-1- carboxylic acid tert-butyl ester | ¹H NMR (400 MHz, DMSO- d6): 9.30-9.15 (6H, br s), 7.22 (2H, m), 6.82 (1H, m), 3.95 (3H, s), 3.50- 3.40(2H, m), 3.03-2.85(4H, m), 2.45-2.35 (2H, m) | — |
| A3-9 | 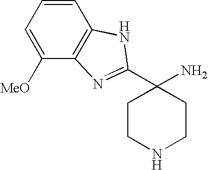 | 4-(4-Methoxy- 1H- benzoimidazol- 2-yl)-piperidin- 4-ylamine | 4-tert- Butoxycarbonyl amino-4-(4- methoxy-1H- benzoimidazol- 2-yl)- piperidine-1- carboxylic acid tert-butyl ester | — | [M + H]⁺ 247 |
| A3-10 | 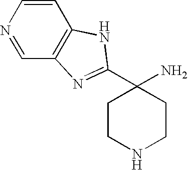 | 4-(1H- Imidazo[4,5- c]pyridin-2-yl)- piperidin-4-yl- amine | 4-tert-Butoxy- carbonylamino- 4-(1H- imidazo[4,5- c]pyridin-2-yl)- piperidine-1- carboxylic acid tert-butyl ester | — | [M + H]⁺ 218 |
| A3-11 | 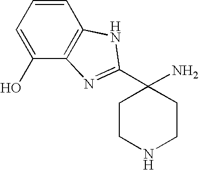 | 4-(4-Hydroxy- 1H- benzoimidazol- 2-yl)-piperidin- 4-ylamine | 4-tert- Butoxycarbonyl amino-4-(4- hydroxy-1H- benzoimidazol- 2-yl)- piperidine-1- carboxylic acid tert-butyl ester | ¹H NMR (400 MHz, DMSO- d6): 9.25(6H, br s), 7.09(2H, m), 6.70(1H, m), 3.50-3.40 (2H, m), 3.05- 2.85(4H, m), 2.48-2.37(2H, m) | |
| A3-12 | 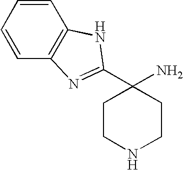 | 4-(1H-benzo- imidazol-2-yl)- piperidin-4- ylamine | 4-tert- Butoxycarbonyl amino-4-(1H- benzoimidazol- 2-yl)- piperidine-1- carboxylic acid tert-butyl ester | — | [M + H]⁺ 217 |
| A3-13 | 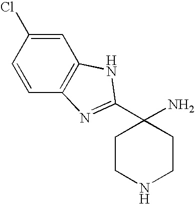 | 4-(6-Chloro-1H- benzoimidazol- 2-yl)-piperidin- 4-ylamine | 4-tert- Butoxycarobnyl amino-4-(6- chloro-1H- benzoimidazol- 2-yl)- piperidine-1- carboxylic acid tert-butyl ester | — | [M + H]⁺ 251 |
| A3-14 | 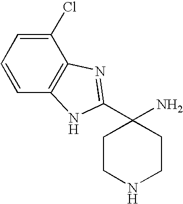 | 4-(4-Chloro-1H- benzoimidazol- 2-yl)-piperidin- 4-ylamine | 4-tert- Butoxycarbonyl amino-4-(4- chloro-1H- benzoimidazol- 2-yl)- piperidine-1- carboxylic acid tert-butyl ester | ¹H NMR (DMSO-d₆) 9.40-9.20(5H, br s), 7.62(1H, d), 7.32(1H, d), 7.29(1H, t), 3.53-3.42(2H, m), 3.07-2.95 (2H, m), 2.94- 2.85(2H, m), 2.48-2.38(2H, m) | |
| A3-15 | 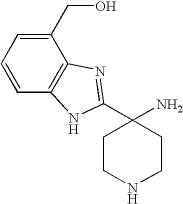 | [2-(4-Amino- piperidin-4-yl)- 1H- benzoimidazol- 4-yl]-methanol | 4-tert- Butoxycarbonyl amino-4-(4- hydroxymethyl- 1H- benzoimidazol- 2-yl)- piperidine-1- carboxylic acid tert-butyl este | ¹H NMR (DMSO-d₆) 9.18(6H, br s), 7.57(1H, d), 7.34-7.25(2H, m), 4.8(2H, s), 3.53-3.46(2H, m), 3.08-2.88 (4H, m), 2.50- 2.37(2H, m) | |
| A3-16 | 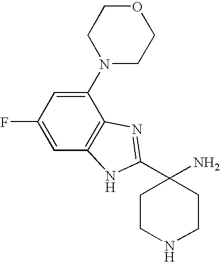 | 4-(6-Fluoro-4- morpholin-4-yl- 1H- benzoimidazol- 2-yl)-piperidin- 4-ylamine | 4-tert- Butoxycarbonyl amino-4-(6- fluoro-4- morpholin-4-yl- 1H- benzoimidazol- 2-yl)- piperidine-1- carboxylic acid tert-butyl ester | | M/z: 320 |

Preparation of Halo-Heterocycles

Preparation 2

### 4-Chloro-7H-pyrrolo[2,3-d]pyrimidine

### 2A. 6-Amino-5-(2,2-diethoxy-ethyl)-2-mercapto-pyrimidin-4-ol

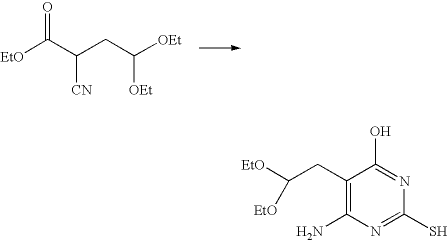

To ethanol (200 ml) was added sodium (2.05 g, 89 mmol) in small portions. The solution was stirred until complete dissolution of the sodium metal. 2-Cyano-4,4-diethoxy-butyric acid ethyl ester (J. Chem. Soc., 1960, 131-138) (9.292 g, 40.5 mmol) was then added as a solution in ethanol (50 ml), followed by addition of thiourea (3.08 g, 40.4 mmol). The solution was heated at 85° C. for 18 hours, and then cooled to room temperature. The solution was concentrated, and saturated aqueous ammonium chloride solution (150 ml) was added. The mixture was stirred at room temperature for 18 hours, after which time the solid was collected by filtration, and washed with water (20 ml) to yield the product (3.376 g, 36%).

### 2B. 6-Amino-5-(2,2-diethoxy-ethyl)-pyrimidin-4-ol

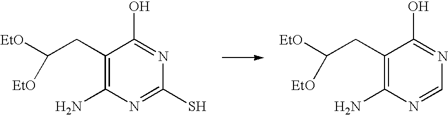

To a suspension of 6-amino-5-(2,2-diethoxy-ethyl)-2-mercapto-pyrimidin-4-ol (1.19 g, 4.6 mmol) in water (50 ml) was added Raney nickel (Aldrich Raney 2800 nickel, 4.8 ml). The mixture was heated at reflux for 1 hour, and then the hot solution was filtered through Celite®. The nickel residue was washed with further water (100 ml), and these washings were filtered through Celite. The aqueous filtrate was evaporated to dryness to yield the title product (0.747 g, 71%).

### 2C. 7H-Pyrrolo[2,3-d]pyrimidin-4-ol

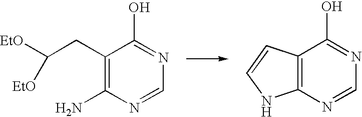

This compound was prepared as described in J. Chem. Soc., 1960, pp. 131-138.

### 2D. 4-Chloro-7H-pyrrolo[2,3-d]pyrimidine

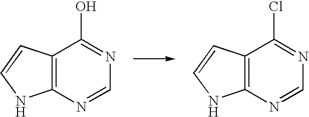

To 7H-pyrrolo[2,3-d]pyrimidin-4-ol (0.425 g, 3.14 mmol) was added phosphorus oxychloride (4 ml). The mixture was heated at reflux for 90 minutes and then cooled to room temperature. The solution was poured onto cracked ice, and extracted with chloroform (3×50 ml) and ethyl acetate (100 ml). The extracts were then dried and concentrated, and the residue obtained triturated with hot ethyl acetate (200 ml) to yield the title compound (0.204 g, 42%).

Preparation 3

### 4-Fluoro-1-triisopropylsilanyl-1H-pyrrolo[2,3-b]pyridine

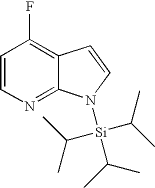

The title compound was prepared according to the method described in Organic Letters (2003), Vol. 5, No. 26, 5023-5025.

Preparation 4

### 4-Fluoro-1,3-dihydro-pyrrolo[2,3-b]pyridin-2-one

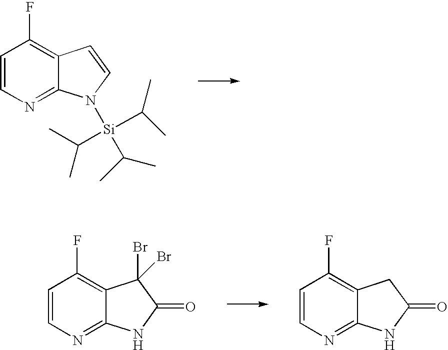

### 4A. 3,3-Dibromo-4-fluoro-1,3-dihydro-pyrrolo[2,3-b]pyridin-2-one

To a solution of 4-fluoro-1-triisopropylsilanyl-1H-pyrrolo[2,3-b]pyridine (Org Lett 2003, 5, 5023-5026, 1.0 g, 3.4 mmol) in tert-butanol (25 ml) was added portionwise pyridine tribromide (3.8 g, 11.97 mmol) and this mixture was stirred at room temperature for 3 days. The solvent was removed in vacuo, water and ethyl acetate was added. the mixture was filtered under suction then the organic layer separated. The aqueous fraction was extracted twice with ethyl acetate then the organic liquors were combined and concentrated. The crude product was purified on a silica Biotage column, eluting with petrol/ethyl acetate to furnish the clean product (312 mg, 29%).

### 4B. 4-Fluoro-1,3-dihydro-pyrrolo[2,3-b]pyridin-2-one

A mixture of 3,3-dibromo-4-fluoro-1,3-dihydro-pyrrolo[2,3-b]pyridin-2-one (312 mg, 1.0 mmol), acetic acid (4.5 ml), zinc dust (658 mg, 10 mmol) and methanol (4.5 ml) was stirred at room temperature for 3 hours. Brine was added and the reaction was extracted with ethyl acetate. The organic liquors were washed with brine, dried (MgSO₄) and concentrated to furnish the title compound (184 mg, contains ˜40% des-fluorinated product). Used thus in further reactions.

Preparation 5

### 4-Chloro-5,7-dihydro-pyrrolo[2,3-d]pyrimidin-6-one

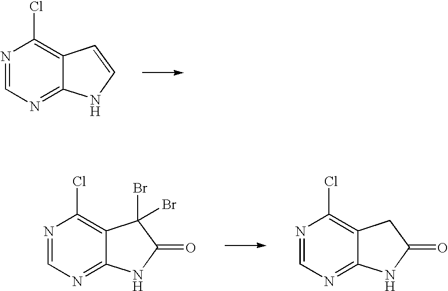

The title compound can be prepared according to the protocols in Preparation 4 using 4-chloro-7H-pyrrolo[2,3-d]pyrimidine.

Preparation 6

### 5-bromo-4-chloro-7H-pyrrolo[2,3-d[pyrimidine

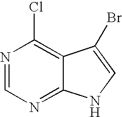

N-Bromosuccinimide (6.84 g, 38.42 mmol) was added portionwise to 4-chloro-7H-pyrrolo[2,3-d]pyrimidine (5 g, 32.56 mmol) in dichloromethane, dry (125 ml) at 20° C. under nitrogen. The resulting suspension was stirred at 20° C. for 1 hour. The reaction mixture was evaporated and the resulting brown solid was triturated with water to give a purple solid which was collected by filtration. The crude solid was triturated with hot MeOH to give a solid which was collected by filtration. The hot trituration was repeated to give 5-bromo-4-chloro-7H-pyrrolo[2,3-d]pyrimidine (5.23 g, 69.1%) as a cream solid.

1H NMR (400.13 MHz, DMSO-d6) δ 7.94 (1H, s), 8.63 (1H, s), 12.95 (1H, s)

MS m/e MH⁺ 232

Preparation 7

### 4,5-dichloro-7H-pyrrolo[2,3-d]pyrimidine

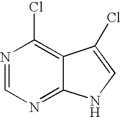

N-Chlorosuccinimide (4.78 g, 35.81 mmol) was added portionwise to a stirred suspension of 4-chloro-7H-pyrrolo[2,3-d]pyrimidine (5 g, 32.56 mmol) in DCM, dry (125 ml) at room temperature. The resulting suspension was stirred for 1 hour then heated to reflux for 5 hours, then allowed to cool down and left to stir at room temperature overnight. The reaction mixture was evaporated and suspended in water (50 mL). The suspension was filtered giving crude product as a grey solid. The solid was suspended in hot methanol and filtered. The solid was then suspended in hot ethyl acetate and filtered to give 4,5-dichloro-7H-pyrrolo[2,3-d]pyrimidine (4.87 g, 80%) as a grey solid.

1H NMR (400.13 MHz, DMSO-d6) δ 7.91 (1H, s), 8.64 (1H, s), 12.87 (1H, s)

MS m/e MH⁺ 188

Preparation 8

### 4-Chloro-5-methyl-7H-pyrrolo[2,3-d]pyrimidine

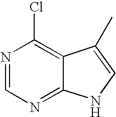

n-Butyllithium (4.08 ml, 6.52 mmol) was added dropwise to 5-bromo-4-chloro-7H-pyrrolo[2,3-d]pyrimidine (689 mg, 2.96 mmol) in tetrahydrofuran (40 ml) at −78° C. over a period of 5 minutes under nitrogen. The resulting suspension was stirred at −78° C. for 30 minutes. Methyl iodide (0.295 ml, 4.74 mmol) was added and the reaction was allowed to warm to ambient temperature. The reaction mixture was diluted with water (25 mL), and extracted with ethyl acetate (50 mL). The organic layer was washed with brine (25 mL) then dried over MgSO4, filtered and evaporated to afford crude product. The crude product was purified by flash silica chromatography, elution gradient 20 to 50% EtOAc in isohexane. Pure fractions were evaporated to dryness to afford 4-chloro-5-methyl-7H-pyrrolo[2,3-d]pyrimidine (244 mg, 49.1%) as a white solid.

1H NMR (400.13 MHz, DMSO-d6) δ 2.42 (3H, d), 7.43 (1H, d), 8.51 (1H, s), 12.22 (1H, s)

MS m/e MH⁺ 168

General Procedure GP4—Reaction of Substituted Piperidine with Halo-Heterocycle

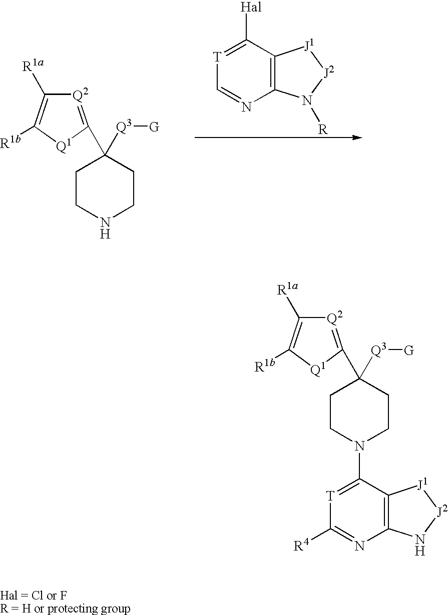

GP4A. Reaction Carried Out Under Microwave Irradiation

A mixture of the piperidine (1 equivalent), halogenated bicycle (e.g. 6-chloro-9H-purine typically 1-2 equivalents), triethylamine (0-10 equivalents) and organic solvent (typically n-butanol or N-methylpyrrolidin-2-one) was irradiated in a sealed microwave vessel to 100-200° C. for 0.25-5 hours. The reaction was typically filtered under suction washing with suitable organic solvents (e.g. methanol, dichloromethane) then concentrated. Optional aqueous work-up or ion exchange chromatography was undertaken followed by purification by silica Biotage column eluting with ethyl acetate/petrol, dichloromethane/acetic acid/methanol/water, or dichloromethane/methanolic ammonia to furnish the pure product.

GP4B. Reaction Carried Out in a Reactivial™

A mixture of the piperidine (0.33 mmol), 4-chloropyrrolo[2,3-d]pyrimidine (or other haloarene, 1-1.5 equivalents), triethylamine (1-6 equivalents) and N-methylpyrrolidinone (5 vols) was stirred for between 1 and 18 hours at 80-110° C. in a Reactivial™ (available from Pierce Chemical Company/Thermo Fisher Scientific Inc.). Upon cooling to room temperature the mixture was diluted with ethyl acetate and water and the organic layer washed three times with water. The organic layer was separated, the solvent removed in vacuo to furnish a crude oil. Alternatively, ion exchange chromatography could be utilised. The material was purified further by column chromatography on silica. Elution with mixtures of dichloromethane, methanol, acetic acid and water (DMAW) afforded the pure N-substituted derivatives as their acetate salts. Mixtures of dichloromethane, methanol and ammonia afforded the free base. If necessary, the product would be further purified by preparative HPLC.

### EXAMPLES 1 TO 32

The intermediate compounds A3-1 to A3-16 were reacted with the appropriate chloro-heterocycle using either procedure GP4A or GP4B to give the compounds of Examples 1 to 32.

| Example | | | | | |
|---|---|---|---|---|---|
| Number | Compound | Chemical Name | Method | N.M.R. Data | M.S. |
| 1 | 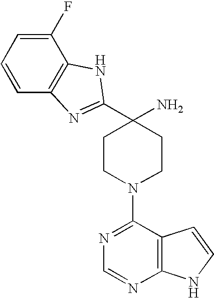 | 4-(7-Fluoro-1H- benzoimidazol- 2-yl)-1-(7H- pyrrolo[2,3- d]pyrimidin-4- yl)-piperidin-4- ylamine | Method GP4B using compounds A3-1 and 4- chloro-7H- pyrrolo[2,3- d]pyrimidine | ¹H NMR (DMSO-d₆) 11.67(1H, br s), 8.15 (1H, s), 7.32(1H, d), 7.18(1H, m), 7.12 (1H, m), 6.92(1H, dd), 6.63(1H, d), 4.28(2H, dm), 3.88 (2H, tm), 2.45(2H, tm), 1.90(3H, s), 1.80(2H, dm) | MS: [M + H]⁺ 352 |
| 2 | 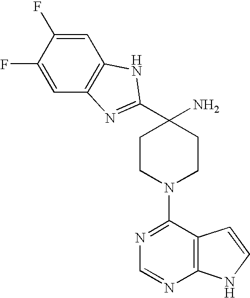 | 4-(5,6-Difluoro- 1H- benzoimidazol- 2-yl)-1-(7H- pyrrolo[2,3- d]pyrimidin-4- yl)-piperidin-4- ylamine | Method GP4B using compounds A3-2 and 4- chloro-7H- pyrrolo[2,3- d]pyrimidine | ¹H NMR (DMSO-d₆) 11.68(1H, br s), 8.15 (1H, s), 7.52(2H, t), 7.18(1H, m), 6.62 (1H, d), 4.25(2H, dm), 3.87(2H, tm), 2.22(2H, tm), 1.90 (3H, s), 1.77(2H, dm) | MS: [M + H]⁺ 370 |
| 3 | 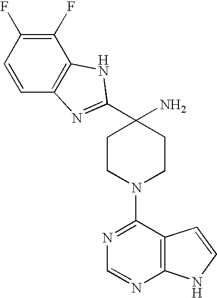 | 4-(6,7-Difluoro- 1H- benzoimidazol- 2-yl)-1-(7H- pyrrolo[2,3- d]pyrimidin-4- yl)-piperidin-4- ylamine | Method GP4B using compounds A3-3 and 4- chloro-7H- pyrrolo[2,3- d]pyrimidine | ¹H NMR (DMSO-d₆) 11.67(1H, br s), 8.16 (1H, s), 7.28(1H, dd), 7.18(2H, m), 6.62(1H, d), 4.28 (2H, dm), 3.88(2H, tm), 2.23(2H, tm), 1.89(3H, s), 1.78 (2H, dm) | MS: [M + H]⁺ 370 |
| 4 | 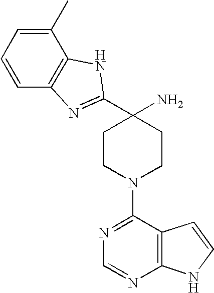 | 4-(7-Methyl-1H- benzoimidazol- 2-yl)-1-(7H- pyrrolo[2,3- d]pyrimidine-4- yl)-piperidin-4- ylamine | Method GP4B using compounds A3-4 and 4- chloro-7H- pyrrolo[2,3- d]pyrimidine | ¹H NMR (DMSO-d₆) 11.67(1H, br s), 8.16 (1H, s), 7.30(1H, d), 7.18(1H, d), 7.03 (1H, t), 6.91(1H, d), 6.64(1H, d), 4.28 (2H, dm), 3.88(2H, tm), 2.47(3H, s), 2.26(2H, tm), 1.91 (3H, s), 1.77(2H, dm) | MS: [M + H]⁺ 348 |
| 5 | 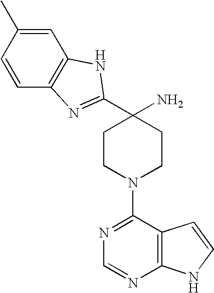 | 4-(6-Methyl-1H- benzoimidazol- 2-yl)-1-(7H- pyrrolo[2,3- d]pyrimidin-4- yl)-piperidin-4- ylamine | Method GP4B using compounds A3-5 and 4- chloro-7H- pyrrolo[2,3- d]pyrimidine | ¹H NMR (DMSO-d₆) 11.68(1H, br s), 8.15 (1H, s), 7.37(1H, d), 7.28(1H, s), 7.18 (1H, d), 6.94(1H, d), 6.63(1H, d), 4.25 (2H, dm), 3.91(2H, tm), 2.38(3H, s), 2.23(2H, tm), 1.90 (3H, s), 1.77(2H, dm) | MS: [M + H]⁺ 348 |
| 6 | 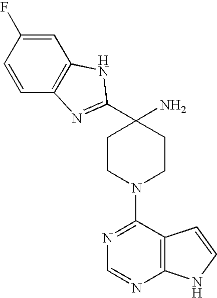 | 4-(6-Fluoro-1H- benzoimidazol- 2-yl)-1-(7H- pyrrolo[2,3- d]pyrimidin-4- yl)-piperidin-4- ylamine | Method GP4B using compounds A3-6 and 4- chloro-7H- pyrrolo[2,3- d]pyrimidine | ¹H NMR (DMSO-d₆) 11.62(1H, br s), 8.15 (1H, s), 7.48(1H, dd), 7.28(1H, dd), 7.18(1H, d), 6.97 (1H, t d), 6.62(1H, d), 4.28(2H, dm), 3.88 (2H, tm), 2.23(2H, tm), 1.88(3H, s), 1.78(2H, dm) | MS: [M + H]⁺ 352 |
| 7 | 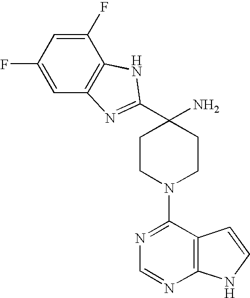 | 4-(5,7-Difluoro- 1H- benzoimidazol- 2-yl)-1-(7H- pyrrolo[2,3- d]pyrimidin-4- yl)-piperidin-4- ylamine | Method GP4B using compounds A3-7 and 4- chloro-7H- pyrrolo[2,3- d]pyrimidine | ¹H NMR (DMSO-d₆) 11.68(1H, br s), 8.14 (1H, s), 7.18(2H, m), 6.96(1H, td), 6.63 (1H, d), 4.28(2H, dm), 3.88(2H, tm), 2.23(2H, tm), 1.85 (3H, s), 1.80(2H, dm) | MS: [M + H]⁺ 370 |
| 8 | 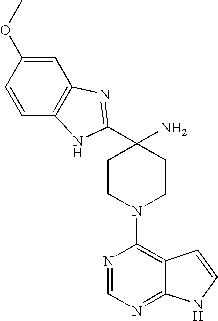 | 4-(5-Methoxy- 1H- benzoimidazol- 2-yl)-1-(7H- pyrrolo[2,3- d]pyrimidin-4- yl)-piperidin-4- ylamine | Method GP4B using compounds A3-8 and 4- chloro-7H- pyrrolo[2,3- d]pyrimidine | ¹H NMR (DMSO-d₆) 11.66(1H, s), 8.15 (1H, s), 7.35(1H, br s), 7.28(1H, s), 7.18 (1H, d), 7.00(1H, br s), 6.75(1H, dd), 6.63(1H, d), 4.25 (2H, dm), 3.89(2H, tm), 3.76(3H, s), 2.23(2H, tm), 1.75 (2H, dm) | M/z: 364 |
| 9 | 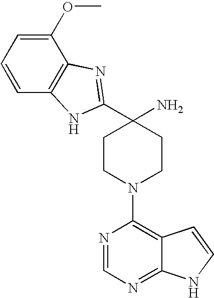 | 4-(4-Methoxy- 1H- benzoimidazol- 2-yl)-1-(7H- pyrrolo[2,3- d]pyrimidin-4- yl)-piperidin-4- ylamine | Method GP4B using compounds A3-9 and 4- chloro-7H- pyrrolo[2,3- d]pyrimidine NB This compound can also exist as the 7- methoxy- tautomer or a mixture thereof. | ¹H NMR (DMSO-d₆) 11.65(1H, br s), 8.15 (1H, s), 7.19(1H, d), 7.10-7.00(2H, m) 6.67(1H, m), 6.63 (1H, d), 4.25(2H, dt), 3.95-3.85(5H, m), 2.23(2H, td), 1.76(2H, dt) | M/z: 364 |
| 10 | 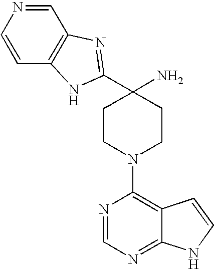 | 4-(1H- Imidazo[4,5- c]pyridin-2-yl)- 1-(7H- pyrrolo[2,3- d]pyrimidin-4- yl-piperidin-4- ylamine | Method GP4B using compounds A3-10 and 4- chloro-7H- pyrrolo[2,3- d]pyrimidine | ¹H NMR (DMSO-d₆) 8.25(1H, d), 8.09 (1H, s), 7.93(1H, d), 7.23(1H, dd), 7.18 (1H, d), 6.65(1H, d), 4.15(2H, m), 3.88 (2H, m), 2.25(2H, m), 1.85(2H, m) | M/z: 335 |
| 11 | 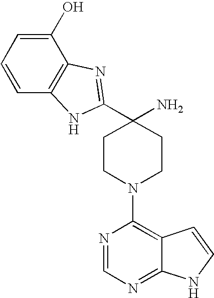 | 2-[4-Amino-1- (7H-pyrrolo[2,3- d]pyrimidin-4- yl)-piperidin-4- yl]-1H- benzoimidazol- 4-ol | Method GP4B using compounds A3-11 and 4- chloro-7H- pyrrolo[2,3- d]pyrimidine | ¹H NMR (DMSO-d₆) 11.67(1H, br s), 8.15 (1H, s), 7.19(1H, d), 6.95-6.88(2H, m) 6.63(1H, d), 6.50 (1H, d), 4.29(2H, dt), 3.87(2H, td), 2.25(2H, td), 1.78 (2H, dt) | M/z: 350 |
| 12 | 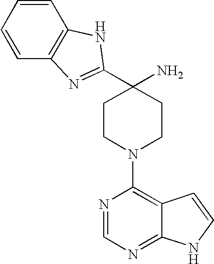 | 4-(1H- benzoimidazol- 2-yl)-1-(7H- pyrrolo[2,3- d]pyrimidin-4- yl)-piperidin-4- ylamine | Method GP4A using compounds A3-12 and 4- chloro-7H- pyrrolo[2,3- d]pyrimidine | ¹H NMR (Me-d3- OD): 8.17(1H, s), 7.62-7.51(2H, m), 7.29-7.19(2H, m), 7.15(1H, d), 6.68 (1H, d), 4.29-4.16 (2H, m), 4.16-4.04 (2H, m), 2.56-2.43 (2H, m), 2.05-1.92 (2H, m) | M/z: 334 |
| 13 | 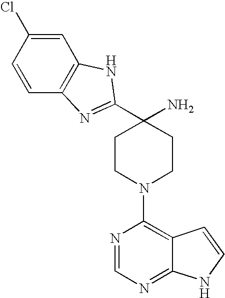 | 4-(6-chloro-1H- benzoimidazol- 2-yl)-1-(7H- pyrrolo[2,3- d]pyrimidin-4- yl)-piperidin-4- ylamine | Method GP4B using compounds A3-13 and 4- chloro-7H- pyrrolo[2,3- d]pyrimidine | 1H NMR (Me-d3- OD): 8.17(1H, s), 7.56(1H, d), 7.52 (1H, d), 7.22(1H, dd), 7.15(1H, d), 6.68(1H, d), 4.32- 4.19(2H, m), 4.14- 4.00(2H, m), 2.54-2.39(2H, m), 2.03-1.92(2H, m) | M/z: 368 |
| 14 | 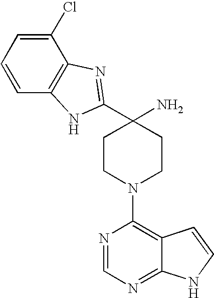 | 4-(4-Chloro-1H- benzoimidazol- 2-yl)-1-(7H- pyrrolo[2,3- d]pyrimidin-4- yl)-piperidin-4- ylamine | Method GP4B using compounds A3-14 and 4-chloro- 7H- pyrrolo[2,3- d]pyrimidine | 1H NMR (400 MHz, DMSO-d6): 11.65(1H, s), 8.64 (1H, t), 8.36(1H, d), 8.14(1H, s), 7.79(1H, dd), 7.47 (1H, d), 7.15(1H, d), 6.55(1H, d), 4.35(2H, d), 4.30- 4.23(2H, m), 3.45- 3.35(2H, m), 2.69 (2H, s), 2.12-2.05 (2H, m), 1.53-1.43 (2H, m) | M/z: 400/ 402 |
| 15 | 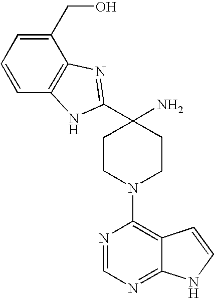 | {2-[4-Amino-1- (7H-pyrrolo[2,3- d]pyrimidin-4- yl)-piperidin-4- yl]-1H-benzo- imidazol-4- yl}-methanol | Method GP4B using compounds A3-15 and 4-chloro- 7H- pyrrolo[2,3- d]pyrimidine | ¹H NMR (DMSO-d₆) 11.65(1H, br s), 8.15 (1H, s), 7.19(1H, d), 7.10-7.00(2H, m) 6.67(1H, m), 6.63 (1H, d), 4.25(2H, dt), 3.95-3.85(5H, m), 2.23(2H, td), 1.76(2H, dt) | M/z: 364 |
| 16 | 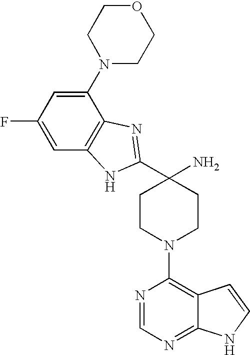 | 4-(6-Fluoro-4- morpholin-4-yl- 1H-benzo- imidazol-2- yl)-1-(7H- pyrrolo[2,3- d]pyrimidin-4- yl)-piperidin-4- ylamine | Method GP4B using compounds A3-16 and 4-chloro- 7H- pyrrolo[2,3- d]pyrimidine | ¹H NMR (DMSO-d₆) 12.10(1H, br s), 11.15(1H, br s), 8.15 (1H, s), 7.18(1H, d), 6.73(1H, d) 6.62 (1H, d), 6.30(1H, d), 4.27(2H, dt), 3.88 (2H, td), 3.78(4H, m), 3.50(4H, m), 2.20(2H, td), 1.78 (2H, dt) | M/z: 437 |
| 17 | 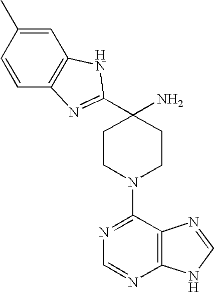 | 4-(6-Methyl-1H- benzoimidazol- 2-yl)-1-(9H- purin-6-yl)- piperidin-4- ylamine | Method GP4B using compounds A3-5 and 6- chloro-9H- purine | ¹H NMR (DMSO-d₆) 8.23(1H, s), 8.12 (1H, s), 7.37(1H, d), 7.27(1H, s), 6.93 (1H, d), 4.80(2H, br s), 4.05(2H, br s), 2.38(3H, s), 2.22 (2H, tm), 1.85(3H, s), 1.77(2H, dm) | [M + H]⁺ 349 |
| 18 | 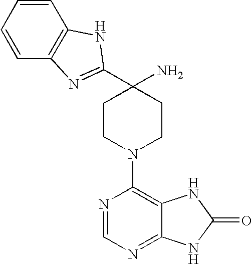 | 6-[4-amino-4- (1H- benzoimidazol- 2-yl)-piperidin- 1-yl]-7,9- dihydro-purin-8- one | Method GP4A using compounds A3-12 and 6- chloro-7,9- dihydro-purin- 8-one | ¹H NMR (DMSO- d6): 8.11(1H, s), 7.53(2H, s), 7.22- 7.10(2H, m), 3.86- 3.66(4H, m), 2.32 (2H, m), 1.83(2H, m) | M/z: 351 |
| 19 | 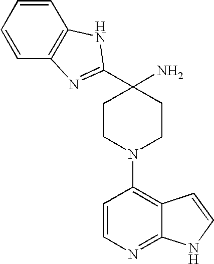 | 4-(1H- benzoimidazol- 2-yl)-1-(1H- pyrrolo[2,3- b]pyridin-4-yl)- piperidin- 4-ylamine | Method GP4B using compounds A3-12 and 4- chloro-1H- pyrrolo[2,3- b]pyridine (Preparation 3) | 1H NMR (Me-d3- OD): 7.94(1H, d), 7.66-7.51(2H, m), 7.31-7.21(2H, m), 7.20(1H, d), 6.57 (1H, d), 6.54(1H, d), 3.83-3.61(4H, m), 2.67-2.53(2H, m), 2.13-1.99(2H, m) | M/z: 333 |
| 20 | 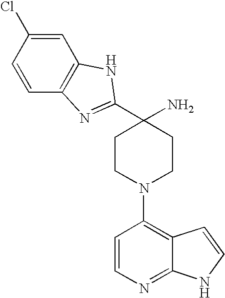 | 4-(6-chloro-1H- benzoimidazol- 2-yl)-1-(1H- pyrrolo[2,3- b]pyridin-4-yl)- piperidin-4- ylamine | Method GP4B using compounds A3-13 and 4- chloro-1H- pyrrolo[2,3- b]pyridine (Preparation 3) | 1H NMR (Me-d3- OD): 7.94(1H, d), 7.58(1H, d), 7.54 (1H, d), 7.24(1H, dd), 7.20(1H, d), 6.57(1H, d), 6.55 (1H, d), 3.80-3.67 (4H, m), 2.64-2.50 (2H, m), 2.10-2.00 (2H, m) | M/z: 367 |
| 21 | 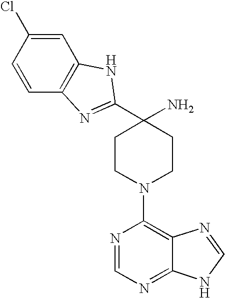 | 4-(6-chloro-1H- benzoimidazol- 2-yl)-1-(9H- purin-6-yl)- piperidin-4- ylamine | Method GP4B using compounds A3-13 and 6- chloro-9H- purine | 1H NMR (Me-d3- OD): 8.24(1H, s), 8.02(1H, s), 7.55 (1H, d), 7.51(1H, d), 7.21(1H, dd), 4.77-4.55(2H, m), 4.36-4.18(2H, m), 2.53-2.38(2H, m), 2.04-1.90(2H, m) | M/z: 369 |
| 22 | 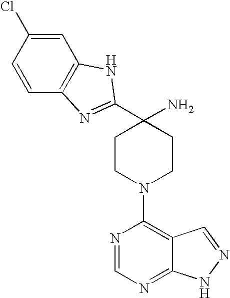 | 4-(6-chloro-1H- benzoimidazol- 2-yl)-1-(1H- pyrazolo[3,4- d]pyrimidin-4- yl)-piperidin-4- ylamine | Method GP4B using compounds A3-13 and 4- chloro-1H- pyrazolo[3,4- d]pyrimidine | 1H NMR (Me-d3- OD): 8.27(2H, s), 7.55(1H, d), 7.51 (1H, d), 7.21(1H, dd), 4.41-4.27(2H, m), 4.16-4.04(2H, m), 2.52-2.40(2H, m), 2.07-1.96(2H, m) | M/z: 369 |
| 23 | 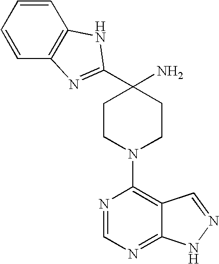 | 4-(1H- benzoimidazol- 2-yl)-1-(1H- pyrazolo[3,4- d]pyrimidin-4- yl)-piperidin-4- ylamine | Method GP4B using compounds A3-12 and 4- chloro-1H- pyrazolo[3,4- d]pyrimidine | 1H NMR (DMSO- d6): 9.81-9.42(2H, br s), 9.11(1H, br s), 8.65(1H, s), 7.76-7.66(2H, m), 7.41-7.30(2H, m), 4.90-4.42(2H, br m), 4.21-4.00(2H, br m), 3.07-2.86 (2H, br m), 2.67- 2.42(2H, br m) | M/z: 335 |
| 24 | 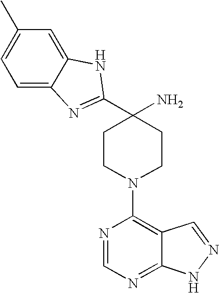 | 4-(6-Methyl-1H- benzoimidazol- 2-yl)-1-(1H- pyrazolo[3,4- d]pyrimidin-4- yl)-piperidin-4- ylamine(acetate salt) | Method GP4A using compounds A3-5 and 4- chloro-1H- pyrazolo[3,4- d]pyrimidine | ¹H NMR (400 MHz, Me-d₃-OD): 8.29(2H, s), 7.50 (1H, d), 7.41(1H, s), 7.12(1H, d), 4.44-4.29(2H, m), 4.12-3.98(2H, m), 2.77-2.62(2H, m), 2.47(3H, s), 2.21- 2.07(2H, m), 2.00 (3H, s). | MS: [M + H]⁺ 349 |
| 25 | 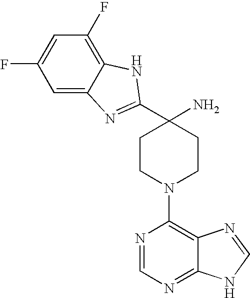 | 4-(5,7-Difluoro- 1H- benzoimidazol- 2-yl)-1-(9H- purin-6-yl)- piperidin-4- ylamine | Method GP4A using compounds A3-7 and 6- chloro-9H- purine | ¹H NMR (400 MHz, Me-d₃-OD): 8.27(1H, s), 8.05 (1H, s), 7.14(1H, dd), 6.95-6.83(1H, m), 5.50(13H, s), 4.56(2H, s), 4.39 (2H, s), 2.72-2.59 (2H, m), 2.15-2.03 (2H, m), 2.03-1.81 (3H, m). | MS: [M + H]⁺ 371 |
| 26 | 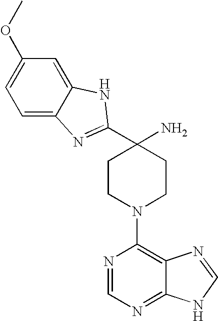 | 4-(6-Methoxy- 1H- benzoimidazol- 2-yl)-1-(9H- purin-6-yl)- piperidin-4- ylamine | Method GP4A using compounds A3-8 and 4- chloro-7H- pyrrolo[2,3- d]pyrimidine | ¹H NMR (400 MHz, Me-d₃-OD): 8.23(1H, s), 8.02 (1H, s), 7.43(1H, d), 7.06(1H, d), 6.86(1H, dd), 4.60 (2H, s), 4.32(2H, s), 3.83(3H, s), 2.53-2.39(2H, m), 1.96(2H, d). | MS: [M + H]⁺ 365 |
| 27 | 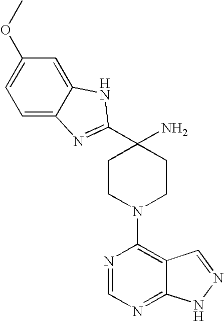 | 4-(6-Methoxy- 1H- benzoimidazol- 2-yl)-1-(1H- pyrazolo[3,4- d]pyrimidin-4- yl)-piperidin-4- ylamine | Method GP4A using compounds A3-8 and 4- chloro-1H- pyrazolo[3,4- d]pyrimidine | ¹H NMR (400 MHz, Me-d₃-OD): 8.27(2H, d), 7.44 (1H, d), 7.06(1H, d), 6.87(1H, dd), 4.28(2H, s), 4.21- 4.06(2H, m), 3.84 (3H, s), 2.54-2.40 (2H, m), 2.09-1.94 (2H, m). | MS: [M + H]⁺ 365 |
| 28 | 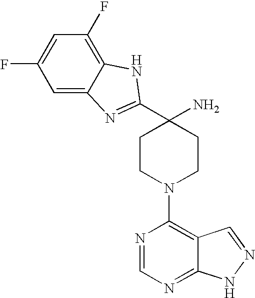 | 4-(5,7-Difluoro- 1H- benzoimidazol- 2-yl)-1-(1H- pyrazolo[3,4- d]pyrimidin-4- yl)-piperidin-4- ylamine | Method GP4A using compounds A3-7 and 4- chloro-1H- pyrazolo[3,4- d]pyrimidine | ¹H NMR (400 MHz, Me-d₃OD): 8.26(2H, d), 7.09 (1H, dd), 6.90-6.77 (1H, m), 4.34(2H, d), 4.18-4.02(2H, m), 2.54-2.38(2H, m), 2.11-1.94(3H, m). | MS: [M + H]⁺ 371 |
| 29 | 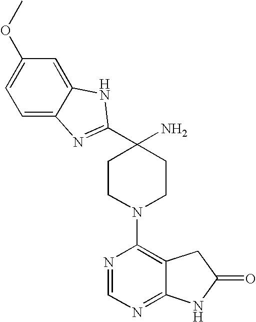 | 4-[4-Amino-4- (6-methoxy-1H- benzoimidazol- 2-yl)-piperidin- 1-yl]-5,7- dihydro- pyrrolo[2,3- d]pyrimidin-6- one (acetate salt) | Method GP4A using compounds A3-8 and 4- chloro-5,7- dihydro- pyrrolo[2,3- d]pyrimidin-6- one | ¹H NMR (400 MHz, Me-d₃-OD): 8.21(1H, s), 7.46 (1H, d), 7.08(1H, d), 6.90(1H, dd), 3.89(2H, d), 3.85 (3H, s), 3.78(2H, d), 2.53-2.41(2H, m), 2.00(3H, s), 1.98-1.88(2H, m). | MS: [M + H]⁺ 380 |
| 30 | 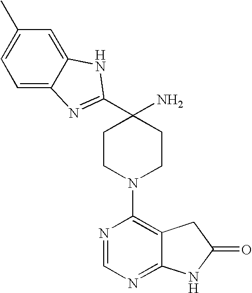 | 4[4-Amino-4- (6-methyl-1H- benzoimidazol- 2-yl)-piperidin- 1-yl]-5,7- dihydro- pyrrolo[2,3- d]pyrimidin-6- one | Method GP4A using compounds A3-5 and 4- chloro-5,7- dihydro- pyrrolo[2,3- d]pyrimidin-6- one | ¹H NMR (400 MHz, Me-d₃-OD): 8.20(1H, s), 7.44 (1H, d), 7.35(1H, s), 7.07(1H, d), 4.02-3.85(4H, m), 3.77(1H, d), 2.46 (3H, s), 2.45-2.31 (2H, m), 1.97-1.84 (2H, m). | MS: [M + H]⁺ 364 |
| 31 | 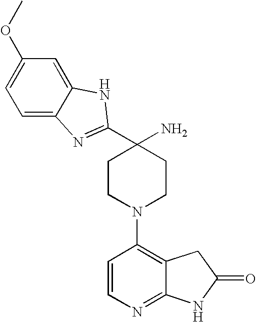 | 4-[4-Amino-4- (6-methoxy-1H- benzoimidazol- 2-yl)-piperidin- 1-yl]-1,3- dihydro- pyrrolo[2,3- b]pyridin-2-one | Method GP4A using compounds A3-8 and 4- fluoro-1,3- dihydro- pyrrolo[2,3- b]pyridin-2- one | ¹H NMR (400 MHz, Me-d₃-OD): 7.80(1H, d), 7.50- 7.41(1H, m), 7.07 (1H, d), 6.89(1H, dd), 6.61(1H, d), 3.97-3.87(1H, m), 3.85(3H, s), 3.76- 3.62(3H, m), 3.62- 3.49(2H, m), 2.55- 2.41(2H, m), 2.06- 1.89(2H, m). | MS: [M + H]⁺ 379 |
| 32 | 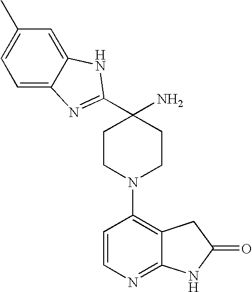 | 4-[4-Amino-4- (6-methyl-1H- benzoimidazol- 2-yl)-piperidin- 1-yl]-1,3- dihydro- pyrrolo[2,3- b]pyridin-2-one | Method GP4A using compounds A3-5 and 4- fluoro-1,3- dihydro- pyrrolo[2,3- b]pyridin-2- one | ¹H NMR (400 MHz, Me-d₃-OD): 8.38(1H, s), 7.82 (1H, d), 7.54-7.44 (1H, m), 7.44-7.36 (1H, m), 7.12(1H, d), 6.61(1H, d), 3.83-3.66(4H, m), 3.53-3.40(2H, m), 2.72-2.58(2H, m), 2.48(3H, s), 2.15- 1.89(2H, m). | MS: [M + H]⁺ 363 |

### EXAMPLE 33

### [4-(1H-benzoimidazol-2-yl)-1-(7H-pyrrolo[2,3-d]pyrimidin-4-yl)-piperidin-4-yl]-methyl-amine

### 33A. 4-(1H-benzoimidazol-2-yl)-4-tert-butoxycarbonylamino-piperidine-1-carboxylic acid tert-butyl ester

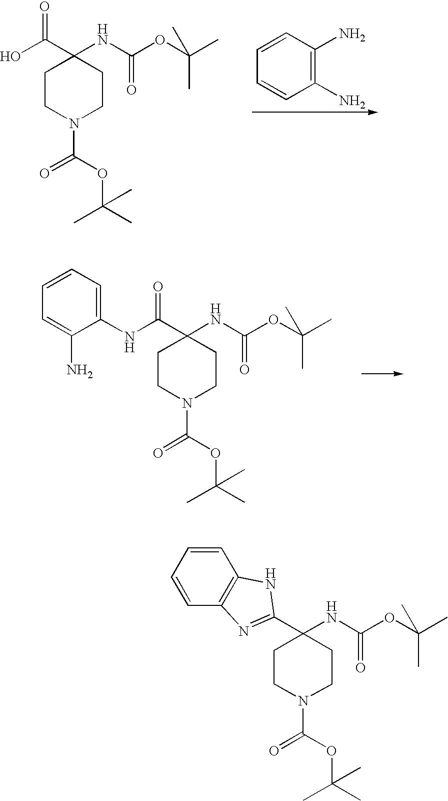

The title compound was prepared from 4-tert-butoxycarbonylamino-piperidine-1,4-dicarboxylic acid mono-tert-butyl ester (3 g, 8.71 mmol) according to General Procedure GP2 and employing cyclisation step GP2C using benzene-1,2-diamine Yield: yellow solid (2.15 g, 97%).

### 33B. 2-(1-tert-butoxycarbonyl-4-tert-butoxycarbonylamino-piperidin-4-yl)benzoimidazole-1-carboxylic acid tert-butyl ester

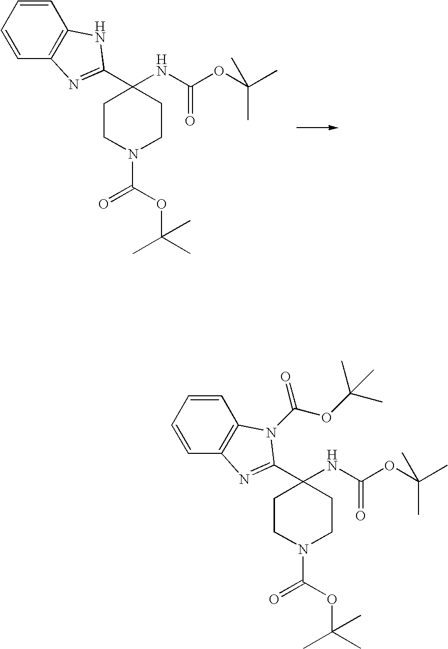

4-(1H-benzoimidazol-2-yl)-4-tert-butoxycarbonylamino-piperidine-1-carboxylic acid tert-butyl ester (1.55 g, 3.72 mmol) was suspended in dichloromethane. 4-dimethylaminopyridine (0.0454 g, 0.372 mmol) and triethylamine (0.507 mL, 3.72 mmol) were added followed by di-tert-butyl dicarbonate (1.62 g, 7.44 mmol). The reaction mixture was stirred at room temperature for 16 hours. The reaction was diluted with dichloromethane and the organic was washed with brine. The organic was separated off and concentrated in vacuo. The residue was purified by flash column chromatography on silica gel eluting with 70/30 petroleum ether/ethyl acetate to afford the title compound as a yellow foam (1.67 g, 87%).

### 33C. 2-[1-tert-butoxycarbonyl-4-(tert-butoxycarbonyl-methyl-amino)-piperidin-4-yl]-benzoimidazole-1-carboxylic acid tert-butyl ester

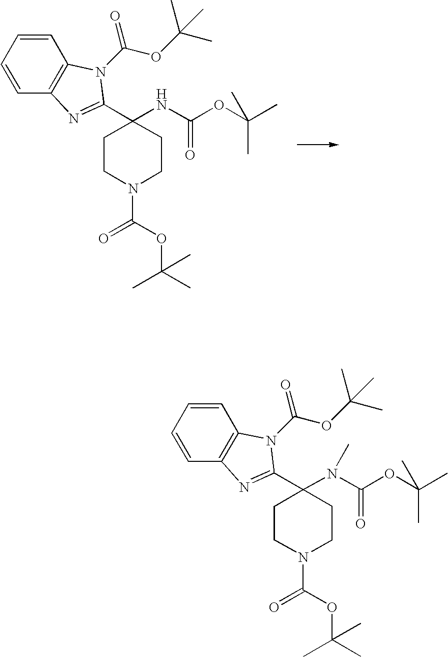

2-(1-tert-butoxycarbonyl-4-tert-butoxycarbonylamino-piperidin-4-yl) benzoimidazole-1-carboxylic acid tert-butyl ester (0.5 g, 0.967 mmol) was dissolved in anhydrous N,N-dimethylformamide (3.6 mL) under nitrogen. The solution was cooled to 0° C. and sodium hydride (0.0472 g, 1.18 mmol, 60% w/w suspension in oil) was added portion-wise. Stirring was continued at 0° C. for 20 minutes and methyl iodide was added drop-wise. The reaction was stirred at 0° C. for 30 minutes and then at room temperature for 16 hours. Water (20 mL) was added and this layer was extracted with ethyl acetate. The organic phase was separated and washed again with water and then brine. The organic phases were separated, dried (MgSO₄) and concentrated in vacuo to afford the title compound (0.529 g, >100%). The product was used without further purification.

### 33D. [4-(1H-benzoimidazol-2-yl)-piperidin-4-yl -methyl-amine

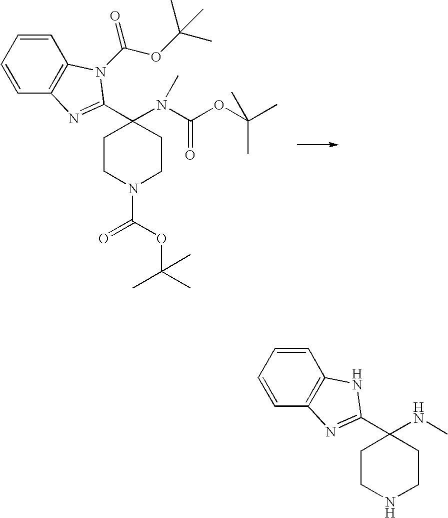

The title compound was prepared using General Procedure 3. Yield: colourless gum (0.109 g, 47%).

### 33E. [4-(1H-benzoimidazol-2-yl)-1-(7H-pyrrolo[2,3-d]pyrimidin-4-yl)-piperidin-4-yl]-methyl-amine

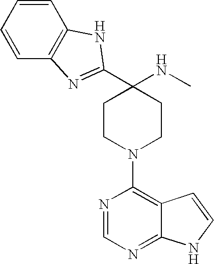

The title compound was prepared by reacting the product of Example 24D with 4-chloro-7H-pyrrolo[2,3-d]pyrimidine (Preparation 1) according to General Procedure GP4B.

M/z: 348; ¹H NMR (CDCl₃): 8.25 (1H, s), 7.68 (1H, d), 7.33-7.13 (3H, m), 7.01 (1H, d), 6.50 (1H, d), 4.22-4.04 (4H, m), 4.04 (3H, s), 2.72-2.56 (2H, m), 2.00-1.8 (2H, m).

### EXAMPLE 34

### C-[4-(1H-Benzoimidazol-2-yl)-1-(7H-pyrrolo[2,3-d]pyrimidin-4-yl)-piperidin-4-yl]-methylamine

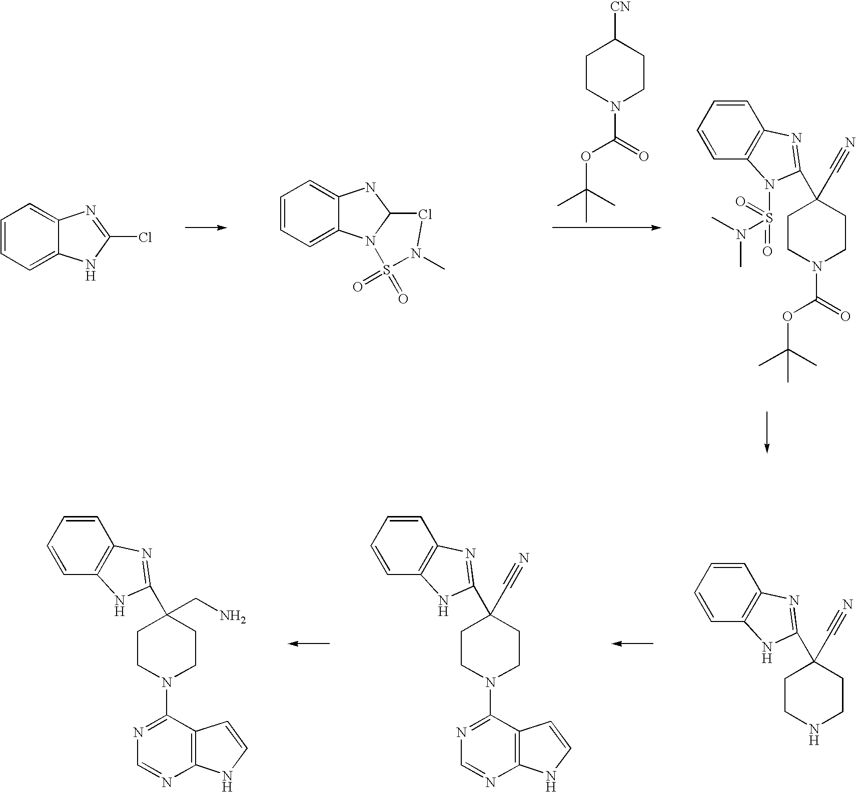

### 34A. 2-Chloro-benzoimidazole-1-sulphonic acid dimethylamide

To a solution of 2-chloro-1H-benzoimidazole (5.0 g, 33 mmol) and dimethylsulphamoyl chloride (3.5 ml, 33 mmol) in dimethylformamide (50 ml) was added 1,4-diazabicyclo[2.2.2]octane (DABCO, 3.7 g, 33 mmol) whilst stirring at room temperature. After 16 hours, water was added and the reaction mixture was extracted with ethyl acetate (200 ml). The organic layer was washed with lithium chloride solution (10% aqueous, ×2) and concentrated brine solution (×1), dried (MgSO₄) and concentrated in vacuo. The resultant oil was purified on a silica Biotage column eluting 0-30% ethyl acetate/petrol, furnishing a colourless oil (7.4 g, 87%).

### 34B. 4-Cyano-4-(1-dimethylsulphamoyl-1H-benzoimidazol-2-yl)-piperidine-1-carboxylic acid tert-butyl ester

2-Chloro-benzoimidazole-1-sulphonic acid dimethylamide was reacted with 1-tert-butoxycarbonyl-4-cyanopiperidine (1 equivalent) in THF in the presence of sodium hexamethyldisilazide at room temperature according to the protocols described in Klapars et al., J. Org. Chem. 2005, 70(24), 10186-10189 to give the title compound as a colourless oil (10.66 g, 86%).

### 34C. 4-(1H-Benzoimidazol-2-yl)-1-(7H-pyrrolo[2,3-d]pyrimidin-4-yl)-piperidine-4-carbonitrile

To a solution of 4-cyano-4-(1-dimethylsulphamoyl-1H-benzoimidazol-2-yl)-piperidine-1-carboxylic acid tert-butyl ester (3.3 g, 7 6 mmol) in dichloromethane (10 ml) was added trifluoroacetic acid (6 ml). The reaction was stirred at room temperature for 30 minutes, extra acid (1 ml) was added then, after a further 10 minutes, the reaction mixture was concentrated in vacuo and re-concentrated from dichloromethane and methanol. To the resulting crude deprotected intermediate was added 4-chloro-(7H-pyrrolo[2,3-d]pyrimidine (1.3 g, 8.36 mmol), triethylamine (4.5 ml) and N-methylpyrrolidine (4.5 ml). The mixture was heated to 160° C. for 90 minutes in a sealed tube using a microwave heater. An extra portion of triethylamine was added (1.5 ml) and the reaction mixture was irradiated again for 90 minutes. Ethyl acetate was then added and the reaction mixture was filtered under suction. The liquors were removed and then the solid was washed with methanol. The organic fractions were purified on a silica Biotage column eluting with a gradient of DMAW 120 to DMAW90 furnishing the product as an oil.

### 34D. C-[4-(1H-Benzoimidazol-2-yl)-1-(7H-pyrrolo[2,3-d]pyrimidin-4-yl)-piperidin-4-yl]-methylamine

A mixture of the nitrile product of Example 34C and Raney nickel (a suspension in water) in an organic solvent (e.g. N,N-dimethylformamide, ethanol and/or tetrahydrofuran), and optionally with added base (e.g. aqueous sodium hydroxide solution or methanolic ammonia), was hydrogenated at atmospheric pressure and at room temperature for 18-96 hours. To achieve full reduction, it was occasionally required to refresh the catalyst during this time. When the requisite volume of hydrogen had been consumed, the reaction mixture was filtered under suction using either a Celite™ pad or glass fibre filter paper before concentrating to give the crude product which was purified by silica column chromatography eluting with mixtures of dichloromethane, methanol, acetic acid and water to give the title compound as the diacetate salt.

M/z: 348; ¹H NMR (Me-d₃-OD) 8.18 (1H, s), 7.62 (2H, m), 7.30 (2H, m), 7.18 (1H, d), 6.68 (1H, d), 4.45 (2H, m), 3.70 (2H, m), 2.60 (2H, m), 2.02 (2H, m), 1.99 (6H, s)

### EXAMPLE 35

### [4-(1H-Benzoimidazol-2-yl)-1-(7H-pyrrolo[2,3-d]pyrimidin-4-yl)-piperidin-4-yl]-methanol

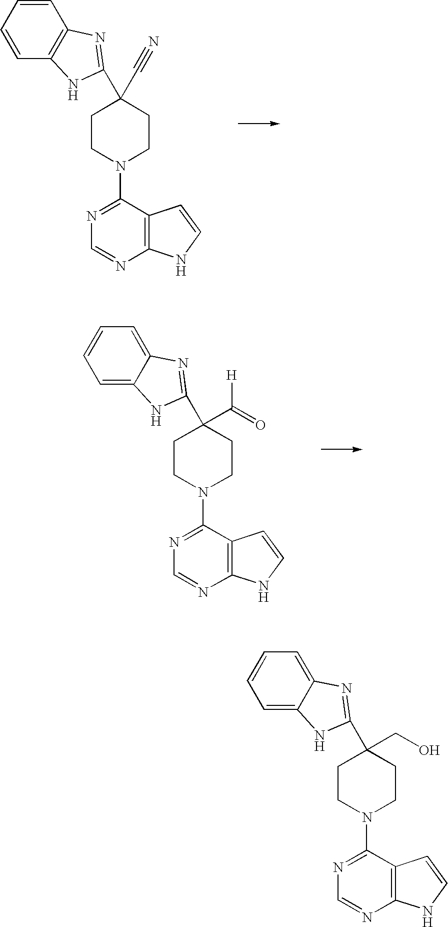

### 35A. 4-(1H-Benzoimidazol-2-yl)-1-(7H-pyrrolo[2,3-d]pyrimidin-4-yl)-piperidine-4-carbaldehyde

4-(1H-Benzoimidazol-2-yl)-1-(9H-purin-6-yl)-piperidine-4-carbonitrile (Example 34C) was subjected to hydrogenation over Raney nickel as described in Example 34D to give a mixture of the corresponding aminomethyl analogue and the title aldehyde (formed by hydrolysis of the imine arising from partial reduction of the nitrile).

### 35B. [4-(1H-Benzoimidazol-2-yl)-1-(7H-pyrrolo[2,3-d]pyrimidin-4-yl)-piperidin-4-yl]-methanol (acetate salt)

To a solution of the aldehyde of Example 35A in tetrahydrofuran was added sodium triacetoxyborohydride (1 to excess equivalents). The mixture was stirred at room temperature overnight then water was added and the mixture extracted with ethyl acetate. After drying the solution and concentrating, the crude material was purified on a silica Biotage column, eluting with mixtures of dichloromethane, methanol, acetic acid and water to give the title compound as the acetate salt.

M/z: 349; ¹H NMR (Me-d₃-OD) 8.13 (1H, s), 7.60 (2H, dd), 7.23 (2H, dd), 7.13 (1H, d), 6.67 (1H, d), 4.61 (2H, m), 3.73 (2H, s), 3.43 (2H, m), 2.60 (2H, m), 2.00 (8H, m)

### EXAMPLE 36

### C-[4-Benzooxazol-2-yl-1-(7H-pyrrolo[2,3-d]pyrimidin-4-yl)-piperidin-4-yl]-methylamine (diacetate salt)

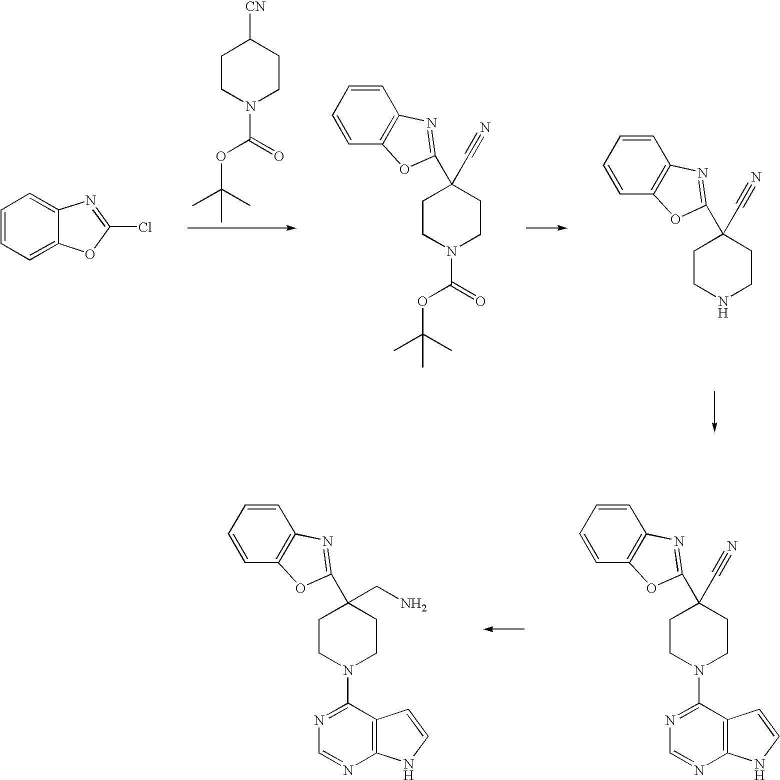

### 36A. 4-Benzooxazol-2-yl-4-cyano-piperidine-1-carboxylic acid tert-butyl ester

The title compound was prepared from 2-chlorobenzooxazole and 1-tert-butoxycarbonyl-4-cyanopiperidine using the general procedure described in Example 34B.

### 36B. 4-Benzooxazol-2-yl-piperidine-4-carbonitrile

The compound of Example 36A was deprotected to give the title compound using General Procedure GP3 described above.

### 36C. 4-Benzooxazol-2-yl-1-(7H-pyrrolo[2,3-d]pyrimidin-4-yl)-piperidine-4-carbonitrile

The product of Example 36B was reacted with 4-chloro-7H-pyrrolo[2,3-d]pyrimidine under the conditions described in General Procedure GP4A above to give the title compound.

### 36D. C-[4-Benzooxazol-2-yl-1-(7H-pyrrolo[2,3-d]pyrimidin-4-yl)-piperidin-4-yl]-methylamine (diacetate salt)

The product of Example 36C was subjected to hydrogenation over Raney nickel using the conditions described in Example 34D to give the title compound as the diacetate salt.

M/z: 349; ¹H NMR (Me-d₃-OD) 8.18 (1H, s), 7.78 (1H, d), 7.69 (1H, d), 7.42 (2H, m), 7.18 (1H, d), 6.67 (1H, d), 4.62 (2H, m), 3.56 (2H, m), 2.62 (2H, m), 1.99 (8H, m)

### EXAMPLE 37

### 4-Benzooxazol-2-yl-1-(7H-pyrrolo[2,3-d]pyrimidin-4-yl)-piperidin-4-ylamine

### 37A. 4-tert-butoxycarbonylamino-4-(2-hydroxy-phenylcarbamoyl)-piperidine-1-carboxylic acid tert-butyl ester

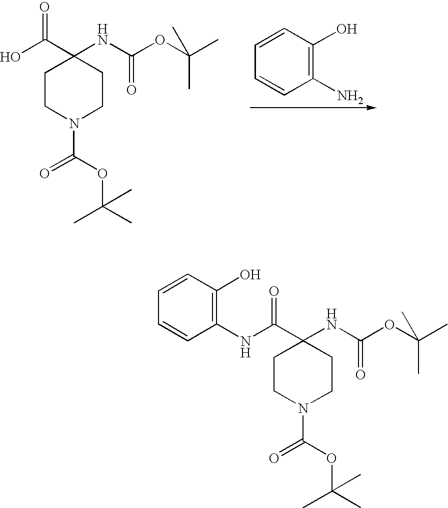

The title compound was prepared from 4-tert-butoxycarbonylamino-piperidine-1,4-dicarboxylic acid mono-tert-butyl ester (1 g, 2.90 mmol) and 2-aminophenol using General Procedure GP2 and the acetic acid cyclisation method GP2B above. Yield: 0.698 g (55%).

### 37B. 4-benzooxazol-2-yl-4-tert-butoxycarbonylamino-piperidine-1-carboxylic acid tert-butyl ester

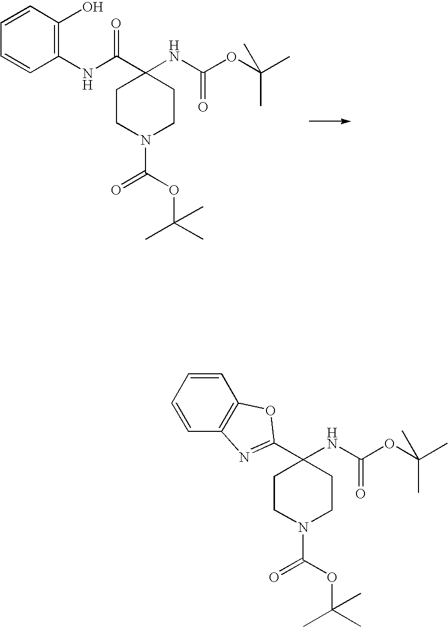

4-tert-butoxycarbonylamino-4-(2-hydroxy-phenylcarbamoyl)-piperidine-1-carboxylic acid tert-butyl ester (0.646 g, 1.48 mmol) was dissolved in anhydrous tetrahydrofuran (4.5 mL) under nitrogen and triphenylphosphine (0.854 g, 3.26 mmol) was added. The solution was cooled to 0° C. and diethylazodicarboxylate (0.513 mL, 3.26 mmol) was added drop-wise.

The reaction mixture was then stirred at room temperature for 16 hours. Solvent was removed in vacuo and the residue was partitioned between ethyl acetate and brine. The organic phase was separated and washed with aqueous 2M NaOH, separated again, dried (MgSO₄) and the solvent was then removed in vacuo. The residue was further purified by flash column chromatography on silica gel eluting with 70/30 petroleum ether/ethyl acetate to afford the title compound as an orange solid (0.493 g, 80%).

### 37C. 4-Benzooxazol-2-yl-piperidin-4-ylamine

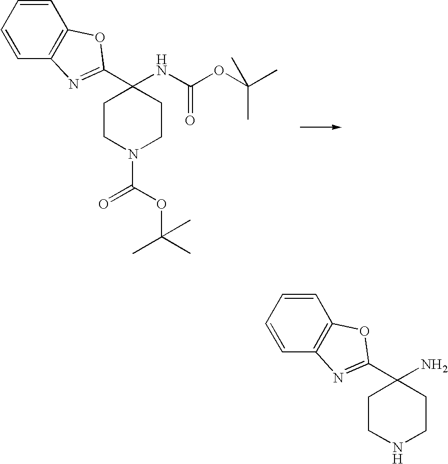

The compound of Example 37B was deprotected to give the title compound using General Procedure GP3 described above. Yield: yellow oil (0.1 g, 39%).

### 37D. 4-Benzooxazol-2-yl-1-(7H-pyrrolo[2,3-d]pyrimidin-4-yl)-piperidin-4-ylamine

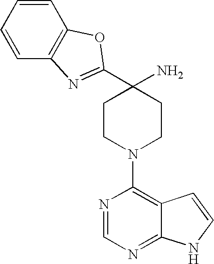

The product of Example 37C was reacted with 4-chloro-7H-pyrrolo[2,3-d]pyrimidine under the conditions described in General Procedure GP4A to give the title compound.

M/z: 335; ¹H NMR (DMSO-d6): 11.68 (1H, s), 8.16 (1H, s), 7.77-7.65 (2H, m), 7.43-7.31 (2H, m), 7.19 (1H, t), 6.64 (1H, d), 4.15-3.93 (4H, m), 2.43 (1H, br s), 2.31-2.17 (2H, m), 1.95-1.84 (2H, d)

### EXAMPLE 38

### 6-(4-amino-4-benzooxazol-2-yl-piperidin-1-yl)-7,9-dihydro-purin-8-one

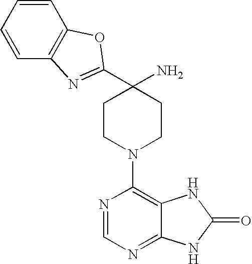

4-Benzooxazol-2-yl-piperidin-4-ylamine (Example 37C) was reacted with 6-chloro-7,9-dihydro-purin-8-one under the conditions described in General Procedure GP4A to give the title compound.

M/z: 352; ¹H NMR (DMSO-d6): 11.44 (1H, br s), 10.74 (1H, br s), 8.10 (1H, s), 7.78-7.66 (2H, m), 7.44-7.31 (2H, m), 3.83-3.66 (4H, m), 2.29-2.15 (2H, m), 1.94-1.80 (2H, m)

### EXAMPLE 39

### 4-(5-Phenyl-1H-imidazol-2-yl)-1-(7H-pyrrolo[2,3-di]pyrimidin-4-yl)-piperidin-4-ylamine

### 39A. 4-tert-butoxycarbonylamino-4-(5-phenyl-1H-imidazol-2-yl)-piperidine-1-carboxylic acid tert-butyl ester

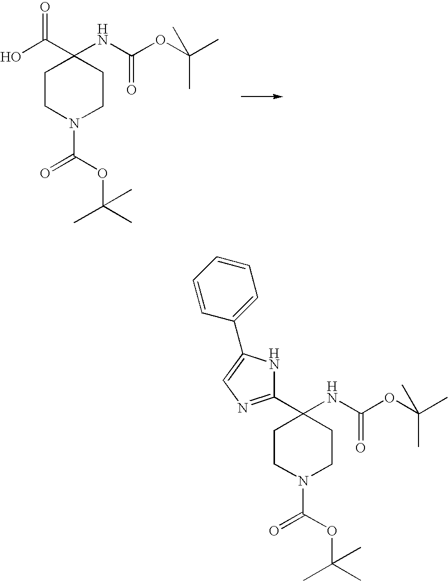

4-tert-butoxycarbonylamino-piperidine-1,4-dicarboxylic acid mono-tert-butyl ester (3 g, 8.71 mmol) and caesium carbonate (1.42 g, 4.36 mmol) were dissolved in anhydrous methanol (26 mL) and the solution was stirred under nitrogen for 2 hours. Solvent was removed in vacuo and the residue was re-dissolved in N,N-dimethylformamide (35 mL). 2-bromoacetophenone (1.73 g, 8.71 mmol) was added and the mixture was stirred under nitrogen for 2 hours. Solvent was removed in vacuo and the residue was re-dissolved in xylenes (44 mL). Ammonium acetate (13.43 g, 174 2 mmol) was added and the mixture was heated at 135° C. with stirring for 4 hours. The reaction mixture was then allowed to stand at room temperature for 48 hours. The reaction mixture was diluted with ethyl acetate and the organic phase was washed with water followed by brine. The organic phase was separated, dried (MgSO₄) then concentrated in vacuo to afford the title compound as a yellow solid (4.80 g, >100%). The product was used without any further purification.

### 39B. 4-(5-phenyl-1H-imidazol-2-yl)-piperidin-4-ylamine

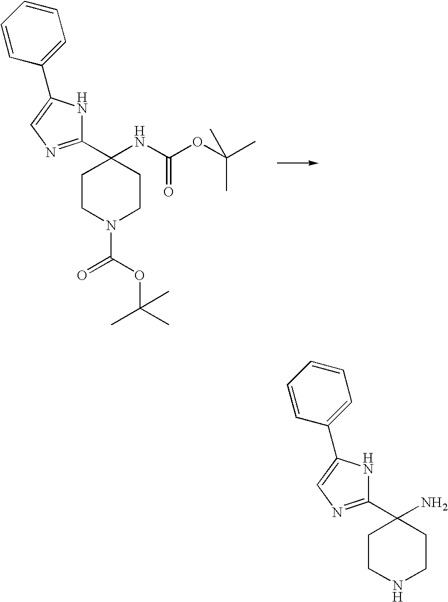

The title compound was prepared by deprotecting the compound of Example 39A using General Procedure GP3 above. Yield: 1.35 g (64%).

### 39C. 4-(5-Phenyl-1H-imidazol-2-yl)-1-(7H-pyrrolo[2,3-d]pyrimidin-4-yl)-piperidin-4-ylamine

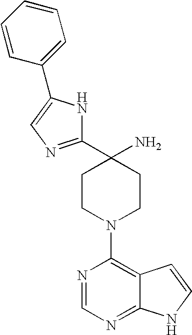

The title compound was prepared by reacting the product of Example 39B with 4-chloro-7H-pyrrolo[2,3-d]pyrimidine under the conditions described in General Procedure GP4B.

M/z: 360; ¹H NMR (Me-d3-OD): 8.16 (1H, s), 7.70 (2H, d), 7.38 (3H, t), 7.32 (1H, s), 7.24 (1H, t), 7.15 (1H, d), 6.67 (1H, d), 4.20-4.04 (4H, m), 2.53-2.39 (2H, m), 2.00-1.86 (2H, m)

### EXAMPLE 40

### 4-(5-Phenyl-oxazol-2-yl)-1-(7H-pyrrolo[2,3-d]pyrimidin-4-yl)-piperidin-4-ylamin

### 40A. 4-chlorocarbonyl-4-(9H-fluoren-9-ylmethoxycarbonylamino)-piperidine-1-carboxylic acid 9H-fluoren-9-ylmethyl ester

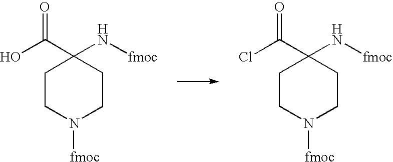

4-(9H-fluoren-9-ylmethoxycarbonylamino)-piperidine-1,4-dicarboxylic acid mono-(9H-fluoren-9-ylmethyl)ester (3 g, 5.09 mmol) was suspended in thionyl chloride (20 mL). After 20 minutes an additional quantity of thionyl chloride (10 mL) was added. Stirring was continued for 2 hours at room temperature. The reaction mixture was concentrated in vacuo and was azeotroped twice with toluene to afford the title compound as a yellow foam. The product was used without further characterization.

### 40B. 4-(9H-fluoren-9-ylmethoxycarbonylamino)-4-(2-hydroxy-2-phenyl-ethylcarbamoyl)-piperidine-1-carboxylic acid 9H-fluoren-9-ylmethyl ester

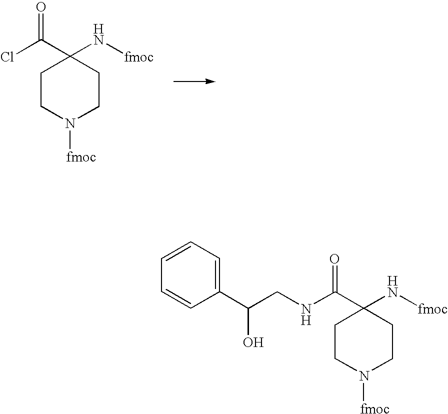

2-Amino-1-phenyl-ethanol (0.698 g, 5.09 mmol) was suspended in anhydrous dichloromethane (15.3 mL) and triethylamine (0.745 mL, 5.34 mmol) was added. This solution was cooled to 0° C. in an ice bath. 4-Chlorocarbonyl-4-(9H-fluoren-9-ylmethoxycarbonylamino)-piperidine-1-carboxylic acid 9H-fluoren-9-ylmethyl ester (assumed to be 3.09 g, 5.09 mmol) was dissolved in anhydrous dichloromethane (10 mL) and the resulting solution was added in a drop-wise manner to the solution of 2-amino-1-phenyl-ethanol. The ice bath was removed and the reaction mixture was stirred at room temperature for 16 hours. The reaction mixture was diluted with dichloromethane and the organic layer was washed with brine. The aqueous layer was separated and extracted with dichloromethane. The organic extracts were combined, dried (MgSO₄) and concentrated in vacuo. The residue was purified by flash column chromatography on silica gel eluting with 70/30 ethyl acetate/petroleum ether to afford the title compound as a white foam (2.34 g, 65%).

### 40C. 4-(9H-fluoren-9-ylmethoxycarbonylamino)-4-(2-oxo-2-phenyl-ethylcarbamoyl)-piperidine-1-carboxylic acid 9H-fluoren-9-ylmethyl ester

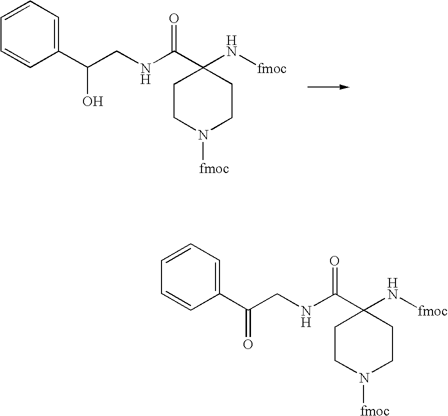

4-(9H-Fluoren-9-ylmethoxycarbonylamino)-4-(2-hydroxy-2-phenyl-ethylcarbamoyl)-piperidine-1-carboxylic acid 9H-fluoren-9-ylmethyl ester (2.34 g, 3.31 mmol) was dissolved in anhydrous dichloromethane (40.6 mL) and pyridinium chlorochromate (1.43 g, 6.61 mmol) was added. The reaction was stirred at room temperature for 16 hours. The reaction mixture was diluted with dichloromethane/methanol and was filtered through Celite in vacuo. The filtrate was concentrated in vacuo. The residue was diluted with dichloromethane and the organic phase was washed twice with brine. The organic phase was separated, dried (MgSO₄) and concentrated in vacuo. The residue was purified by flash column chromatography on silica gel eluting with 60/40 ethyl acetate/petroleum ether to afford the title compound as a white foam (1.85 g, 79%).

### 40D. 4-(9H-fluoren-9-ylmethoxycarbonylamino)-4-(5-phenyl-oxazol-2-yl)-piperidine-1-carboxylic acid 9H-fluoren-9-ylmethyl ester

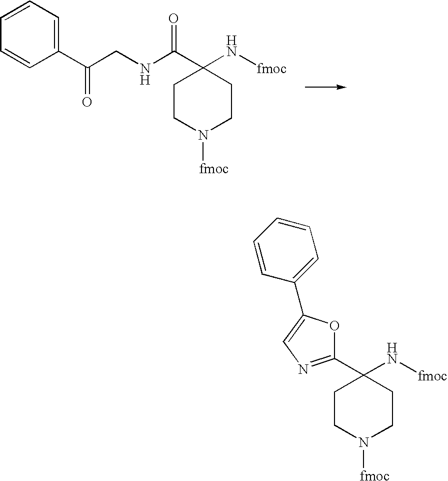

4-(9H-Fluoren-9-ylmethoxycarbonylamino)-4-(2-oxo-2-phenyl-ethylcarbamoyl)-piperidine-1-carboxylic acid 9H-fluoren-9-ylmethyl ester (1.85 g, 2.62 mmol) was dissolved in phosphorus (III) oxychloride (19 mL) and the reaction was heated at 110° C. with stirring for 3 hours. Upon cooling, the reaction was quenched by drop-wise addition of ice-water (with vigorous stirring). Dichloromethane was added and the vigorous stirring was continued. The mixture was transferred to a separating funnel and the aqueous phase was separated. The aqueous phase was extracted again with dichloromethane, and the organic extracts were combined, dried (MgSO₄) and concentrated in vacuo. The residue was purified by flash column chromatography on silica gel eluting with 60/40 petroleum ether/ethyl acetate followed by 90/10 ethyl acetate/methanol to afford the title compound as a yellow foam.

### 40E. 4-(5-phenyl-oxazol-2-yl)-piperidin-4-ylamine

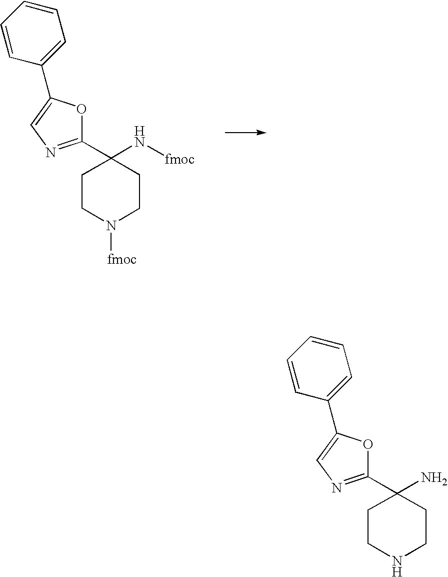

4-(9H-Fluoren-9-ylmethoxycarbonylamino)-4-(5-phenyl-oxazol-2-yl)-piperidine-1-carboxylic acid 9H-fluoren-9-ylmethyl ester (assumed to be 1.22 g, 2.62 mmol) was dissolved in dichloromethane (28 mL) and piperidine (5.18 mL, 52.4 mmol) was added followed by 1,8-diazabicyclo[5.4.0]undec-7-ene (10 drops). The reaction mixture was stirred at room temperature for 1 hour and then concentrated in vacuo. The residue was purified on an acidic ion-exchange column to afford the title compound as a yellow gum (0.084 g, 13%).

### 40F. 4-(5-Phenyl-oxazol-2-yl)-1-(7H-pyrrolo[2,3-d]pyrimidin-4-yl)-piperidin-4-ylamine

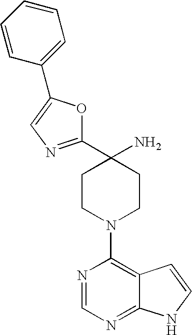

The title compound was prepared by reacting the product of Example 40E with 4-chloro-7H-pyrrolo[2,3-d]pyrimidine under the conditions described in General Procedure GP4B.

M/z: 361; ¹H NMR (Me-d3-OD): 8.17 (1H, s), 7.75 (2H, d), 7.46 (3H, t), 7.37 (1H, t), 7.15 (1H, d), 6.67 (1H, d), 4.41-4.28 (2H, m), 3.96-3.81 (2H, m), 2.57-2.43 (2H, m), 2.07-1.93 (2H, m)

### EXAMPLE 41

### 4-(5-phenyl-1H-imidazol-2-yl)-1-(1H-pyrrolo[2,3-b]pyridin-4-yl)-piperidin-4-ylamine

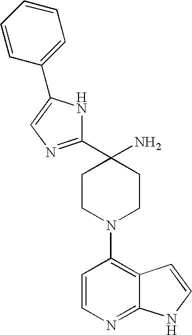

The title compound was prepared by reacting the product of Example 39B with 4-chloro-1H-pyrrolo[2,3-b]pyridine (Preparation 2) under the conditions described in General Procedure GP4B.

M/z: 359; ¹H NMR (DMSO-d6): 11.35 (1H, br s), 7.93 (1H, d), 7.76 (2H, d), 7.53 (1H, br s), 7.33 (2H, t), 7.21 (1H, d), 7.18 (1H, t), 6.48 (1H, d), 6.44 (1H, d), 3.71-3.51 (4H, m), 2.47-2.30 (2H, m), 1.93-1.75 (2H, m)

### EXAMPLE 42

### 4-(4-Phenyl-1H-benzoimidazol-2-yl)-1-(7H-pyrrolo[2,3-d]pyrimidin-4-yl)-piperidin-ylamine

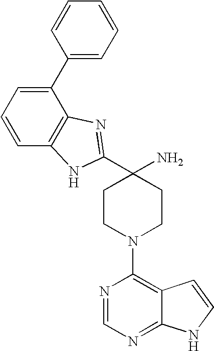

A mixture of 4-(4-chloro-1H-benzoimidazol-2-yl)-1-(7H-pyrrolo[2,3-d]pyrimidin-4-yl)-piperidin-4-ylamine (Example 14) (50 mg; 0.13 mmol), phenylboronic acid (35 mg; 2 equiv), potassium carbonate (115 mg; 6 equiv) and bis(tri-t-butyl-phosphine)palladium (0) (4 mg; 5 mol%) in ethanol (1 ml), toluene (1 ml), water (1 ml) was heated at 135° C. for 60 minutes in a CEM Discover microwave synthesizer. After cooling, the reaction mixture was partitioned between ethyl acetate and water and the insoluble solid was removed by filtration. The ethyl acetate layer was separated, dried (Na₂SO₄), filtered, evaporated then purified on a silica Biotage column eluting with DMAW240 then DMAW90. Product-containing fractions were combined and evaporated then re-evaporated from methanol (×2) to give 8 mg of the title compound as a cream solid. M/z: 410 ¹H NMR (DMSO-d₆) 11.66 (1H, s), 8.15 (1H, s), 7.35 (1H, br s), 7.28 (1H, s), 7.18 (1H, d), 7.00 (1H, br s), 6.75 (1H, dd), 6.63 (1H, d), 4.25 (2H, dm), 3.89 (2H, tm), 3.76 (3H, s), 2.23 (2H, tm), 1.75 (2H, dm)

### EXAMPLE 43

### 4-(4-Pyrrolidin-1-ylmethyl-1H-benzoimidazol-2-yl)-1-(7H-pyrrolo[2,3-d]pyrimidin-4-yl)-piperidin-4-ylamine

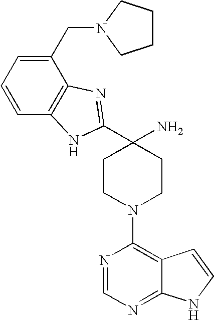

### 43A. 2-[4-amino-1-(7H-pyrrolo[2,3-d]pyrimidin-4-yl)-piperidin-4-yl]-1H-benzoimidazole-4-carbaldehyde

A solution of {2-[4-amino-1-(7H-pyrrolo[2,3-d]pyrimidin-4-yl)-piperidin-4-yl]-1H-benzoimidazol-4-yl}-methanol (Example 15) (115 mg; 0.27 mmol) in methanol (3 ml) was treated with DCM (15 ml) followed by managanese dioxide (475 mg; 20 equiv) then stirred at room temperature overnight. The reaction was incomplete as indicated by LC/MS. Further managanese dioxide was added until the reaction was complete. The reaction was filtered through Celite and the filtrate evaporated to give 75 mg of 2-[4-amino-1-(7H-pyrrolo[2,3-d]pyrimidin-4-yl)-piperidin-4-yl]-1H-benzoimidazole-4-carbaldehyde which was used without further purification or characterization.

### 43B. 4-(4-Pyrrolidin-1-ylmethyl-1H-benzoimidazol-2-yl)-1-(7H-pyrrolo[2,3-d]pyrimidin-4-yl)-piperidin-4-ylamine

A suspension of 2-[4-amino-1-(7H-pyrrolo[2,3-d]pyrimidin-4-yl)-piperidin-4-yl)-piperidin-4-yl]-1H-benzoimidazole-4-carbaldehyde in DCM (10 ml) was treated with acetic acid (30 ul; 2.2 equiv), pyrrolidine (40 ul; 2.2 equiv) and sodium triacetoxyborohydride (200 mg; 4.4 equiv) then heated at 35° C. overnight. The reaction was evaporated then purified on a silica Biotage column eluting with DMAW90 then DMAW60. Production containing fractions were combined and evaporated then re-evaporated from methanol (×2). The residue was partitioned between ethyl acetate and saturated NaHCO₃ solution. The ethyl acetate layer was dried (Na₂SO₄), filtered and evaporated to give 15 mg of the title compound as a white solid.

### EXAMPLE 44

### (4-(1H-benzo[d]imidazol-2-yl)-1-(9H-purin-6-yl)piperidin-4-yl)methanamine

### 44A. 2-chloro-N,N-dimethyl-1H-benzo[d]imidazole-1-sulfonamide

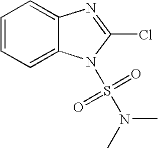

A solution of 1,4-diazabicyclo[2.2.2]octane (7.35 g, 65.54 mmol) in DMF (40 ml) was added to a stirred solution of 2-chloro-1H-benzo[d]imidazole (10 g, 65.54 mmol) and dimethylsulfamoyl chloride (7.04 ml, 65.54 mmol) in DMF (60 ml). The resulting suspension was stirred at ambient temperature for 3 hours. The reaction mixture was diluted with water (200 mL), extracted with ethyl acetate (200 ml) and washed sequentially with 10% aqueous sodium chloride (200 mL) and saturated brine (100 mL). The organic layer was dried over MgSO4, filtered and evaporated to afford crude product. The crude product was purified by flash silica chromatography, elution gradient 10 to 35% EtOAc in isohexane. Pure fractions were evaporated to dryness to afford 2-chloro-N,N-dimethyl-1H-benzo[d]imidazole-1-sulfonamide (12.33 g, 72.4%) as a colourless oil which solidified on standing.

1H NMR (400.13 MHz, CDCl3) δ 3.02 (6H, s), 7.35-7.38 (2H, m), 7.67-7.70 (1H, m), 7.91-7.94 (1H, m)

MS m/e MH⁺ 260

### 44B. tert-Butyl 4-cyano-4-(1-(N,N-dimethylsulfamoyl)-1H-benzo[d]imidazol-2-yl)piperidine-1-carboxylate

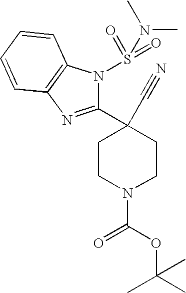

Sodium bis(trimethylsilyl)amide 1M in THF (43.1 ml, 43.12 mmol) was added dropwise to 2-chloro-N,N-dimethyl-1H-benzo[d]imidazole-1-sulfonamide (11.2 g, 43.12 mmol) and tert-butyl 4-cyanopiperidine-1-carboxylate (9.07 g, 43.12 mmol) in toluene (60 ml) at 0° C. over a period of 15 minutes under nitrogen. The resulting solution was stirred at 20° C. for 3 hours. The reaction mixture was diluted with EtOAc (200 mL), and washed sequentially with 2M HCl (100 mL) and saturated brine (100 mL). The organic layer was dried over MgSO4, filtered and evaporated to afford crude product. The crude product was purified by flash silica chromatography, elution gradient 20% EtOAc in isohexane. Pure fractions were evaporated to dryness to afford tert-butyl 4-cyano-4-(1-(N,N-dimethylsulfamoyl)-1H-benzo[d]imidazol-2-yl)piperidine-1-carboxylate (15.81 g 85%).

1H NMR (400.13 MHz, CDCl3) δ 1.46 (9H, s), 2.20-2.28 (2H, m), 2.75 (2H, d), 3.03 (6H, s), 3.33 (2H, d), 4.19 (2H, s), 7.41 (2H, q), 7.73-7.80 (2H, m)

MS m/e M−H 432

### 44C. 4-(1H-benzo[d]imidazol-2-yl)piperidine-4-carbonitrile

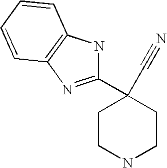

Hydrogen chloride 4M in dioxane (104 ml, 415.20 mmol) was added to tert-butyl 4-cyano-4-(1-(N,N-dimethylsulfamoyl)-1H-benzo[d]imidazol-2-yl)piperidine-1-carboxylate (15 g, 34.60 mmol). The resulting solution was stirred at ambient temperature for 24 hours. The reaction mixture was filtered, washing with diethyl ether. The crude product was purified by ion exchange chromatography, using an SCX column. The desired product was eluted from the column using 7M NH3/MeOH and pure fractions were evaporated to dryness to afford 4-(1H-benzo[d]imidazol-2-yl)piperidine-4-carbonitrile (4.79 g, 61.2%) as a white solid.

1H NMR (400.13 MHz, DMSO-d6) δ 2.07-2.14 (2H, m), 2.25 (2H, d), 2.80-2.95 (2H, m), 3.01-3.06 (2H, m), 7.20-7.24 (2H, m), 7.58 (2H, s)

MS m/e MH⁺ 227

### 44D. (4-(1H-Benzo[d]imidazol-2-yl)piperidin-4-yl)methanamine

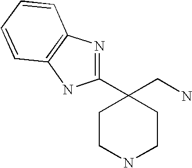

Borane-tetrahydrofuran complex (17.68 ml, 17.68 mmol) was added to 4-(1H-benzo[d]imidazol-2-yl)piperidine-4-carbonitrile (1 g, 4.42 mmol) in THF (20 ml) over a period of 5 minutes under nitrogen. The resulting solution was stirred at ambient temperature for 24 hours. The reaction mixture was quenched with methanol (25 ml) and 4M HCl in dioxane (25 ml) then heated at reflux for 4 hours. The crude product was purified by ion exchange chromatography, using an SCX column. The desired product was eluted from the column using 7M NH3/MeOH and pure fractions were evaporated to dryness to afford (4-(1H-benzo[d]imidazol-2-yl)piperidin-4-yl)methanamine (0.828 g, 81%) as a yellow gum.

MS m/e MH⁺ 231

### 44E. (4-1H-benzo[d]imidazol-2-yl)-1-(9H-purin-6-yl)piperidin-4-yl)methanamine

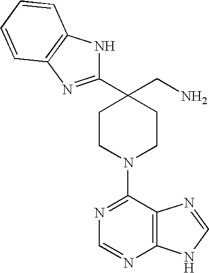

N-Ethyldiisopropylamine (0.109 ml, 0.63 mmol) was added to 6-chloro-9H-purine (81 mg, 0.52 mmol) and (4-(1H-benzo[d]imidazol-2-yl)piperidin-4-yl)methanamine (120 mg, 0.52 mmol) in butan-1-ol (2 ml). The resulting solution was stirred at 60° C. for 3 hours. The reaction mixture was filtered and the solid washed with methanol and diethyl ether to give impure product. The crude product was purified by preparative HPLC (Waters XBridge Prep C18 OBD column, 5μ silica, 19 mm diameter, 100 mm length), using decreasingly polar mixtures of water (containing 1% NH₃) and MeCN as eluents. Fractions containing the desired compound were evaporated to dryness to afford (4-(1H-benzo[d]imidazol-2-yl)-1-(9H-purin-6-yl)piperidin-4-yl)methanamine (143 mg, 79%) as a white solid.

¹H NMR (400.13 MHz, DMSO-d6+d4AcOH) δ 1.85-1.91 (2H, m+AcOH), 2.44-2.48 (2H, m), 3.23 (2H, s), 3.89 (2H, s), 4.71 (2H, s), 7.18-7.20 (2H, m), 7.55-7.59 (2H, m), 8.12 (1H, s), 8.23 (1H, s)

MS m/e MH⁺ 349

### EXAMPLE 45

### (4-(1H-benzo[d]imidazol-2-yl)-1-(5-chloro-7H-pyrrolo[2,3-d]pyrimidin-4-yl)piperidin-4-yl)methanamine

### 45A. 1-(4-(1H-benzo[d]imidazol-2-yl)piperidin-4-yl)-N-(diphenylmethylene)-methanamine

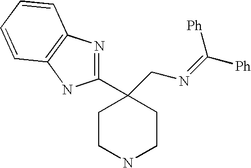

(4-(1H-Benzo[d]imidazol-2-yl)piperidin-4-yl)methanamine (987 mg, 4.29 mmol) (Example 44D, benzophenone imine (0.719 ml, 4.29 mmol) and p-toluenesulfonic acid (221 mg, 1.29 mmol) were added to DCM (40 ml) and stirred at 25° C. for 1 day. The reaction mixture was diluted with saturated NaHCO₃ (25 mL), and extracted with DCM (50 mL). The organics were washed with saturated brine (25 mL). The organic layer was dried over MgSO4, filtered and evaporated to afford crude product. The crude product was purified by flash silica chromatography, elution 0 to 10% MeOH in DCM then 10% methanol in DCM with ammonia. Pure fractions were evaporated to dryness to afford 1-(4-(1H-benzo[d]imidazol-2-yl)piperidin-4-yl)-N-(diphenylmethylene)methanamine (532 mg, 31.5%) as a white foam.

¹H NMR (400.13 MHz, CDCl3) δ 1.71-1.77 (2H, m), 2.35-2.40 (2H, m), 2.77-2.83 (2H, m), 2.96-3.02 (2H, m), 3.62 (2H, s), 7.08-7.10 (2H, m), 7.21-7.24 (2H, m), 7.39-7.46 (7H, m), 7.61 (2H, t), 7.78-7.80 (1H, m), 10.89 (1H, s)

MS m/e MH⁺ 395

### 45B. (4-(1H-benzo[d]imidazol-2-yl)-1-(5-chloro-7H-pyrrolo[2,3-d]pyrimidin-4-yl)piperidin-4-yl)methanamine

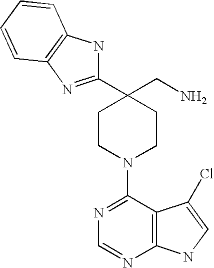

N-Ethyldiisopropylamine (0.132 ml, 0.76 mmol) was added to 4,5-dichloro-7H-pyrrolo[2,3-d]pyrimidine (119 mg, 0.63 mmol) (Preparation 7) and 1-(4-(1H-benzo[d]imidazol-2-yl)piperidin-4-yl)-N-(diphenylmethylene)methanamine (250 mg, 0.63 mmol) in butan-1-ol (4 ml). The resulting solution was stirred at 110° C. for 2 hours. Hydrogen chloride 6N in isopropanol (1.056 ml, 6.34 mmol) and water (1 ml) were added and the reaction was heated for a further 10 minutes. The crude product was purified by ion exchange chromatography, using an SCX column. The product was eluted from the column using 7M NH3/MeOH and product containing fractions were evaporated to dryness. The crude product was purified by preparative HPLC (Waters XBridge Prep C18 OBD column, 5μ silica, 19 mm diameter, 100 mm length), using decreasingly polar mixtures of water (containing 1% NH3) and MeCN as eluents. Fractions containing the desired compound were evaporated to dryness to afford (4-(1H-benzo[d]imidazol-2-yl)-1-(5-chloro-7H-pyrrolo[2,3-d]pyrimidin-4-yl)piperidin-4-yl)methanamine (81 mg, 33.5%) as a white solid.

¹H NMR (400.13 MHz, DMSO-d6) δ 1.86-1.93 (2H, m), 2.44-2.52 (2H, d), 2.82 (2H, s), 3.21 (2H, s), 4.03-4.06 (2H, m), 7.12-7.15 (2H, m), 7.45 (1H, s), 7.51-7.54 (2H, m), 8.22 (1H, s)

MS m/e MH⁺ 382

### EXAMPLE 46

### 4-1H-benzo[d]imidazol-2-yl)-1-(3-bromo-1H-pyrazolo[3,4-d]pyrimidin-4-yl)piperidin-4-yl)methanamine

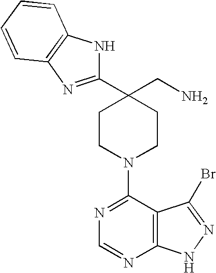

N-Ethyldiisopropylamine (0.046 ml, 0.26 mmol) was added to 1-(4-(1H-benzo[d]imidazol-2-yl)piperidin-4-yl)-N-(diphenylmethylene)methanamine (86 mg, 0.22 mmol) (Example 45A) and 3-bromo-4-chloro-1H-pyrazolo[3,4-d]pyrimidine (50.9 mg, 0.22 mmol) in butan-1-ol (4 ml). The resulting solution was stirred at 20° C. for 4 hours. Hydrogen chloride 6N in isopropanol (0.363 ml, 2.18 mmol) and water (1 ml) were added and the reaction stirred for 18 hours. The crude product was purified by ion exchange chromatography, using an SCX column. The crude product was eluted from the column using 7M NH3/MeOH. The crude product was purified by preparative HPLC (Waters XBridge Prep C18 OBD column, 5μ silica, 19 mm diameter, 100 mm length), using decreasingly polar mixtures of water (containing 1% NH3) and MeCN as eluents. Fractions containing the desired compound were evaporated to dryness to afford desired product as a yellow gum. The product was purified by flash silica chromatography, elution gradient 0 to 20% MeOH in DCM with ammonia. Pure fractions were evaporated to dryness to afford (4-(1H-benzo[d]imidazol-2-yl)-1-(3-bromo-1H-pyrazolo[3,4-d]pyrimidin-4-yl)piperidin-4-yl)methanamine (23.00 mg, 24.69%) as a white solid.

¹H NMR (400.13 MHz, DMSO-d6) δ 1.85-1.91 (2H, m), 2.84 (2H, s), 3.37 (2H, s), 4.28-4.32 (2H, m), 7.13-7.16 (2H, m), 7.52-7.55 (2H, m), 8.29 (1H, s)

MS m/e MH⁺ 427

### EXAMPLE 47

### 1H-benzo[d]imidazol-2-yl)-1-(5-methyl-7H-pyrrolo[2,3-d]pyrimidin-4-yl)piperidin-4-yl)methanamine

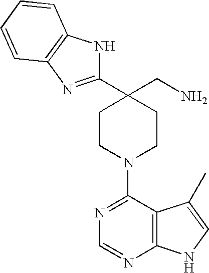

N-Ethyldiisopropylamine (0.132 ml, 0.76 mmol) was added to 4-chloro-5-methyl-7H-pyrrolo[2,3-d]pyrimidine (106 mg, 0.63 mmol) (Preparation 8) and 1-(4-(1H-benzo[d]imidazol-2-yl)piperidin-4-yl)-N-(diphenylmethylene)methanamine (250 mg, 0.63 mmol) (Example 45A) in butan-1-ol (4 ml). The resulting solution was stirred at 110° C. for 2 hours. Hydrogen chloride 6N in isopropanol (1.056 ml, 6.34 mmol) and water (1 ml) were added and the reaction was heated for a further 10 minutes. The crude product was purified by ion exchange chromatography, using an SCX column. The product was eluted from the column using 7M NH3/MeOH and product containing fractions were evaporated to dryness. The crude product was purified by preparative HPLC (Waters XBridge Prep C18 OBD column, 5μ silica, 19 mm diameter, 100 mm length), using decreasingly polar mixtures of water (containing 1% NH3) and MeCN as eluents. Fractions containing the desired compound were evaporated to dryness to afford (4-(1H-benzo[d]imidazol-2-yl)-1-(5-methyl-7H-pyrrolo[2,3-d]pyrimidin-4-yl)piperidin-4-yl)methanamine (66.0 mg, 28.8%) as a colourless gum.

¹H NMR (400.13 MHz, DMSO-d6) δ 1.85-1.92 (2H, m), 2.37-2.37 (3H, s), 2.46 (2H, d), 2.83 (2H, s), 3.10-3.17 (2H, m), 3.84 (2H, d), 7.04 (1H, s), 7.12-7.15 (2H, m), 7.51-7.54 (2H, m), 8.16 (1H, s), 11.45 (1H, s)

MS m/e MH⁺ 362

### EXAMPLE 48

### (4-(1H-benzo[d]imidazol-2-yl)-1-(5-bromo-7H-pyrrolo[2,3-d]pyrimidin-4-yl)-piperidin-4-yl)methanamine

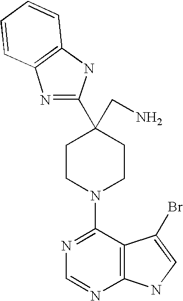

N-Ethyldiisopropylamine (0.227 ml, 1.30 mmol) was added to 5-bromo-4-chloro-7H-pyrrolo[2,3-d]pyrimidine (252 mg, 1.09 mmol) (Preparation 6) and (4-(1H-benzo[d]imidazol-2-yl)piperidin-4-yl)methanamine (250 mg, 1.09 mmol) (Example 44D) in butan-1-ol (4 ml). The resulting solution was stirred at 20° C. for 5 days. The reaction mixture was filtered and the filtrate was purified by ion exchange chromatography, using an SCX column. The desired product was eluted from the column using 7M NH3/MeOH. The crude product was purified by preparative HPLC (Waters XBridge Prep C18 OBD column, 5μ silica, 19 mm diameter, 100 mm length), using decreasingly polar mixtures of water (containing 0.1% TFA) and MeCN as eluents then repeated using decreasingly polar mixtures of water (containing 1% NH3) and MeCN as eluents Fractions containing the desired compound were evaporated to dryness to afford (4-(1H-benzo[d]imidazol-2-yl)-1-(5-bromo-7H-pyrrolo[2,3-d]pyrimidin-4-yl)piperidin-4-yl)methanamine (32.0 mg, 6.91%) as a yellow solid.

1H NMR (500.13 MHz, DMSO-d6) δ]1.97-2.03 (2H, m), 2.97 (2H, s), 3.38 (2H, m), 3.99-4.04 (2H, m), 7.13-7.16 (2H, m), 7.39 (1H, d), 7.52-7.55 (2H, m), 8.23 (1H, s)

MS m/e MH⁺ 426

### EXAMPLE 49

### 4-(1H-benzo[d]imidazol-2-yl)-1-(9H-purin-6-yl)piperidine-4-carbonitrile

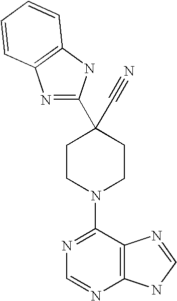

N-Ethyldiisopropylamine (1.324 ml, 7.60 mmol) was added to 6-chloro-9H-purine (587 mg, 3.80 mmol) and 4-(1H-benzo[d]imidazol-2-yl)piperidine-4-carbonitrile (860 mg, 3.80 mmol) (Example 44C) in NMP (10 ml). The resulting solution was stirred at 110° C. for 2 hours. The crude product was purified by ion exchange chromatography, using an SCX column. The desired product was eluted from the column using 7M NH3/MeOH and fractions were evaporated to dryness. The crude product was triturated with DCM to give 4-(1H-benzo[d]imidazol-2-yl)-1-(9H-purin-6-yl)piperidine-4-carbonitrile (422 mg, 32.2%).

1H NMR (400.13 MHz, DMSO-d6) δ 2.30-2.38 (2H, m), 2.46 (2H, d), 3.63 (2H, s), 5.39 (2H, s), 7.20-7.24 (2H, m), 7.58 (2H, s), 8.19 (1H, s), 8.29 (1H, s), 12.91 (1H, s)

MS m/e MH⁺ 345

### EXAMPLE 50

### 1-(7H-Pyrrolo[2,3-d]pyrimidin-4-yl)-4-[6-(trifluoromethyl)-1H-benzimidazol-2-yl]piperidin-4-amine

### 50A. 4-tert-butoxycarbonylamino-1-(7H-pyrrolo[2,3-d]pyrimidin-4-yl)-piperidine-4-carboxylic acid ethyl ester

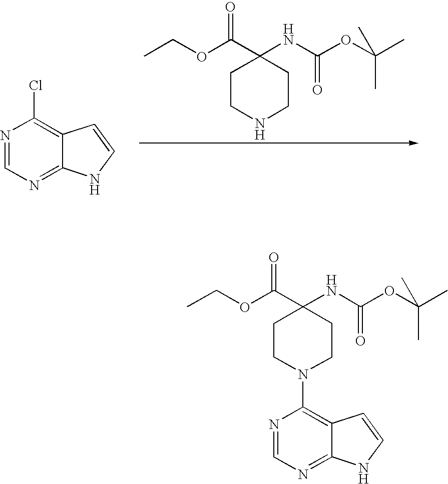

4-tert-butoxycarbonylamino-piperidine-4-carboxylic acid ethyl ester (5 g, 19.4 mmol*) was dissolved in N-methylpyrrolidinone (41 mL) and triethylamine (2.9 mL, 21.3 mmol) was added followed by 4-chloro-7H-pyrrolo[2,3-d]pyrimidine (3.27 g, 21.3 mmol). The resulting mixture was heated at 110° C. under nitrogen for 4 hours. The reaction mixture was allowed to stand for 64 hours. The reaction mixture was diluted with ethyl acetate and the organic was washed three times with water. The organic was separated off, dried (MgSO₄) and concentrated in vacuo. The residue was purified by flash column chromatography on silica gel, eluting with 50/50 ethyl acetate/petroleum ether to afford the title compound as a yellow oil (9.70 g, >100%).

Commercially available from Astatech (catalogue number: 55743)

### 50B. 4-tert-Butoxycarbonylamino-1-(7H-pyrrolo[2,3-d]pyrimidin-4-yl)-piperidine-4-carboxylic acid

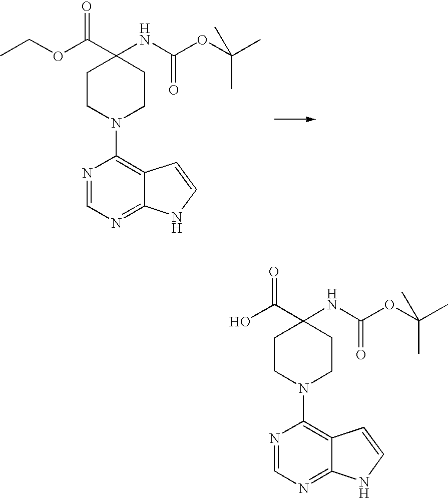

4-tert-Butoxycarbonylamino-1-(7H-pyrrolo[2,3-d]pyrimidin-4-yl)-piperidine-4-carboxylic acid ethyl ester (7.28 g, 19.4 mmol) was dissolved in a 1:1 mixture of ethanol and tetrahydrofuran (100 mL in total). A solution of sodium hydroxide (3.88 g, 97 mmol) in water (50 mL) was made up and this was added to the solution of 4-tert-butoxycarbonylamino-1-(7H-pyrrolo[2,3-d]pyrimidin-4-yl)-piperidine-4-carboxylic acid ethyl ester. The resulting mixture was stirred at 60° C. for 18 hours. The reaction was allowed to cool and was concentrated in vacuo. The residue was dissolved in water (100 mL) and was acidified cautiously with conc. HCl to pH 4-5 with ice-cooling. The aqueous was extracted four times with ethyl acetate, each time ensuring an aqueous pH of 4-5. The organics were combined, dried (MgSO₄) and concentrated in vacuo to afford the title compound as a yellow gum (7.3 g, >100%). The product was used without further purification.

### 50C. 1-(7H-Pyrrolo[2,3-d]pyrimidin-4-yl)-4-[6-(trifluoromethyl)-1H-benzimidazol-2-yl]piperidin-4-amine

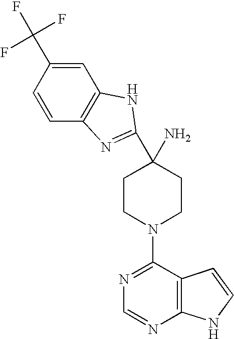

A solution of HATU (419 mg, 11 mmol), in DMA (2 mL) was added to a mixture of 4-tert-butoxycarbonylamino-1-(7H-pyrrolo[2,3-d]pyrimidin-4-yl)-piperidine-4-carboxylic acid (361 mg, 1 mmol), DIPEA (0.522 mL, 3 mmol) and 4-(trifluoromethyl)benzene-1,2-diamine (194 mg, 1.1 mmol) in DMA (3 mL). The resulting mixture was stirred at room temperature for 1 hour, then heated to 60° C. and stirred overnight. The reaction mixture was then purified by ion exchange chromatography, using an SCX column. The crude product were eluted from the column using 7N methanolic ammonia, and concentrated in vacuo. Concentrated aqueous hydrochloric acid (10 mL) was added to the crude material, and the resulting mixture stirred and heated at 85° C. for 48 hours. The reaction mixture was then diluted with water, and again purified by ion exchange chromatography, using an SCX column. The column was washed with water, methanol, DCM, and finally more methanol. The product was recovered from the column with 7N methanolic ammonia, then concentrated in vacuo. The resulting crude material was purified by flash silica chromatography, eluting with 0-10% 7N methanolic ammonia in DCM.

1H NMR (400.13 MHz, DMSO-d₆) δ 1.81 (2H, d), 2.20-2.29 (2H, m), 3.85-3.91 (2H, m), 4.26-4.33 (2H, m), 6.63 (1H, d), 7.18 (1H, d), 7.46 (1H, d), 7.68 (1H, d), 7.85 (1H, s), 8.16 (1H, s), 11.65 (1H, s).

MS m/e MH⁺ 402.

### EXAMPLES 51 TO 54

Examples 51 to 54 were prepared by the method described in Example 50.

### EXAMPLE 51

### 4-(6,7-dimethyl-1H-benzimidazol-2-yl)-1-(7H-pyrrolo[2,3-d]pyrimidin-4-yl)piperidin-4-amine

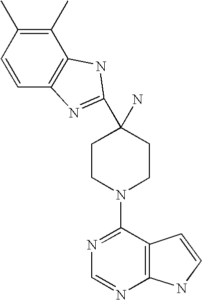

¹H NMR (400.13 MHz, DMSO-d₆) δ 1.75-1.81 (2H, m), 2.23-2.28 (2H, m), 2.30 (3H, s), 2.42 (3H, s), 3.85-3.92 (2H, m), 4.25-4.31 (2H, m), 6.63 (1H, d), 6.93 (1H, d), 7.16-7.21 (2H, m), 8.15 (1H, s), 11.65 (1H, s).

MS m/e MH⁺ 362.

### EXAMPLE 52

### 4-(6-chloro-5-fluoro-1H-benzimidazol-2-yl)-1-(7H-pyrrolo[2,3-d]pyrimidin-4-yl)piperidin-4-amine

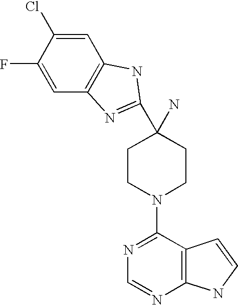

¹H NMR (399.9 MHz, DMSO-d₆) δ 1.79 (2H, d), 2.19-2.25 (2H, m), 3.84-3.91 (2H, m), 4.26-4.31 (2H, m), 6.63 (1H, d), 7.18 (1H, d), 7.50-7.53 (1H, m), 7.67-7.68 (1H, m), 8.16 (1H, s), 11.66 (1H, s).

MS m/e MH⁺ 386.

### EXAMPLE 53

### 4-(5,6-Dichloro-1H-benzoimidazol-2-yl)-1-(7H-pyrrolo[2,3-d]pyrimidin-4-yl)-piperidin-4-ylamine

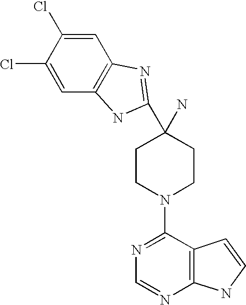

1H NMR (400.13 MHz, DMSO-d₆) δ 1.76-1.80 (2H, m), 2.17-2.24 (2H, m), 3.83-3.89 (2H, m), 4.29 (2H, d), 6.62 (1H, d), 7.17-7.18 (1H, m), 7.74 (2H, s), 8.15 (1H, s), 11.65 (1H, s).

MS m/e MH⁺ 402.

Note—this example was further purified by preparative LCMS (Waters XBridge Prep C18 OBD column, 5μ silica, 19 mm diameter, 100 mm length), using decreasingly polar mixtures of water (containing 1% NH3) and MeCN as eluents. Fractions containing the desired compound were evaporated to dryness.

### EXAMPLE 54

### 4-(1-methylbenzimidazol-2-yl)-1-(7H-pyrrolo[2,3-d]pyrimidin-4-yl)piperidin-4-amine

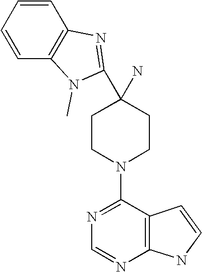

¹H NMR (400.13 MHz, DMSO-d₆) δ 1.92-1.95 (2H, m), 2.34-2.42 (4H, m), 4.06 (2H, s), 4.11 (3H, s), 6.62-6.63 (1H, m), 7.14-7.18 (2H, m), 7.22 (1H, t), 7.50 (1H, d), 7.58 (1H, d), 8.14 (1H, s), 11.64 (1H, s).

MS m/e MH⁺ 348.

### EXAMPLE 55

### 4-(3H-imidazo[5,4-b]pyridin-2-yl)-1-(7H-pyrrolo[2,3-d]pyrimidin-4-yl)piperidin-4-amine

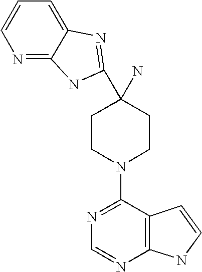

¹H NMR (400.13 MHz, DMSO-d₆) δ 1.78-1.82 (2H, m), 2.22-2.29 (2H, m), 3.85-3.92 (2H, m), 4.26-4.30 (2H, m), 6.62-6.63 (1H, m), 7.15-7.19 (2H, m), 7.88-7.90 (1H, m), 8.15 (1H, s), 8.26-8.27 (1H, m), 11.64 (1H, s).

MS m/e MH⁺ 335.

### EXAMPLE 56

### 4-(5-imidazol-1-yl-1H-benzimidazol-2-yl)-1-(7H-pyrrolo[2,3-d]pyrimidin-4-yl)piperidin-4-amine

### 56A. 4-Imidazol-1-ylbenzene-1,2-diamine

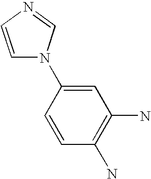

5-(1H-Imidazol-1-yl)-2-nitroaniline (500 mg, 2.45 mmol) (obtained from AP building blocks) and palladium (10% 0n C) (50 mg, 0.05 mmol) in ethanol (100 mL) were stirred under an atmosphere of hydrogen at 1 atm and 25° C. for 24 hours. The reaction mixture was filtered through Celite, and the filtrate concentrated and dried under vacuum to give 4-(1H-imidazol-1-yl)benzene-1,2-diamine (400 mg, 94%) as a purple solid.

MS m/e MH⁺ 175.

### 56B. 4-(5-imidazol-1-yl -1H-benzimidazol-2-yl)-1-(7H-pyrrolo[2,3-d]pyrimidin-4-yl)piperidin-4-amine

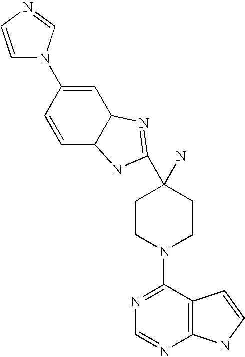

The title compound was prepared by reacting 4-[(2-methylpropan-2-yl)oxycarbonyl-amino]-1-(7H-pyrrolo[2,3-d]pyrimidin-4-yl)piperidine-4-carboxylic acid with 4-imidazol-1-ylbenzene-1,2-diamine using the method described in Example 50.

¹H NMR (400.13 MHz, DMSO-d6) δ 1.77-1.85 (2H, m), 2.24-2.30 (2H, m), 3.88-3.95 (2H, m), 4.25-4.31 (2H, m), 6.63 (1H, d), 7.10 (1H, s), 7.18 (1H, d), 7.38 (1H, dd), 7.60 (1H, d), 7.67 (1H, t), 7.72 (1H, s), 8.16 (1H, t), 8.17 (1H, s), 11.67 (1H, s)

MS m/e WH⁺ 400.

### EXAMPLE 57

### 4-(5-chloro-6-methyl -1H-benzo[d]imidazol-2-yl)-1-(7H-pyrrolo[2,3-d]pyrimidin-4-yl)piperidin-4-amine

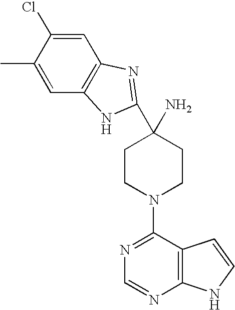

4-(tert-Butoxycarbonylamino)-1-(7H-pyrrolo[2,3-d]pyrimidin-4-yl)piperidine-4-carboxylic acid (500 mg, 1.38 mmol), 4-chloro-3-methylbenzene-1,2-diamine (325 mg, 2.08 mmol), 1-(3-Dimethylaminopropyl)-3-ethylcarbodiimide hydrochloride (398 mg, 2.08 mmol) and 1-Hydroxybenzotriazole (206 mg, 1.52 mmol) were added to DMF (15 mL) and heated at 60° C. for 5 hours. The resulting black solution was evaporated to dryness and used in the next reaction without any further purification.

tert-butyl-4-(2-amino-4-chloro-5-methylphenylcarbamoyl)-1-(7H-pyrrolo[2,3d]pyrimidin-4-yl)piperidin-4-ylcarbamate (0.690 g, 1.38 mmol) and cone HCl (15 ml, 493.68 mmol) were heated at 100° C. overnight. The resulting black solution was passed through a 20 g SCX-2 column. The crude product was purified by preparative HPLC using decreasingly polar mixtures of water (containing 1% NH3) and MeCN as eluents. Fractions containing the desired compound were evaporated to dryness to afford 4-(5-chloro-6-methyl-1H-benzo[d]imidazol-2-yl)-1-(7H-pyrrolo[2,3d]pyrimidin-4-yl)piperidin-4-amine (0.035 g, 0.09 mmol, 6.64%) as a white solid. m/z (ESI+) (M+H)+=382; 1H NMR (400.132 MHz, DMSO) δ 1.78-1.75 (m, 2H), 2.21 (ddd, 2H), 2.39 (s, 3H), 3.44-3.18 (brm, 2H), 3.89-3.84 (m, 2H), 4.28-4.25 (m, 2H), 6.62 (s, 1H), 7.17 (s, 1H), 7.43 (s, 1H), 7.52 (s, 1H), 8.15 (s, 1H).

### EXAMPLE 58

### 4-(5-Chloro-7-methyl-1H-benzo[d]imidazol-2-yl)-1-(7H-pyrrolo[2,3-d]pyrimidin-4-yl)piperidin-4-amine

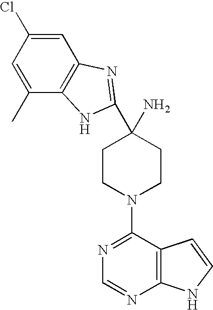

1-(tert-Butoxycarbonyl)-4-(tert-butoxycarbonylamino)piperidine-4-carboxylic acid (1.000 g, 2.90 mmol), O-(7-Azabenzotriazol-1-yl)-N,N,N′,N′-tetramethyluronium hexafluorophosphate (1.435 g, 3.77 mmol), 5-chloro-3-methyl-1,2-phenylenediamine (0.455 g, 2.90 mmol) and N-ethyldiisopropylamine (0.976 g, 7.55 mmol) were added to DMA (35 mL) and stirred for 10 minutes at ambient temperture. The reaction was then heated at 60° C. for 48 hours. The reaction was quenched with 2.0 NaOH (50 ml), extracted with diethyl ether (3×100 ml), dried (MgSO4) and the solvent removed in vacuo to yield crude tert-butyl 4-(2-amino-5-chloro-3-methylphenylcarbamoyl)-4-(tert-butoxycarbonylamino)piperidine-1-carboxylate (71.3%) as purple gum. This was used in the next step without any further purification.

4-(tert-butoxycarbonylamino)-1-(7H-pyrrolo[2,3-d]pyrimidin-4-yl)piperidine-4-carboxylic acid (0.500 g, 1.38 mmol), O-(7-azabenzotriazol-1-yl)-N,N,N′,N′-tetramethyluronium hexafluorophosphate (0.684 g, 1.80 mmol), 5-chloro-3-methyl-1,2-phenylenediamine (0.238 g, 1.52 mmol) and N-ethyldiisopropylamine (0.622 ml, 3.60 mmol) were dissolved in DMF (15 mL) and stirred at room temperature for 15 minutes, the reaction was then heated at 60° C. for 16 hours. The reaction was evaporated to dryness, quenched with 2.0N NaOH (25 ml), extracted with ethyl acetate (3×75 ml), dried (MgSO4) and solvent removed invacuo to yield a yellow gum. This was dissolved in Hydrogen chloride (10 ml, 100.00 mmol) and heated at 100° C. overnight, the resulting solution was evaporated to dryness and passed through a 20 g SCX column. The crude product was purified by preparative HPLC using decreasingly polar mixtures of water (containing 1% NH3) and MeCN as eluents. Fractions containing the desired compound were evaporated to dryness to afford 4-(5-chloro-7-methyl-1H-benzo[d]imidazol-2-yl)-1-(7H-pyrrolo[2,3-d]pyrimidin-4-yl)piperidin-4-amine (0.069 g, 0.18 mmol, 13.06%) as a white solid. m/z (ES+) (M+H)+=382; HPLC tR=1.64; 1H NMR (400.132 MHz, CDCl3) δ 1.54-1.51 (2H, m), 1.66 (3H, s), 2.24-2.16 (4H, m), 3.66-3.61 (2H, m), 4.13-4.09 (2H, m), 6.19 (1H, d), 6.69 (1H, s), 6.71 (1H, d), 7.97 (1H, s).

### EXAMPLE 59

### 2-(4-amino-1-(7H-pyrrolo[2,3-d]pyrimidin-4-yl)piperidin-4-yl)-N,N-diethyl-1H-benzo[d]imidazole-5-sulfonamide

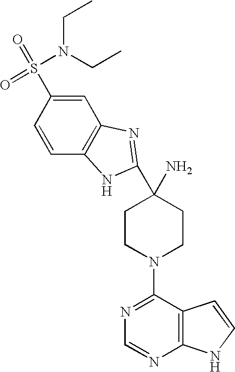

4-(tert-butoxycarbonylamino)-1-(7H-pyrrolo[2,3-d]pyrimidin-4-yl)piperidine-4-carboxylic acid (0.500 g, 1.38 mmol), O-(7-Azabenzotriazol-1-yl)-N,N,N′,N′-tetramethyluronium hexafluorophosphate (0.684 g, 1.80 mmol), 3,4-Diamino-N,N-diethyl-benzenesulfonamide (0.370 g, 1.52 mmol) and N-Ethyldiisopropylamine (0.622 ml, 3.60 mmol) were dissolved in DMF (15 mL) and stirred at room temperature for 15 minutes, the reaction was then heated at 60° C. for 16 hours. The reaction mixture was quenched with 2M NaOH (25 mL), extracted with DCM (3×50 mL), the organic layer was dried over MgSO4, filtered and evaporated to afford brown solid. This was dissolved in Hydrogen chloride (15 mL, 150.00 mmol) and heated at 100° C. overnight. The reaction mixture was evaporated to dryness and redissolved in water (10 mL), this was passed through a 20 g SCX column to afford crude product. The crude product was purified by preparative HPLC using decreasingly polar mixtures of water (containing 1% NH3) and MeCN as eluents. Fractions containing the desired compound were evaporated to dryness to afford 2-(4-amino-1-(7H-pyrrolo[2,3-d]pyrimidin-4-yl)piperidin-4-yl)-N,N-diethyl-1H-benzo[d]imidazole-5-sulfonamide (12 mg, 1.851%) as a beige solid. m/z (ESI+) (M+H)+469=; HPLC tR=1.24 min. 1H NMR (400.132 MHz, DMSO) δ 0.97 (6H, t), 1.89-1.85 (2H, m), 2.38-2.27 (2H, m), 3.09 (4H, q), 3.98-3.90 (2H, m), 4.12-4.08 (2H, m), 6.58 (1H, s), 7.13 (1H, s), 7.55 (1H, d), 7.66 (1H, d), 7.89 (1H, s), 8.11 (1H, s), 11.64 (1H, s).

### EXAMPLE 60

### 4-(5-(4-methylpiperazin-1-yl)-1H-benzo[d]imidazol-2-yl)-1-(7H-pyrrolo[2,3-d]pyrimidin-4-yl)piperidin-4-amine

### 60A. tert-Butyl 4-(2-amino-4-(4-methylpiperazin-1-yl)phenylcarbamoyl)-4-(tert-butoxycarbonylamino)piperidine-1-carboxylate

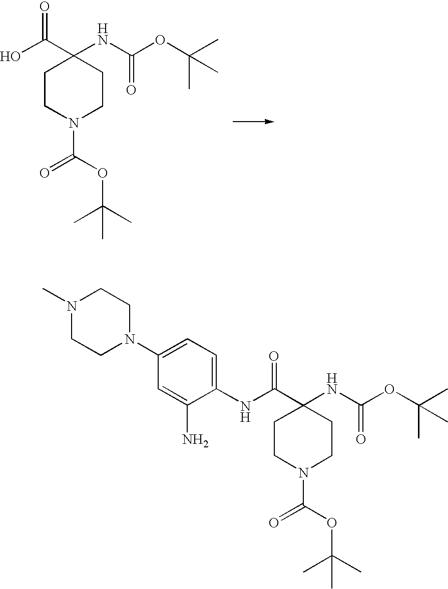

1-(tert-Butoxycarbonyl)-4-(tert-butoxycarbonylamino)piperidine-4-carboxylic acid (0.800 g, 2.32 mmol), N-(3-dimethylaminopropyl)-N′-ethylcarbodiimide hydrochloride (0.490 g, 2.56 mmol), 1-hydroxybenzotriazole (0.345 g, 2.56 mmol) and 4-(4-methylpiperazino)-1,2-benzenediamine (0.527 g, 2.56 mmol) were dissolved in DMF (35 mL) and heated at 60° C. overnight. The reaction mixture was quenched with 2M NaOH (50 mL), extracted with DCM (3×75 mL), the organic layer was dried over MgSO4, filtered and evaporated to afford black gum. This was used in the next step without any further purification. m/z (ESI)+(M+H)+=532; HPLC tR=1.85.

### 60B. 4-(5-(4-methylpiperazin-1-yl)-1H-benzo[d]imidazol-2-yl)piperidin-4-amine

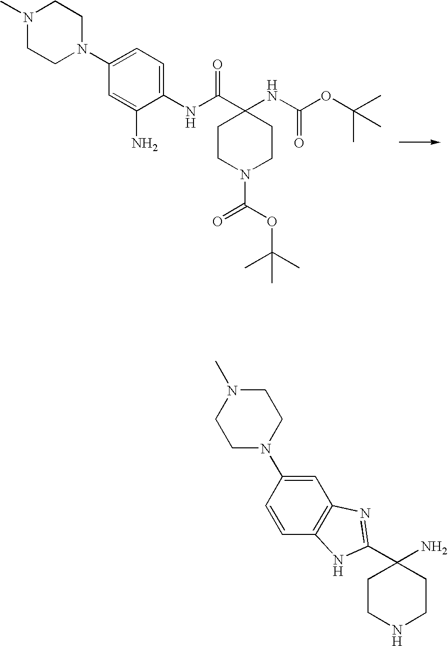

tert-butyl 4-(2-amino-4-(4-methylpiperazin-1-yl)phenylcarbamoyl)-4-(tert-butoxy carbonylamino)piperidine-1-carboxylate (1.000 g, 1.88 mmol) and Hydrogen chloride (15 ml, 150.00 mmol) were heated at 98° C. over the weekend. The reaction was evaporated to dryness and passed through a 20 g SCX column, the obtained black gum was used in the next step without any further purification. m/z (ESI+)+=315; HPLC tR=1.24.

### 60C. 4-(5-(4-methylpiperazin-1-yl)-1H-benzo[d]imidazol-2-yl)-1-(7H-pyrrlo[2,3-d]pyrimidin-4-yl)piperidin-4-amine

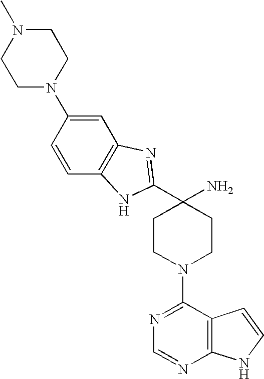

4-(5-(4-methylpiperazin-1-yl)-1H-benzo[d]imidazol-2-yl)piperidin-4-amine (0.200 g, 0.64 mmol), N-ethyldiisopropylamine (0.110 mL, 0.64 mmol) and 6-chloropurine (0.098 g, 0.64 mmol) were dissolved in DMA (35 mL) and heated at 90° C. overnight. The reaction was evaporated to dryness and the reaction mixture was quenched with 2M NaOH (50 mL), extracted with DCM (3×75 mL), the organic layer was dried over MgSO₄, filtered and evaporated to afford black gum. The crude product was purified by preparative HPLC using decreasingly polar mixtures of water (containing 1% NH₃) and MeCN as eluents. Fractions containing the desired compound were evaporated to dryness to afford 44544-methylpiperazin-1-yl)-1H-benzo[d]imidazol-2-yl)-1-(7H-pyrrolo[2,3-d]pyrimidin-4-yl)piperidin-4-amine (12 mg, 4.37%) as a yellow solid. m/z (ESI+) (M+H)+=432; HPLC tR=1.14 min; 1H NMR (400.132 MHz, DMSO) δ 1.70-1.66 (2H, m), 2.21-2.13 (2H, m), 3.02-2.97 (4H, m), 3.20 (3H, s), 3.25-3.21 (6H, m), 3.85-3.80 (2H, m), 4.17-4.14 (2H, m), 6.55 (1H, s), 6.86-6.74 (2H, m), 7.10 (1H, s), 8.08 (1H, s), 11.57 (1H, s),

### EXAMPLE 61

### 1-(7H-Pyrrolo[2,3-d]pyrimidin-4-yl)-4-(5-(trifluoromethoxy)-1H-benzo[d]imidazol-2-yl)piperidin-4-amine

### 61A. 4-(trifluoromethoxy)benzene-1,2-diamine

2-Nitro-4-(trifluoromethoxy)aniline (2.000 g, 9.00 mmol) and palladium (0.096 g, 0.90 mmol) were dissolved in ethanol (25 mL) and stirred under a balloon of hydrogen for 48 hours. The reaction was filtered and evaporated to dryness to afford 4-(trifluoromethoxy)-benzene-1,2-diamine (1.392 g, 7.24 mmol, 92%) as a black oil; m/z (ESI+) (M+H)+=; HPLC tR=1.53 min. 1H NMR (400.132 MHz, CDCl3) δ 3.41 (4H, vbrs), 6.60-6.58 (2H, m), 6.67 (1H, d).

### 61B. 1-(7H-Pyrrolo[2,3-d]pyrimidin-4-yl)-4-(5-(trifluoromethoxy)-1H-benzo[d]imidazol-2-yl)piperidin-4-amine

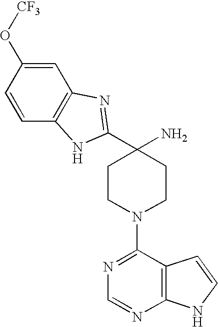

4-(Trifluoromethoxy)benzene-1,2-diamine (0.292 g, 1.52 mmol), 4-(tert-butoxycarbonylamino)-1-(7H-pyrrolo[2,3-d]pyrimidin-4-yl)piperidine-4-carboxylic acid (Example 50B) (0.5 g, 1.38 mmol), O-(7-azabenzotriazol-1-yl)-N,N,N′,N′-tetramethyluronium hexafluorophosphate (0.684 g, 1.80 mmol) and N-ethyldiisopropylamine (0.616 mL, 3.60 mmol) were dissolved in DMF (15 mL) and heated at 60° C. overnight. The resulting solution was evaporated to dryness, quenched with 2M NaOH (20 mL), extracted with DCM (3×50 mL), the organic layer was dried over MgSO4, filtered and evaporated to afford black gum. This was treated with Hydrogen chloride (15 mL, 150.00 mmol) and heated at 100° C. overnight. The resulting solution was diluted with water (30 mL) and passed through a 20 g SCX column the resulting crude product was purified by flash silica chromatography, elution gradient 10 to 20% MeOH in DCM. Pure fractions were evaporated to dryness to afford 1-(7H-pyrrolo[2,3-d]pyrimidin-4-yl)-4-(5-(trifluoromethoxy)-1H-benzo[d]imidazol-2-yl)piperidin-4-amine (36 mg, 6.23%) as a white solid. m/z (ESI+) (M+H)+=418; HPLC tR=1.68 min; 1H NMR (400.132 MHz, DMSO) δ 1.74-1.70 (2H, m), 2.20-2.14 (2H, m), 3.28-3.17 (2H, m), 3.84-3.79 (2H, m), 4.23-4.20 (2H, m), 6.56 (1H, d), 7.05 (1H, d), 7.11 (1H, d), 7.41 (1H, s), 7.50 (1H, d), 8.09 (1H, s), 11.58 (1H, s), 12.62-12.09 (1H, m).

### EXAMPLE 62

### 4-(5-Isopropyl-1H-benzo[d]imidazol-2-yl)-1-(7H-pyrrolo[2,3-d]pyrimidin-4-yl)piperidin-4-yl)piperidin-4-amine

### 62A. 4-Isopropylbenzene-1,2-diamine

4-Isopropyl-2-nitroaniline (0.830 g, 4.61 mmol) and palladium (0.049 g, 0.46 mmol) were dissolved in ethanol (25 mL) and stirred under a balloon of hydrogen for 48 hours. The reaction was filtered and evaporated to dryness to afford 4-isopropylbenzene-1,2-diamine (0.62 g, 90%) as a black solid; m/z (ESI+) (M+H)+=150; HPLC tR=1.55 min; 1H NMR (400.132 MHz, CDCl3) δ 1.22 (6H, d), 2.79 (1H, septet), 3.36 (4H, brs), 6.62-6.60 (2H, m), 6.67 (1H, d).

### 62B. 4(5-isopropyl-1H-benzo[d]imidazol-2-yl)-1-(7H-pyrrolo[2,3-d]pyrimidin-4-yl)piperidin-4-amine

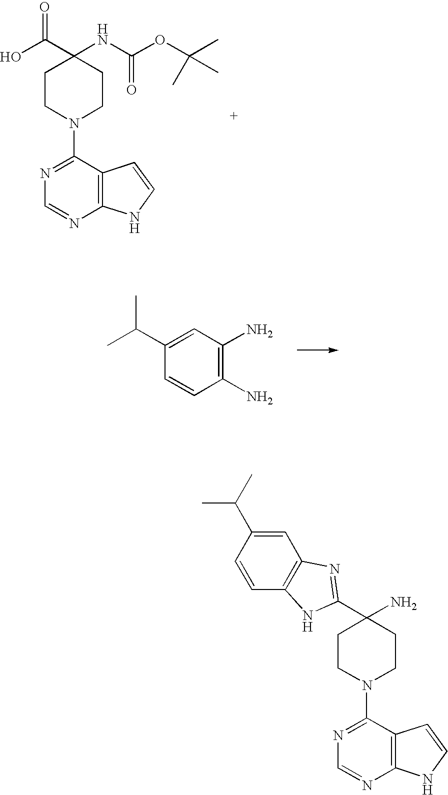

4-(tert-Butoxycarbonylamino)-1-(7H-pyrrolo[2,3-d]pyrimidin-4-yl)piperidine-4-carboxylic acid (0.500 g, 1.38 mmol), O-(7-azabenzotriazol-1-yl)-N,N,N′,N′-tetramethyluronium hexafluorophosphate (0.684 g, 1.80 mmol), 4-isopropylbenzene-1,2-diamine (0.210 g, 1.40 mmol) and N-ethyldiisopropylamine (0.622 ml, 3.60 mmol) were dissolved in DMF (15 mL) and stirred at room temperature for 15 minutes, the reaction was then heated at 60° C. for 16 hours. The reaction was evaporated to dryness, quenched with 2.0N NaOH (25 ml), extracted with ethyl acetate (3×75 ml), dried (MgSO4) and solvent removed invacuo to yield a yellow gum. This was dissolved in hydrogen chloride (10 ml, 100.00 mmol) and heated at 100 oC overnight, the resulting solution was evaporated to dryness and passed through a 20 g SCX column. The crude product was purified by preparative HPLC using decreasingly polar mixtures of water (containing 1% NH3) and MeCN as eluents. Fractions containing the desired compound were evaporated to dryness to afford 4-(5-isopropyl-1H-benzo[d]imidazol-2-yl)-1-(7H-pyrrolo[2,3-d]pyrimid-in-4-yl)piperidin-4-amine (16.5 mg, 3.18%) as a white solid. m/z (ESI+) (M+H)+=375; HPLC tR=1.68 min.

### EXAMPLE 63

### 63A. 4-(4-methoxyphenoxy)benzene-1,2-diamine

4-(4-methoxyphenoxy)-2-nitroaniline (2.000 g, 7.69 mmol) and 4-(4-methoxyphenoxy)-2-nitroaniline (2.000 g, 7.69 mmol) were dissolved in Ethanol (25 mL) and stirred under a balloon of hydrogen for 48 hours. The reaction was filtered and evaporated to dryness to afford a black solid. The crude residue was triturated with Et20 to give a solid which was collected by filtration and dried under vacuum to give 4-(4-methoxyphenoxy)benzene-1,2-diamine (1.4 g, 79%) as a brown solid. m/z (ESI+) (M+H)+=230; HPLC tR=1.62 min;

1H NMR (400.132 MHz, CDCl3) δ 3.36 (4H, s), 3.82 (3H, s), 6.35 (1H, dd), 6.40 (1H, d), 6.67 (1H, d), 6.88-6.84 (2H, m), 6.96-6.93 (2H, m).

### 63B. tert-Butyl 4-(tert-butoxycarbonylamino)-4-(5-(4-methoxyphenoxy)-1H-benzo[d]imidazol-2-yl)piperidine carboxylate

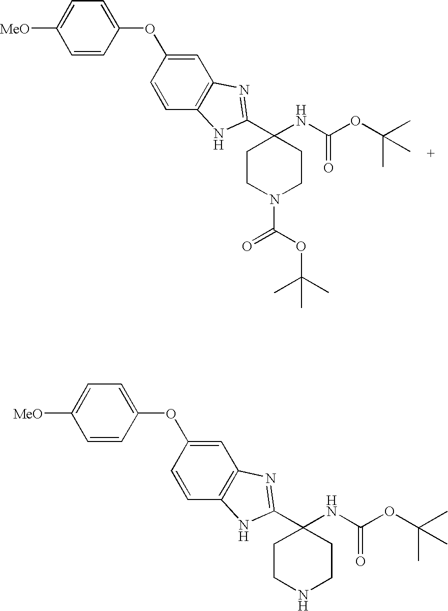

1-(tert-Butoxycarbonyl)-4-(tert-butoxycarbonylamino)piperidine-4-carboxylic acid (1.000 g, 2.90 mmol),4-(4-methoxyphenoxy)benzene-1,2-diamine (0.802 g, 3.48 mmol), O-(7-azabenzotriazol-1-yl)-N,N,N′,N′-tetramethyluronium hexafluorophosphate (1.435 g, 3.77 mmol) and N-ethyldiisopropylamine (1.338 mL, 7.55 mmol) were added to DMF (50 mL) and stirred at 55° C. overnight. The reaction was evaporated to dryness, quenched with 2M NaOH (50 mL), extracted with diethyl ether (3×100 mL), the organic layer was dried over MgSO4, filtered and evaporated to afford black gum. The crude product was purified by flash silica chromatography, elution gradient 0 to 3% MeOH in DCM. Pure fractions were evaporated to dryness to afford the amide as a brown residue. This was dissolved in toluene (40 ml) and p-toluenesulfonic acid (0.047 mL, 0.29 mmol) was added and the reaction was heated at reflux overnight. The reaction mixture was quenched with 2M NaOH (50 mL), extracted with diethyl ether (2×75 mL), the organic layer was dried over MgSO4, filtered and evaporated to afford tert-butyl 4-(tert-butoxycarbonylamino)-4-(5-(4-methoxyphenoxy)-1H-benzo[d]imidazol-2-yl)piperidine-1-carboxylate (0.935 g, 59.8%) and tert-butyl 4-(5-(4-methoxyphenoxy)-1H-benzo[d]imidazol-2-yl)piperidin-4-ylcarbamate (0.165 g, 12.96%) as an orange solid. m/z (ESI+) (M+H)+=539; HPLC tR 2.59=min.

### 63C. 4-(5-(4-methoxyphenoxy)-1H-benzo[d]imidazol-2-yl)piperidin-4-amine

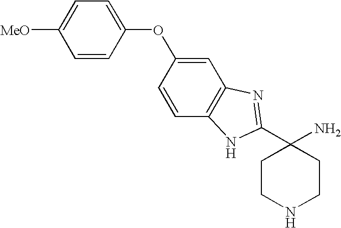

tert-butyl 4-(tert-butoxycarbonylamino)-4-(5-(4-methoxyphenoxy)-1H-benzo[d]imidazol-2-yl)piperidine-1-carboxylate (0.935 g, 1.74 mmol) and tert-butyl 4-(5-(4-methoxyphenoxy)-1H-benzo[d]imidazol-2-yl)piperidin-4-ylcarbamate (0.165 g, 0.38 mmol) were dissolved in Acetonitrile (15 mL), to this was added Hydrogen chloride (15 mL, 90.00 mmol) and the reaction was stirred for 2 hours to afford a white solid. The precipitate was collected by filtration, washed with MeCN (10 mL) and air dried to afford 4-(5-(4-methoxyphenoxy)-1H-benzo[d]imidazol-2-yl)piperidin-4-amine (0.870 g) as a tan solid, which was used without further purification.

### 63D. 4-(5-(4-methoxyphenoxy)-1H-benzo[d]imidazol-2-yl)-1-(9H-purin-6-yl)piperidin-4-amine

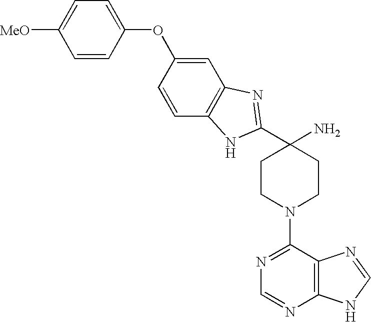

4-(5-(4-Methoxyphenoxy)-1H-benzo[d]imidazol-2-yl)piperidin-4-amine dihydrochloride (0.230 g, 0.56 mmol), 6-chloropurine (0.086 g, 0.56 mmol) and N-ethyldiisopropylamine (0.361 g, 2.80 mmol) were dissolved in DMF (50 mL) and heated at 80° C. for 2 hours. The recation was evaporated to dryness and passed through a 10 g SCX column. The crude product was purified by preparative HPLC using decreasingly polar mixtures of water (containing 1% formic acid) and MeCN as eluents. Fractions containing the desired compound were evaporated to dryness to afford 4-(5-(4-methoxyphenoxy)-1H-benzo[[d]imidazol-2-yl)-1-(9H-purin-6-yl)piperidin-4-amine (0.041 g, 16.06%) a solid; m/z (ESI+) (M+H)+=456; HPLC tR=1.28 min; 1H NMR (400.132 MHz, DMSO) δ 1.73-1.70 (2H, m), 2.19-2.13 (2H, m), 3.67 (3H, s), 4.09-3.94 (2H, m), 4.81-4.57 (2H, m), 6.76 (1H, d), 6.86 (4H, s), 6.96 (1H, s), 7.40 (1H, d), 8.04 (1H, s), 8.15 (1H, s).

### EXAMPLE 64

### 4-(5-(4-Methoxyphenoxy)-1H-benzo[d]imidazol-2-yl)-1-(7H-pyrrolo[2,3-d]pyrimidin-4-yl)piperidin-4-amine

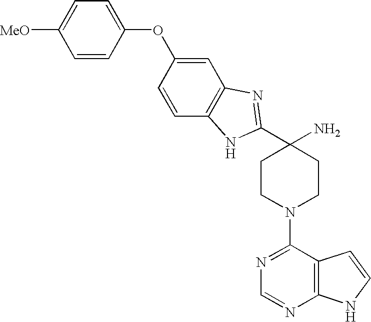

4-(5-(4-methoxyphenoxy)-1H-benzo[d]imidazol-2-yl)piperidin-4-amine dihydrochloride (0.64 g, 1.56 mmol), 6-Chloro-7-deazapurineand N-Ethyldiisopropylamine (1.385 mL, 7.78 mmol) were dissolved in DMF (30 mL) and heated at 80° C. for 2 hours. The recation was evaporated to dryness and passed through a 10 g SCX column. On dissolving for preperative HPLC a brown solid crashed out, this was filtered and dried. The remaing crude product was purified by preparative HPLC using decreasingly polar mixtures of water (containing 1% formic acid) and MeCN as eluents this was further purified by preparative HPLC using decreasingly polar mixtures of water (containing 1% NH3) and MeCN as eluents. Fractions containing the desired compound were evaporated to dryness to afford 4-(5-(4-methoxyphenoxy)-1H-benzo[d]imidazol-2-yl)-1-(7H-pyrrolo[2,3-d]pyrimidin-4-yl)piperidin-4-amine (0.168 g, 23.70%) as a white solid. m/z (ESI+) (M+H)+=456; HPLC tR=1.88 min; 1H NMR (400.132 MHz, DMSO) δ 1.72-1.68 (2H, m), 2.19-2.12 (2H, m), 3.66 (3H, s), 3.84-3.79 (2H, m), 4.21-4.17 (2H, m), 6.55 (1H, d), 6.76-6.74 (1H, m), 6.95 (4H, s), 7.10 (1H, d), 7.39 (1H, d), 8.08 (1H, s), 11.58 (1H, s).

### EXAMPLE 65

### 1-(7H-Pyrrolo]2,3-d]pyrimidin-4-yl)-4-(5-(trifluoromethylthio)-1H-benzo[d]imidazol-2-yl)piperidin-4-amine

### 65A. 4-(5-(trifluoromethylthio)-1H-benzo]d]imidazol-2-yl)piperidin-4-amine dihydrochloride

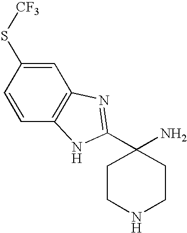

1-(tert-Butoxycarbonyl)-4-(tert-butoxycarbonylamino)piperidine-4-carboxylic acid (1.000 g, 2.90 mmol),4-(trifluoromethylthio)benzene-1,2-diamine (0.665 g, 3.19 mmol), O-(7-Azabenzotriazol-1-yl)-N,N,N′,N′-tetramethyluronium hexafluorophosphate (1.435 g, 3.77 mmol) and N-ethyldiisopropylamine (1.338 mL, 7.55 mmol) were added to DMF (50 mL) and stirred at 55° C. overnight. The reaction was evaporated to dryness, quenched with 2M NaOH (50 mL), extracted with diethyl ether (3×100 mL), the organic layer was dried over MgSO₄, filtered and evaporated to afford black gum. The crude product was purified by flash silica chromatography, elution gradient 0 to 3% MeOH in DCM. Pure fractions were evaporated to dryness to afford the amide as a brown residue (0.35 g). This was dissolved in Toluene (40 ml) and p-Toluenesulfonic acid (0.047 mL, 0.29 mmol) was added and the reaction was heated at reflux over the weekend. LCMS indicated product, mono deboc and di deboc material. The reaction was evaporated to dryness dissolved in acetonitrile and treated with Hydrogen chloride (10 mL, 60.00 mmol), the reaction was stirred for 1 hour. The precipitate was collected by filtration, washed with MeCN (15 mL) and air dried to afford 4-(5-(trifluoromethylthio)-1H-benzo[d]imidazol-2-yl)piperidin-4-amine dihydrochloride (0.240 g, 21.23%) as a white solid, which was used without further purification. m/z (ESI+) (M+H)+=316; HPLC tR=1.87 min.

### 65B. 1-(7H-Pyrrolo[2,3-d]pyrimidin-4-yl)-4-(5-(trifluoromethylthio)-1H-benzo[d]imidazol-2-yl)piperidin-4-amine

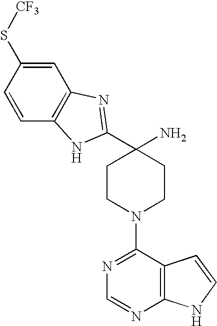

4-(5-(Trifluoromethylthio)-1H-benzo[d]imidazol-2-yl)piperidin-4-amine dihydrochloride (0.24 g, 0.62 mmol), N-ethyldiisopropylamine (0.533 mL, 3.08 mmol) and 6-chloro-7-deazapurine (0.099 g, 0.65 mmol) were dissolved in DMF (30 mL) and heated at 85° C. overnight. The reaction was evaporated to dryness and the crude product was purified by ion exchange chromatography, using an SCX column. The desired product was eluted from the column using 7M NH3/MeOH and pure fractions were evaporated to dryness to afford a yellow gum. The crude product was purified by flash silica chromatography, elution gradient 0 to 10% MeOH in DCM. Pure fractions were evaporated to dryness to afford 1-(7H-pyrrolo[2,3-d]pyrimidin-4-yl)-4-(5-(trifluoromethylthio)-1H-benzo[d]imidazol-2-yl)piperidin-4-amine (0.058 g, 21.70%) as a tan solid; m/z (ESI+) (M+H)+=434; HPLC tR=1.82 min; 1H NMR (400.132 MHz, CDCl3) δ 1.92-1.88 (2H, m), 2.64-2.57 (2H, m), 4.04-3.99 (2H, m), 4.45-4.41 (2H, m), 6.53 (1H, d), 7.07 (1H, d), 7.28 (1H, s), 7.53 (1H, d), 7.63-7.59 (1H, m), 7.95 (1H, s), 8.34 (1H, s), 10.56 (1H, s).

### EXAMPLE 66

### (4-(5-chloro-1H-benzo[d]imidazol-2-yl)-1-(9H-purin-6-yl)piperidin-4-yl)methanamine

### 66A. 5-chloro-1H-benzo[d]imidazol-2(3H)-one

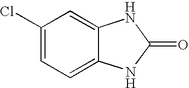

4-Chloro-1,2-phenylenediamine (20 g, 140.27 mmol) was dissolved in THF (250 mL), to this was added portionwise 1,1′-Carbonyldiimidazole (27.3 g, 168.32 mmol) and the reaction was stirred overnight at 25° C. The black reaction was evaporated to dryness, quenched with 2.0N HCl (100 mL) and filtered. The obtained solid was dissolved in methanol and passed through a 50 g SCX column, to afford 5-chloro-1H-benzo[d]imidazol-2(3H)-one (14.6 g, 61.7%) as a yellow solid. m/z (ESI+) (M+H)+=169; HPLC tR=1.14 min; 1H NMR (399.902 MHz, DMSO) δ 6.91 (1H, d), 6.97-6.94 (2H, m), 10.74 (2H, s).

### 66B. 2,5-dichloro-1H-benzo[d]imidazole

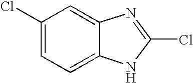

5-chloro-1H-benzo[d]imidazol-2(3H)-one (13.00 g, 77.11 mmol) was added to Phosphorus oxychloride (108 ml, 1156.72 mmol) and heated at reflux for 4 hours The reaction was evaporated to dryness, the obtained brown gum was quenched with ice (50 g) and acetonitrile (100 ml), the resulting solution was filtered and the liquor was basified with NaHCO3. The acetonitrile was evaporated and the solid was dissolved in ethanol, the reaction mixture was filtered through celite, dried (MgSO4) and solvent evaporated to afford 2,5-dichloro-1H-benzo[d]imidazole (5.34 g, 37.0%) as a yellow solid; 1H NMR (400.132 MHz, DMSO) δ 7.20 (1H, dd), 7.47 (1H, d), 7.54 (1H, d).

### 66C. 2,5-dichloro-N,N-dimethyl-1H-benzo[d]imidazole-1-sulfonamide and 2,6-dichloro-N,N-dimethyl-1H-benzo[d]imidazole-1-sulfonamide

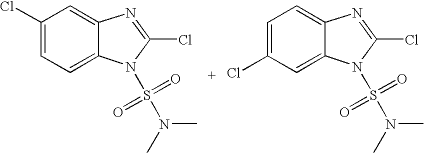

2,5-dichloro-1H-benzo[d]imidazole (5.34 g, 28.55 mmol) and 1,4-Diazabicyclo[2.2.2]octane (2.81 mL, 28.55 mmol) were dissolved in DMF (200 mL), to this was added Dimethylsulfamoyl chloride (3.07 mL, 28.55 mmol) and the reaction was stirred overnight. The reaction was filtered and solvent evaporated to dryness. The reaction mixture was quenched with water (100 mL), extracted with diethyl ether (3×200 mL), the organic layer was dried over MgSO4, filtered and evaporated to afford yellow solid. The crude product was purified by flash silica chromatography, elution gradient 100% DCM. Pure fractions were evaporated to dryness to afford as a yellow solid. This was dissolved in DCM/diethyl ether, the solvent was slowly evaporated until solid was visible in the flask, this was then stirred for 1 hour to afford 2,5-dichloro-N,N-dimethyl-1H-benzo[d]imidazole-1-sulfonamide (7.40 g, 88%) as a white solid; m/z (ESI+) (M+H)+=294; HPLC tR=2.35 min; 1H NMR (400.132 MHz, CDCl3) δ 3.04 (3H, s), 3.06 (3H, s), 7.36 (1H, ddd), 7.62 (0.5H, d), 7.69 (0.5H, d), 7.87 (0.5H, d), 7.97 (0.5H, d).

### 66D. tert-Butyl 4-(5-chloro-1-(N,N-dimethylsulfamoyl)-1H-benzo[d]imidazol-2-yl)-4-cyanopiperidine-1-carboxylate

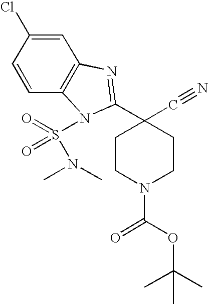

Lithium diisopropylamide (2.62 ml, 5.23 mmol) was dissolved in THF (50 mL) and cooled to −78° C., to this was slowly added 1-N-Boc-4-cyanopiperidine (1 g, 4.76 mmol) in THF (5.0 ml) and the reaction was stirred for 10 minutes at -78° C. before the rapid addition of 2,5-dichloro-N,N-dimethyl-1H-benzo[d]imidazole-1-sulfonamide (1.399 g, 4.76 mmol). The reaction was stirred for 30 minutes at −78° C. before being warmed to 25° C. and stirred overnight. The reaction mixture was quenched with saturated NH4Cl (50 mL), extracted with diethyl ether (3×75 mL), the organic layer was dried over MgSO4, filtered and evaporated to afford an orange gum. The crude product was purified by flash silica chromatography, elution gradient 20 to 100% Et20 in isohexane. Pure fractions were evaporated to dryness to afford tert-butyl 4-(5-chloro-1-(N,N-dimethylsulfamoyl)-1H-benzo[d]imidazol-2-yl)-4-cyanopiperidine-1-carboxylate (1.440 g, 64.7%) as a yellow gum. The columed gum was triturated with 20% Et₂O in iso-hexane to give a solid which was collected by filtration and dried under vacuum to give tert-butyl 4-(5-chloro-1-(N,N-dimethylsulfamoyl)-1H-benzo[d]imidazol-2-yl)-4-cyanopiperidine-1-carboxylate (1.440 g, 64.7%) as a white solid. m/z (ESI+) (M+H)+=468; HPLC tR=3.02 min; 1H NMR (400.132 MHz, CDCl3) δ 1.49 (9H, s), 2.24 (2H, ddd), 2.76-2.73 (2H, m), 3.07 (6H, s), 3.36-3.30 (2H, m), 4.27-4.16 (2H, m), 7.40 (1H, dd), 7.69 (1H, d), 7.80 (1H, d).

### 66E. 4-(5-chloro-1H-benzo[d]imidazol-2-yl)piperidine-4-carbonitrile

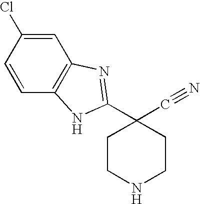

tert-Butyl 4-(5-chloro-1-(N,N-dimethylsulfamoyl)-1H-benzo[d]imidazol-2-yl)-4-cyanopiperidine-1-carboxylate (1.45 g, 3.10 mmol) was added to acetonitrile (10 mL) and 6.0 N Hydrochloric acid in propan-2-ol (25.8 mL, 154.92 mmol). The reaction was heated at 50° C. until the solid dissolved. The reaction was then stirred at 25° C. overnight to afford a white solid. The precipitate was collected by filtration, washed with MeCN (50 mL) and dried under vacuum to afford 4-(5-chloro-1H-benzo[d]imidazol-2-yl)piperidine-4-carbonitrile (1.200 g, 116%) as a white solid, which was used without further purification; m/z (ESI+) (M+H)+=261; HPLC tR=1.25 min.

### 66F. 4-(5-chloro-1H-benzo[d]imidazol-2-yl)-1-(9H-purin-6-yl)piperidine-4-carbonitrile

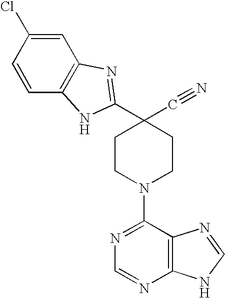

4-(5-chloro-1H-benzo[d]imidazol-2-yl)piperidine-4-carbonitrile hydrochloride (0.35 g, 1.18 mmol), 6-Chloropurine (0.182 g, 1.18 mmol) and Triethylamine (0.492 mL, 3.53 mmol) were dissolved in DMA (40 mL) and heated at 80° C. for 2 hours. The reaction was evaporated to dryness, the crude product was purified by ion exchange chromatography, using an SCX column. The desired product was eluted from the column using 7M NH3/MeOH to afford a brown solid. The crude solid was triturated with MeOH to give a solid which was collected by filtration and dried under vacuum to give 4-(5-chloro-1H-benzo[d]imidazol-2-yl)-1-(9H-purin-6-yl)piperidine-4-carbonitrile (0.350 g, 78%) as a white solid ; m/z (ESI+) (M+H)+=379; HPLC tR=1.27 min; 1H NMR (400.132 MHz, DMSO) δ 2.29-2.22 (2H, m), 2.40-2.37 (2H, m), 3.56-3.50 (2H, m), 5.41-5.29 (2H, m), 7.18 (1H, d), 8.12 (1H, s), 8.22 (1H, s).

### 66G. (4-(5-chloro-1H-benzo[d]imidazol-2-yl)-1-(9H-purin-6-yl)piperidin-4-yl)methanamine

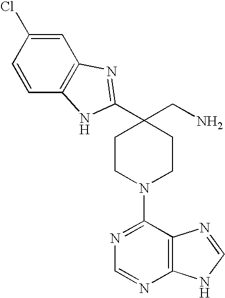

4-(5-Chloro-1H-benzo[d]imidazol-2-yl)-1-(9H-purin-6-yl)piperidine-4-carbonitrile (0.35 g, 0.92 mmol) and platinum(IV) oxide (83% Pt (0.210 g, 0.92 mmol) were dissolved in acetic acid (30 mL) and subjected to hydrogenation. The reaction mixture was then filtered and evaporated to dryness and the crude product was purified by ion exchange chromatography, using an SCX column. The desired product was eluted from the column using 7M NH3/MeOH afford a brown residue. This was purified by flash silica chromatography, elution gradient 10% MeOH in DCM and finally 10% 7.0 N NH3 in methanol in DCM. Fractions were evaporated to dryness to afford (4-(5-chloro-1H-benzo[d]imidazol-2-yl)-1-(9H-purin-6-yl)piperidin-4-yl)methanamine (0.130 g, 36.8%) as a brown residue. The crude residue was triturated with hot MeOH to give a solid which was collected by filtration and dried under vacuum to give (4-(5-chloro-1H-benzo[d]imidazol-2-yl)-1-(9H-purin-6-yl)piperidin-4-yl)methanamine (0.130 g, 36.8%) as a tan solid ; m/z (ESI+) M+=383; HPLC tR=1.27 min; 1H NMR (400.132 MHz, DMSO) δ 1.74-1.67 (2H, m), 2.35-2.32 (2H, m), 2.73 (2H, s), 5.11-4.76 (2H, m), 7.10 (1H, dd), 7.47 (1H, d), 7.51 (1H, d), 8.03 (1H, s), 8.13 (1H, s).

### EXAMPLE 67

### 67A. 4-(5-chloro-1H-benzo[d]imidazol-2-yl)-1-(7H-pyrrolo[2,3-d]pyrimidin-4-yl)piperidine-4-carbonitrile

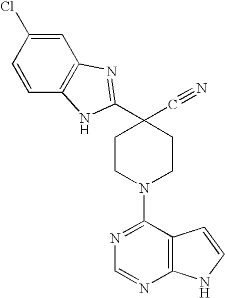

4-(5-Chloro-1H-benzo[d]imidazol-2-yl)piperidine-4-carbonitrile hydrochloride (0.35 g, 1.18 mmol), 6-chloro-7-deazapurine (0.181 g, 1.18 mmol) and triethylamine (0.492 mL, 3.53 mmol) were dissolved in DMA (15 mL) and heated at 80° C. for 2 hours. The reaction was evaporated to dryness, the crude product was purified by ion exchange chromatography, using an SCX column. The desired product was eluted from the column using 7M NH3/MeOH to afford a brown solid. The crude solid was purified by flash silica chromatography, elution gradient 0 to 5% MeOH in DCM, the obtained solid was triturated with MeOH to give a solid which was collected by filtration and dried under vacuum to give 4-(5-chloro-1H-benzo[d]imidazol-2-yl)-1-(7H-pyrrolo[2,3-d]pyrimidin-4-yl)piperidine-4-carbonitrile (0.260 g, 58.4%) as a white solid ; m/z (ESI+) (M+H)+=377; HPLC tR=1.69 min; 1H NMR (400.132 MHz, DMSO) δ 2.36-2.29 (2H, m), 2.47-2.43 (2H, m), 3.56-3.50 (2H, m), 4.78-4.75 (2H, m), 6.69 (1H, s), 7.29-7.24 (2H, m), 7.77-7.48 (2H, m), 8.22 (1H, s), 11.76 (1H, s), 13.04 (1H, s).

### 67B. (4-(5-chloro-1H-benzo[d]imidazol-2-yl)-1-(7H-pyrrolo[2,3-d]pyrimidin-4-yl)piperidin-4-yl)methanamine

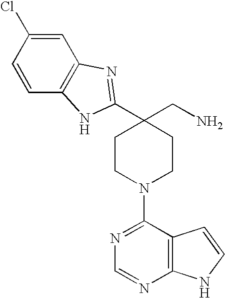

4-(5-chloro-1H-benzo[d]imidazol-2-yl)-1-(7H-pyrrolo[2,3-d]pyrimidin-4-yl)piperidine-4-carbonitrile (0.26 g, 0.69 mmol) was subjected to hydrogenation over platinum(IV) oxide catalyst as described in Example 66G. The reaction mixture was then filtered, evaporated to dryness and the crude product was purified by ion exchange chromatography, using an SCX column. The desired product was eluted from the column using 7M NH3/MeOH to afford a dark solid which was purified by preparative HPLC (Waters XBridge Prep C18 OBD column, 5μ silica, 50 mm diameter, 150 mm length), using decreasingly polar mixtures of water (containing 1% NH3) and MeCN as eluents. Fractions containing the desired compound were evaporated to dryness to afford (4-(5-chloro-1H-benzo[d]imidazol-2-yl)-1-(7H-pyrrolo[2,3-d]pyrimidin-4-yl)piperidin-4-yl)methanamine (0.041 g, 15.60%) as a white solid; m/z (ESI+) (M+H)+=381; HPLC tR=1.55 min; 1H NMR (400.132 MHz, DMSO) δ 1.74-1.68 (2H, m), 2.35-2.32 (2H, m), 2.74 (2H, s), 3.31-3.23 (2H, m), 4.39-4.35 (2H, m), 6.52 (1H, d), 7.11-7.08 (2H, m), 7.51 (1H, s), 8.06 (1H, s), 11.56 (1H, s).

### EXAMPLE 68

### Preparation of 4-(5-Phenyl-1H-benzo[d]imidazol-2-yl)-1-(7H-pyrrolo[2,3-d]pyrimidin-4-yl)piperidin-4-amine

### 68A. tert-Butyl 4-(2-amino-4-bromophenylcarbamoyl)-1-(7H-pyrrolo[2,3-d]pyrimidin-4-yl)piperidin-4-ylcarbamate and tert-Butyl 4-(2-amino-5-bromophenylcarbamoyl)-1-(7H-pyrrolo[2,3-d]pyrimidin-4-yl)piperidin-4-ylcarbamate

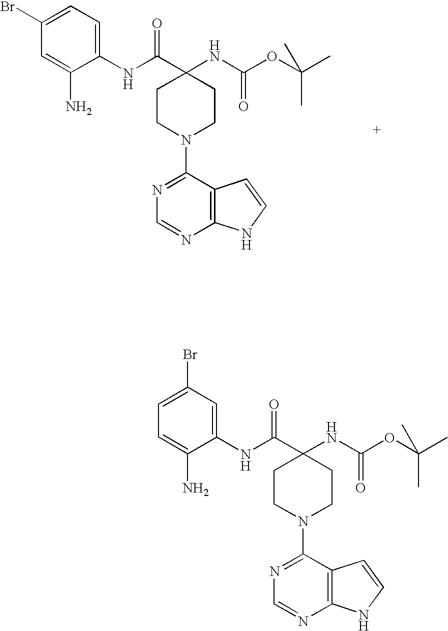

1-Hydroxybenzotriazole (4.11 g, 30.44 mmol) was added to 4-(tert-butoxycarbonylamino)-1-(7H-pyrrolo[2,3-d]pyrimidin-4-yl)piperidine-4-carboxylic acid (10 g, 27.67 mmol) (Example 50B), 2-amino-4-bromophenylamine (7.76 g, 41.51 mmol) and 1-ethyl-3-(3-dimethylaminopropyl)carbodiimide hydrochloride (7.96 g, 41.51 mmol) in N,N-dimethylformamide (50 mL) at ambient temperature. The resulting slurry was stirred at 60° C. for 18 hours. The reaction mixture was cooled to room temperature then partitioned between water (500 ml) and ethyl acetate (500 ml). The aqueous layer was re-extracted with ethyl acetate (250 ml) then the combined organics were filtered and washed sequentially with water (250 mL), saturated Na2CO3 (250 mL), and saturated brine (250 mL). The organic layer was dried over MgSO4, filtered and evaporated to afford crude product. The crude product was purified by flash silica chromatography, elution gradient 0 to 5% MeOH in DCM. Fractions containing product were evaporated to dryness then redissolved in DCM and ether added until precipitation started to occur. The mixture was left for 1 hr to allow complete precipitation of the product then the precipitate was collected by filtration, washed with DCM and air dried to afford tert-butyl 4-(2-amino-4-bromophenylcarbamoyl)-1-(7H-pyrrolo[2,3-d]pyrimidin-4-yl)piperidin-4-ylcarbamate compound with tert-butyl 4-(2-amino-5-bromophenylcarbamoyl)-1-(7H-pyrrolo[2,3-d]pyrimidin-4-yl)piperidin-4-ylcarbamate (3.25 g, 22.11%) as a beige solid; NMR Spectrum: Major Isomer 1H NMR (399.902 MHz, DMSO) δ 1.35 (9H, s), 1.98 (4H, m), 3.58 (2H, m), 4.19 (2H, m), 5.06 (2H, s), 6.56 (2H, m), 6.75 (2H, m), 7.10 (1H, m), 7.29 (1H, s), 8.07 (1H, s), 8.96 (1H, s), 11.59 (1H, s) Minor Isomer 1H NMR (399.902 MHz, DMSO) δ 1.35 (9H, s), 1.98 (4H, m), 3.58 (2H, m), 4.18 (2H, m), 4.95 (2H, s), 6.56 (2H, m), 7.00 (2H, m), 7.10 (1H, m), 7.29 (1H, s), 8.07 (1H, s), 9.00 (1H, s), 11.59 (1H, s);

Mass Spectrum: M+H⁺0 530, 532.

### 68B. 4-(5-bromo-1H-benzo[d]imidazol-2-yl)-1-(7H-pyrrolo[2,3-d]pyrimidin-4-yl)piperidin-4-amine

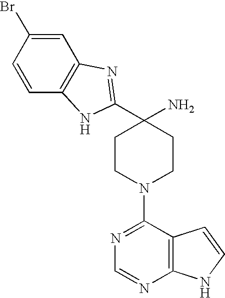

4M HCl in dioxane (44.2 mL, 176.84 mmol) was added to a mixture of tert-butyl 4-(2-amino-4-bromophenylcarbamoyl)-1-(7H-pyrrolo[2,3-d]pyrimidin-4-yl)piperidin-4-ylcarbamate compound with tert-butyl 4-(2-amino-5-bromophenylcarbamoyl)-1-(7H-pyrrolo[2,3-d]pyrimidin-4-yl)piperidin-4-ylcarbamate (4.69 g, 8.84 mmol) in NMP (40 mL) at ambient temperature. The resulting solution was stirred at 90° C. for 7 hours. The reaction mixture was cooled to room temperature, diluted with MeOH then purified by ion exchange chromatography, using an SCX column. The crude product was eluted from the column using 2M NH3/MeOH and fractions were evaporated to dryness to afford a brown gum. The crude product was purified by flash silica chromatography, elution gradient 0 to 10% 2M ammonia in MeOH in DCM. Pure fractions were evaporated to dryness to afford 4-(5-bromo-1H-benzo[d]imidazol-2-yl)-1-(7H-pyrrolo[2,3-d]pyrimidin-4-yl)piperidin-4-amine (3.09 g, 85%) as a beige solid; NMR Spectrum: 1H NMR (399.902 MHz, DMSO) δ 1.78 (2H, m), 2.23 (2H, m), 3.87 (2H, m), 4.29 (2H, m), 6.63 (1H, m), 7.18 (1H, s), 7.27 (1H, m), 7.46 (1H, m), 7.68 (1H, s), 8.16 (1H, s), 11.66 (1H, s), 12.32 (1H, s); Mass Spectrum: M+H⁺ 414.

### 68C. tert-Butyl 4-(4-(5-bromo-1H-benzo[d]imidazol-2-yl)-4-(tert-butoxycarbonylamino)piperidin-1-yl)-7H-pyrrolo[2,3-d]pyrimidine-7-carboxylate

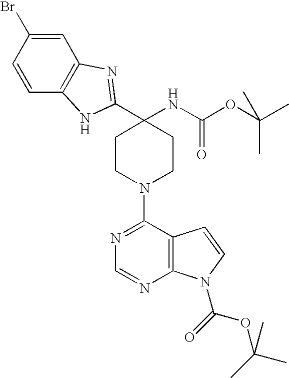

Di-tert-butyl dicarbonate (16.99 g, 77.86 mmol) was added to 445-bromo-1H-benzo[d]imidazol-2-yl)-1 47H-pyrrolo[2,3-d]pyrimidin-4-yl)piperidin-4-amine (3.21 g, 7.79 mmol) in THF (50 mL). The resulting solution was stirred at ambient temperature for 72 hours. The reaction mixture was evaporated then purified by flash silica chromatography, elution gradient 0 to 5% MeOH in DCM. Pure fractions were evaporated to dryness to afford tert-butyl 4-(4-(5-bromo-1H-benzo[d]imidazol-2-yl)-4-(tert-butoxycarbonylamino)piperidin-1-yl)-7H-pyrrolo[2,3-d]pyrimidine-7-carboxylate (3.94 g, 83%) as a grey solid; Mass Spectrum: M+H⁺ 614.

### 68D. 4-(5-phenyl-1H-benzo[d]imidazol-2-yl)-1-(7H-pyrrolo[2,3-d]pyrimidin-4-yl)piperidin-4-amine

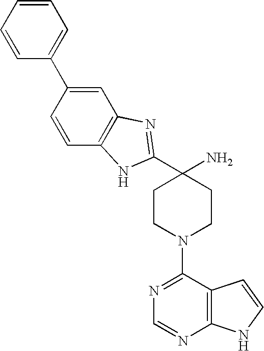

tert-Butyl 4-(4-(5-bromo-1H-benzo[d]imidazol-2-yl)-44tert-butoxycarbonyl-amino)piperidin-1-yl)-7H-pyrrolo[2,3-d]pyrimidine-7-carboxylate (200 mg, 0.33 mmol), phenylboronic acid (51.8 mg, 0.42 mmol) and tri-potassium orthophosphate (347 mg, 1.63 mmol) were suspended in a mixture of dioxane (3 mL) and water (0.750 mL) and bubbled with nitrogen for 5 minutes. Tetrakis(triphenylphosphine)-palladium(0) (18.87 mg, 0.02 mmol) was added and the reaction was heated to 110° C. for 30 minutes in a microwave reactor and cooled to RT. The reaction mixture was acidified with acetic acid and added to a SCX column. The protected crude product was eluted from the column using 2M ammonia in MeOH and solvents were evaporated to dryness. The residues were dissolved in a mixture of DCM (2 mL) and TFA (2 mL) and stirred at ambient temperature for 45 minutes. The reaction mixture was again added to a SCX column. The desired crude product was eluted from the column using 2M ammonia in MeOH and solvents were evaporated to dryness to afford the crude product. The crude product was purified by preparative HPLC using decreasingly polar mixtures of water (containing 1% NH3) and MeCN as eluents. Fractions containing the desired compound were evaporated to dryness to afford 4-(5-phenyl-1H-benzo[d]imidazol-2-yl)-1-(7H-pyrrolo[2,3-d]pyrimidin-4-yl)piperidin-4-amine (41.0 mg, 30.7%) as a white solid; NMR Spectrum: 1H NMR (399.902 MHz, DMSO) δ 1.80 (2H, m), 2.27 (2H, m), 3.91 (2H, m), 4.28 (2H, m), 6.64 (1H, m), 7.19 (1H, m), 7.33 (1H, m), 7.45 (3H, m), 7.57 (1H, m), 7.67 (2H, m), 7.74 (1H, m), 8.17 (1H, s), 11.66 (1H, s), 12.23 (1H, br s); Mass Spectrum: M+H⁺ 410.

### EXAMPLES 69 TO 78

The compounds of Examples 69 to 78 were prepared in an identical manner to the compound of Example 68, using the intermediate tert-butyl 4-(4-(5-bromo-1H-benzo[d]imidazol-2-yl)-4-(tert-butoxycarbonylamino)piperidin-1-yl)-7H-pyrrolo[2,3-d]pyrimidine-7-carboxylate

| Ex. | | Chemical | NMR | Mass |
|---|---|---|---|---|
| No. | Structure | Name | Data | Spectrum |
| 69 | 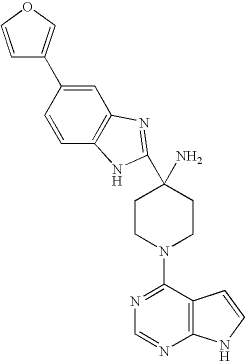 | 4-(5-furan-3-yl-1H- benzimidazol-2-yl)- 1-(7H-pyrrolo[2,3- d]pyrimidin-4- yl)piperidin-4-amine | 1H NMR (399.902 MHz, DMSO) δ 1.79 (2 H, m), 2.26 (2 H, m), 3.91 (2 H, m), 4.27 (2 H, m), 6.64 (1 H, m), 6.96 (1 H, m), 7.18 (1 H, m), 7.40 (1 H, m), 7.48 (1 H, m), 7.70 (2 H, m), 8.12 (1 H, s), 8.16 (1 H, s), 11.66 (1 H, s), 12.15 (1 H, s) NH₂ missing | M + H⁺ 400 |
| 70 | 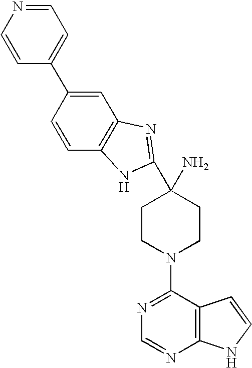 | 4-(5-pyridin-4-yl-1H- benzimidazol-2-yl)- 1-(7H-pyrrolo[2,3- d]pyrimidin-4- yl)piperidin-4-amine | 1H NMR (700.034 MHz, DMSO) δ 2.12 (2 H, m), 2.68 (2 H, m), 3.93 (2 H, m), 4.20 (2 H, m), 6.69 (1 H, m), 7.25 (1 H, m), 7.74-8.15 (5 H, m), 8.22 (1 H, s), 8.67 (2 H, m), 8.86 (2 H, s), 11.80 (1 H, s), 13.04 (1 H, s) | M + H⁺ 411 |
| 71 | 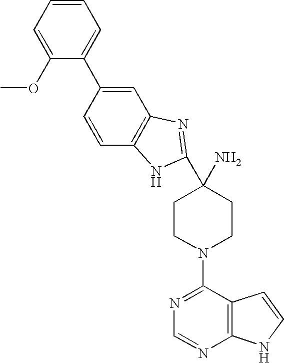 | 4-[5-(2- methoxyphenyl)-1H- benzimidazol-2-yl]- 1-(7H-pyrrolo[2,3- d]pyrimidin-4- yl)piperidin-4-amine | 1H NMR (399.902 MHz, DMSO) δ 1.72 (2 H, m), 2.19 (2 H, m), 3.68 (3 H, s), 3.84 (2 H, m), 4.20 (2 H, m), 6.56 (1 H, m), 6.95 (1 H, m), 7.03 (1 H, m), 7.10 (1 H, m), 7.14 (1 H, m), 7.23 (2 H, m), 7.45 (2 H, m), 8.08 (1 H, s), 11.57 (1 H, s), 12.08 (1 H, s) NH₂ missing | M + H⁺ 440 |
| 72 | 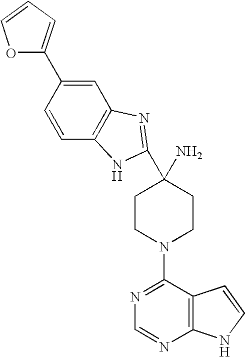 | 4-[5-furan-2-yl)-1H- benzimidazol-2-yl]- 1-(7H-pyrrolo[2,3- d]pyrimidin-4- yl)piperidin-4-amine | 1H NMR (399.902 MHz, DMSO) δ 1.72 (2 H, m), 2.17 (2 H, m), 3.82 (2 H, m), 4.21 (2 H, m), 6.50 (1 H, m), 6.56 (1 H, m), 6.76 (1 H, m), 7.11 (1 H, m), 7.44 (2 H, m), 7.63 (1 H, m), 7.71 (1 H, m), 8.08 (1 H, s), 11.58 (1 H, s), 12.16 (1 H, s) NH₂ missing | M + H⁺ 400 |
| 73 | 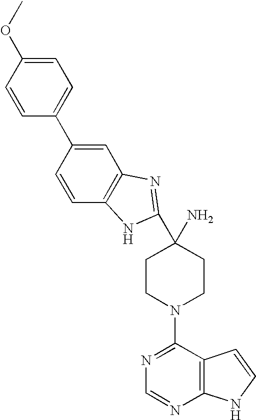 | 4-[5-(4- methoxyphenyl)-1H- benzimidazol-2-yl]- 1-(7H-pyrrolo[2,3- d]pyrimidin-4- yl)piperidin-4-amine | 1H NMR (399.902 MHz, DMSO) δ 1.80 (2 H, m), 2.27 (2 H, m), 3.80 (3 H, s), 3.91 (2 H, m), 4.28 (2 H, m), 6.64 (1 H, m), 7.02 (2 H, d), 7.19 (1 H, m), 7.38 (1 H, m), 7.53-7.67 (4 H, m), 8.16 (1 H, s), 11.66 (1 H, s), 12.16 (1 H, s) NH₂ missing | M + H⁺ 440 |
| 74 | 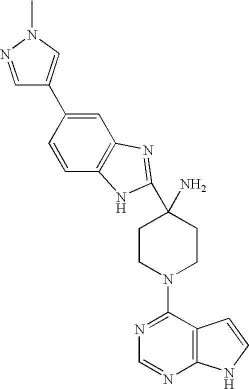 | 4-[5-(1- methylpyrazol-4-yl)- 1H-benzimidazol-2- yl]-1-(7H- pyrrolo[2,3- d]pyrimidin-4- yl)piperidin-4-amine | 1H NMR (399.902 MHz, DMSO) δ 1.79 (2 H, m), 2.25 (2 H, m), 3.90 (5 H, m), 4.27 (2 H, m), 6.64 (1 H, m), 7.18 (1 H, m), 7.34 (1 H, m), 7.46 (1 H, m), 7.62 (1 H, m), 7.81 (1 H, s), 8.06 (1 H, s), 8.16 (1 H, s), 11.65 (1 H, s), 12.09 (1 H, s) NH₂ missing | M + H⁺ 414 |
| 75 | 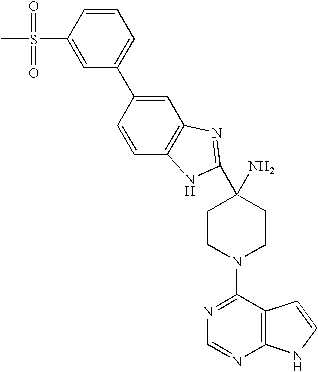 | 4-[5-(3- methylsulfonylphenyl)- 1H-benzimidazol-2- yl]-1-(7H- pyrrolo[2,3- d]pyrimidin-4- yl)piperidin-4-amine | 1H NMR (399.902 MHz, DMSO) δ 1.92 (3 H, s), 2.08 (2 H, m), 2.62 (2 H, m), 4.01 (2 H, m), 4.16 (2 H, m), 6.69 (1 H, m), 7.25 (1 H, m), 7.65- 8.09 (6 H, m), 8.19 (1 H, s), 8.22 (1 H, s), 11.77 (1 H, s), 12.86 (1 H, s) NH₂ missing | M + H⁺ 488 |
| 76 | 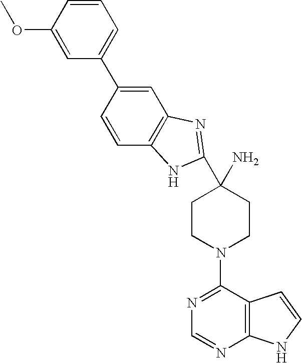 | 4-[5-(3- methoxyphenyl)-1H- benzimidazol-2-yl]- 1-(7H-pyrrolo[2,3- d]pyrimidin-4- yl)piperidin-4-amine | 1H NMR (399.902 MHz, DMSO) δ 1.72 (2 H, m), 2.19 (2 H, m), 3.75 (3 H, s), 3.84 (2 H, m), 4.20 (2 H, m), 6.56 (1 H, m), 6.82 (1 H, m), 7.11 (2 H, m), 7.15 (1 H, m), 7.29 (1 H, m), 7.36 (1 H, m), 7.47 (1 H, m), 7.66 (1 H, m), 8.09 (1 H, s), 11.58 (1 H, s), 12.14 (1 H, s) NH₂ missing | M + H⁺ 440 |
| 77 | 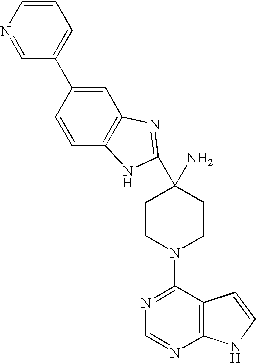 | 4-(5-pyridin-3-yl-1H- benzimidazol-2-yl)- 1-(7H-pyrrolo[2,3- d]pyrimidin-4- yl)piperidin-4-amine | 1H NMR (700.034 MHz, DMSO) δ 2.16 (2 H, m), 2.72 (2 H, m), 3.95 (2 H, m), 4.23 (2 H, m), 6.78 (1 H, s), 7.32 (1 H, s), 7.64 (2 H, m), 7.77 (1 H, m), 7.98 (1 H, s), 8.28 (2 H, m), 8.63 (1 H, m), 8.89 (2 H, s), 9.01 (1 H, m), 12.05 (1 H, s) Imidazole NH missing | M + H⁺ 411 |
| 78 | 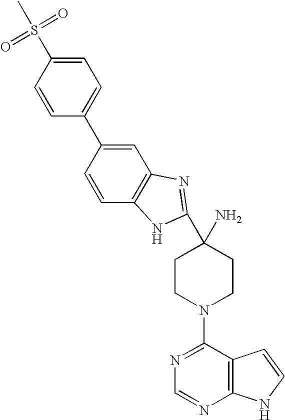 | 4-[5-(4- methylsulfonylphenyl)- 1H-benzimidazol-2- yl]-1-(7H- pyrrolo[2,3- d]pyrimidin-4- yl)piperidin-4-amine | 1H NMR (399.902 MHz, DMSO) δ 1.82 (2 H, m), 2.28 (2 H, m), 3.25 (3 H, s), 3.91 (2 H, m), 4.29 (2 H, m), 6.65 (1 H, m), 7.19 (1 H, m), 7.54 (1 H, m), 7.62 (1 H, m), 7.87 (1 H, m), 7.97 (4 H, m), 8.17 (1 H, s), 11.67 (1 H, s), 12.30 (1 H, s) NH2 missing | M + H⁺ 400 |

Preparation of Biaryl and Heteroaryl-Aryl Intermediates B-1 to B-6

Preparation B-1

### N-(2-Nitro-6-pyridin-3-ylphenyl)acetamide

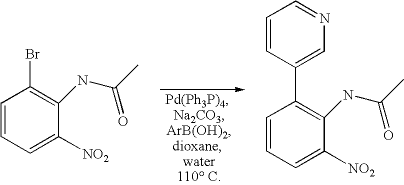

N-(2-Bromo-6-nitrophenyl)acetamide (485 mg, 1.87 mmol), 3-(4,4,5,5-tetramethyl-1,3,2-dioxaborolan-2-yl)pyridine (461 mg, 2.25 mmol) and sodium carbonate (794 mg, 7.49 mmol) were stirred in dioxane (35 ml) and water (7.3 ml). The mixture was purged with nitrogen for 15 minutes and then Pd(Ph₃P)₄ (44 mg, 0.04 mmol) added. The mixture was heated at 110° C. for 2 h, cooled, filtered and concentrated. The residue was taken up in DCM washed with brine, dried (MgSO₄) and concentrated. Silica column chromatography using increasingly polar mixtures of MeOH in DCM) gave the title compound (386 mg, 80%) as a brown gum.

M/z: [M+H]⁺ 258; ¹H NMR (DMSO-d₆) 1.85 (3H, s), 7.50-7.53 (1H, m), 7.62 (1H, t), 7.75-7.81 (2H, m), 7.98-8.00 (1H, m), 8.58 (1H, d), 8.62-8.63 (1H, m), 9.87 (1H, s)

By following the method described in Preparation B-1, the intermediates B-2 to B-6 were formed.

| Prep. | | Chemical | | | |
|---|---|---|---|---|---|
| No. | Structure | Name | Precursor | N.M.R. Data | M.S. |
| B-2 | 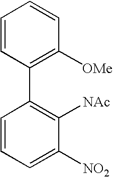 | N-(2′- methoxy-3- nitrobiphenyl- 2- yl)acetamide | 2-methoxy- phenylboronic acid | 1H NMR (DMSO-d₆) 1.81 (3 H, s), 3.69 (3 H, s), 7.03-7.07 (1 H, m), 7.12-7.17 (2 H, m), 7.41-7.45 (1 H, m), 7.49 (1 H, t), 7.57 (1 H, d), 7.86-7.88 (1 H, m), 9.41 (1 H, s) | [M + H]⁺ 285 |
| B-3 | 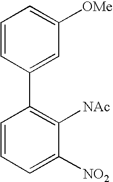 | N-(3′- methoxy-3- nitrobiphenyl- 2- yl)acetamide | 3-methoxy- phenylboronic acid | 1H NMR (DMSO-d₆) 1.86 (3 H, s), 3.80 (3 H, s), 6.93-6.95 (2 H, m), 6.99-7.02 (1 H, m), 7.37-7.41 (1 H, m), 7.57 (1 H, t), 7.69-7.71 (1 H, m), 7.91-7.93 (1 H, m), 9.75 (1 H, s) | [M + H]⁺ 285 |
| B-4 | 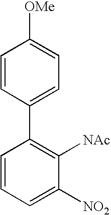 | N-(4′- methoxy-3- nitrobiphenyl- 2- yl)acetamide | 4-methoxy- phenylboronic acid | 1H NMR (DMSO-d₆) 1.87 (3 H, s), 3.82 (3 H, s), 7.03-7.05 (2 H, m), 7.31-7.34 (2 H, m), 7.54 (1 H, t), 7.64-7.67 (1 H, m), 7.86-7.89 (1 H, m), 9.71 (1 H, s) | [M + H]⁺ 285 |
| B-5 | 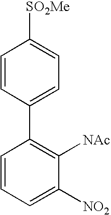 | N-[4′-(methyl- sulfonyl)-3- nitrobiphenyl- 2-yl]- acetamide | 4-(methane- sulfonyl)phenyl- boronic acid | 1H NMR (DMSO-d₆) 1.87 (3 H, s), 3.26 (3 H + H2O, s), 7.61- 7.66 (3 H, m), 7.73-7.76 (1 H, m), 7.99-8.07 (3 H, m), 9.86 (1 H, s) | [M + H]⁺ 334 |
| B-6 | 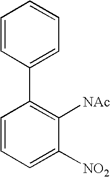 | N-(3- nitrobiphenyl- 2-yl)- acetamide | phenylboronic acid | 1H NMR (DMSO-d₆) 1.85 (3 H, s), 7.37-7.51 (5 H, m), 7.58 (1 H, t), 7.68-7.70 (1 H, m), 7.91-7.94 (1 H, m), 9.75 (1 H, s) | [M + H]⁺ 256 |

De-Acetylation of Biaryl and Heteroaryl-Aryl Intermediates B-1 to B-6

Preparation C-1

### (2-Nitro-6-pyridin-3-ylphenyl)amine

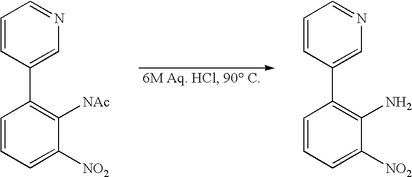

N-(2-Nitro-6-pyridin-3-ylphenyl)acetamide (386 mg, 1.50 mmol) (Intermediate B-1) was suspended in MeOH (5 ml) and 6M hydrochloric acid (10 ml) added. The mixture was heated at 90° C. overnight. The mixture was cooled, neutralised with 2N NaOH and extracted with DCM (×2). The combined extracts were dried (MgSO₄) and concentrated to give the title compound (259 mg, 80%) as a yellow oil that crystallised on standing. M/z: [M+H]⁺ 216; ¹H NMR (DMSO-d₆) 6.78-6.82 (1H, m), 6.89 (2H, s), 7.38-7.40 (1H, m), 7.52-7.56 (1H, m), 7.85-7.88 (1H, m), 8.09-8.12 (1H, m), 8.60-8.61 (1H, m), 8.65-8.67 (1H, m)

By following the method described in Preparation C-1, the biaryl intermediates C-2 to C-6 were formed.

| Prep. | | Chemical | | | |
|---|---|---|---|---|---|
| No. | Structure | Name | Precursor | N.M.R. Data | M.S. |
| C-2 | 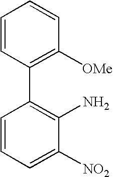 | (2′-methoxy- 3- nitrobiphenyl- 2-yl)amine | N-(2′- methoxy-3- nitrobiphenyl- 2- yl)acetamide | 1H NMR (DMSO-d₆) 3.76 (3 H, s), 6.54 (2 H, s), 6.72- 6.76 (1 H, m), 7.07- 7.11 (1 H, m), 7.17-7.21 (2 H, m), 7.27-7.29 (1 H, m), 7.45- 7.50 (1 H, m), 8.03- 8.06 (1 H, m) | [M + H]⁺ 245 |
| C-3 | 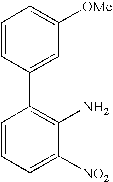 | (3′-methoxy- 3- nitrobiphenyl- 2-yl)amine | N-(3′- methoxy-3- nitrobiphenyl- 2- yl)acetamide | 1H NMR (DMSO-d₆) 3.82 (3 H, s), 6.75-6.80 (3 H, m), 6.97-7.05 (3 H, m), 7.36- 7.38 (1 H, m), 7.45 (1 H, t), 8.05-8.08 (1 H, m) | — |
| C-4 | 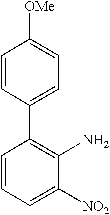 | (4′-methoxy- 3- nitrobiphenyl- 2-yl)amine | N-(4′- methoxy-3- nitrobiphenyl- 2- yl)acetamide | 1H NMR (DMSO-d₆) 3.83 (3 H, s), 6.70 (2 H, s), 6.74- 6.78 (1 H, m), 7.07- 7.11 (2 H, m), 7.31-7.37 (3 H, m), 8.02-8.05 (1 H, m) | — |
| C-5 | 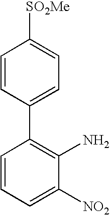 | [4′- (methyl- sulfonyl)-3- nitrobiphenyl- 2-yl]amine | N-[4′- (methyl- sulfonyl)-3- nitrobiphenyl- 2- yl)acetamide | 1H NMR (DMSO-d₆) 3.26 (3 H + H₂O, s), 6.79- 6.83 (1 H, m), 6.88 (2 H, s), 7.38-7.41 (1 H, m), 7.70- 7.72 (2 H, m), 8.05- 8.13 (3 H, m) | [M − H]⁻ 291 |
| C-6 | 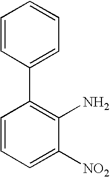 | (3- nitrobiphenyl- 2-yl)amine | N-(3- nitrobiphenyl- 2- yl)acetamide | 1H NMR (DMSO-d₆) 6.71 (2 H, s), 6.77-6.81 (1 H, m), 7.34-7.36 (1 H, m), 7.41- 7.49 (3 H, m), 7.52- 7.56 (2 H, m), 8.05-8.08 (1 H, m) | — |

Reduction of Biaryl and Heteroaryl-Aryl Intermediates C-1 to C-6

Preparation D-1

### 3-Pyridin-3-ylbenzene-1,2-diamine

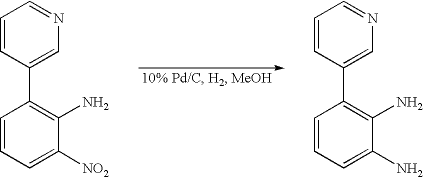

(2-Nitro-6-pyridin-3-ylphenyl)amine (255 mg, 1.18 mmol) was suspended in methanol (20 ml) and the system purged with nitrogen. 10% Pd/C (26 mg, 10% by mass) was added and the mixture stirred under a hydrogen atmosphere overnight. The system was purged with nitrogen and the catalyst filtered off The filtrate was concentrated to give the title compound (218 mg, 100%) as brown solid that was used immediately in the next step.

By following the method in Preparation D-1, the biaryl intermediates D-2 to D-6 were formed.

| Prep. | | Chemical | | | |
|---|---|---|---|---|---|
| No. | Structure | Name | Precursor | N.M.R. Data | M.S. |
| D-2 | 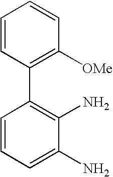 | 2′-methoxy- biphenyl- 2,3-diamine | (2′-methoxy- 3-nitro- biphenyl-2- yl)amine | 1H NMR (DMSO-d₆) 3.73 (3 H, s), 6.28-6.30 (1 H, m), 6.47 (1 H, t), 6.56-6.58 (1 H, m), 6.99-7.03 (1 H, m), 7.08- 7.12 (2 H, m), 7.33- 7.37 (1 H, m) | [M + H]⁺ 215 |
| D-3 | 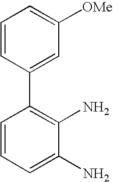 | 3′-methoxy- biphenyl- 2,3-diamine | (3′-methoxy- 3-nitro- biphenyl-2- yl)amine | 1H NMR (DMSO-d₆) 3.79 (3 H, s), 4.07 (2 H, s), 4.56 (2 H, s), 6.37-6.39 (1 H, m), 6.50 (1 H, t), 6.57-6.59 (1 H, m), 6.89-6.91 (2 H, m), 6.94- 6.96 (1 H, m), 7.34- 7.38 (1 H, m) | [M + H]⁺ 215 |
| D-4 | 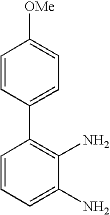 | 4′-methoxy- biphenyl- 2,3-diamine | (4′-methoxy- 3-nitro- biphenyl-2- yl)amine | 1H NMR (DMSO-d₆) 3.80 (3 H, s), 4.47 (4 H, s), 6.35- 6.38 (1 H, m), 6.50 (1 H, t), 6.56-6.59 (1 H, m), 6.99- 7.03 (2 H, m), 7.28- 7.32 (2 H, m) | [M + H]⁺ 215 |
| D-5 | 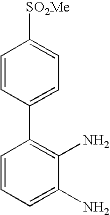 | 4′-(methyl- sulfonyl)- biphenyl- 2,3-diamine | [4′-(methyl- sulfonyl)-3- nitro- biphenyl-2- yl]amine | 1H NMR (DMSO-d₆) 3.25 (3 H, s), 4.24 (2 H, s), 4.64- 4.67 (2 H, s), 6.38- 6.41 (1 H, m), 6.54 (1 H, t), 6.61-6.63 (1 H, m), 7.66- 7.68 (2 H, m), 7.96- 7.99 (2 H, m) | [M + H]⁺ 263 |
| D-6 | 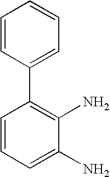 | biphenyl- 2,3-diamine | (3-nitro- biphenyl-2- yl)amine | 1H NMR (DMSO-d₆) 4.05 (2 H, s), 4.57 (2 H, s), 6.36- 6.38 (1 H, m), 6.51 (1 H, t), 6.57-6.60 (1 H, m), 7.31- 7.35 (1 H, m), 7.35- 7.40 (2 H, m), 7.43-7.47 (2 H, m) | [M + H]⁺ 185 |

### EXAMPLE 79

### 4-(4-Pyridin-3-yl-1H-benzimidazol-2-yl)-1-(7H-pyrrolo[2,3-d]pyrimidin-4-amine

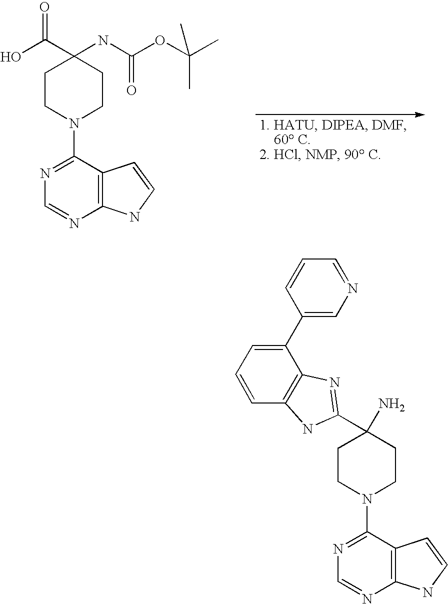

3-Pyridin-3-ylbenzene-1,2-diamine (220 mg, 1.19 mmol), 4-[(tert-butoxycarbonyl)amino]-1-(7H-pyrrolo[2,3-d]pyrimidin-4-yl)piperidine-4-carboxylic acid (430 mg, 1.19 mmol) and HATU (679 mg, 1.79 mmol) were dissolved in DMF (6 ml). N,N-Diisopropylethylamine (623 μl, 3.57 mmol) was added and the mixture heated at 60° C. for 3 h. The mixture was cooled and stirred at RT overnight. The solution was absorbed onto an SCX column, washed with methanol and eluted with ammonia in methanol. Product containing fractions were concentrated. The crude product was dissolved in NMP (5 ml) and 4M HCl in dioxane (5 ml) added. The mixture was heated at 90° C. for 4 h and then allowed to cool to RT overnight. The solution was absorbed onto an SCX column, washed with methanol and eluted with ammonia in methanol. Product containing fractions were concentrated. The residue was purified by reverse phase HPLC (Xbridge column, 19×10 mm, 5 micron C18. Modifier=1% 0.880 ammonia in water/acetonitrile) to give the title compound (90 mg, 18%) as a white solid.

M/z: [M+H]⁺ 411; 1H NMR (DMSO-d₆) 1.84 (2H, d), 2.24-2.31 (2H, m), 3.88-3.95 (2H, m), 4.28-4.33 (2H, m), 6.64 (1H, d), 7.18 (1H, d), 7.28 (1H, t), 7.43 (1H, d), 7.49-7.54 (2H, m), 8.16 (1H, s), 8.44 (1H, brs), 8.55 (1H, d), 9.28 (1H, brs), 11.65 (1H, s)

### EXAMPLES 80 TO 84

By following the method of Example 79, the compounds of Examples 80 to 84 were formed.

| Example | | Chemical | | | |
|---|---|---|---|---|---|
| No. | Structure | Name | Precursor | N.M.R. Data | M.S. |
| 80 | 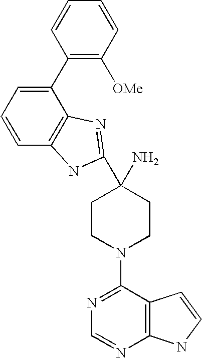 | 4-[4-(2- methoxy- phenyl)-1H- benzimidazol- 2-yl]-1- (7H- pyrrolo[2,3- d]pyrimidin- 4- yl)piperidin- 4-amine | 2′-methoxy- biphenyl- 2,3-diamine | 1H NMR (DMSO-d₆) 1.78 (2 H, d), 2.27 (2 H, m), 3.77 (3 H, brs), 3.88 (2 H, t), 4.28 (2 H, d), 6.63 (1 H, d), 7.09 (2 H, brs), 7.15-7.19 (3 H, m), 7.40-7.51 (3 H, m), 8.14 (1 H, s), 11.64 (1 H, s) | [M + H]⁺ 440 |
| 81 | 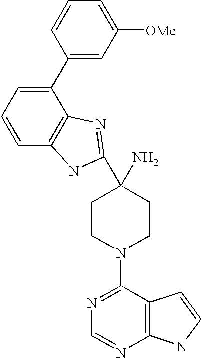 | -[4-(3- methoxy- phenyl)-1H- benzimidazol- 2-yl]-1- (7H- pyrrolo[2,3- d]pyrimidin- 4- yl)piperidin- 4-amine | 3′-methoxy- biphenyl- 2,3-diamine | 1H NMR (DMSO-d₆) 1.82 (2 H, d), 2.25-2.32 (2 H, m), 3.78 (3 H, s), 3.91 (2 H, t), 4.30 (2 H, m), 6.64 (1 H, d), 6.92 (1 H, d), 7.17-7.19 (1 H, m), 7.23 (1 H, t), 7.38 (2 H, m), 7.47- 7.92 (3 H, brm), 8.15 (1 H, s), 11.65 (1 H, s) | [M + H]⁺ 440 |
| 82 | 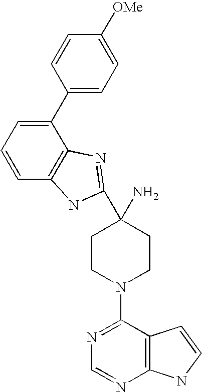 | -[4-(4- methoxy- phenyl)-1H- benzimidazol- 2-yl]-1- (7H- pyrrolo[2,3- d]pyrimidin- 4- yl)piperidin- 4-amine | 4′-methoxy- biphenyl- 2,3-diamine | 1H NMR (DMSO-d₆) 1.82 (2 H, d), 2.24- 2.31 (2 H, m), 3.82 (3 H, s), 3.90 (2 H, t), 4.30- 4.35 (2 H, m), 6.64 (1 H, d), 7.05 (2 H, d), 7.17-7.22 (2 H, m), 7.28 (1 H, s), 7.42 (1 H, d), 8.06 (2 H, brs), 8.15 (1 H, s), 11.65 (1 H, s) | [M + H]⁺ 440 |
| 83 | 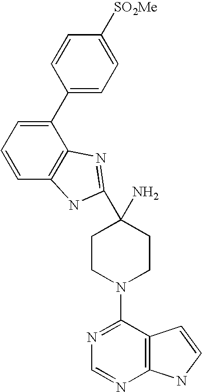 | 4-{4-[4- (methyl- sulfonyl)phenyl]- 1H- benzimidazol- 2-yl}-1- (7H- pyrrolo[2,3- d]pyrimidin- 4- yl)piperidin- 4-amine | 4′-(methyl- sulfonyl)- biphenyl- 2,3-diamine | 1H NMR (DMSO-d₆) 1.84 (2 H, d), 2.24-2.31 (2 H, m), 3.27 (3 H, s), 3.87-3.94 (2 H, m), 4.31-4.36 (2 H, m), 6.64- 6.65 (1 H, m), 7.17- 7.19 (1 H, m), 7.30 (1 H, t), 7.46 (1 H, d), 7.56 (1 H, d), 8.01 (2 H, d), 8.16 (1 H, s), 8.34 (2 H, brs), 11.65 (1 H, s) | [M + H]⁺ 488 |
| 84 | 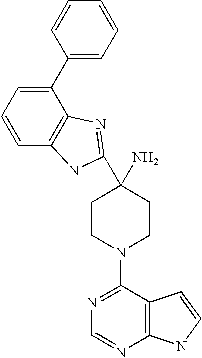 | 4-(4-phenyl- 1H- benzimidazol- 2-yl)-1- (7H- pyrrolo[2,3- d]pyrimidin- 4- yl)piperidin- 4-amine | biphenyl- 2,3-diamine | 1H NMR (DMSO-d₆) 1.80-1.86 (2 H, m), 2.25- 2.31 (2 H, m), 3.91 (2 H, t), 4.28-4.34 (2 H, m), 6.64 (1 H, d), 7.18 (1 H, t), 7.24 (1 H, t), 7.34- 7.37 (2 H, m), 7.46- 7.51 (3 H, m), 8.05 (2 H, brs), 8.16 (1 H, s), 11.65 (1 H, s). | [M + H]⁺ 410 |

### EXAMPLE 85

### 4-(7-Bromo-1H-benzo[d]imidazol-2-yl)-1-(7H-pyrrolo[2,3-d]pyrimidin-4-yl)piperidin-amine

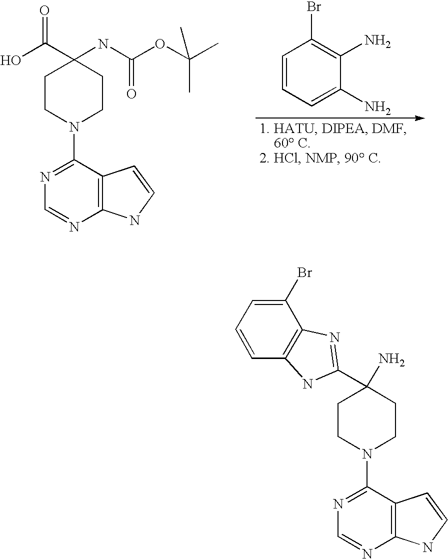

### 85A. tert-Butyl 4-(2-amino-3-bromophenylcarbamoyl)-1-(7H-pyrrolo[2,3-d]pyrimidin-4-yl)piperidin-4-ylcarbamate

4-(tert-Butoxycarbonylamino)-1-(7H-pyrrolo[2,3-d]pyrimidin-4-yl)piperidine-4-carboxylic acid (1.7 g, 4.70 mmol), 3-bromobenzene-1,2-diamine dihydrochloride (1.467 g, 5.64 mmol) and HATU (2.68 g, 7.06 mmol) were dissolved in DMF (15 mL). DIPEA (4.11 mL, 23.52 mmol) was added and the reaction mixture heated at 65° C. for 4 h. The mixture was cooled and concentrated. The residue was dissolved in methanol and absorbed onto and SCX column, washed with methanol and eluted with ammonia in methanol. Product containing fractions were concentrated. The crude product was purified by flash silica chromatography, eluting with 2.5% MeOH in DCM. Pure fractions were evaporated to dryness to afford the title compound (0.388 g, 15.55%) as a white solid.

M/z: [M+H]⁺ 532; 1H NMR (DMSO-d₆) 1.45 (9H, s), 2.01-2.14 (4H, m), 3.66-3.72 (2H, m), 4.24-4.28 (2H, m), 4.98 (2H, s), 6.50 (1H, t), 6.62-6.64 (1H, m), 6.92 (1H, d), 7.17-7.19 (1H, m), 7.27-7.30 (1H, m), 7.42 (1H, s), 8.15 (1H, s), 9.27 (1H, s), 11.66 (1H, s)

### 85B. 4-(7-Bromo-1H-benzo[d]imidazol-2-yl)-1-(7H-pyrrolo[2,3-d]pyrimidin-4-yl)piperidin-4-amine

tert-Butyl 4-(2-amino-3-bromophenylcarbamoyl)-1-(7H-pyrrolo[2,3-d]pyrimidin-4-yl)piperidin-4-ylcarbamate (370 mg, 0.70 mmol) was dissolved in NMP (3.5 mL) and 4M HCl in dioxane (3.5 ml) added. The reaction mixture was heated at 90° C. overnight. The mixture was cooled and concentrated. The crude product was purified by ion exchange chromatography, using an SCX column. The desired product was eluted from the column using 7M NH3/MeOH and pure fractions were evaporated to dryness to afford the title compound (267 mg, 93%) as a tan coloured foam.

M/z: [M+H]⁺ 414; 1H NMR (DMSO-d₆) 1.81 (2H, d), 2.21-2.29 (2H, m), 3.83-3.90 (2H, m), 4.31-4.36 (2H, m), 6.65 (1H, d), 7.09 (1H, t), 7.18 (1H, d), 7.34-7.36 (1H, m), 7.49 (1H, d), 8.16 (1H, s), 11.66 (1H, s).

### EXAMPLE 86

### 4-(4-(1-Methyl-1H-pyrazol-4-yl)-1H-benzo[d]imidazol-2-yl)-1(7H-pyrrolo[2,3-d]pyrimidin-4-yl)piperidin-4-amine

### 86A. tert-Butyl 5-bromo-1′-[7-(tert-butoxycarbonyl)-7H-pyrrolo[2,3-d]pyrimidin-4-yl]-1-oxospiro[imidazo[1,5-a]benzimidazole-3,4′-piperidine]-2(1H)-carboxylate

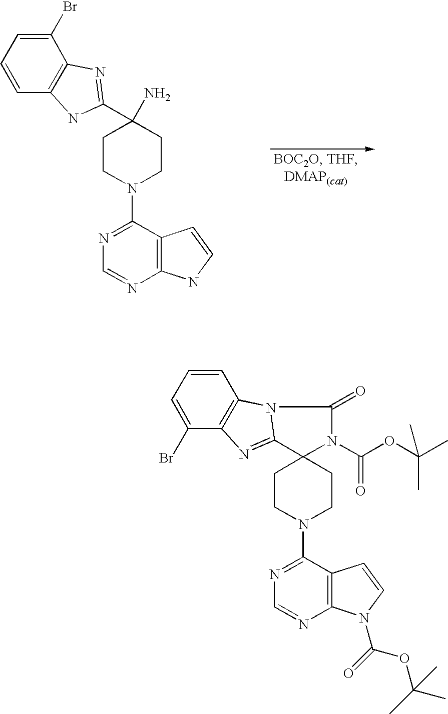

(4-Bromo-1H-benzo[d]imidazol-2-yl)-1-(7H-pyrrolo[2,3-d]pyrimidin-4-yl)piperidin-4-amine (130 mg, 0.32 mmol), di-tert-butyl dicarbonate (0.326 mL, 1.42 mmol) and 4-dimethylaminopyridine (0.385 mg, 3.15 μmol) were stirred in THF (10 mL) for 2 h at room temperature. The mixture was concentrated and the crude product was purified by flash silica chromatography, eluting with 0.5% 7N ammonia in methanol in DCM. Pure fractions were evaporated to dryness to afford the title compound (167 mg, 83%) as a white foam.

M/z: [M+H]⁺ 640; 1H NMR (DMSO-d₆) 1.34 (9H, s), 1.63 (9H, s), 2.20 (2H, d), 2.68-2.77 (2H, m), 3.99 (2H, t), 4.82 (2H, d), 6.98 (1H, d), 7.41 (1H, t), 7.60 (1H, d), 7.70-7.72 (1H, m), 7.91-7.93 (1H, m), 8.42 (1H, s)

### 86B. 4-(4-(1-Methyl-1H-pyrazol-4-yl)-1H-benzo[d]imidazol-2-yl)-1-(7H-pyrrolo[2,3-d]pyrimidin-4-yl)piperidin-4-amine

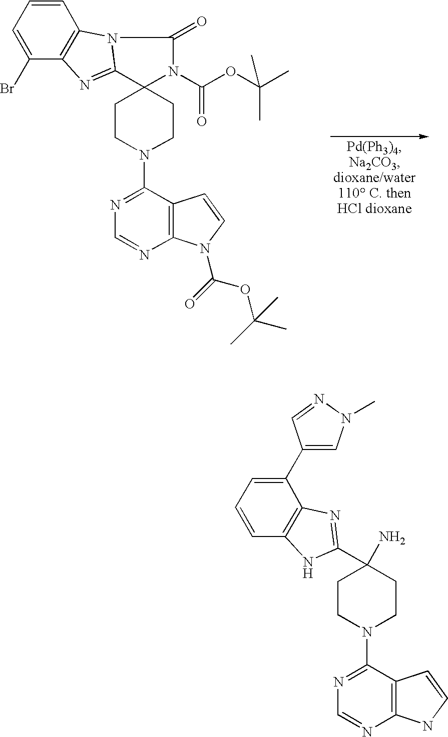

Butyl 5-bromo-1′-[7-(tert-butoxycarbonyl)-7H-pyrrolo[2,3-d]pyrimidin-4-yl]-1-oxospiro[imidazo[1,5-a]benzimidazole-3,4′-piperidine]-2(1H)-carboxylate (160 mg, 0.25 mmol), 1-methyl-4-(4,4,5,5-tetramethyl-1,3,2-dioxaborolan-2-yl)-1H-pyrazole (62 mg, 0.30 mmol) and sodium carbonate (105 mg, 0.99 mmol) were stirred in dioxane (5 mL) and water (1 mL). The mixture was purged with nitrogen for 15 min. Tetrakis(triphenylphosphine)palladium (0) (5.74 mg, 4.97 μmol) was added and the mixture heated at 110° C. for 30 min in a microwave. The mixture was cooled, filtered and concentrated. The residue was dissolved in dioxane and 4M HCl in dioxane (10 ml) added. The mixture was stirred for 2 h and concentrated. The residue was dissolved in methanol, absorbed on to an SCX column, washed with methanol and eluted with ammonia in methanol. Product containing fractions were concentrated. The crude product was purified by flash silica chromatography, eluting with 5% 7N ammonia in MeOH in DCM gave the title compound (54.0 mg, 52.6%) as a white solid.

M/z: [M+H]⁺ 414; 1H NMR (DMSO-d₆) 1.85 (2H, d), 2.28-2.35 (2H, m), 3.95 (5H, m(, 4.29-4.35 (2H, m), 6.66 (1H, d), 7.13 (1H, t), 7.18-7.19 (1H, m), 7.29 (1H, d), 7.38 (1H, d), 8.17 (1H, s), 8.20 (1H, s), 8.51 (1H, s), 11.65 (1H, s), 12.20 (1H, brs).

### EXAMPLES 87 AND 88

By following the method of Example 86B, the compounds of Examples 87 and 88 were formed.

| Example | | Chemical | | | |
|---|---|---|---|---|---|
| No. | Structure | Name | Precursor | N.M.R. Data | M.S. |
| 87 | 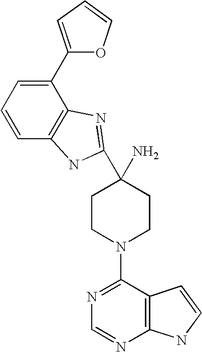 | 4-[7-(2- furyl)-1H- benzimidazol- 2-yl]-1- (7H- pyrrolo[2,3- d]pyrimidin- 4- yl)piperidin- 4-amine | 2-(2-furyl)- 4,4,5,5- tetramethyl- 1,3,2- dioxaborolane | 1H NMR (DMSO-d₆) 1.83-1.90 (2 H, m), 2.28- 2.35 (2 H, m), 3.96 (2 H, t), 4.28-4.34 (2 H, m), 6.66 (2 H, d), 7.18-7.20 (1 H, m), 7.23 (1 H, d), 7.42 (1 H, d), 7.51- 7.53 (2 H, m), 7.76 (1 H, s), 8.17 (1 H, s), 11.66 (1 H, s), 12.30 (1 H, brS). | [M + H]⁺ 400 |
| 88 | 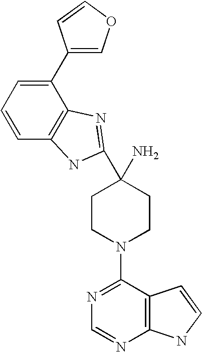 | 4-[7-(3- furyl)-1H- benzimidazol- 2-yl]-1- (7H- pyrrolo[2,3- d]pyrimidin- 4- yl)piperidin- 4-amine | 2-(3-furyl)- 4,4,5,5- tetramethyl- 1,3,2- dioxaborolane | 1H NMR (DMSO-d₆) 1.83-1.87 (2 H, m), 2.27- 2.34 (2 H, m), 3.93- 4.00 (2 H, m), 4.26-4.32 (2 H, m), 6.66 (1 H, m), 7.16-7.23 (3 H, m), 7.36-7.42 (2 H, m), 7.75 (1 H, s), 8.16 (1 H, s), 8.67 (1 H, s), 11.65 (1 H, s), 12.28 (1 H, brS) | [M + H]⁺ 400 |

### EXAMPLE 89

### (4-(5-Bromo-1H-benzo[d]imidazol-2-yl)-1-(7H-pyrrolo[2,3-d]pyrimidin-4-yl)piperidin-4-yl)methanamine

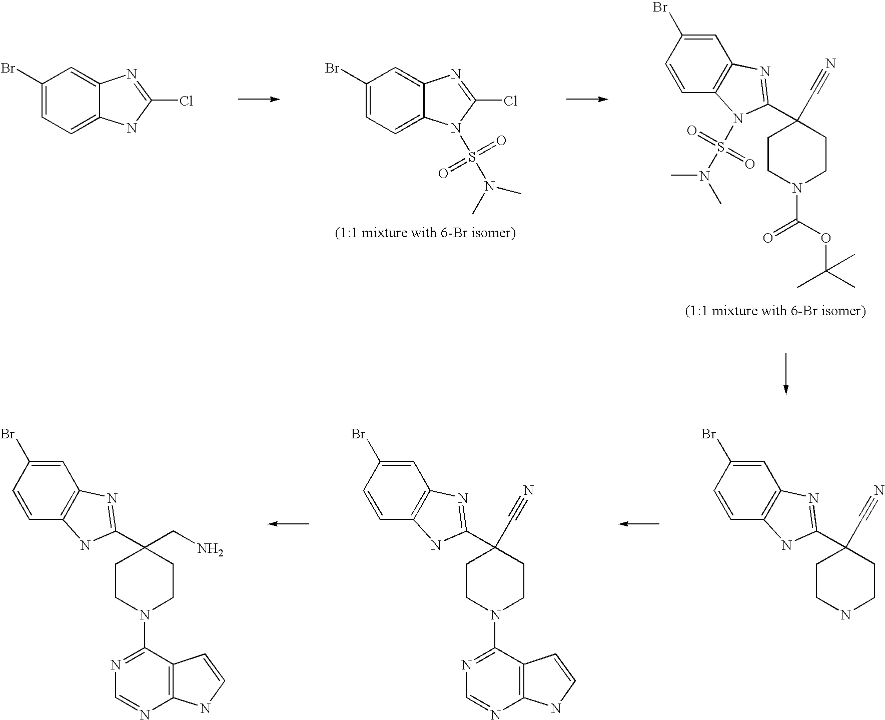

### 89A. 5-Bromo-2-chloro-N,N-dimethyl-1H-benzimidazole-1-sulfonamide and 6-bromo-2-chloro-N,N-dimethyl-1H-benzimidazole-1-sulfonamide (1:1 mixture)

The title compound was prepared as a 1:1 mixture of isomers from 5-bromo-2-chlorobenzimidazole using the procedure described in Example 34A.

M/z: [M+H]⁺ 340; 1H NMR (DMSO-d₆) 3.01 and 3.03 (both 3H, s), 7.58-7.62 (1H, m), 7.70 and 7.83 (both 0.5H, d), 7.97-8.00 (1H, m).

### 89B. tert-Butyl 4-{5-bromo-1-[(dimethylamino)sulfonyl]-1H-benzimidazol-2-yl}-4-cyanopiperidine-1-carboxylate and tert-butyl 4-{6-bromo-1-[(dimethylamino)sulfonyl]-1H-benzimidazol-2-yl}-4-cyanopiperidine-1-carboxylate (1:1 mixture)

The title compound was prepared as a 1:1 mixture of isomers from 5-bromo-2-chloro-N,N-dimethyl-1H-benzimidazole-1-sulfonamide and 6-bromo-2-chloro-N,N-dimethyl-1H-benzimidazole-1-sulfonamide (1:1 mixture) and 1-tert-butoxycarbonyl-4-cyanopiperidine using the procedure described in Example 34B.

M/z: [M+H]⁺ 512; 1H NMR (DMSO-d₆) 1.43 (9H, s), 2.22 (2H, t), 2.62 (2H, d), 3.00 and 3.02 (both 3H, s), 3.13 (2H, m), 4.09 (2H, d), 7.63-7.67 (1H, m), 7.78 and 7.81 (both 0.5H, d), 7.88 and 8.09 (both 0.5H, d).

### 89C. 4-(5-Bromo-1H-benzo[d]imidazol-2-yl)piperidine-4-carbonitrile

In a different deprotection reaction to that described in Example 34C, the mixture of tert-butyl 4-(5-bromo-1-(N,N-dimethylsulfamoyl)-1H-benzo[d]imidazol-2-yl)-4-cyanopiperidine-1-carboxylate compound and tert-butyl 4-(6-bromo-1-(N,N-dimethylsulfamoyl)-1H-benzo[d]imidazol-2-yl)-4-cyanopiperidine-1-carboxylate (1:1 mixture) (500 mg, 0.98 mmol) was dissolved in dioxane (5 mL), 4M HCl in dioxane (5 ml) was added and the mixture was stirred for 4 h. The mixture was then concentrated and the residue dissolved in methanol/water, absorbed onto an SCX column, washed with methanol and eluted with ammonia in methanol. The product containing fractions were concentrated. The crude product was purified by flash silica chromatography, elution with 7.5% 7N ammonia in methanol in DCM gave the title compound (173 mg, 58.1%) as a white solid.

M/z: [M+H]⁺ 307; 1H NMR (DMSO-d₆) 2.06-2.13 (2H, m), 2.23 (2H, d), 2.79-2.86 (2H, m), 3.01-3.06 (2H, m), 7.36-7.39 (1H, m), 7.55 (1H, d), 7.79 (1H,

### 89D. 4-(5-Bromo-1H-benzo[d]imidazol-2-yl)-1-(7H-pyrrolo[2,3-d]pyrimidin-4-yl)piperidine-4-carbonitrile

4-(5-Bromo-1H-benzo[d]imidazol-2-yl)piperidine-4-carbonitrile (170 mg, 0.56 mmol), 4-chloro-7H-pyrrolo[2,3-d]pyrimidine (94 mg, 0.61 mmol) and triethylamine (0.37 ml, 2.65 mmol) were dissolved in NMP (5 mL) and sealed into a microwave tube. The reaction mixture was heated to 160° C. for 90 minutes in the microwave reactor and cooled to RT. The crude product was purified by ion exchange chromatography, using an SCX column. The desired product was eluted from the column using 7M NH3/MeOH and product containing fractions were concentrated to give an oil which formed a solid when concentrated from DCM. This was suspended in DCM, filtered and dried on the filter to give the title compound (71.4%) as a beige solid.

M/z: [M+H]⁺ 424; 1H NMR (DMSO-d₆) 2.29-2.37 (2H, m), 2.45 (2H, d), 3.54 (2H, t), 4.77 (2H, d), 6.69-6.70 (1H, m), 7.25-7.26 (1H, m), 7.37-7.39 (1H, m), 7.50-7.60 (1H, m), 7.75-7.85 (1H, m), 8.22 (1H, s), 11.78 (1H, s), 13.04 (1H, s)

### 89E. (4-(5-Bromo-1H-benzo[d]imidazol-2-yl)-1-(7H-pyrrolo[2,3-d]pyrimidin-4-yl)piperidin-4-yl)methanamine

4-(5-Bromo-1H-benzo[d]imidazol-2-yl)-1-(7H-pyrrolo[2,3-d]pyrimidin-4-yl)piperidin-4-carbonitrile (160 mg, 0.38 mmol) and platinum(IV) oxide (16 mg, 0.07 mmol) in acetic acid (50 mL) were stirred under an atmosphere of hydrogen at 5 bar and 25° C. for 16 hours. The catalyst was filtered off and the filtrate concentrated. The residue was dissolved in methanol, absorbed on to an SCX column, washed with methanol and eluted with ammonia in methanol. Product containing fractions were concentrated. The crude product was purified by preparative HPLC (sunfire C18 column, 5μ silica, 19 mm diameter, 100 mm length), using decreasingly polar mixtures of water (containing 0.1% TFA) and MeCN as eluents. The resulting product was then further purified using preparative HPLC (Xbridge column, 5μ silica, 19 mm diameter, 100 mm length), using decreasingly polar mixtures of water (containing 1% NH3) and MeCN as eluents. Fractions containing the desired compound were evaporated to dryness to afford the title compound (59.0 mg, 36.5%) as a brown solid.

M/z: [M+H]⁺ 428; 1H NMR (DMSO-d₆) 1.92-1.97 (2H, m), 2.43-2.47 (2H, m), 3.22 (2H, s), 3.69-3.74 (2H, m), 4.19-4.23 (2H, m), 6.54 (1H, d), 7.09-7.10 (1H, d), 7.29-7.31 (1H, m), 7.48-7.49 (1H, d), 7.70-7.71 (1H, d), 8.14 (1H, s).

### EXAMPLE 90

### 4-(7-(benzyloxy)-1H-benzo[d]imidazol-2-yl)-1-(7H-pyrrolo[2,3-d]pyrimidin-4-yl)piperidin-4-amine

### 90A. tert-butyl 4-(2-amino-3-(benzyloxy)phenylcarbamoyl)-1-(7H-pyrrolo[2,3-d]pyrimidin-4-yl)piperidin-4-ylcarbamate and tert-butyl 4-(2-amino-6-(benzyloxy)phenylcarbamoyl)-1-(7H-pyrrolo[2,3-d]pyrimidin-4-yl)piperidin-4-ylcarbamate (7:3 regioisomeric mixture)

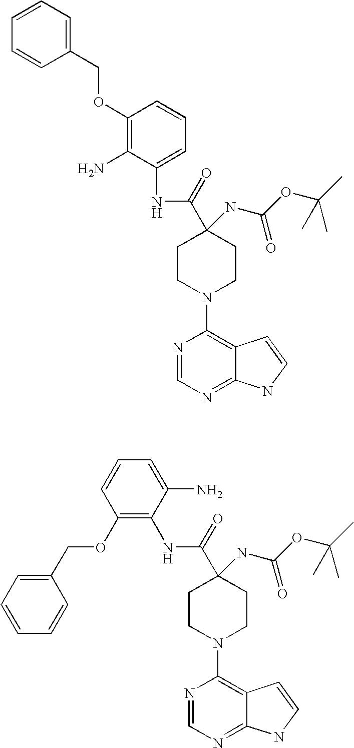

O-(7-Azabenzotriazol-1-yl)-N,N,N′,N′-tetramethyluronium hexafluorophosphate (1.063 g, 2.79 mmol) was added in one portion to a stirred suspension of 4-(tert-butoxycarbonylamino)-1-(7H-pyrrolo[2,3-d]pyrimidin-4-yl)piperidine-4-carboxylic acid (1 g, 2.77 mmol) and N-Ethyldiisopropylamine (0.574 mL, 3.32 mmol) in NMP (5 mL). The mixture was warmed to 50° C. to achieve dissolution. The mixture was cooled and 3-(benzyloxy)benzene-1,2-diamine (0.593 g, 2.77 mmol) was added in one portion. The dark solution was stirred for 65 hours at room temperature. The mixture was partitioned between ethyl acetate and water. The organic layer was washed three times with water and then brine. The organic solution was dried over magnesium sulfate, filtered and evaporated. The residue was purified by MPLC on silica using gradient elution (1% methanol/DCM to 10% methanol/DCM). Product containing fractions were combined to give a pink foam which was shown to be a 7:3 regioisomeric mixture of tert-butyl 4-(2-amino-3-(benzyloxy)phenylcarbamoyl)-1-(7H-pyrrolo[2,3-d]pyrimidin-4-yl)piperidin-4-ylcarbamate and tert-butyl 4-(2-amino-6-(benzyloxy)phenylcarbamoyl)-1-(7H-pyrrolo[2,3-d]pyrimidin-4-yl)piperidin-4-ylcarbamate (1.230 g, 80%). m/z (ESI+) (M+H)⁺=558.5; HPLC t_{R}=2.21 min (28%), 2.41 min (72%); ¹H NMR (399.9 MHz, DMSO-d₆) δ 1.44 (9H, s), 1.91-1.98 (0.6H, m), 2.06-2.20 (3.4H, m), 3.64-3.77 (2H, m), 3.97-4.32 (1.4H, m), 4.95 (0.6H, s), 5.13 (1.4H, s), 6.28 (0.3H, d), 6.32 (0.3H, d), 6.53 (0.70H, t), 6.56-6.60 (1H, m), 6.75 (0.7H, s), 6.82 (0.7H, d), 7.22-7.43 (5.6H, m), 7.50 (1.4H, d), 8.22 (1H, s), 8.62 (0.3H, s), 9.12 (0.7H, s), 12.00 (1H, s).

### 90B. 4-(7-(benzyloxy)-1H-benzo[d]imidazol-2-yl)-1-(7H-pyrrolo[2,3-d]pyrimidin-4-yl)piperidin-4-amine

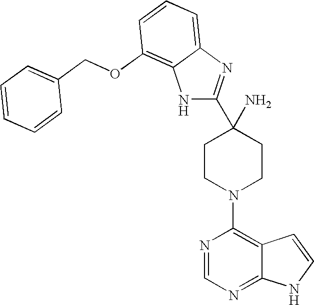

A mixture of tert-butyl 4-(2-amino-3-(benzyloxy)phenylcarbamoyl)-1-(7H-pyrrolo[2,3-d]pyrimidin-4-yl)piperidin-4-ylcarbamate and tert-butyl 4-(2-amino-6-(benzyloxy)phenylcarbamoyl)-1-(7H-pyrrolo[2,3-d]pyrimidin-4-yl)piperidin-4-ylcarbamate (1.58 g, 2.83 mmol) was treated with hydrogen chloride (4M in dioxane) (7.4 mL, 29.60 mmol) and NMP (7.5 mL). The resulting solution was stirred at 95° C. for 16 hours. The mixture was concentrated under reduced pressure and the residue was partitioned between DCM and sodium bicarbonate solution. The organic layer was concentrated under reduced pressure and the residue was purified by MPLC on silica using gradient elution (2% methanol/DCM to 1% n-propylamine/15% methanol/DCM). The desired product, 4-(7-(benzyloxy)-1H-benzo[d]imidazol-2-yl)-1-(7H-pyrrolo[2,3-d]pyrimidin-4-yl)piperidin-4-amine (0.646 g, 51.9%), was thus isolated as a tan dry film.

m/z (ESI+) (M+H)+=440.5; HPLC t_{R}=1.94 min; ¹H NMR (399.9 MHz, DMSO-d6) δ 1.78 (2H, d), 2.20-2.30 (4H, m), 3.88 (2H, t), 4.23-4.32 (2H, m), 5.30 (2H, s), 6.63 (1H, d), 6.76 (1H, br, s), 6.99-7.14 (2H, m), 7.17 (1H, d), 7.31-7.44 (3H, m), 7.52 (2H, br, s). 8.15 (1H, s), 11.64 (1H, s), 12.15 (1H, br, s).

### EXAMPLE 91

### 4-(4-pyrimidin-5-yl-1H-benzimidazol-2-yl)-1-(7H-pyrrolo[3,2-e]pyrimidin-4-yl)piperidin-4-amine

### 91A. tert-butyl 1′-[7-1(2-methylpropan-2-yl)oxycarbonyl]pyrrolo[3,2-e]pyrimidin-4-yl]-1-oxo-5-phenylmethoxyspirolimidazo[3,4-a]benzimidazole-3,4′-piperidine]-2-carboxylate

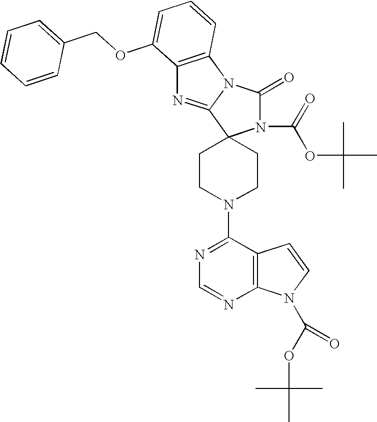

Di-tert-butyl dicarbonate (1.1 g, 5.04 mmol) in THF (5 mL) was added to a suspension of 4-(7-(benzyloxy)-1H-benzo[d]imidazol-2-yl)-1-(7H-pyrrolo[2,3-d]pyrimidin-4-yl)piperidin-4-amine (554 mg, 1.26 mmol) in THF (5 mL). 4-Dimethylaminopyridine (23.10 mg, 0.19 mmol) was added and the solution was stirred for 2 hours at room temperature. The mixture was concentrated under reduced pressure and the residue was purified by MPLC on silica using gradient elution (15% ethyl acetate/isohexane to 50% ethyl acetate/isohexane). The desired product, tert-butyl 1′-[7-[(2-methylpropan-2-yl)oxycarbonyl]pyrrolo[3,2-e]pyrimidin-4-yl]-1-oxo-5-phenylmethoxyspiro[imidazo[3,4-a]benzimidazole-3,4′-piperidine]-2-carboxylate (719 mg, 86%), was thus isolated as a colourless dry film. m/z (ESI+) (M+H)⁺=666.6; HPLC t_{R}=3.38 min; ¹H NMR (399.9 MHz, CDCl₃) δ 1.46 (9H, s), 1.67 (9H, s), 1.89 (2H, d), 2.97-3.09 (2H, m), 4.23 (2H, t), 4.83 (2H, d), 5.49 (2H, s), 6.55 (1H, d), 6.93 (1H, d), 7.29-7.44 (5H, m), 7.50 (2H, d), 7.57 (1H, d), 8.57 (1H, s).

### 91B. tert-butyl 5-hydroxy-1′-[7-[(2-methylpropan-2-yl)oxycarbonyl]pyrrolo[3,2-e]pyrimidin-4-yl]-1-oxospiro[imidazo[3,4-a]benzimidazole-3,4′-piperidine]-2-carboxylate

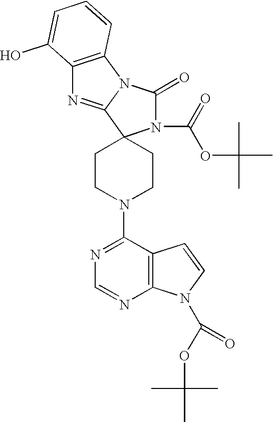

Palladium on carbon (10% w/w) (1.5 g, 1.41 mmol) was added in one portion to tert-butyl 1′-[7-[(2-methylpropan-2-yl)oxycarbonyl]pyrrolo[3,2-e]pyrimidin-4-yl]-1-oxo-5-phenylmethoxyspiro[imidazo[3,4-a]benzimidazole-3,4′-piperidine]-2-carboxylate (8.9 g, 13.37 mmol) in ethanol (150 mL) and THF (50 mL) at room temperature. The resulting solution was stirred vigorously under an atmosphere of hydrogen for 4 hours. The mixture was filtered through celite, washing the catalyst with THF (100 mL). The filtrate was concentrated under reduced pressure to give tert-butyl 5-hydroxy-1′-[7-[(2-methylpropan-2-yl)oxycarbonyl]pyrrolo[3,2-e]pyrimidin-4-yl]-1-oxospiro[imidazo[3,4-a]benzimidazole-3,4′-piperidine]-2-carboxylate (6.96 g, 90%) as a colourless solid. m/z (ESI+) (M+H)⁺=576.6; HPLC t_{R}=2.40 min. ¹H NMR (399.9 MHz, DMSO-d₆) δ 1.34 (9H, s), 1.63 (9H, s), 2.12 (2H, d), 2.69-2.79 (2H, m), 3.97-4.07 (2H, m), 4.80 (2H, d), 6.82-6.88 (1H, m), 6.97 (1H, d), 7.24 (1H, t), 7.29-7.34 (1H, m), 7.59 (1H, d), 8.41 (1H, s), 10.18 (1H, s).

### 91C. tert-butyl 1′-[7-[(2-methylpropan-2-yl)oxycarbonyl]pyrrolo[3,2-e]pyrimidin-4-yl]-1-oxo-5-(trifluoromethylsulfonyloxy)spiro[imidazo[3,4-a]benzimidazole-3,4′-piperidine]-2-carboxylate

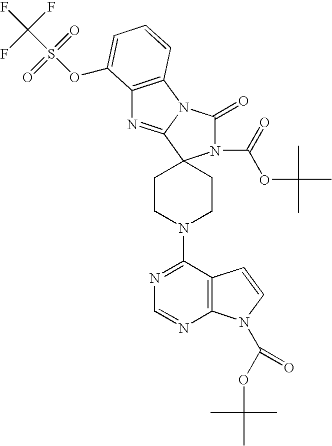

Trifluoromethanesulfonic anhydride (0.026 mL, 0.16 mmol) was added to a mixture of tert-butyl 5-hydroxy-1′-[7-[(2-methylpropan-2-yl)oxycarbonyl]pyrrolo[3,2-e]pyrimidin-4-yl]-1-oxospiro[imidazo[3,4-a]benzimidazole-3,4′-piperidine]-2-carboxylate (75 mg, 0.13 mmol) and N-ethyldiisopropylamine (0.045 mL, 0.26 mmol) in DCM (1 mL). The mixture was stirred for 2 hours at room temperature. The mixture was partitioned between DCM and sodium bicarbonate solution. The organic solution was dried with magnesium sulfate, filtered, and concentrated under reduced pressure. The residue was purified by MPLC on silica using gradient elution (10% ethyl acetate/DCM to 20% ethyl acetate/DCM). The desired product, tert-butyl 1′-[7-[(2-methylpropan-2-yl)oxycarbonyl]pyrrolo[3,2-e]pyrimidin-4-yl]-1-oxo-5-(trifluoromethylsulfonyloxy)spiro[imidazo[3,4-a]benzimidazole-3,4′-piperidine]-2-carboxylate (53.0 mg, 57.5%), was thus isolated as a yellow dry film. m/z (ESI+) (M+H)⁺=708.6; HPLC t_{R}=3.41 min; ¹H NMR (399.9 MHz, CDCl₃) δ 1.45 (9H, s), 1.68 (9H, s), 1.88 (2H, d), 2.97-3.09 (2H, m), 4.17 (2H, t), 4.86 (2H, d), 6.57 (1H, d), 7.38 (1H, d), 7.44-7.50 (2H, m), 8.00 (1H, d),

### 91D. 4-(4-Pyrimidin-5-yl-1H-benzimidazol-2-yl)-1-(7H-pyrrolo[3,2-e]pyrimidin-4-yl)piperidin-4-amine

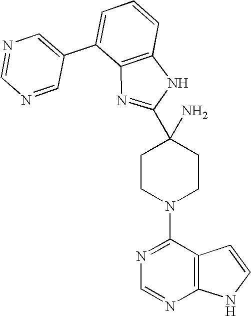

A stream of nitrogen was passed through a mixture of pyrimidine-5-boronic acid (38 mg, 0.3 mmol), tert-butyl 1′-[7-[(2-methylpropan-2-yl)oxycarbonyl]pyrrolo[3,2-e]pyrimidin-4-yl]-1-oxo-5-(trifluoromethylsulfonyloxy)spiro[imidazo[3,4-a]benzimidazole-3,4′-piperidine]-2-carboxylate (170 mg, 0.24 mmol), and sodium carbonate (102 mg, 0.96 mmol) in dioxan (2.4 mL) and water (0.5 mL). After 15 minutes tetrakistriphenyl-phosphine palladium (0) (5.5 mg) was added and the reaction was heated by microwave irradiation at 130° C. for 10 minutes. The mixture was cooled to room temperature and treated with sodium hydroxide solution (2M, 0.5 mL) and tetrabutylammonium bromide (20 mg).The mixture was again heated by microwave irradiation at 130° C. for 10 minutes. The mixture was cooled and the organic layer was separated off and concentrated under reduced pressure. The residue was dissolved with TFA (3 mL) and the solution was allowed to stand overnight. The solution was concentrated under reduced pressure and loaded onto a 5 g SCX-2 cartridge with methanol. The cartridge was washed with methanol and elution of product was achieved with 2M methanolic ammonia. Product containing fractions were concentrated to dryness to give a brown foam. The foam was treated with acetonitrile (ca. 6 mL) and placed in an ultrasound bath for 15 minutes and then heated to 50° C. The insoluble material was removed by filtration. A solid crystallised from the filtrate on standing and this was isolated by filtration and dried in vacuo to afford 4-(4-pyrimidin-5-yl-1H-benzimidazol-2-yl)-1-(7H-pyrrolo[3,2-e]pyrimidin-4-yl)piperidin-4-amine (18.2 mg) as a near colourless solid. m/z (ESI+) (M+H)⁺=412.5; HPLC t_{R}=1.43 min; ¹H NMR (399.9 MHz, DMSO-d₆) δ 1.81-1.88 (2H, m), 2.23-2.34 (2H, m), 3.88-3.97 (2H, m), 4.25-4.32 (2H, m), 6.62-6.66 (1H, m), 7.16-7.18 (1H, m), 7.32 (1H, t), 7.53-7.60 (2H, m), 8.16 (1H, s), 9.16 (1H, s), 9.53 (2H, br, s), 11.65 (1H, s).

### EXAMPLES 92 TO 96

The compounds of Examples 92 to 96 were synthesised in a manner similar to that used for Example 91 using the boronate/boronic acid starting material shown in the table below in place of pyrimidine-5-boronic acid.

| Example | Structure | | | |
|---|---|---|---|---|
| 92 | 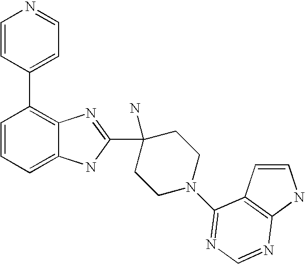 | pyridine-4- boronic acid pinacol cyclic ester | 4-(4-pyridin-4-yl- 1H-benzimidazol-2- yl)-1-(7H- pyrrolo[3,2- e]pyrimidin-4- yl)piperidin-4- amine | ¹H NMR (399.9 MHz, DMSO-d₆) δ 1.82-1.87 (2 H, m), 2.24-2.33 (2 H, m), 3.86-3.95 (2 H, m), 4.29-4.38 (2 H, m), 6.65 (1 H, d), 7.18 (1 H, d), 7.30 (1 H, t), 7.51-7.59 (2 H, m), 8.12-8.22 (2 H, m), 8.62-8.67 (2 H, m), 11.66 (1 H, s) Mass found: 411.2 Retention time: 1.44 |
| 93 | 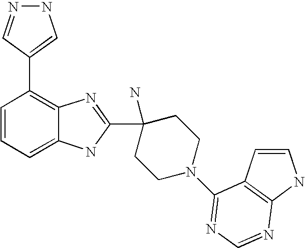 | 4-(4,4,5,5- tetramethyl- 1,3,2- dioxaborolan-2- yl)-pyrazole-1- carboxylic acid tert-butyl ester | 4-[4-(1H-pyrazol-4- yl)-1H- benzimidazol-2-yl]- 1-(7H-pyrrolo[3,2- e]pyrimidin-4- yl)piperidin-4- amine | ¹H NMR (399.9 MHz, DMSO-d₆) δ 1.80-1.90 (2 H, m), 2.26-2.39 (2 H, m), 3.92-4.04 (2 H, m), 4.20-4.32 (2 H, m), 6.64 (1 H, d), 7.10- 7.20 (2 H, m), 7.29 (1 H, d), 7.41 (1 H, d), 8.15 (1 H, s), 8.17-8.69 (2 H, m), 11.62 (1 H, s) Mass found: 400.5 Retention time: 1.44 |
| 94 | 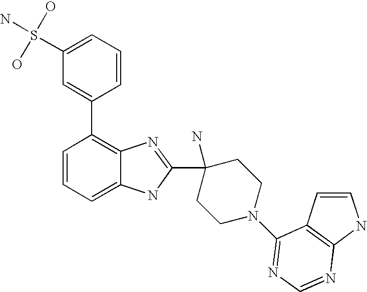 | methyl 3- boronobenzene- sulfonamide | 3-[2-[4-amino-1- (7H-pyrrolo[3,2- e]pyrimidin-4- yl)piperidin-4-yl]- 1H-benzimidazol-4- yl]benzenesulfonamide | ¹H NMR (399.9 MHz, DMSO-d₆) δ 1.80-1.89 (2 H, m), 2.27-2.35 (2 H, m), 3.92-4.02 (2 H, m), 4.20-4.31 (2 H, m), 6.64 (1 H, d), 7.16- 7.20 (1 H, m), 7.26-7.45 (4 H, m), 7.54 (1 H, d), 7.67 (1 H, t), 7.81 (1 H, d), 8.16 (1 H, s), 8.37 (1 H, br, s), 8.53 (1 H, br, s), 11.65 (1 H, s) Mass found: 489.1 Retention time: 1.4 |
| 95 | 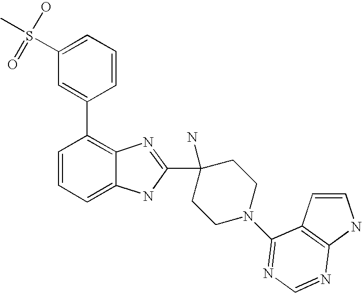 | 3-(methyl- sulfonyl) phenylboronic acid | 4-[4-(3- methylsulfonyl- phenyl)-1H- benzimidazol-2-yl]- 1-(7H-pyrrolo[3,2- e]pyrimidin-4- yl)piperidin-4- amine | ¹H NMR (399.9 MHz, DMSO-d₆) δ 1.80-1.85 (2 H, m), 2.24-2.35 (2 H, m), 3.23 (3 H, s), 3.93- 4.02 (2 H, m), 4.21- 4.28 (2 H, m), 6.63 (1 H, d), 7.18 (1 H, d), 7.30 (1 H, t), 7.49 (1 H, d), 7.55 (1 H, d), 7.76 (1 H, t), 7.89 (1 H, d), 8.15 (1 H, s), 8.43 (1 H, br, s), 8.73 (1 H, br, s), 11.65 (1 H, s) Mass found: 488.2 Retention time: 1.25 |
| 96 | 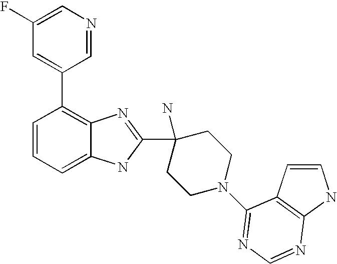 | 5-fluoro-3- pyridinyl boronic acid | 4-[4-(5- fluoropyridin-3-yl)- 1H-benzimidazol-2- yl]-1-(7H- pyrrolo[3,2- e]pyrimidin-4- yl)piperidin-4- amine | ¹H NMR (399.9 MHz, DMSO-d₆) δ 1.82-1.87 (2 H, m), 2.23-2.33 (2 H, m), 3.88-3.96 (2 H, m), 4.27-4.33 (2 H, m), 6.65 (1 H, d), 7.18 (1 H, d), 7.30 (1 H, t), 7.50-7.59 (2 H, m), 8.16 (1 H, s), 8.48 (1 H, br, s), 8.55 (1 H, d), 9.24 (1 H, br, s), 11.65 (1 H, s) Mass found: 429.2 Retention time: 1.59 |

### EXAMPLE 97

### (4-(5-methyl-1H-benzo[d]imidazol-2-yl)-1-(7H-pyrrolo[2,3-d]pyrimidin-4-yl)piperidin-4-yl)methanamine

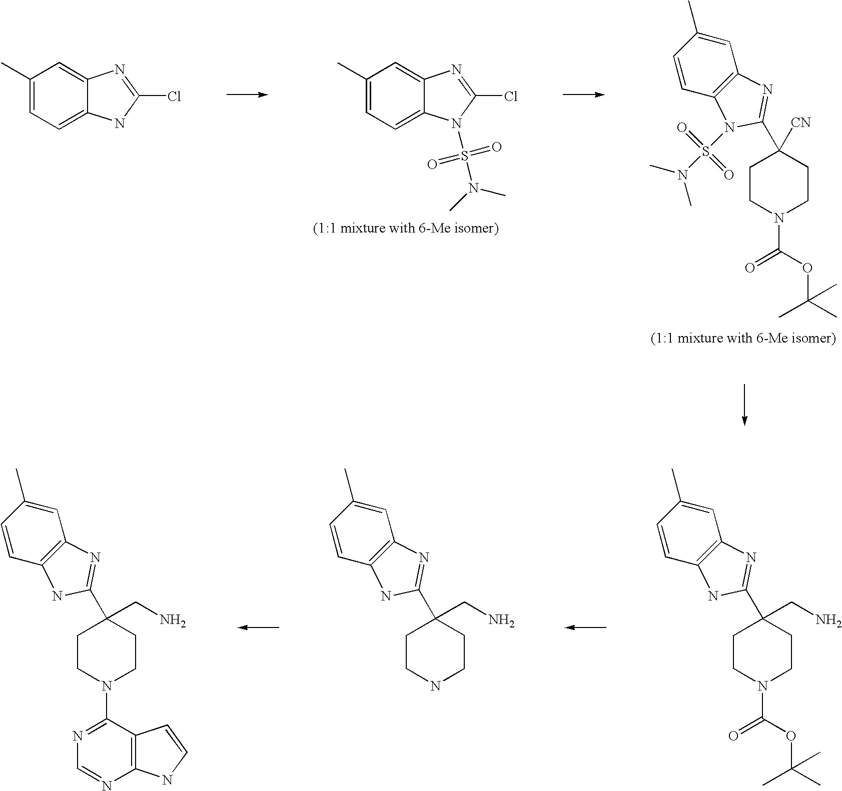

### 97A. 2-chloro-N,N,5-trimethyl-1H-benzo[d]imidazole-1-sulfonamide and 2-chloro-N,N,6-benzo[d]imidazole-1-sulfonamide (1:1 mixture)

The trimethyl-1H title compound was prepared as a 1:1 mixture of isomers from 2-chloro-5-methylbenzimidazole using the procedure described in Example 34A. M/z: [M+H]⁺=274; 1H NMR (DMSO-d₆) 2.43 and 2.47 (each 1.5H, s), 2.98 and 3.00 (each 3H, s), 7.22-7.28 (1H, m), 7.51-7.51 and 7.69-7.70 (each 0.5H, m), 7.59 and 7.75 (each 0.5H, d).

### 97B. tert-butyl 4-cyano-4-(1-(N,N-dimethylsulfamoyl)-5-methyl-1H-benzo[d]imidazol-2-yl)piperidine-1-carboxylate and tert-butyl 4-cyano-4-(1-(N,N-dimethylsulfamoyl)-6-methyl-1H-benzo[d]imidazol-2-yl)piperidine-1-carboxylate (1:1 mixture)

The title compound was prepared as a 1:1 mixture of isomers from 2-chloro-N,N,5-trimethyl-1H-benzo[d]imidazole-1-sulfonamide and 2-chloro-N,N,6-trimethyl-1H-benzo[d]imidazole-1-sulfonamide (1:1 mixture) and 1-tert-butoxycarbonyl-4-cyanopiperidine using the procedure described in Example 34B. M/z: [M+H]⁺=448; 1H NMR (DMSO-d₆) 1.42 (9H, s), 2.16-2.21 (2H, m), 2.44 and 2.55 (each 1.5H, s), 2.62 (2H, d), 2.97 and 2.98 (each 3H, s), 3.08-3.19 (2H, m), 4.07 (2H, d), 7.27-7.33 (1H, m), 7.57-7.61 (1H, m), 7.67-7.70 (1H, m)

### 97C. tert-butyl 4-(aminomethyl)-4-(5-methyl-1H-benzo[d]imidazol-2-yl)piperidine-1-carboxylate

The title compound was prepared from tert-butyl 4-cyano-4-(1-(N,N-dimethylsulfamoyl)-5-methyl-1H-benzo[d]imidazol-2-yl)piperidine-1-carboxylate and tert-butyl 4-cyano-4-(1-(N,N-dimethylsulfamoyl)-6-methyl-1H-benzo[d]imidazol-2-yl)piperidine-1-carboxylate using the procedure described in Example 34D. M/z: [M+H]⁺=345; 1H NMR (DMSO-d₆) 1.40 (9H, s), 1.57-1.64 (2H, m), 2.24 (2H, d), 2.40 (3H, s), 2.74 (2H, s), 2.91 (2H, s), 3.71-3.75 (2H, m), 6.94-6.97 (1H, m), 7.29 (1H, s), 7.38 (1H, d)

### 97D. (4-(5-methyl-1H-benzo[d]imidazol-2-yl)piperidin-4-yl)methanamine

The title compound was prepared from tert-butyl 4-(aminomethyl)-4-(5-methyl-1H-benzo[d]imidazol-2-yl)piperidine-1-carboxylate using the procedure described in Example 89C. M/z: [M+H]⁺=245; 1H NMR (DMSO-d₆) 1.54-1.61 (2H, m), 2.23-2.26 (2H, m), 2.40 (3H, s), 2.52 (2H+DMSO, m), 2.71 (2H, s), 2.81-2.86 (2H, m), 6.93-6.95 (1H, m), 7.28 (1H, s), 7.37 (1H, d)

### 97E. (4-(5-methyl-1H-benzo[d]imidazol-2-yl)-1-(7H-pyrrolo[2,3-d]pyrimidin-4-yl)piperidin-4-yl)methanamine

(4-(5-Methyl-1H-benzo[d]imidazol-2-yl)piperidin-4-yl)methanamine (80 mg, 0.33 mmol), 4-chloro-7H-pyrrolo[2,3-d]pyrimidine (50.3 mg, 0.33 mmol) and N-ethyldiisopropylamine (0.068 mL, 0.39 mmol) in butan-1-ol (2 mL) were heated at 60° C. overnight. The mixture was heated at 75° C. for a day and then 95° C. overnight. The mixture was cooled, absorbed onto an SCX column washed with methanol and eluted with ammonia in methanol. Product containing fractions were concentrated. The crude product was purified by preparative HPLC (Waters XBridge Prep C18 OBD column, 5μ silica, 19 mm diameter, 100 mm length), using decreasingly polar mixtures of water (containing 1% NH3) and MeCN as eluents. Fractions containing the desired compound were evaporated to dryness to afford (4-(5-methyl-1H-benzo[d]imidazol-2-yl)-1-(7H-pyrrolo[2,3-d]pyrimidin-4-yl)piperidin-4-yl)methanamine (35.8 mg, 30.3%) as a white solid. M/z: [M+H]⁺=362; 1H NMR (DMSO-d₆) 1.74-1.81 (2H, m), 2.40-2.43 (5H, m), 2.79 (2H, s), 3.30-3.38 (2H, m), 4.43-4.47 (2H, m), 6.59 (1H, d), 6.95-6.98 (1H, m), 7.17 (1H, d), 7.31 (1H, s), 7.41 (1H, s), 8.13 (1H, s), 11.64 (1H, s)

### EXAMPLE 98

### (1-(5-bromo-7H-pyrrolo[2,3-d]pyrimidin-4-yl)-4-(5-methyl-1H-benzo[d]imidazol-2-yl)piperidin-4-yl)methanamine

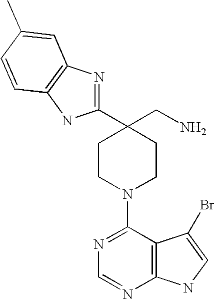

(1-(5-bromo-7H-pyrrolo[2,3-d]pyrimidin-4-yl)-4-(5-methyl-1H-benzo[d]imidazol-2-yl)piperidin-4-yl)methanamine was prepared from (445-Methyl-1H-benzo[d]imidazol-2-yl)piperidin-4-yl)methanamine (as described in Example 97D) and 5-bromo-4-chloro-7H-pyrrolo[2,3-d]pyrimidine (as described in Preparation 6) using the procedure described in Example 97E. M/z: [M+H]+=442; 1H NMR (DMSO-d₆) 1.88-1.95 (2H, m), 2.40-2.46 (5H, m), 2.82 (2H, s), 3.18 (2H, t), 3.97-4.01 (2H, m), 6.95-6.98 (1H, m), 7.31 (1H, s), 7.39-7.42 (1H, d), 7.51 (1H, s), 8.22 (1H, s).

### EXAMPLE 99

### (4-(4-phenyl-1H-benzo[d]imidazol-2-yl)-1(7H-pyrrolo[2,3-d]pyrimidin-4-yl)piperidin-4-yl)methanamine

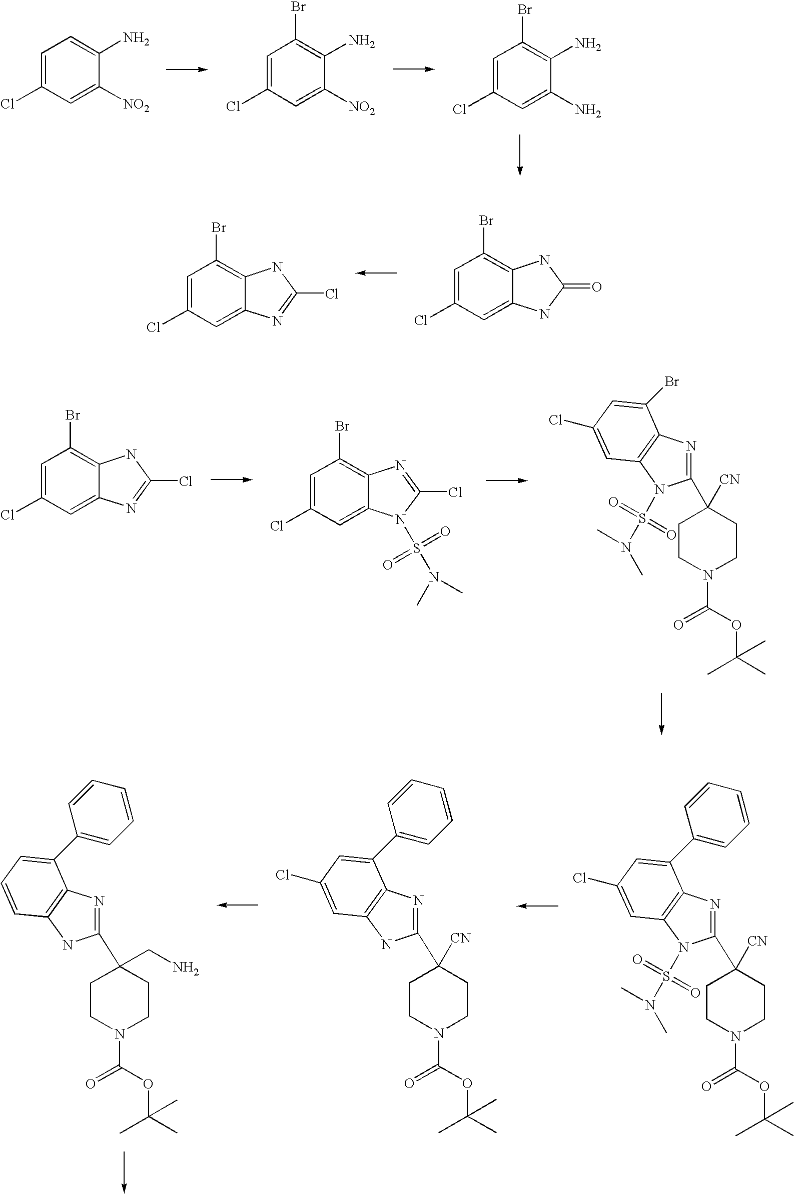

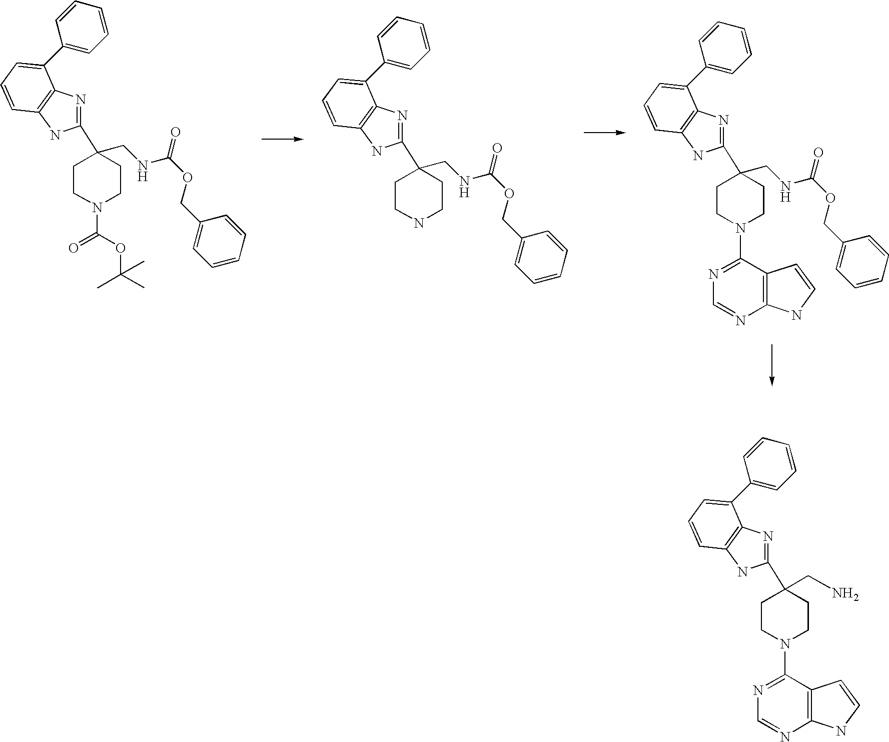

### 99A. 2-bromo-4-chloro-6-nitroaniline

4-Chloro-2-nitroaniline (10 g, 57.95 mmol) was dissolved in acetic acid (90 ml). Bromine (3.12 mL, 60.85 mmol) in acetic acid (30 ml) was added at a rate so that the temperature of the reaction was maintained at 15-20° C. The reaction mixture was stirred for 3 h and then concentrated. The yellow solid was dissolved in DCM, washed with saturated sodium hydrogen carbonate (×2), dried (MgSO₄) and concentrated. The crude product was purified by flash silica chromatography, eluting with 1.5:1 isohexane:DCM. Pure fractions were evaporated to dryness to afford 2-bromo-4-chloro-6-nitroaniline (9.13 g, 62.7%) as a yellow crystalline solid. 1H NMR (DMSO-d₆) 7.27 (2H, s), 8.02 (1H, d), 8.09 (1H, d).

### 99B. 4-bromo-6-chloro-1H-benzo[d]limidazol-2(3H)-one

3-Bromo-5-chlorobenzene-1,2-diamine (6.35 g, 28.67 mmol) was dissolved in chloroform (80 mL) and 1,1′-carbonyldiimidazole (6.97 g, 43.01 mmol) added. The mixture was heated at reflux for 1 h. Further 1,1′-carbonyldiimidazole (6.97 g, 43.01 mmol) was added and the mixture heated for 1 h. The mixture was cooled and filtered to give 4-bromo-6-chloro-1H-benzo[d]imidazol-2(3H)-one (5.68 g, 80%) as a white solid. M/z: [M+H]⁺=247; 1H NMR (DMSO-d₆) 6.97 (1H, d), 7.21 (1H, d), 11.07 (1H, s), 11.22 (1H, s).

### 99C. 7-bromo-2,5-dichloro-1H-benzo[d]imidazole

4-Bromo-6-chloro-1H-benzo[d]imidazol-2(3H)-one (5.68 g, 22.95 mmol) was suspended in phosphorous oxychloride (40 mL) and DMF (0.75 ml) added. The mixture was heated at reflux for 7 h. The mixture was cooled and stood overnight. The mixture was concentrated and azeotroped (×2) with toluene. The residue was taken up in DCM (100 ml) with the aid of a little methanol. The solution was filtered to remove solid and the filtrate washed with saturated aqueous sodium hydrogen carbonate, dried (MgSO₄) and concentrated to give 7-bromo-2,5-dichloro-1H-benzo[d]imidazole (2.88 g, 47.2%) as an orange solid. M/z: [M+H]⁺=266; 1H NMR (DMSO-d₆) 7.56 (1H, d), 7.62 (1H, brs), 13.88 (1H, brs)

### 99D. 4-bromo-2,6-dichloro-N,N-dimethyl-1H-benzo[d]imidazole-1-sulfonamide

The title compound was prepared from 7-Bromo-2,5-dichloro-1H-benzo[d]imidazole using the procedure described in Example 34A. M/z: [M+H]⁺=374; 1H NMR (DMSO-d₆) 3.04 (6H, s), 7.81 (1H, d), 7.87 (1H, d). Note: this is a single isomer, assigned as the isomer shown based on likely steric and electronic reasons but no spectroscopic proof is available.

### 99E. tert-butyl 4-(4-bromo-6-chloro-1-(N,N-dimethylsulfamoyl)-1H-benzo[d]imidazol-2-yl)-4-cyanopiperidine-1-carboxylate

The title compound was prepared from 4-bromo-2,6-dichloro-N,N-dimethyl-1H-benzo[d]imidazole-1-sulfonamide and 1-tert-butoxycarbonyl-4-cyanopiperidine using the procedure described in Example 34B. M/z: [M+H]⁺=548; 1H NMR (DMSO-d₆) 1.43 (9H, s), 2.23-2.26 (2H, m), 2.62 (2H, d), 3.05 (6H, s), 3.09-3.18 (2H, d), 7.76 (1H, d), 7.87 (1H, d)

### 99F. tert-butyl 4-(6-chloro-1-(N,N-dimethylsulfamoyl)-4-phenyl-1H-benzo[d]imidazol-2-yl)-4-cyanopiperidine-1-carboxylate

The title compound was prepared from phenylboronic acid and tert-butyl 4-(4-bromo-6-chloro-1-(N,N-dimethylsulfamoyl)-1H-benzo[d]imidazol-2-yl)-4-cyanopiperidine-1-carboxylate using the procedure described in Preparation B1. M/z: [M+H]⁺=544; 1H NMR (DMSO-d₆) 1.42 (9H, s), 2.20-2.28 (2H, m), 2.63 (2H, d), 3.06 (6H, s), 3.09-3.16 (2H, m), 4.07-4.11 (2H, m), 7.45-7.49 (1H, m), 7.52-7.57 (2H, m), 7.71-7.73 (2H, m), 8.00-8.03 (2H, m).

### 99G. tert-butyl 4-(6-chloro-4-phenyl-1H-benzo[d]imidazol-2-yl)-4-cyanopiperidine-1-carboxylate

Tert-butyl 4-(6-chloro-1-(N,N-dimethylsulfamoyl)-4-phenyl-1H-benzo[d]imidazol-2-yl)-4-cyanopiperidine-1-carboxylate (220 mg, 0.40 mmol) was dissolved in dioxane (5 mL) and 4M HCl in dioxane (5.00 mL) added. The mixture was stirred for 3 h and concentrated. The crude product was purified by ion exchange chromatography, using an SCX column. The desired product was eluted from the column using 7M NH3/MeOH and pure fractions were evaporated to dryness to afford 4-(6-chloro-4-phenyl-1H-benzo[d]imidazol-2-yl)piperidine-4-carbonitrile (133 mg, 98%) as a white foam. M/z: [M+H]⁺=337; 1H NMR (DMSO-d₆) 2.13-2.17 (2H, m), 2.27 (2H, d), 2.80-2.87 (2H, m), 3.03-3.08 (2H, m), 7.42-7.45 (2H, m), 7.51-7.54 (2H, m), 7.59 (1H, d), 8.01 (2H, s).

4-(6-chloro-4-phenyl-1H-benzo[d]imidazol-2-yl)piperidine-4-carbonitrile (130 mg, 0.39 mmol) was dissolved in THF (5 mL) and di-tert-butyl dicarbonate (84 mg, 0.39 mmol) added. The mixture was stirred for 1.5 h and concentrated. The crude product was purified by flash silica chromatography, eluting with 0.75% MeOH in DCM. Pure fractions were evaporated to dryness to afford tert-butyl 4-(6-chloro-4-phenyl-1H-benzo[d]imidazol-2-yl)-4-cyanopiperidine-1-carboxylate (161 mg, 95%) as a white foam. M/z: [M+H]⁺=337; 1H NMR (DMSO-d₆) 1.44 (9H, s), 2.15-2.22 (2H, m), 2.33-2.38 (2H, m), 3.16 (2H, s), 4.04-4.07 (2H, m), 7.42-7.58 (5H, m), 8.10 (2H, d), 13.17 (1H, s)

### 99H. tert-butyl 4-(aminomethyl)-4-(4-phenyl-1H-benzo[d]imidazol-2-yl)piperidine-1-carboxylate

The title compound was prepared from tert-butyl 4-(6-chloro-4-phenyl-1H-benzo[d]imidazol-2-yl)-4-cyanopiperidine-1-carboxylate using the procedure described in Example 34D. M/z: [M+H]⁺=407; 1H NMR (DMSO-d₆) 1.40 (9H, s), 1.60-1.67 (2H, m), 2.30 (2H, d), 2.81 (2H, s), 3.02 (2H, s), 3.73-3.76 (2H, m), 7.24 (1H, t), 7.33-7.39 (2H, m), 7.46-7.51 (3H, m), 8.05 (2H, d)

### 99I. tert-butyl 4-((benzyloxycarbonylamino)methyl)-4-(4-phenyl-1H-benzo[d]imidazol-2-yl)piperidine-1-carboxylate

Benzyl chloroformate (0.048 mL, 0.34 mmol) was added to tert-butyl 4-(aminomethyl)-4-(4-phenyl-1H-benzo[d]imidazol-2-yl)piperidine-1-carboxylate (115 mg, 0.28 mmol) and N,N-diisopropylethylamine (0.074 mL, 0.42 mmol) in DCM (6 mL). The mixture was stirred for 1 h, washed with brine, dried (MgSO₄) and concentrated. The crude product was purified by flash silica chromatography, eluting with 1.5% MeOH/DCM. Pure fractions were evaporated to dryness to afford tert-butyl 4-((benzyloxycarbonylamino)methyl)-4-(4-phenyl-1H-benzo[d]imidazol-2-yl)piperidine-1-carboxylate (162 mg, 106%) as a white foam. M/z: [M+H]⁺=541; 1H NMR (DMSO-d₆) 1.40 (9H, s), 1.61-1.68 (2H, m), 2.30-2.35 (2H, m), 2.86-2.96 (2H, m), 3.35 (2H, d), 3.76-3.79 (2H, m), 4.94 (2H, s), 7.23-7.49 (11H, m), 8.11-8.13 (2H, m), 12.36 (1H, s).

### 99J. benzyl (4-(4-phenyl-1H-benzo[d]imidazol-2-yl)piperidin-4-yl)methylcarbamate

The title compound was prepared from tert-butyl 4-((benzyloxycarbonylamino)methyl)-4-(4-phenyl-1H-benzo[d]imidazol-2-yl)piperidine-1-carboxylate using the procedure described in Example 89C. M/z: [M+H]⁺=441; 1H NMR (DMSO-d₆+d4 AcOH) 1.89-1.93 (2H, m), 2.50-2.53 (2H+DMSO, m), 2.85-2.92 (2H, m), 3.26-3.29 (2H, m), 3.40 (2H, s), 4.95 (2H, s), 7.23-7.30 (6H, m), 7.35-7.39 (2H, m), 7.46-7.52 (3H, m), 8.01 (2H, s)

### 99K. benzyl (4-(4-phenyl-1H-benzo[d]imidazol-2-yl)-1-(7H-pyrrolo[2,3-d]pyrimidin-4-yl)piperidin-4-yl)methylcarbamate

Benzyl (444-phenyl-1H-benzo[d]imidazol-2-yl)piperidin-4-yl)methylcarbamate (107 mg, 0.24 mmol), 4-chloro-7H-pyrrolo[2,3-d]pyrimidine (44.8 mg, 0.29 mmol) and N-ethyldiisopropylamine (0.085 mL, 0.49 mmol) were heated at 120° C. overnight. The mixture was cooled and absorbed onto an SCX column, washed with methanol and eluted with ammonia in methanol. Product containing fractions were concentrated. The crude product was purified by flash silica chromatography, elution gradient 2.5-5% MeOH in DCM. Pure fractions were evaporated to dryness to afford benzyl (4-(4-phenyl-1H-benzo[d]imidazol-2-yl)-1-(7H-pyrrolo[2,3-d]pyrimidin-4-yl)piperidin-4-yl)methylcarbamate (94 mg, 69.4%) as a white foam. M/z: [M+H]⁺=558; 1H NMR (DMSO-d₆+d4 AcOH) 1.77-1.86 (2H, m), 2.50-2.54 (2H+DMSO, m), 3.32-3.42 (4H, m), 4.47-4.51 (2H, m), 4.93 (2H, s), 6.60 (1H, d), 7.17 (1H, d), 7.21-7.28 (7H, m), 7.32-7.39 (2H, m), 7.4 5-7.49 (3H, m), 8.10-8.15 (3H, m)

### 99L. (4-(4-phenyl-1H-benzo[d]imidazol-2-yl)-1-(7H-pyrrolo[2,3-d]pyrimidin-4-yl)piperidin-4-yl)methanamine

Benzyl (4-(4-phenyl-1H-benzo[d]imidazol-2-yl)-1-(7H-pyrrolo[2,3-d]pyrimidin-4-yl)piperidin-4-yl)methylcarbamate (90 mg, 0.16 mmol) was dissolved in MeOH (15 mL) and a few drops of AcOH added. The system was purged with nitrogen. 10% Pd/C (45 mg, 50% by mass) was added and the mixture stirred under a hydrogen atmosphere for 4 h. The mixture was filtered and the filtrate absorbed onto a SCX column, washed with methanol and elute with ammonia in methanol. Product containing fractions were concentrated. The crude product was purified by flash silica chromatography, eluting with 5% 7N ammonia in methanol in DCM. Pure fractions were evaporated to dryness to afford (4-(4-phenyl-1H-benzo[d]imidazol-2-yl)-1-(7H-pyrrolo[2,3-d]pyrimidin-4-yl)piperidin-4-yl)methanamine (55.0 mg, 80%) as a white solid. M/z: [M+H]⁺=424; 1H NMR (DMSO-d₆) 1.78-1.85 (2H, m), 2.42-2.48 (2H, m), 2.86 (2H, s), 3.43-3.52 (2H, m), 4.42-4.46 (2H, m), 5.05 (2H, brs), 6.61 (1H, d), 7.16 (1H, d), 7.25 (1H, t), 7.33-7.39 (2H, m), 7.48-7.52 (3H, m), 8.06 (2H, d), 8.14 (1H, t), 11.63 (1H, s)

### EXAMPLE 100

### 1H-pyrazol-4-yl)-1H-benzo[d]imidazol-2-yl)-1-(7H-pyrrolo[2,3-d]pyrimidin-4-yl)piperidin-4-yl)methanamine

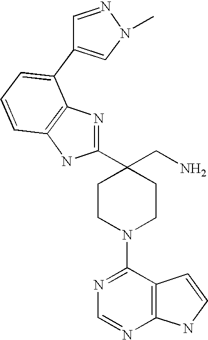

The title compound was prepared using the synthetic route described in Example 99, starting from the product of Example 99E.

### 100A. tert-butyl 4-(6-chloro-1-(N,N-dimethylsulfamoyl)-4-(1-methyl-1H-pyrazol-4-yl)-1H-benzo[d]imidazol-2-yl)-4-cyanopiperidine-1-carboxylate

The title compound was prepared from 1-methyl-4-(4,4,5,5-tetramethyl-1,3,2-dioxaborolan-2-yl)-1H-pyrazole and tert-butyl 4-(4-bromo-6-chloro-1-(N,N-dimethylsulfamoyl)-1H-benzo[d]imidazol-2-yl)-4-cyanopiperidine-1-carboxylate (as described in Example 99E) using the procedure described in Preparation B-1. M/z: [M+H]⁺=548; 1H NMR (DMSO-d₆) 1.44 (9H, s), 2.26-2.35 (2H, m), 2.68-2.71 (2H, m), 3.03 (6H, s), 3.16-3.21 (2H, m), 3.95 (3H, s), 4.11-4.15 (2H, m), 7.52 (1H, d), 7.81 (1H, d), 8.33 (1H, d), 8.60 (1H, s).

### 100B. tert-butyl 4-(6-chloro-4-(1-methyl-1H-pyrazol-4-yl)-1H-benzo[d]imidazol-2-yl)-4-cyanopiperidine-1-carboxylate

The title compound was prepared from tert-butyl 4-(6-chloro-1-(N,N-dimethylsulfamoyl)-4-(1-methyl-1H-pyrazol-4-yl)-1H-benzo[d]imidazol-2-yl)-4-cyanopiperidine-1-carboxylate using the procedure described in Example 99G. M/z: [M+H]⁺=441; 1H NMR (DMSO-d₆) 1.45 (9H, s), 2.19-2.26 (2H, m), 2.40 (2H, d), 3.18-3.19 (2H, m), 3.94 (3H, s), 4.06 (2H, d), 7.38 (1H, d), 7.55 (1H, d), 8.33 (1H, s), 8.61 (1H, s), 13.06 (1H, s).

### 100C. tert-butyl 4-(aminomethyl)-4-(4-(1-methyl-1H-pyrazol-4-yl)-1H-benzo[d]imidazol-2-yl)piperidine-1-carboxylate

The title compound was prepared from tert-butyl 4-(6-chloro-4-(1-methyl-1H-pyrazol-4-yl)-1H-benzo[d]imidazol-2-yl)-4-cyanopiperidine-1-carboxylate using the procedure described in Example 34D. M/z: [M+H]⁺=411; 1H NMR (DMSO-d₆) 1.41 (9H, s), 1.63-1.70 (2H, m), 2.31-2.35 (2H, m), 2.82 (2H, s), 3.00 (2H, brs), 3.74-3.80 (2H, m), 3.93 (3H, s), 7.13 (1H, t), 7.28-7.30 (1H, m), 7.37-7.39 (1H, m), 8.22 (1H, s)

### 100D. tert-butyl 4-((benzyloxycarbonylamino)methyl)-4-(4-(1-methyl-1H-pyrazol-4-yl)-1H-benzo[d]imidazol-2-yl)piperidine-1-carboxylate

The title compound was prepared from tert-butyl 4-(aminomethyl)-4-(4-(1-methyl-1H-pyrazol-4-yl)-1H-benzo[d]imidazol-2-yl)piperidine-1-carboxylate using the procedure described in Example 991. M/z: [M+H]⁺=545; 1H NMR (DMSO-d₆) 1.40 (9H, s), 1.63-1.71 (2H, m), 2.35 (2H, d), 2.93 (2H, brs), 3.36 (2H, d), 3.78-3.82 (2H, m), 3.92 (3H, s), 4.95 (2H, s), 7.14 (1H, t), 7.22-7.34 (7H, m), 7.39-7.41 (1H, m), 8.24 (1H, m), 8.51 (1H, s), 12.26 (1H, s)

### 100E. benzyl (4-(4-(1-methyl-1H-pyrazol-4-yl)-1H-benzo[d]imidazol-2-yl)piperidin-4-yl)methylcarbamate

The title compound was prepared from tert-butyl 4-((benzyloxycarbonylamino)methyl)-4-(4-(1-methyl-1H-pyrazol-4-yl)-1H-benzo[d]imidazol-2-yl)piperidine-1-carboxylate using the procedure described in Example 89C. M/z: [M+H]⁺=445; 1H NMR (DMSO-d₆) 1.62-1.69 (2H, m), 2.30 (2H, d), 2.53-2.61 (2H, m), 2.85 (2H, d), 3.32-3.35 (2H+H₂O, m), 3.90-3.92 (3H, m), 4.94 (2H, s), 7.13 (1H, t), 7.20-7.33 (7H, m), 7.38 (1H, d), 8.24 (1H, d), 8.51 (1H, d), 12.13 (1H, s).

### 100F. benzyl (4-(4-(1-methyl-1H-pyrazol-4-yl)-1H-benzo[d]imidazol-2-yl)-1-(7H-pyrrolo[2,3-d]pyrimidin-4-yl)piperidin-4-yl)methylcarbamate

The title compound was prepared from benzyl (4-(4-(1-methyl-1H-pyrazol-4-yl)-1H-benzo[d]imidazol-2-yl)piperidin-4-yl)methylcarbamate using the procedure described in Example 99K. M/z: [M+H]⁺=562; 1H NMR (DMSO-d₆) 1.80-1.89 (2H, m), 2.52-2.55 (2H+DMSO, m), 3.32-3.43 (4H+H₂O, m), 3.93 (3H, s), 4.53 (2H, d), 4.94 (2H, s), 6.62 (1H, d), 7.14-7.35 (9H, m), 7.40-7.42 (1H, m), 8.15 (1H, s), 8.27 (1H, d), 8.54 (1H, s), 11.65 (1H, s), 12.34 (1H, s).

### 100G. 1H-pyrazol-4-yl)-1H-benzo[d]imidazol-2-yl)-1-(7H-pyrrolo[2,3-d]pyrimidin-4-yl)piperidin-4-yl)methanamine

The title compound was prepared from benzyl (4-(4-(1-methyl-1H-pyrazol-4-yl)-1H-benzo[d]imidazol-2-yl)-1-(7H-pyrrolo[2,3-d]pyrimidin-4-yl)piperidin-4-yl)methylcarbamate using the procedure described in Example 99L. M/z: [M+H]⁺=428; 1H NMR (DMSO-d₆) 1.80-1.88 (2H, m), 2.46-2.52 (2H+DMSO, m), 2.88 (2H, s), 3.43-3.50 (2H, m), 3.94 (3H, s), 4.44-4.50 (2H, m), 6.62 (1H, d), 7.12-7.18 (2H, m), 7.30 (1H, d), 7.38 (1H, d), 8.14 (1H, s), 8.25 (1H, s), 8.52 (1H, s), 11.64 (1H, s).

### EXAMPLE 101

### [4-[4-(oxolan-3-yl)-1H-benzimidazol-2-yl]-1-(7H-pyrrolo[3,2-e]pyrimidin-4-yl)piperidin-4-yl]methanamine

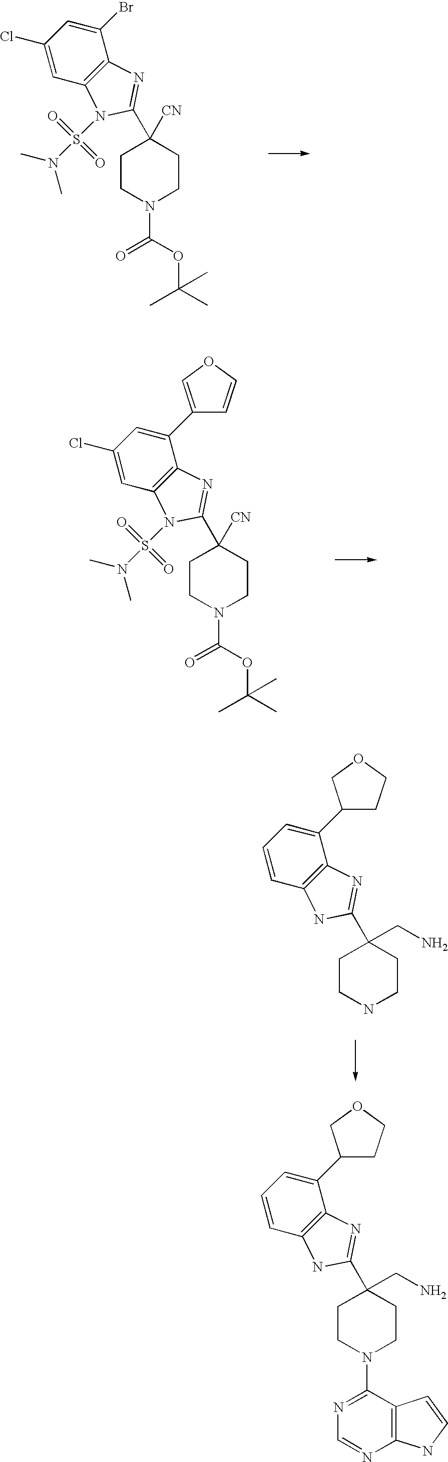

### 101A. tert-butyl 4-(6-chloro-1-(N,N-dimethylsulfamoyl )-4-(furan-3-yl)-1H-benzo[d]imidazol-2-yl)-4-cyanopiperidine-1-carboxylate

The title compound was prepared from 2-(furan-3-yl)-4,4,5,5-tetramethyl-1,3,2-dioxaborolane and tert-butyl 4-(4-bromo-6-chloro-1-(N,N-dimethylsulfamoyl )-1H-benzo[d]imidazol-2-yl)-4-cyanopiperidine-1-carboxylate (as described in Example 99E) using the procedure described in Preparation B1. M/z: [M+H]⁺=534; 1H NMR (DMSO-d₆) 1.43 (9H, s), 2.26-2.35 (2H, m), 2.69 (2H, d), 3.04 (6H, s), 3.10-3.20 (2H, m), 4.11-4.14 (2H, m), 7.37-7.38 (1H, m), 7.62 (1H, d), 7.86 (2H, m), 8.70-8.71 (1H, m).

### 101B. (4-(4-(tetrahydrofuran-3-yl)-1H-benzo[d]imidazol-2-yl)piperidin-4-yl)methanamine

Tert-butyl 4-(6-chloro-1-(N,N-dimethylsulfamoyl )-4-(furan-3-yl)-1H-benzo[d]imidazol-2-yl)-4-cyanopiperidine-1-carboxylate (435 mg, 0.81 mmol) was dissolved in ethanol (19.5 mL), THF (5.9 mL) and 2N NaOH (2.9 mL). Raney(R) nickel, 50% slurry in water, (710 mg, 8.29 mmol) was added and the mixture stirred under a hydrogen atmosphere for 20 h. The system was purged with nitrogen and 10% Pd/C (44 mg, 10% by mass) added. The mixture was stirred under a hydrogen atmosphere for 24 h. The mixture was filtered and concentrated. The residue was taken-up in dioxane (5 ml) and 4M HCl in dioxane (5 ml) added. The mixture was stirred for 1 h and then concentrated. The crude product was purified by ion exchange chromatography, using an SCX column. The desired product was eluted from the column using 7M NH3/MeOH and pure fractions were evaporated to dryness to afford (4-(4-(tetrahydrofuran-3-yl)-1H-benzo[d]imidazol-2-yl)piperidin-4-yl)methanamine (198 mg, 81%) as a white solid. M/z: [M+H]⁺=301; 1H NMR (DMSO-d₆) 1.57-1.64 (2H, m), 2.14-2.38 (4H, m), 2.54-2.60 (2H, m), 2.75 (2H, s), 2.81-2.89 (2H, m), 3.73 (1H, t), 3.85-3.89 (1H, m), 3.91-3.95 (1H, m), 3.97-4.04 (1H, m), 4.15 (1H, t), 7.02 (1H, d), 7.08 (1H, t), 7.32-7.35 (1H, m).

### 101C. [4-[4-(oxolan-3-yl)-1H-benzimidazol-2-yl]-1-(7H-pyrrolo[3,2-e]pyrimidin-4-yl)piperidin-4-yl]methanamine

(4-(4-(tetrahydrofuran-3-yl)-1H-benzo[d]imidazol-2-yl)piperidin-4-yl)methanamine (100 mg, 0.33 mmol) and 4-chloro-7H-pyrrolo[2,3-d]pyrimidine (51 mg, 0.33 mmol) were dissolved in butan-1-ol (2.5 ml). N,N-Diisopropylethylamine (69u1, 0.39 mmol) was added and the mixture heated at 60° C. for 64 h. The mixture was absorbed onto an SCX column, washed with MeOH and eluted with ammonia in MeOH. Product containing fractions were concentrated and purified by preparative, reverse phase HPLC (Waters XBridge Prep C18 OBD column, 5μ silica, 19 mm diameter, 100 mm length), using decreasingly polar mixtures of water (containing 1% NH3) and MeCN as eluents to give [4-[4-(oxolan-3-yl)-1H-benzimidazol-2-yl]-1-(7H-pyrrolo[3,2-e]pyrimidin-4-yl)piperidin-4-yl]methanamine (20 mg, 15%) as a white solid. M/z: [M+H]⁺=418; 1H NMR (DMSO-d₆ @ 373K) 1.84-1.89 (2H, m), 2.17-2.23 (1H, m), 2.34-2.40 (1H, m), 2.44-2.47 (2H, m), 2.91 (2H, s), 3.52-3.58 (2H, m), 3.79 (1H, t), 3.86-3.90 (1H, m), 3.96 (1H, t), 3.99-4.04 (1H, m), 4.17 (1H, t), 4.32-4.37 (2H, m), 6.54 (1H, d), 7.01 (1H, d), 7.06-7.09 (2H, m), 7.33-7.35 (1H, m), 8.12 (1H, s), 11.25 (1H, brs).

### EXAMPLE 102

### 4-(7-benzyloxy)-1H-benzo[d]imidazol-2-yl)-1-(9H-purin-6-yl)piperidin-4-amine

### 102A. Methyl 4-(tert-butoxycarbonylamino)-1-(9H-purin-6-yl)piperidine-4-carboxylate

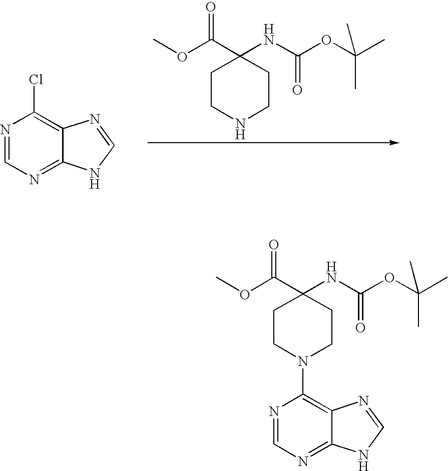

A mixture of 6-chloro-9H-purine (5.41 g, 35 mmol), methyl 4-(tert-butoxycarbonyl-amino)piperidine-4-carboxylate (10.40 g, 40.25 mmol)* and triethylamine (24.39 mL, 175.00 mmol) in ethanol (175 mL) was heated to reflux for 100 minutes under nitrogen. The resulting mixture was filtered and the filtrate evaporated. The crude product was purified by flash silica chromatography, elution gradient 2 to 8% MeOH in DCM. Pure fractions were evaporated to dryness to afford the title compound (10.84 g, 82%) as a white solid. m/z (ESI+) M+=377; HPLC t_{R}=1.37 min; 1H NMR (399.9 MHz, DMSO-d6) δ 1.41 (9H, s), 1.89-1.93 (2H, m), 2.01-2.05 (2H, m), 3.61 (4H, s), 4.92 (2H, s), 7.47 (1H, s), 8.12 (1H, s), 8.22 (1H, s), 13.02 (1H, s).

* Commercially available from Astatech (catalogue number: 55743)

### 102B. 4-[(2-Methylpropan-2-yl)oxycarbonylamino]-1-(9H-purin-6-yl)piperidine-4-carboxylic acid

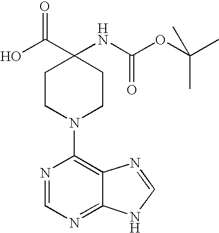

Sodium hydroxide 2M aqueous (37.7 mL, 75.48 mmol) was added in one portion to methyl 4-(tert-butoxycarbonylamino)-1-(9H-purin-6-yl)piperidine-4-carboxylate (9.47 g, 25.16 mmol) in THF (70 mL) and MeOH (45 mL) at 20° C. under nitrogen. The resulting solution was stirred at 20° C. for 18 hours, then acidified to pH3.5 with 2M aqueous HCl. The mixture was washed sequentially four times with DCM (100 mL). The combined organic layers were washed with saturated brine and the organic layer was dried over MgSO4, filtered and evaporated to afford the title compound (9.50 g, >100%) as a white dry film. m/z (ESI+) (M+H)+=363; HPLC t_{R}=1.13 min; 1H NMR (399.9 MHz, DMSO-d6) δ 1.41 (9H, s), 1.84-1.91(2H, m), 2.00-2.06 (2H, m), 3.55-3.61 (2H, m), 4.90-4.97 (2H, m, 7.30 (1H, s), 8.11 (1H, s), 8.21 (1H, s), 13.00 (1H, s).

### 102C. 3:1 regioisomeric mixture of tert-butyl 4-(2-amino-3-(benzyloxy)phenylcarbamoyl)-1-(9H-purin-6-yl)piperidin-4-ylcarbamate and tert-butyl 4-(2-amino-6-(benzyloxy)phenylcarbamoyl)-1-(9H-purin-6-yl)piperidin-4-ylcarbamate

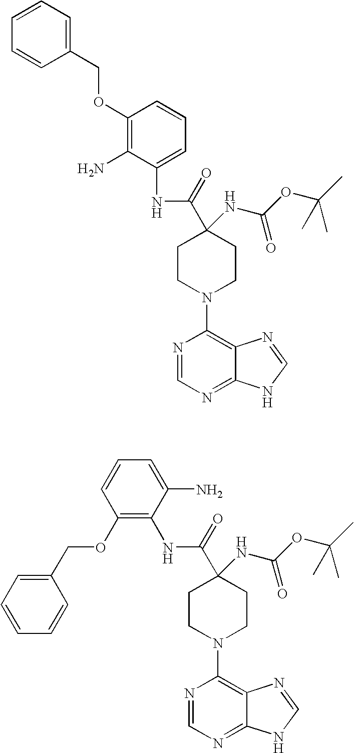

N,N-Diisopropylethylamine (5.94 mL, 34.07 mmol) was added in one portion to a stirred suspension of 4-(tert-butoxycarbonylamino)-1-(9H-purin-6-yl)piperidine-4-carboxylic acid (10.29 g, 28.40 mmol) and O-(7-Azabenzotriazol-1-yl)-N,N,N′,N′-tetramethyluronium hexafluorophosphate (10.90 g, 28.68 mmol) in NMP (60 mL). The mixture was warmed to 50° C. for 15 minutes under nitrogen and cooled to 20° C. 3-(benzyloxy)benzene-1,2-diamine (6.08 g, 28.40 mmol) was added in one portion and the resulting solution was stirred at 20° C. for 60 hours. More O-(7-Azabenzotriazol-1-yl)-N,N,N′,N′-tetramethyluronium hexafluorophosphate (2.72 g, 7.17 mmol) and N,N-Diisopropylethylamine (1.98 mL, 10.45 mmol) was added and the mixture was heated to 55° C. for 1 hour and cooled. The reaction mixture was diluted with EtOAc (200 mL), and washed sequentially with water (3×100 mL) and saturated brine (100 mL). The organic layer was dried over MgSO4, filtered and evaporated to afford crude product which was purified by flash silica chromatography, elution gradient 1 to 10% MeOH in DCM. Pure fractions were evaporated to dryness to afford a pink foam which was shown to be a 3:1 regioisomeric mixture of tert-butyl N-[4-[(2-amino-3-phenylmethoxyphenyl)carbamoyl]-1-(9H-purin-6-yl)piperidin-4-yl]carbamate and tert-butyl N-[4-[(2-amino-6-phenylmethoxyphenyl)carbamoyl]-1-(9H-purin-6-yl)piperidin-4-yl]carbamate (10.22 g, 64%). m/z (ESI+) (M+H)+=559; HPLC t_{R}=1.76 min (21%), 2.00 min (75%); 1H NMR (399.9 MHz, DMSO-d6) δ 1.44 (9H, d), 1.96 (0.5H, m), 2.08 (3.5H, t), 3.78 (2H, m), 4.85 (2H, s), 4.95 (0.5H, s), 5.13 (1.5H, s), 6.27 (0.25H, d), 6.32 (0.25H, d), 6.53 (0.75H, t), 6.64 (0.75H, s), 6.81-6.83 (0.25H, m), 7.22-7,25 (0.75H, m), 7.31-7.42 (3.75H, m), 7.50 (1.5H, d), 8.15 (1H, m), 8.24 (1H, m), 8.57 (0.25H, s), 9.08 (0.75H, s)

### 102D. 4-(7-(benzyloxy)-1H-benzo[d]imidazol-2-yl)-1-(9H-purin-6-yl)piperidin-4-amine

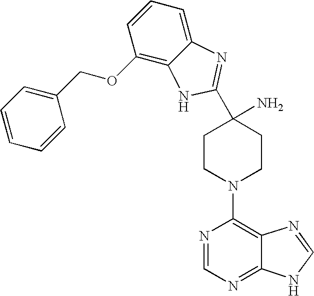

A solution of tert-butyl 4-(2-amino-3-(benzyloxy)phenylcarbamoyl)-1-(7H-pyrrolo[2,3-d]pyrimidin-4-yl)piperidin-4-ylcarbamate and tert-butyl 4-(2-amino-6-(benzyloxy)phenylcarbamoyl)-1-(7H-pyrrolo[2,3-d]pyrimidin-4-yl)piperidin-4-ylcarbamate (10.21 g, 18.28 mmol) in NMP (46 mL) was treated in one portion with hydrogen chloride (4M in dioxane) (45.70 mL, 182 8 mmol) and the resulting solution was stirred at 95° C. for 3 hours. All solvent was removed by evaporation at 95° C. under reduced pressure. The residue was taken into DMF (15 mL), diluted with DCM (45 mL) and added to a silica MPLC chromatography column, elution gradient 2 to 20% of a 10% methanolic solution of aq NH4OH in DCM. Pure fractions were evaporated to dryness to afford 4-(7-(benzyloxy)-1H-benzo[d]imidazol-2-yl)-1-(9H-purin-6-yl)piperidin-4-amine (5.61 g, 69.7 %) as a white solid. m/z (ESI+) (M+H)+=441; HPLC tR=1.26 min; 1H NMR (399.9 MHz, DMSO-d6) δ 1.78 (2H, d), 2.22-2.28 (2H, m), 4.02 (2H, s), 4.84 (2H, s), 5.30 (2H, s), 6.75 (1H, s), 7.02 (1H, t), 7.09 (1H, s), 7.33 (1H, m), 7.40 (2H, t), 7.51 (2H, s), 8.11 (1H, s), 8.21-8.23 (1H, s).

### EXAMPLE 103

### 4-(4-Phenyl-1H-benzimidazol-2-yl)-1-(9H-purin-6-yl)piperidin-4-amine

### 103A. tert-Butyl 1′-[9-[(2-methylpropan-2-yl)oxycarbonyl]purin-6-yl]-1-oxo-5-phenylmethoxyspiro[imidazo[3,4-a]benzimidazole-3,4′-piperidine]-2-carboxylate

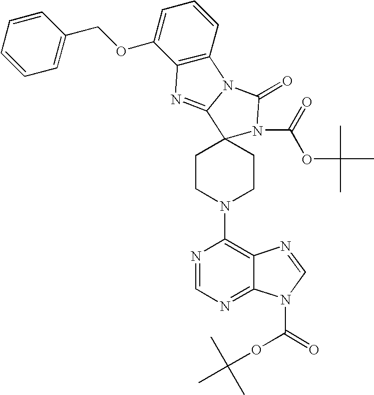

A solution of di-tert-butyl dicarbonate (10.98 g, 50.31 mmol) in THF (50 mL) was added to a stirred suspension of 4-(7-(benzyloxy)-1H-benzo[d]imidazol-2-yl)-1-(9H-purin-6-yl)piperidin-4-amine (Example 102D) (5.54 g, 12.58 mmol) in THF (50 mL) at 20° C. 4-dimethylaminopyridine (0.230 g, 1.89 mmol) was added and the resulting suspension was stirred at 20° C. for 2 hours under nitrogen. The mixture was concentrated under reduced pressure and the residue was purified by flash silica chromatography, elution gradient 15 to 50% EtOAc in DCM. Pure fractions were evaporated to dryness to afford tert-butyl 1′-[9-[(2-methylpropan-2-yl)oxycarbonyl]purin-6-yl]-1-oxo-5-phenylmethoxyspiro-[imidazo[3,4-a]benzimidazole-3,4′-piperidine]-2-carboxylate (6.46 g, 77%) as a white dry film. m/z (ESI+) (M+H)+=667; HPLC tR=2.55 min; 1H NMR (399.9 MHz, CDCl3) δ 1.47 (9H, s), 1.70 (9H, s), 1.90 (2H, d), 3.00-3.07 (2H, m), 4.15 (2H, m), 5.35 (1H, m), 5.50 (2H, s), 5.89 (1H, m), 6.93 (1H, d), 7.29-7.32 (2H, m), 7.35-7.39 (2H, m), 7.49-7.51 (2H, m), 7.56-7.58 (1H, m), 8.19 (1H, s), 8.52 (1H, s).

### 103B. tert-butyl 5-hydroxy-1′-[9-[(2-methylpropan-2-yl)oxycarbonyl]purin-6-yl]-1-oxospiro[imidazo[3,4-a]benzimidazole-3,4′-piperidine]-2-carboxylate

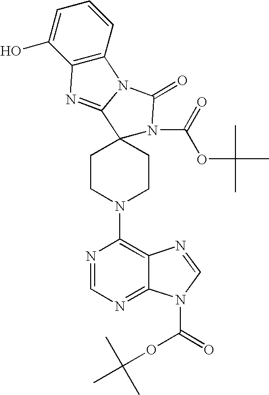

tert-Butyl 1′-[9-[(2-methylpropan-2-yl)oxycarbonyl]purin-6-yl]-1-oxo-5-phenylmethoxyspiro[imidazo[3,4-a]benzimidazole-3,4′-piperidine]-2-carboxylate (6.4 g, 9.60 mmol) and palladium 30% on carbon (0.100 g, 0.94 mmol) in ethyl acetate (200 mL) and ethanol (50 mL) was stirred under hydrogen at 20° C. for 70 hours. DCM (100 mL) and MeOH (50 mL) were added and the mixture was stirred for a further 10 minutes, filtered and evaporated to afford tert-butyl 5-hydroxy-1′-[9-[(2-methylpropan-2-yl)oxycarbonyl]purin-6-yl]-1-oxospiro[imidazo[3,4-a]benzimidazole-3,4′-piperidine]-2-carboxylate (5.14 g, 100%) as a white solid, which was used without further purification. m/z (ESI+) (M+H)+=577; HPLC tR=2.65 min. 1H NMR (399.9 MHz, DMSO-d6) δ 1.26 (1H, d), 1.34 (9H, s), 1.65 (9H, s), 2.15 (2H, d), 2.70-2.78 (2H, m), 4.05 (1H, t), 6.85-6.87 (1H, m), 7.24 (1H, t), 7.30-7.33 (1H, m), 8.46 (1H, s), 8.58 (1H, s).

### 103C. tert-Butyl 1′-[9-[(2-methylpropan-2-yl)oxycarbonyl]purin-6-yl]-1-oxo-5-(trifluoromethylsulfonyloxy)spiro[imidazo[3,4-a]benzimidazole-3,4′-piperidine]-2-carboxylate

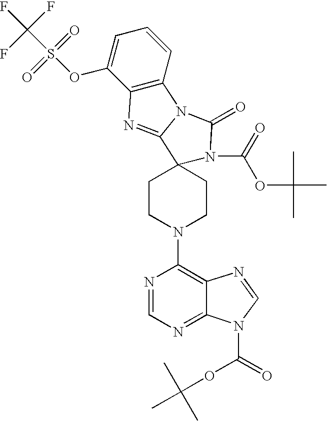

Trifluoromethanesulfonic anhydride (1.779 mL, 10.57 mmol) was added dropwise to a stirred suspension of tert-butyl 1′-[9-[(2-methylpropan-2-yl)oxycarbonyl]purin-6-yl-1-oxo-5-phenylmethoxyspiro[imidazo[3,4-a]benzimidazole-3,4′-piperidine]-2-carboxylate (5.08 g, 8.81 mmol) and N-ethyldiisopropylamine (3.07 mL, 17.62 mmol) in DCM (50 mL) at 0° C., over a period of 1 minute under nitrogen. The resulting mixture was stirred at 20° C. for 30 minutes. The reaction was incomplete and further trifluoromethanesulfonic anhydride (0.60 mL, 3.52 mmol) was added and the mixture was stirred at 20° C. for a further 15 minutes. The reaction mixture was diluted with DCM (50 mL), and washed with saturated NaHCO3 (100 mL). The organic layer was dried over MgSO4, filtered and evaporated to afford crude product. The crude product was purified by flash silica chromatography, elution gradient 4 to 20% EtOAc in DCM. Pure fractions were evaporated to dryness to afford tert-butyl 1′-[9-[(2-methylpropan-2-yl)oxycarbonyl]purin-6-yl]-1-oxo-5-(trifluoromethylsulfonyloxy)spiro[imidazo[3,4-a]benzimidazole-3,4′-piperidine]-2-carboxylate (3.14 g, 50.3%) as an orange dry film.

m/z (ESI+) (M+H)+=709;l HPLC tR=2.62 min;

1H NMR (399.9 MHz, CDCl3) δ 1.46 (9H, s), 1.71 (9H, s), 1.89-1.92 (2H, m), 3.04-3.05 (2H, m), 4.10 (2H, s), 5-40-5.90 (2H, d), 7.37-7.39 (1H, m), 7.48 (1H, t), 7.99-8.01 (1H, m), 8.20 (1H, s), 8.54 (1H, s).

### 103D. 4-(4-phenyl-1H-benzimidazol-2-yl)-1-(9H-purin-6-yl)piperidin-4-amine

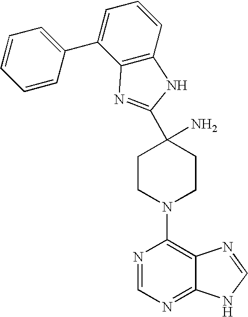

A stream of nitrogen was passed through a mixture of tert-butyl 1′-[9-[(2-methylpropan-2-yl)oxycarbonyl]purin-6-yl]-1-oxo-5-(trifluoromethylsulfonyloxy)spiro[imidazo[3,4-a]benzimidazole-3,4′-piperidine]-2-carboxylate (0.213 g, 0 3 mmol), phenylboronic acid (73.2 mg, 0.60 mmol) and cesium fluoride (0.183 g, 1.20 mmol) in 1,4-dioxane (3 mL) and water (0.03 mL). After 10 minutes, tetrakis(triphenylphosphine)palladium(0) (28 mg, 24.00 μmol) was added and the mixture was sealed into a microwave tube. The reaction was heated to 150° C. for 20 minutes in the microwave reactor and cooled to RT. 50% aq NaOH (0.1 mL) was added and the mixture was heated to 130° C. for 10 minutes in the microwave reactor and cooled to RT. Water (10 mL) was added and the mixture was acidified to pH 8 with 2M aq HCl. The mixture was washed sequentially with a 10:1 mixture of EtOAc and MeOH 10:1 (3×10 mL) and the combined organic layer was dried briefly over MgSO4 and evaporated. The residue was dissolved in TFA (3 mL) and stood overnight, then diluted with MeOH (5 mL) and loaded onto a 5 g SCX-2 cartridge. The cartridge was washed with MeOH and the product eluted with 2N NH3 in MeOH. The eluent was evaporated and the crude product was dissolved in DMF (1 mL), diluted with DCM (10 mL) and loaded onto a 12 g silica Redisep cartridge for purification by chromatography eluting with 10 to 100% in DCM of a 10% methanolic solution of aq NH4OH. Fractions containing product were combined to afford 4-(4-phenyl-1H-benzimidazol-2-yl)-1-(9H-purin-6-yl)piperidin-4-amine (99 mg, 59%) as a dry film.

m/z (ESI+) (M+H)⁺=411; HPLC t_{R}=1.94 min;

1H NMR (399.9 MHz, DMSO-d6) δ 1.82 (2H, d), 2.07-2.09 (2H, m), 4.07 (2H, s), 4.84 (2H, s), 7.24 (1H, t), 7.34 (2H, m), 7.48 (3H, m), 8.03 (2H, m), 8.11 (1H, s), 8.21-8.23 (1H, s).

### EXAMPLES 104 TO 106

The compounds of Examples 104 to 106 were synthesised in a manner similar to that used for Example 103D using the boronic acid starting material shown in the table below in place of phenylboronic acid.

| Example | Structure | Precursor | Chemical Name | N.M.R. Data | M.S. |
|---|---|---|---|---|---|
| 104 | 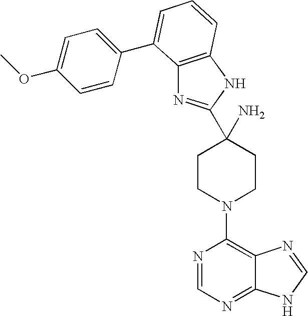 | 4-Methoxy- phenylboronic acid | 4-[4-(4- methoxyphenyl)- 1H-benzimidazol- 2-yl]-1-(9H-purin- 6-yl)piperidin-4- amine | 1H NMR (399.9 MHz, DMSO-d6) δ 1.80-1.83 (2 H, m), 2.24-2.30 (2 H, m), 3.81 (3 H, s), 4.07 (2 H, s), 4.92 (2 H, s), 7.05 (2 H, d), 7.20 (1 H, t), 7.28 (1 H, d), 7.42 (1 H, d), 8.09 (2 H, m), 8.12 (1 H, s), 8.22 (1 H, s) | MS: [M + H]+ 441 Retention time: 1.73 |
| 105 | 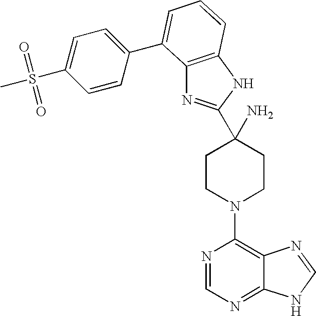 | 4-(Methane- sulfonyl)- boronic acid | 4-[4-(4- methylsulfonyl- phenyl)-1H- benzimidazol-2- yl]-1-(9H-purin-6- yl)piperidin-4- amine | 1H NMR (399.9 MHz, DMSO-d6) δ 1.83 (2 H, d), 2.23-2.30 (2 H, m), 3.28 (3 H, s), 4.06 (2 H, s), 4.89 (2 H, s), 7.29 (1 H, t), 7.46 (1 H, m), 7.56 (1 H, d), 8.01 (2 H, d), 8.12 (1 H, s), 8.22 (1 H, s), 8.34 (2 H, m) | MS: [M + H]+ 489 Retention time: 1.72 |
| 106 | 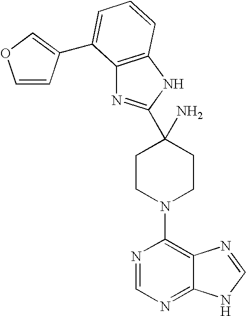 | Furan-3- boronic acid | 4-(4-furan-3-yl- 1H-benzimidazol- 2-yl)-1-(9H-purin- 6-yl)piperidin-4- amine | 1H NMR (399.9 MHz, DMSO-d6) δ 1.83-1.86 (2 H, m), 2.27-2.31 (2 H, m), 4.15 (2 H, s), 4.80 (2 H, s), 7.17 (1 H, t), 7.22 (1 H, s), 7.36 (1 H, d), 7.41 (1 H, d), 7.75 (1 H, s), 8.12 (1 H, s), 8.23 (1 H, s), 8.66 (1 H, s) | MS: [M + H]+ 401 Retention time: 1.84 |

### EXAMPLE 107

### 4-(7-(benzyloxy)-1H-benzo[d]imidazol-2-yl)-1-(1H-pyrazolo[3,4-d]pyrimidin-4-yl)piperidin-4-amine

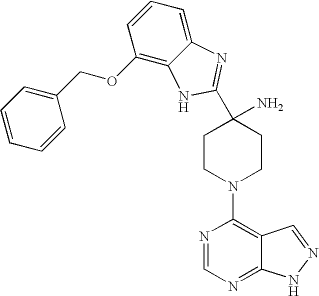

### 107A. Methyl 4-(tert-butoxycarbonylamino)-1-(1H-pyrazolo[3,4-d]pyrimidin-4-yl)piperidine-4-carboxylate

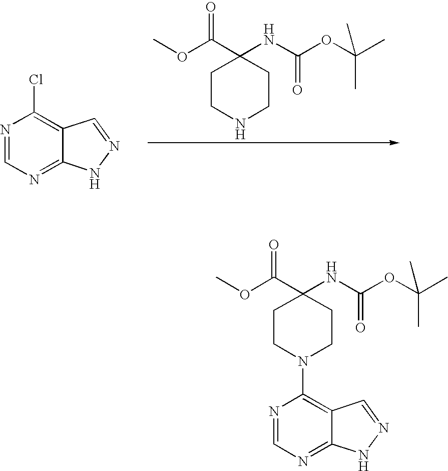

Triethylamine (25.3 mL, 181.81 mmol) was added in one portion to a stirred suspension of methyl 4-(tert-butoxycarbonylamino)piperidine-4-carboxylate (10.80 g, 41.82 mmol) and 4-chloro-1H-pyrazolo[3,4-d]pyrimidine (5.62 g, 36.36 mmol)** in ethanol (175 mL) at 20° C. under nitrogen. The resulting solution was stirred at 20° C. for 30 minutes and at 50° C. for 10 minutes. The solution was filtered and the filtrate was evaporated. The crude product was purified by flash silica chromatography, elution gradient 2 to 8% MeOH in DCM. Pure fractions were evaporated to dryness to afford methyl 4-(tert-butoxycarbonylamino)-1-(1H-pyrazolo[3,4-d]pyrimidin-4-yl)piperidine-4-carboxylate (9.94 g, 72.6%) as a cream dry film. m/z (ESI+) M+=377; HPLC tR=1.24 min;

1H NMR (399.9 MHz, DMSO-d6) δ 1.41 (9H, s), 1.92-1.97 (2H, m), 2.07-2.10 (2H, m), 3.57 (2H, m), 3.63 (3H, s), 4.34 (1H, s), 7.53 (1H, s), 8.24 (1H, s), 8.30 (1H, s), 13.52 (1H, s).

Commercially available from Chontech (catalogue number: 07044)

### 107B. 4-(tert-butoxycarbonylamino)-1-(1H-pyrazolo[3,4-d]pyrimidin-4-yl)piperidine-4-carboxylic acid

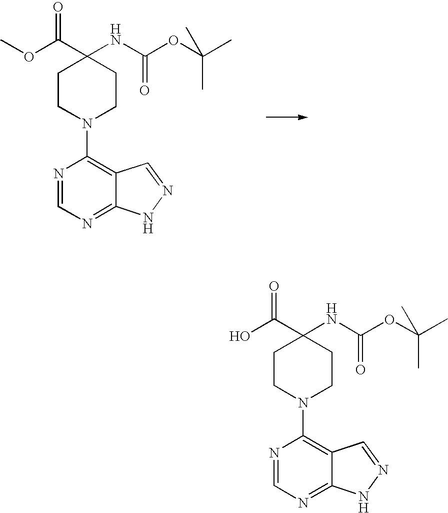

Sodium hydroxide 2M aqueous (39.4 mL, 78.74 mmol) was added in one portion to methyl 4-(tert-butoxycarbonylamino)-1-(1H-pyrazolo[3,4-d]pyrimidin-4-yl)piperidine-4-carboxylate (9.88 g, 26.25 mmol) in THF (75 mL) and MeOH (45 mL) at 20° C. under nitrogen. The resulting solution was stirred at 20° C. for 20 hours, then acidified to pH3.5 with 2M aqueous HCl. The mixture was washed sequentially with DCM (3×100 mL). The combined organic layers were washed with saturated brine (100 mL) and the organic layer was dried over MgSO4, filtered and evaporated to afford 4-(tert-butoxycarbonylamino)-1-(1H-pyrazolo[3,4-d]pyrimidin-4-yl)piperidine-4-carboxylic acid (9.18 g, 97%) as a white dry film. m/z (ESI+) (M+H)+=363; HPLC tR=1.37 min.

1H NMR (399.9 MHz, DMSO-d6) δ 1.41 (9H, s), 1.94 (2H, t), 2.09 (2H, s), 3.55-3.63 (2H, m), 4.34 (2H, s), 7.37 (1H, s), 8.24 (1H, s), 8.30 (1H, s).

### 107C. 3:1 regioisomeric mixture of tert-butyl 4-(2-amino-3-(benzyloxy)phenylcarbamoyl)-1-(1H-pyrazolo[3,4-d]pyrimidin-4-yl)piperidin-4-ylcarbamate and tert-butyl 4-(2-amino-6-(benzyloxy)phenylcarbamoyl)-1-(1H-pyrazolo[3,4-d]pyrimidin-4-yl)piperidin-4-ylcarbamate.

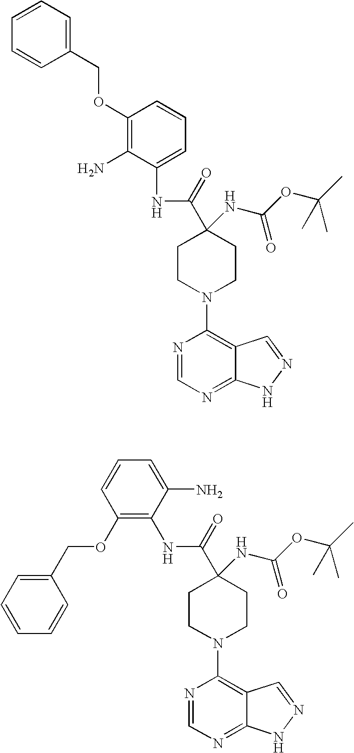

N,N-Diisopropylethylamine (6.58 mL, 37.75 mmol) was added in one portion to a stirred suspension of 4-(tert-butoxycarbonylamino)-1-(1H-pyrazolo[3,4-d]pyrimidin-4-yl)piperidine-4-carboxylic acid (9.12 g, 25.17 mmol) and O-(7-Azabenzotriazol-1-yl)-N,N,N′,N′-tetramethyluronium hexafluorophosphate (10.53 g, 27.68 mmol) in NMP (60 mL). The mixture was warmed to 50° C. for 15 minutes under nitrogen and cooled to 20° C. 3-(benzyloxy)benzene-1,2-diamine (5.39 g, 25.17 mmol) was added in one portion and the resulting solution was stirred at 20° C. for 70 hours. The reaction mixture was diluted with EtOAc (200 mL), and washed sequentially with water (3×100 mL) and saturated brine (100 mL). The organic layer was dried over MgSO4, filtered and evaporated to afford crude product which was purified by flash silica chromatography, elution gradient 1 to 10% MeOH in DCM. Fractions containing product were were evaporated to dryness to afford a pink foam which was shown to be an 82% pure 3:1 regioisomeric mixture of tert-butyl 4-(2-amino-3-(benzyloxy)phenylcarbamoyl)-1-(1H-pyrazolo[3,4-d]pyrimidin-4-yl)piperidin-4-ylcarbamate and tert-butyl 4-(2-amino-6-(benzyloxy)phenylcarbamoyl)-1-(1H-pyrazolo[3,4-d]pyrimidin-4-yl)piperidin-4-ylcarbamate. (8.1 g, 58% of 82% pure). m/z (ESI+) (M+H)⁺=559; HPLC t_{R}=2.21 min (28%), 2.41 min (72%); 1H NMR (399.9 MHz, DMSO-d6) δ 1.44 (9H, d), 1.96 (0.5H, m), 2.13 (1.5H, s), 3.72 (2H, d), 4.31 (1H, m), 4.46 (1H, m), 4.94 (0.5H, s), 5.13 (1.5H, s), 6.28 (0.25H, d), 6.32 (0.25H, d), 6.51 (0.75H, t), 6.62 (0.75H, s), 6.81 (0.75H, d), 6.90 (0.25H, m), 7.22-7.43 (6H, m), 7.50 (1.5H, d), 8.15 (0.25H, s), 8.25 (1H, m), 8.32 (0.75H, s), 8.61 (0.25H, s), 9.10 (0.75H, s), 13.51 (1H, s)

### 107D. 4-(7-(benzyloxy)-1H-benzo[d]imidazol-2-yl)-1-(1H-pyrazolo[3,4-d]pyrimidin-4-yl)piperidin-4-amine

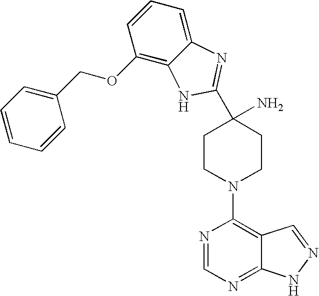

Hydrogen chloride 4M in dioxane (36.2 mL, 145.00 mmol) was added in one portion to a stirred solution of tert-butyl 4-(2-amino-3-(benzyloxy)phenylcarbamoyl)-1-(1H-pyrazolo[3,4-d]pyrimidin-4-yl)piperidin-4-ylcarbamate (8.1 g, 14.50 mmol) and tert-butyl 4-(2-amino-6-(benzyloxy)phenylcarbamoyl)-1-(1H-pyrazolo[3,4-d]pyrimidin-4-yl)piperidin-4-ylcarbamate (0.00 μmol) in NMP (40 mL) at 20° C. under nitrogen. The resulting solution was stirred at 95° C. for 3 hours. The reaction mixture was evaporated to dryness and partitioned between in DCM (150 mL) and sat NaHCO3 (100 mL). The aqueous layer was washed sequentially with DCM (2×100 mL) and the combined organic layers were dried over MgSO4 and evaporated. The crude product was purified by flash silica chromatography, elution gradient 2 to 20% of a 10% methanolic colution of NH4OH in DCM. Pure fractions were evaporated to dryness to afford 4-(7-(benzyloxy)-1H-benzo[d]imidazol-2-yl)-1-1H-pyrazolo[3,4-d]pyrimidin-4-yl)piperidin-4-amine (3.93 g, 61.5%) as a cream dry film. m/z (ESI+) (M+H)+=441; HPLC tR=1.84 min.

1H NMR (399.9 MHz, DMSO-d6) δ 1.82-1.85 (2H, m), 2.25-2.29 (2H, m), 3.91-3.95 (2H, t), 4.30 (2H, m), 5.30 (2H, s), 6.76 (1H, s), 7.03 (1H, t), 7.12 (1H, m), 7.33-7.45 (3H, m), 7.52 (2H, s), 8.23-8.26 (1H, m), 8.32 (1H, s), 13.50 (1H, s).

### EXAMPLE 108

### 4(4-phenyl -1H-benzo[d]imidazol-2-yl)-1-(1H-pyrazolo[3,4-d]pyrimidin-4-yl)piperidin-4-amine

### 108A. tert-butyl 1′-[2-[(2-methylpropan-2-yl)oxycarbonyl]pyrazolo[4,3-e]pyrimidin-4-yl]-1-oxo-5-phenylmethoxyspiro[imidazo[3,4-a]benzimidazole-3,4′-piperidine]-2-carboxylate

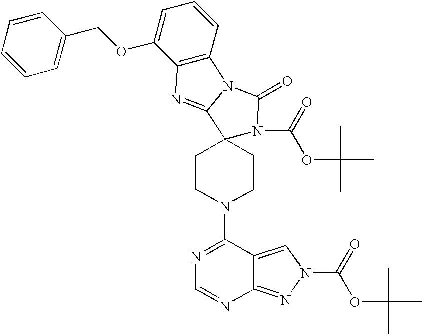

The title compound was prepared from 4-(7-(benzyloxy)-1H-benzo[d]imidazol-2-yl)-1-(1H-pyrazolo[3,4-d]pyrimidin-4-yl)piperidin-4-amine using the procedure described in Example 103A. m/z (ESI+) (M+H)+=667; HPLC tR=2.23 min;

1H NMR (399.9 MHz, DMSO-d6) δ 1.38 (9H, s), 1.64 (9H, s), 2.23 (2H, d), 2.72-2.78 (2H, m), 4.04 (1H, m), 4.85 (2H, m), 5.42 (2H, s), 7.09-7.11 (1H, m), 7.32-7.43 (4H, m), 7.48-7.52 (3H, m), 8.50 (1H, s), 8.68 (1H, s).

### 108B. tert-Butyl 5-hydroxy-1′-[2-[(2-methylpropan-2-yl)oxycarbonyl]pyrazolo[4,3-e]pyrimidin-4-yl]-1-oxospiro[imidazo-[3,4-a]benzimidazole-3,4′-piperidine]-2-carboxylate

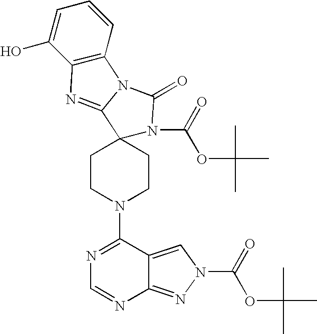

The title compound was prepared from tert-butyl 1′-[2-[(2-methylpropan-2-yl)oxycarbonyl]pyrazolo[4,3-e]pyrimidin-4-yl]-1-oxo-5-phenylmethoxyspiro[imidazo[3,4-a]benzimidazole-3,4′-piperidine]-2-carboxylate using the procedure described in Example 103B.

m/z (ESI+) (M+H)+=577; HPLC tR=1.23 min.

1H NMR (399.9 MHz, DMSO-d6) δ 1.37 (9H, s), 1.65 (9H, s), 2.22 (2H, d), 2.73-2.78 (2H, m), 4.07 (2H, m), 4.47 (2H, m), 6.84-6.87 (1H, m), 7.24 (1H, t), 7.32 (1H, d), 8.52 (1H, s), 8.70 (1H, s).

### 108C. tert-Butyl 1′-[2-[2-methylpropan-2-yl)oxycarbonyl]pyrazolo[4,3-e]pyrimidin-4-yl]-1-oxo-5-(trifluoromethylsulfonyloxy)spiro[imidazo[3,4-a]benzimidazole-3,4′-piperidine]-2-carboxylate

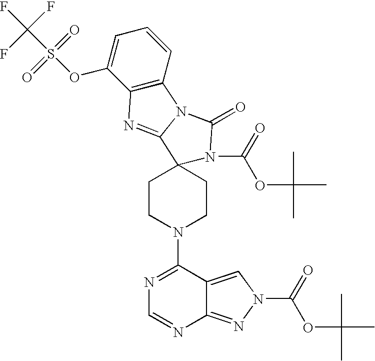

The title compound was prepared from tert-butyl 5-hydroxy-1′-[2-[(2-methylpropan-2-yl)oxycarbonyl]pyrazolo[4,3-e]pyrimidin-4-yl]-1-oxospiro[imidazo[3,4-a]benzimidazole-3,4′-piperidine]-2-carboxylate using the procedure described in Example 103C. m/z (ESI+) (M+H)+=709; HPLC tR=2.27 min; 1H NMR (399.9 MHz, DMSO-d6) δ 1.36 (9H, s), 1.64 (9H, s), 2.31-2.34 (2H, m), 2.72-2.78 (2H, m), 4.04 (2H, m), 4.94 (2H, m), 7.60-7.65 (2H, m), 8.02-8.05 (1H, m), 8.53 (1H, s), 8.71 (1H, s).

### 108D. 4-(7-(benzyloxy)-1H-benzo[d]imidazol-2-yl)-1-(1H-pyrazolo[3,4-d]pyrimidin-4-yl)piperidin-4-amine

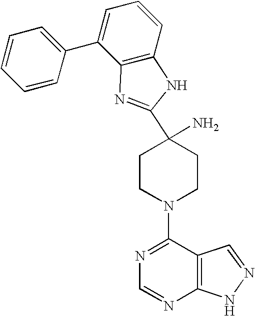

The title compound was prepared from tert-butyl 1′-[2-[(2-methylpropan-2-yl)oxycarbonyl]pyrazolo[4,3-e]pyrimidin-4-yl]-1-oxo-5-(trifluoromethylsulfonyloxy)-spiro[imidazo[3,4-a]benzimidazole-3,4′-piperidine]-2-carboxylate and phenylboronic acid using the procedure described in Example 103D. m/z (ESI+) (M+H)+=411; HPLC tR=1.95 min;

1H NMR (399.9 MHz, DMSO-d6) δ 1.86-1.90 (2H, d), 2.27-2.31 (2H, m), 3.93-3.97 (2H, t), 4.35 (2H, s), 7.24 (1H, t), 7.35 (2H, m), 7.48-7.50 (3H, m), 8.04 (2H, m), 8.25 (1H, s), 8.33 (1H, s).

### EXAMPLES 109 TO 111

The compounds of Examples 109 to 111 were synthesised in a manner similar to that used for Example 103D and Example 108D using the boronic acid starting material shown in the table below in place of phenylboronic acid.

| Example | Structure | Precursor | Chemical Name | N.M.R. Data | M.S. |
|---|---|---|---|---|---|
| 109 | 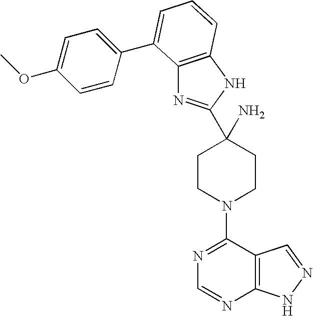 | 4-Methoxy- phenylboronic acid | 4-[4-(4- methoxyphenyl)- 1H- benzo[d]imidazol- 2-yl)-1-(1H- pyrazolo[3,4- d]pyrimidin-4- yl)piperidin-4- amine | 1H NMR (399.9 MHz, DMSO-d6) δ 1.88 (2 H, d), 2.27-2.31 (2 H, m), 3.81 (3 H, s), 3.93-3.98 (2 H, t), 4.36 (2 H, m), 7.04 (2 H, d), 7.21 (1 H, t), 7.28 (1 H, s), 7.43 (1 H, d), 8.07 (1 H, m), 8.25 (1 H, s), 8.34 (1 H, s), 13.50 (1 H, s) | MS: [M + H]+ 441 Retention time: 1.98 |
| 110 | 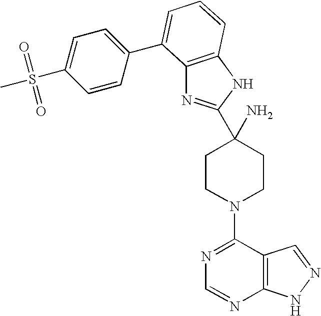 | 4-(Methane- sulfonyl)- boronic acid | 4-[4-(4- methylsulfonyl- phenyl)-1H- benzo[d]imidazol- 2-yl)-1-(1H- pyrazolo[3,4- d]pyrimidin-4- yl)piperidin-4- amine | 1H NMR (399.9 MHz, DMSO-d6) δ 1.87-1.91 (2 H, m), 2.28-2.32 (2 H, m), 3.28 (3 H, s), 3.95-4.00 (2 H, t), 4.37 (2 H, m), 7.30 (1 H, t), 7.45 (1 H, m), 7.57 (1 H, d), 8.01 (2 H, d), 8.25 (1 H, s), 8.34 (3 H, m) | MS: [M + H]+ 489 Retention time: 1.74 |
| 111 | 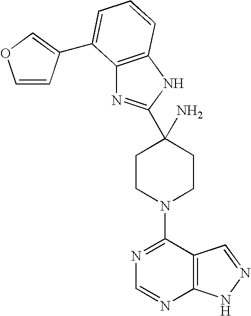 | Furan-3- boronic acid | 4-(4-furan-3-yl- 1H- benzo[d]imidazol- 2-yl)-1-(1H- pyrazolo[3,4- d]pyrimidin-4- yl)piperidin-4- amine | 1H NMR (399.9 MHz, DMSO-d6) δ 1.88-1.92 (2 H, m), 2.30-2.35 (2 H, m), 3.99-4.05 (2 H, t), 4.32 (2 H, m), 7.18-7.22 (2 H, m), 7.37-7.42 (2 H, m), 7.75 (1 H, s), 8.26 (1 H, s), 8.35 (1 H, s), 8.67 (1 H, s) | MS: [M + H]+ 401 Retention time: 1.87 |

### EXAMPLE 112

### 4-[7-(2-methylsulfonylphenyl)-1H-benzimidazol-2-yl]-1-(7H-pyrrolo[3,2-e]pyrimidin-4-yl)piperidin-4-amine

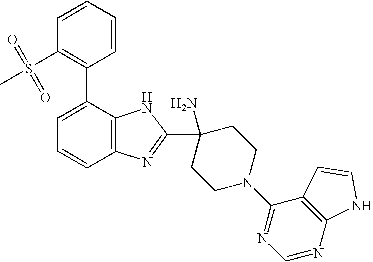

The title compound was synthesised according to the method described in Example 91D using (2-methylsulfonyl)phenylboronic acid in place of pyrimidine-5-boronic acid.

¹H NMR (399.9 MHz, DMSO-d₆) δ 1.70-1.78 (2H, m), 2.10-2.21 (2H, m), 2.90 (3H, br, s), 3.80 (2H, t), 4.27 (2H, d), 6.58-6.62 (1H, m), 7.04 (1H, d), 7.12-7.25 (2H, m), 7.42 (1H, d), 7.46-7.60 (1H, m), 7.65-7.79 (2H, m), 8.08-8.15 (2H, m), 11.63 (1H, s).

M/z: [M+H]⁺488;

### EXAMPLE 113

### 4-(4-(3-(aminomethyl)-4-fluorophenyl)-1H-benzo[d]imidazol-2-yl)-1-(7H-pyrrolo[2,3-d]pyrimidin-4-yl)piperidin-4-amine

### 113A. tert-butyl 2-fluoro-5-(4,4,5,5-tetramethyl-1,3,2-dioxaborolan-2-yl)benzylcarbamate

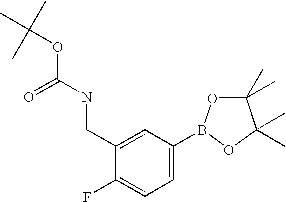

Bis(pinacolato)diboron (2.83 g, 11.13 mmol), tert-butyl 5-bromo-2-fluorobenzylcarbamate (2.82 g, 9.27 mmol) (CAS no. 491836-84-1, prepared by a method very similar to that described in WO 2005094822) and potassium acetate (4.55 g, 46.36 mmol) were suspended in 1,4-dioxane (25 mL) at 22° C. under nitrogen. A stream of nitrogen was passed through the mixture for 10 minutes. 1,1′-Bis(diphenylphosphino)ferrocene-palladium dichloride (0.191 g, 0.23 mmol) was added and the mixture was heated at reflux for 1.5 hours. The mixture was concentrated and the residue was treated with DCM (500 mL). The insoluble material was removed by filtration through a plug of silica. The silica was washed through with further DCM. The combined filtrates were concentrated and the residue purified by flash silica chromatography, eluting with 10 to 30% ethyl acetate in isohexane. Pure fractions were evaporated to dryness to afford tert-butyl 2-fluoro-5-(4,4,5,5-tetramethyl-1,3,2-dioxaborolan-2-yl)benzylcarbamate (2.74 g, 84%) as a colourless gum.

¹H NMR (399.9 MHz, CDCl₃) δ 1.33 (12H, s), 1.46 (9H, s), 4.37 (2H, s), 4.83 (1H, s), 7.00-7.05 (1H, m), 7.68-7.72 (1H, m), 7.77 (1H, d).

M/z: [M+H-C₄H₈]+296

### 113B. 4-(4-(3-(aminomethyl)-4-fluorophenyl)-1H-benzo[d]imidazol-2-yl)-1-(7H-pyrrolo[2,3-d]pyrimidin-4-yl)piperidin-4-amine

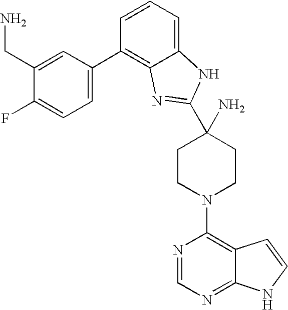

The title compound was synthesised according to the method used to prepare Example 91D using tert-butyl 2-fluoro-5-(4,4,5,5-tetramethyl-1,3,2-dioxaborolan-2-yl)benzylcarbamate in place of pyrimidine-5-boronic acid.

¹H NMR (500.13 MHz, DMSO-d₆) δ 1.82-1.89 (2H, m), 2.32-2.38 (2H, m), 3.86 (2H, s), 3.94-4.01 (2H, m), 4.22-4.28 (2H, m), 6.57 (1H, d), 7.09 (1H, d), 7.15 (1H, dd), 7.21 (1H, t), 7.32 (1H, d, 7.45 (1H, dd), 7.96 (1H, br, s), 8.13 (1H, d), 8.16 (1H, s), 11.25 (1H, br, s).

M/z: [M+H]⁺ 457

### EXAMPLE 114

### 4-(4-(2-(aminomethyl)phenyl )-1H-benzo[d]imidazol-2-yl)-1-(7H-pyrrolo[2,3-d]pyrimidin-4-yl)piperidin-4-amine

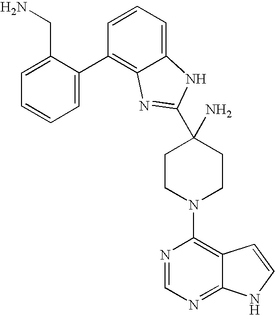

The title compound was synthesised according to the method used to prepare Example 91D using 2-((tert-butoxycarbonylamino)methyl)phenylboronic acid in place of pyrimidine-5-boronic acid.

¹H NMR (399.9 MHz, DMSO-d₆) δ 1.74-1.83 (2H, m), 2.14-2.25 (2H, m), 3.57 (2H, s), 3.83 (2H, t), 4.24-4.33 (2H, m), 6.61 (1H, d), 7.02 (1H, d), 7.16 (1H, d), 7.19-7.29 (2H, m), 7.31-7.38 (1H, m), 7.42 (1H, t), 7.52 (1H, d), 7.60 (1H, d), 8.14 (1H, s), 11.64 (1H, br, s);

M/z: [M+H]+439

### EXAMPLE 115

### 4-(4-(3-(methylsulfonylmethyl)phenyl)-1H-benzo[d]imidazol-2-yl)-1-(7H-pyrrolo[2,3-d]pyrimidin-4-yl)piperidin-4-amine

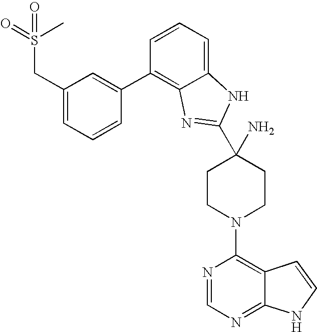

tert-Butyl 1′-[7-[(2-methylpropan-2-yl)oxycarbonyl]pyrrolo[3,2-e]pyrimidin-4-yl]-1-oxo-5-(trifluoromethylsulfonyloxy)spiro[imidazo[3,4-a]benzimidazole-3,4′-piperidine]-2-carboxylate (Example 91C) (150 mg, 0.21 mmol) was added to a mixture of bis(pinacolato)diboron (81 mg, 0.32 mmol) and potassium acetate (146 mg, 1.48 mmol) in dioxane (1.5 mL). A stream of nitrogen was bubbled through the mixture for 5 minutes. (1,1′-bis(diphenylphosphino)ferrocene)-dichloropalladium(II) (8.72 mg, 10.60 μmol) was added and the mixture was stirred and heated by microwave for 15 minutes at 135° C. The mixture was cooled, then treated with sodium carbonate (100 mg, 0.94 mmol), 1-bromo-3-(methylsulfonylmethyl)benzene (74 mg, 0.30 mmol), and water (0.5 mL). A stream of nitrogen was passed through the reaction mixture for 10 minutes and tetrakistriphenyl-phosphine (10 mg) was added. The mixture was heated by microwave for 10 minutes at 135° C. The mixture was cooled and treated with 50% aqueous sodium hydroxide (0.1 mL). The mixture was heated by microwave for 10 minutes at 135° C., then cooled to room temperature. The organic layer was concentrated under reduced pressure and the residue was dissolved with TFA (3 mL). The resulting solution was allowed to stand at room temperature for 1 hour. The solution was concentrated under reduced pressure and the crude product was purified by ion exchange chromatography, using an SCX column. The column was washed with methanol after the product was loaded and the desired product was eluted from the column using 2M ammonia in methanol. Pure fractions were evaporated to dryness to afford crude product as a brown foam. This material was further purified by preparative LCMS (Waters XBridge Prep C18 OBD column, 5μ silica, 19 mm diameter, 100 mm length), using decreasingly polar mixtures of water (containing 0.1% ammonia) and acetonitrile as eluents. Fractions containing the desired compound were evaporated to dryness to afford 4-(4-(3-(methylsulfonylmethyl)phenyl)-1H-benzo[d]-imidazol-2-yl)-1-(7H-pyrrolo[2,3-d]pyrimidin-4-yl)piperidin-4-amine (16 mg, 15%) as beige solid.

¹H NMR (399.9 MHz, DMSO-d₆) δ 1.77-1.86 (2H, m), 2.23-2.34 (2H, m), 2.94 (3H, s), 3.85-3.95 (2H, m), 4.27-4.36 (2H, m), 4.55 (2H, s), 6.62-6.65 (1H, m), 7.16-7.18 (1H, m), 7.26 (1H, t), 7.30-7.36 (1H, m), 7.38-7.43 (1H, m), 7.47-7.54 (2H, m), 8.05 (2H, br, s), 8.15 (1H, s), 11.65 (1H, s);

M/z: [M+H]⁺ 502

### EXAMPLE 116

### 4-(4-(2-(methylsulfonylmethyl)phenyl)-1H-benzo[d]imidazol-2-yl)-1-(7H-pyrrolo[2,3-d]pyrimidin-4-yl)piperidin-4-amine

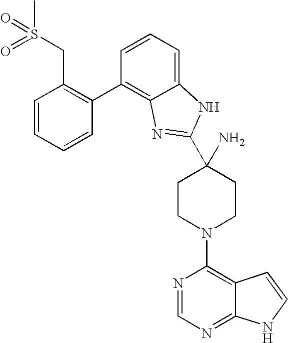

The title compound was synthesised according to the method described in Example 115 using 1-bromo-2-(methylsulfonylmethyl)benzene in place of 1-bromo-3-(methylsulfonyl-methyl)benzene.

¹H NMR (399.9 MHz, DMSO-d₆) δ 1.73-1.82 (2H, d), 2.16-2.27 (2H, m), 2.52 (3H, obscured by DMSO signal), 3.81-3.92 (2H, m), 4.22-4.31 (2H, m), 4.46 (2H, s), 6.61 (1H, d), 7.09 (1H, d), 7.16 (1H, d), 7.24 (1H, t), 7.38-7.64 (5H, m), 8.13 (1H, s), 11.65 (1H, br, s);

M/z: [M+H]⁺ 502.

Biological Activity

### EXAMPLE 117

Measurement of PKA Kinase Inhibitory Activity (IC₅₀)

Compounds of the invention can be tested for PK inhibitory activity using the PKA catalytic domain from Upstate Biotechnology (#14-440) and the 9 residue PKA specific peptide (GRTGRRNSI), also from Upstate Biotechnology (#12-257), as the substrate. A final concentration of 1 nM enzyme is used in a buffer that includes 20 mM MOPS pH 7.2, 40 μM ATP/γ³³P-ATP and 50 mM substrate. Compounds are added in dimethylsulphoxide (DMSO) solution to a final DMSO concentration of 2.5%. The reaction is allowed to proceed for 20 minutes before addition of excess orthophosphoric acid to quench activity. Unincorporated γ³³P-ATP is then separated from phosphorylated proteins on a Millipore MAPH filter plate. The plates are washed, scintillant is added and the plates are then subjected to counting on a Packard Topcount.

The % inhibition of the PKA activity is calculated and plotted in order to determine the concentration of test compound required to inhibit 50% of the PKA activity (IC₅₀).

Following the protocol described above, the IC₅₀ values of the compounds of Examples 1, 2, 3, 4, 6, 8, 12, 17, 18, 35 and 37 have been found to be less than 1 μM.

### EXAMPLE 118

Measurement of PKB Kinase Inhibitory Activity (IC₅₀)

The inhibition of protein kinase B (PKB) activity by compounds can be determined essentially as described by Andjelkovic et al. (Mol. Cell. Biol. 19, 5061-5072 (1999)) but using a fusion protein described as PKB-PIF and described in full by Yang et al (Nature Structural Biology 9, 940-944 (2002)). The protein is purified and activated with PDK1 as described by Yang et al. The peptide AKTide-2T (H-A-R-K-R-E-R-T-Y-S-F-G-H-H-A-OH) obtained from Calbiochem (#123900) is used as a substrate. A final concentration of 0.6 nM enzyme is used in a buffer that includes 20 mM MOPS pH 7.2, 30 μM ATP/γ³³P-ATP and 25 82 M substrate. Compounds are added in DMSO solution to a final DMSO concentration of 2.5%. The reaction is allowed to proceed for 20 minutes before addition of excess orthophosphoric acid to quench activity. The reaction mixture is transferred to a phosphocellulose filter plate where the peptide binds and the unused ATP is washed away. After washing, scintillant is added and the incorporated activity measured by scintillation counting.

The % inhibition of the PKB activity is calculated and plotted in order to determine the concentration of test compound required to inhibit 50% of the PKB activity (IC₅₀).

Following the protocol described above, the IC₅₀ values of the compounds of Examples 1, 2, 3, 4, 5, 6, 7, 8, 9, 10, 12, 17, 18, 19, 20, 34, 36, 37, 39 and 41 have been found to be less than 0.1 μM whilst the compounds of Examples 33, 35, 38 and 40 each have IC₅₀ values of less than 1 μM.

### EXAMPLE 119

In Vitro MDA-MB-468 Human Breast Ddenocarcinoma GSK-3 Phosphorylation Assay

This assay determines the ability of test compounds to inhibit phosphorylation of Serine-9 residue in Glycogen Synthase Kinase-3beta (GSK-313) as a surrogate marker of cellular PKB (Akt) activity, as assessed using Acumen Explorer Fluorescent Plate-Reader technology. A MDA-MB-468 human breast adenocarcinoma cell line (LGC Promochem, Teddington, Middlesex, UK, Catalogue No. HTB-132) was routinely maintained in Dulbecco's modified Eagle's growth medium (DMEM; Invitrogen Limited, Paisley, UK Catalogue No. 11966-025) containing 10% heat-inactivated foetal calf serum (FCS; Sigma, Poole, Dorset, UK, Catalogue No. F0392) and 1% L-glutamine (Gibco, Catalogue No. 25030-024) at 37° C. with 5% CO₂ up to a confluency of 70-90%.

For the phosphorylation assay, the cells were detached from the culture flask using Trypsin-EDTA (Invitrogen Limited, Catalogue No. 25300-062) and seeded into the wells of a black transparent-bottom Corning Costar Polystyrene 96 well plate (Fisher Scientific UK, Loughborough, Leicestershire, UK; Catalogue No. 3904 and DPS-130-020K) at a density of 5000 cells per well in 100 μl of complete growth media. The cells were incubated overnight at 37° C. with 5% CO₂ to allow them to adhere.

On day 2, the cells were treated with test compounds and incubated for 2 hours at 37° C. with 5% CO₂. Test compounds were prepared as 10mM stock solutions in DMSO and dosed directly to required concentration into test wells using non-contact (acoustic dispensing of multiple 2.5 nl droplets directly into assay wells) ECHO dosing technology (Labcyte Inc Sunnyvale, Calif., USA). Each plate contained control wells without test compound.

20 μl of fixing buffer (Phosphate Buffered Saline (PBS) containing 10% formaldehyde; Sigma; Catalogue No. F1635) was then added to each well to give a final well concentration 1.6%. Plates were then incubated for 30 minutes at room temperature prior to the fixative being removed. Each well was washed once with 250 μl of PBS and then 50 μl PBS added to each well. PBS was then aspirated and cells permeabilised and blocked by incubating each well with 50 μl of permeabilisation/blocking buffer (PBS containing 0.5% Tween 20 (Sigma; Catalogue No. P5927) and 5% Marvel Milk Powder (Andrews Pharmacy Ltd, Macclesfield, Cheshire, UK; Catalogue No. APC100199)) for 1 hour at room temperature prior to staining.

Following removal of Perm/Block buffer, 50 μl of primary anti-phospho-GSK-3β antibody (Cell Signalling Technology (New England Biolabs (Uk) Ltd.), Hitchin, Hertfordshire, UK; Catalogue No.9336 diluted 1:400 in Blocking buffer (PBS containing 5% Marvel and 0.05% Tween/Polysorbate 20) was added to each well and incubated overnight at 4° C.

Each well was washed three times in 250 μl of wash buffer (PBS containing 0.05% polysorbate 20), and cells incubated for 1 hour at room temperature with 50 μl of secondary fluorescently-labelled anti-rabbit Alexa Fluor 488 antibody (Molecular Probes, Invitrogen Limited, Catalogue No. A11008) diluted 1:750 in blocking buffer. Plates were washed three times in 250 μl of wash buffer and stored containing 50 μl of PBS at 4° C. until required.

Plates were analysed using an Acumen Explorer Plate-reader to quantify level of fluorescent signal that represents quantity of phosphorylated-GSK-313. Active compounds caused a decrease in phospho-GSK-3β phosphorylation relative to the maximum (undosed) control for each assay, which is measured by the number of phosphorylated objects per well, and enabled potency of PKB (Akt) inhibitors to be determined

IC₅₀ calculation—IC₅₀ is the concentration of compound required to give 50% effect over the range of activity affected by the compound, between maximum (no compound) and minimum (excess level of compound) response control data. IC₅₀ values were determined by fitting background corrected, dose response assay data to a 4 parameter logistic curve fit equation model with the minimum response set to zero. This was done using an in-house developed algorithm within the Origin graphing software package (OriginLab Corporation, Northampton, Mass., USA).

Compounds of the invention were tested in the above assay and the mean IC₅₀ values are set out in the table below. Unless indicated otherwise, the data refer to the title compounds in the examples.

| Example Number | Cell pGSK3b IC₅₀ (μM) |
|---|---|
| 6 | 0.56 |
| 7 | 0.94 |
| 11 | 0.16 |
| 13 | 0.66 |
| 17 | 0.32 |
| 21 | 0.25 |
| 22 | 0.77 |
| 44 | 0.12 |
| 45 | 0.087 |
| 46 | 0.097 |
| 47 | 0.17 |
| 48 | 0.21 |
| 50 | 0.48 |
| 51 | 0.8 |
| 52 | 1.3 |
| 53 | 1.8 |
| 54 | >2.6 |
| 55 | 2.9 |
| 56 | 5.5 |
| 57 | >2.1 |
| 58 | 1.4 |
| 59 | 1.9 |
| 60 | 8.6 |
| 61 | 0.62 |
| 62 | 0.58 |
| 63 | >0.96 |
| 64 | 0.38 |
| 65 | 0.42 |
| 69 | 1.6 |
| 70 | 1.6 |
| 71 | 2.5 |
| 72 | 3 |
| 73 | 3.3 |
| 74 | 3.7 |
| 75 | 5.1 |
| 76 | 6.4 |
| 77 | 12 |
| 78 | 16 |
| 79 | 0.085 |
| 80 | 0.064 |
| 81 | 0.14 |
| 82 | 0.12 |
| 83 | 0.047 |
| 85 | 0.35 |
| 86 | 0.13 |
| 87 | 0.22 |
| 88 | 0.052 |
| 91 | 0.052 |
| 92 | 0.033 |
| 93 | 0.25 |
| 94 | 0.047 |
| 95 | 0.049 |
| 96 | 0.044 |
| 97 | 0.085 |
| 98 | 0.18 |
| 99 | 0.015 |
| 100 | 0.014 |
| 101 | 0.0093 |
| 34D | 0.086 |
| 42 & 84 | <0.031 |
| 66G | 0.11 |
| 67B | 0.062 |
| 68B | 0.46 |
| 68D | 2.4 |
| 89E | 0.081 |
| 103 | 0.033 |
| 104 | 0.12 |
| 105 | 0.2 |
| 106 | 0.052 |
| 108 | 0.35 |
| 109 | 0.65 |
| 110 | 0.49 |
| 111 | 0.11 |
| 112 | 0.3 |
| 113 | 0.038 |
| 115 | 0.27 |
| 116 | 0.047 |

Individual IC₅₀ values were not included in the calculation of the mean IC₅₀ value if they were seen to be obvious outliers, i.e. not within approximately three-fold of the IC₅₀ of two other sets of data for the same compound

### EXAMPLE 120

Anti-Proliferative Activity

The anti-proliferative activities of compounds of the invention are determined by measuring the ability of the compounds to inhibition of cell growth in a number of cell lines. Inhibition of cell growth is measured using the Alamar Blue assay (Nociari, M. M, Shalev, A., Benias, P., Russo, C. Journal of Immunological Methods 1998, 213, 157-167). The method is based on the ability of viable cells to reduce resazurin to its fluorescent product resorufin. For each proliferation assay cells are plated onto 96 well plates and allowed to recover for 16 hours prior to the addition of inhibitor compounds for a further 72 hours. At the end of the incubation period 10% (v/v) Alamar Blue is added and incubated for a further 6 hours prior to determination of fluorescent product at 535 nM ex/590 nM em. In the case of the non-proliferating cell assay cells are maintained at confluence for 96 hour prior to the addition of inhibitor compounds for a further 72 hours. The number of viable cells is determined by Alamar Blue assay as before. All cell lines are obtained from ECACC (European Collection of cell Cultures) or ATCC.

In particular, compounds of the invention were tested against the PC3 cell line (ATCC Reference: CRL-1435) derived from human prostate adenocarcinoma. Many compounds of the invention were found to have IC₅₀ values of less than 25 μM in this assay and preferred compounds have IC₅₀ values of less than 15 μM.

### EXAMPLE 121

Assay for hERG Activity

The activity of compounds of the invention against the hERG K⁺ ion channel was determined using the assay described in the article by M. H. Bridgland-Taylor et al., Journal of Pharmcaological and Toxicological Methods, 54 (2006), 189-199.

The majority of the the compounds tested in the hERG assay had IC₅₀ values of greater than 10 μM, and some had IC₅₀ values of greater than 50 μM.

Pharmaceutical Formulations

### EXAMPLE 122

(i) Tablet Formulation

A tablet composition containing a compound of the formula (I) is prepared by mixing 50 mg of the compound with 197 mg of lactose (BP) as diluent, and 3 mg magnesium stearate as a lubricant and compressing to form a tablet in known manner.

(ii) Capsule Formulation

A capsule formulation is prepared by mixing 100 mg of a compound of the formula (I) with 100 mg lactose and filling the resulting mixture into standard opaque hard gelatin capsules.

(iii) Injectable Formulation I

A parenteral composition for administration by injection can be prepared by dissolving a compound of the formula (I) (e.g. in a salt form) in water containing 10% propylene glycol to give a concentration of active compound of 1.5% by weight. The solution is then sterilised by filtration, filled into an ampoule and sealed.

(iv) Injectable Formulation II

A parenteral composition for injection is prepared by dissolving in water a compound of the formula (I) (e.g. in salt form) (2 mg/ml) and mannitol (50 mg/ml), sterile filtering the solution and filling into sealable 1 ml vials or ampoules.

(v) Injectable Formulation III

A formulation for i.v. delivery by injection or infusion can be prepared by dissolving the compound of formula (I) (e.g. in a salt form) in water at 20 mg/ml. The vial is then sealed and sterilised by autoclaving.

(vi) Injectable Formulation IV

A formulation for i.v. delivery by injection or infusion can be prepared by dissolving the compound of formula (I) (e.g. in a salt form) in water containing a buffer (e.g. 0.2 M acetate pH 4.6) at 20 mg/ml. The vial is then sealed and sterilised by autoclaving.

(vii) Subcutaneous Injection Formulation

A composition for sub-cutaneous administration is prepared by mixing a compound of the formula (I) with pharmaceutical grade corn oil to give a concentration of 5 mg/ml. The composition is sterilised and filled into a suitable container.

(viii) Lyophilised Formulation

Aliquots of formulated compound of formula (I) are put into 50 ml vials and lyophilized. During lyophilisation, the compositions are frozen using a one-step freezing protocol at (−45° C.). The temperature is raised to −10° C. for annealing, then lowered to freezing at −45° C., followed by primary drying at +25° C. for approximately 3400 minutes, followed by a secondary drying with increased steps if temperature to 50° C. The pressure during primary and secondary drying is set at 80 millitor.

Equivalents

The foregoing examples are presented for the purpose of illustrating the invention and should not be construed as imposing any limitation on the scope of the invention. It will readily be apparent that numerous modifications and alterations may be made to the specific embodiments of the invention described above and illustrated in the examples without departing from the principles underlying the invention. All such modifications and alterations are intended to be embraced by this application.

1-120. (canceled)

121. A compound of the formula (I): 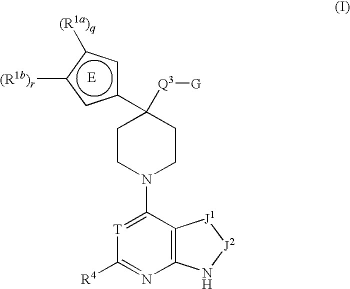
or salts, tautomers or N-oxides thereof, wherein
the ring E is a five membered heteroaryl ring containing 1, 2, 3 or 4 heteroatoms selected from O, N and S provided that no more than 1 heteroatom may be other than N;
q and r are each is 0 or 1;
T is N or a group CR⁵;
J¹-J² represents a group selected from N═C(R⁶), (R⁷)C═N, (R⁸)N—C(O), (R⁸)₂C—C(O), N═N and (R⁷)C═C(R⁶);
Q³ is a bond or a saturated hydrocarbon linker group containing from 1 to 3 carbon atoms; wherein the carbon atoms of the linker group may optionally bear one or more substituents selected from fluorine and hydroxy, provided that the hydroxy group when present is not located at a carbon atom α with respect to the G group;
G is selected from NR²R³, CN and OH;
R^{1a} and R^{1b} are the same or different and each is hydrogen or a substituent R¹⁰; or R^{1a} and R^{1b} together with the carbon atoms or heteroatoms to which they are attached form a 5 or 6-membered aryl or heteroaryl ring, wherein the aryl or heteroaryl rings are optionally substituted by one or more substituents R¹⁰; provided that when R^{1a} and R^{1b} are each hydrogen or R¹⁰, then the heteroaryl ring E is other than a thiophene or furan ring;
R² and R³ are independently selected from hydrogen; C₁₋₄ hydrocarbyl and C₁₋₄ acyl wherein the hydrocarbyl and acyl groups are optionally substituted by one or more substituents selected from fluorine, hydroxy, cyano, amino, methylamino, dimethylamino, methoxy and a monocyclic or bicyclic aryl or heteroaryl group;
or R² and R³ together with the nitrogen atom to which they are attached form a cyclic group selected from an imidazole group and a saturated monocyclic heterocyclic group having 4-7 ring members and optionally containing a second heteroatom ring member selected from O and N;
or one of R² and R³ together with the nitrogen atom to which they are attached and one or more atoms from the group Q³ form a saturated monocyclic heterocyclic group having 4-7 ring members and optionally containing a second heteroatom ring member selected from O and N; and
R⁴, R⁶ an R⁸ are each independently selected from hydrogen, halogen, C₁₋₅ saturated hydrocarbyl, cyano, CONH₂, CF₃ and NH₂;
R⁵ and R⁷ are each independently selected from hydrogen, halogen, C₁₋₅ saturated hydrocarbyl, cyano and CF₃; and
R¹⁰ is selected from halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and C₁₋₈ hydrocarbyl optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{c} is selected from hydrogen and C₁₋₄ hydrocarbyl; and
X¹ is O, S or NR^{c} and X² is ═O, ═S or ═NR^{c}.

122. A compound according to claim 121 wherein J¹-J² is a group N═C(R⁶).

123. A compound according to claim 121 wherein J¹-J² is a group (R⁷)C═N.

124. A compound according to claim 121 wherein J¹-J² is a group (R⁸)N—C(O).

125. A compound according to claim 121 wherein J¹-J² is a group (R⁸)₂C—C(O).

126. A compound according to claim 121 wherein J¹-J² is a group (R⁷)C═C(R⁶) wherein (i) R⁶ is selected from hydrogen, chlorine, fluorine and methyl; and R⁷ is selected from hydrogen, chlorine, fluorine and methyl; or (ii) R⁶ is selected from hydrogen, chlorine, fluorine and methyl; and R⁷ is selected from hydrogen, bromine, chlorine, fluorine and methyl.

127. A compound according to claim 121 wherein Q³ is a bond or a group (CH₂)ₐ wherein a is 1, 2 or 3.

128. A compound according to claims 121 wherein G is NR²R³.

129. A compound according to claim 121 having the general formula (II): 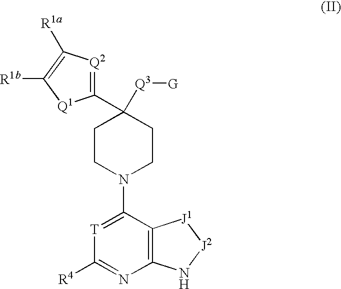
or salts, tautomers or N-oxides thereof, wherein
T is N or a group CR⁵;
J¹-J² represents a group selected from N═C(R⁶), (R⁷)C═N, (R⁸)N—C(O), (R⁸)₂C—C(O), N═N and (R⁷)C═C(R⁶);
Q¹ is NH, S or O;
Q² is N or CH;
Q³ is a bond or a saturated hydrocarbon linker group containing from 1 to 3 carbon atoms; wherein the carbon atoms of the linker group may optionally bear one or more substituents selected from fluorine and hydroxy, provided that the hydroxy group when present is not located at a carbon atom α with respect to the G group;
G is selected from NR²R³, CN and OH;
R^{1a} and R^{1b} are the same or different and each is hydrogen or a substituent R¹⁰; or R^{1a} and
R^{1b} together with the carbon atoms to which they are attached form a 6-membered aryl or heteroaryl ring, wherein the aryl or heteroaryl rings are optionally substituted by one or more substituents R¹⁰; provided that when Q¹ is S or O and R^{1a} and R^{1b} are each hydrogen or R¹⁰, then Q² is N;
R² and R³ are independently selected from hydrogen; C₁₋₄ hydrocarbyl and C₁₋₄ acyl wherein the hydrocarbyl and acyl groups are optionally substituted by one or more substituents selected from fluorine, hydroxy, cyano, amino, methylamino, dimethylamino, methoxy and a monocyclic or bicyclic aryl or heteroaryl group;
or R² and R³ together with the nitrogen atom to which they are attached form a cyclic group selected from an imidazole group and a saturated monocyclic heterocyclic group having 4-7 ring members and optionally containing a second heteroatom ring member selected from O and N;
or one of R² and R³ together with the nitrogen atom to which they are attached and one or more atoms from the group Q³ form a saturated monocyclic heterocyclic group having 4-7 ring members and optionally containing a second heteroatom ring member selected from O and N; and
R⁴, R⁶ and R⁸ are each independently selected from hydrogen, halogen, C₁₋₅ saturated hydrocarbyl, cyano, CONH₂, CF₃ and NH₂;
R⁵ and R⁷ are each independently selected from hydrogen, halogen, C₁₋₅ saturated hydrocarbyl, cyano and CF₃; and
R¹⁰ is selected from halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a}-R^{b} wherein R^{a} is a bond, O, CO, X¹C(X²), C(X²)X¹, X^{I}C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and C₁₋₈ hydrocarbyl optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{c} is selected from hydrogen and C₁₋₄ hydrocarbyl; and
X¹ is 0, S or NR^{c} and X² is ═O, ═S or ═NR^{c}.

130. A compound according to claim 129 having the formula (III): 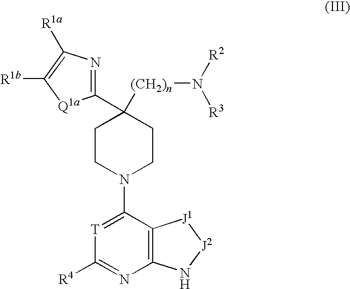
or salts, tautomers or N-oxides thereof, wherein Q^{1a} is NH or O; n is 0, 1 or 2; and R^{1a}, R^{1b}, R², R³, R⁴, T, J¹ and J² are as defined in any one of the preceding claims.

131. A compound according to claim 130 having the formula (IV): 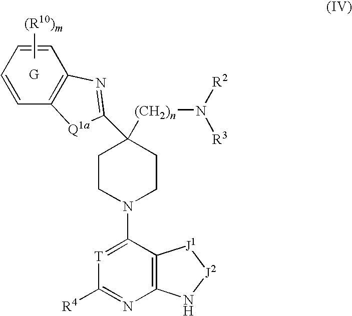
or salts, tautomers or N-oxides thereof, wherein Q^{1a} is NH or O; the ring G is a benzene or pyridine ring, m is 0, 1 or 2; n is 0, 1 or 2; and R², R³, R⁴, R¹⁰, T, J¹ and J² are as defined in any one of the preceding claims.

132. A compound according to claim 131 having the formula (IVa): 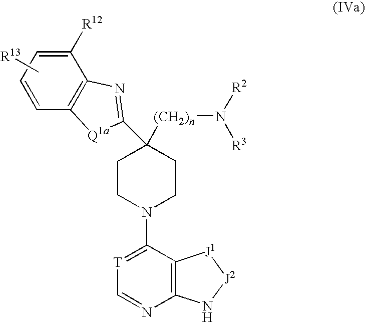
or salts, tautomers or N-oxides thereof, wherein R¹² is selected from:
hydrogen;
hydroxy;
halogen (e.g. fluorine and chlorine);
cyano;
amino;
mono-C₁₋₄-alkylamino or di-C₁₋₄-alkylamino wherein the C₁₋₄-alkyl moieties of the mono-C₁₋₄-alkylamino and di-C₁₋₄-alkylamino groups are optionally substituted by hydroxy (other than α-hydroxy), C₁₋₂ alkoxy, amino, mono-C₁₋₂-alkylamino, di-C₁₋₂-alkylamino, C₁₋₂ acylamino;
optionally substituted C₁₋₄ alkyl (e.g. methyl);
optionally substituted C₁₋₄ alkoxy (e.g. methoxy) groups; wherein the optional substituents for the C₁₋₄ alkyl and C₁₋₄ alkoxy groups are selected from halogen (e.g. fluorine), hydroxy, C₁₋₂ acyloxy, C₁₋₂ alkoxy (e.g. methoxy), amino, mono-C₁₋₄-alkylamino, di-C₁₋₄-alkylamino and C₁₋₄ acylamino; and
a group (CH₂)ₜ-R^{cyc″}, wherein t is 0, 1 or 2;
a 5-6 membered aryl or heteroaryl ring optionally substituted by C₁₋₄ alkyl, C₁₋₄ alkoxy, halogen, hydroxy, C₁₋₄ acyloxy, cyano, trifluoromethyl, trifluoromethoxy, difluoromethoxy, amino, mono-C₁₄₋-alkylamino, di-C₁₋₄-alkylamino or C₁₋₄ acylamino; or
a 3-7 membered non-aromatic carbocyclic or heterocyclic ring optionally substituted by C₁₋₄ alkyl, C₁₋₄ alkoxy, halogen, hydroxy, C₁₋₄ acyloxy, cyano, trifluoromethyl, trifluoromethoxy, difluoromethoxy, amino, mono-C₁₋₄-alkylamino, di-C₁₋₄-alkylamino; C₁₋₄ acylamino or C₁₋₄ alkoxycarbonyl; and R¹³ is selected from:
hydrogen;
hydroxy;
halogen (e.g. fluorine and chlorine);
cyano;
trifluoromethyl;
trifluoromethoxy;
difluoromethoxy;
amino;
methylamino;
dimethylamino;
methyl; and
methoxy.

133. A compound according to claim 130 having the formula (IVb): 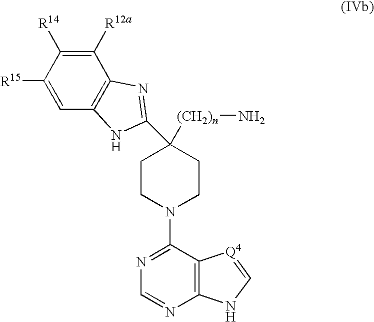
or salts, tautomers or N-oxides thereof, wherein Q⁴ is N or CH; n is 0 or 1; R^{12a} is selected from hydrogen, fluorine and chlorine; R¹⁴ is selected from hydrogen, fluorine, chlorine, methyl and methoxy; and R¹⁵ is selected from hydrogen and fluorine; provided that at least one _{of R}^{12a}, R¹⁴ and R¹⁵ is hydrogen.

134. A compound according to claim 130 having the formula)(IV⁰): 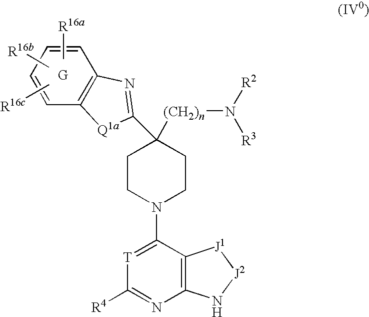
or salts, tautomers or N-oxides thereof, wherein n, T, J¹-J², R², R³ and R⁴ are as defined in any one of the preceding claims, Q^{1a} is NH or O, and R^{16a} is selected from:
hydrogen;
hydroxy;
halogen (e.g. fluorine, chlorine and bromine);
cyano;
amino;
mono-C₁₋₄-alkylamino or di-C₁₋₄-alkylamino wherein the C₁₋₄-alkyl moieties of the mono-C₁₋₄-alkylamino and di-C₁₋₄-alkylamino groups are optionally substituted by hydroxy (other than a-hydroxy), C₁₋₂ alkoxy, amino, mono-C₁₋₂-alkylamino, di-C₁₋₂-alkylamino, C₁₋₂ acylamino;
optionally substituted C₁₋₄ alkyl (e.g. methyl);
optionally substituted C₁₋₄ alkoxy (e.g. methoxy) groups;
optionally substituted C₁₋₄ alkylthio; wherein the optional substituents for the C₁₋₄ alkyl, C₁₋₄ alkoxy and C₁₋₄ alkylthio groups are selected from halogen (e.g. fluorine), hydroxy, C₁₋₂ acyloxy, C₁₋₂ alkoxy (e.g. methoxy), amino, mono-C₁₋₄-alkylamino, di-C₁₋₄-alkylamino and C₁₋₄ acylamino;
a group R^{cyc};
a group O—R^{cyc};
a group (O)ᵣ—(CH₂)ₜ-R^{cyc′}, wherein r is 0 or 1; t is 0, 1 or 2;
a 5-6 membered aryl or heteroaryl ring optionally substituted by C₁₋₄ alkyl, C₁₋₄ alkoxy, halogen, hydroxy, C₁₋₄ acyloxy, cyano, trifluoromethyl, trifluoromethoxy, difluoromethoxy, amino, mono-C₁₋₄-alkylamino, di-C₁₋₄-alkylamino, C₁₋₄ acylamino, aminosulphonyl, mono-C₁₋₄-alkylaminosulphonyl, di-C₁₋₄-alkylaminosulphonyl or C₁₋₄-alkylsulphonyl; or
a 3-7 membered non-aromatic carbocyclic or heterocyclic ring optionally substituted by C₁₋₄ alkyl, C₁₋₄ alkoxy, halogen, hydroxy, C₁₋₄ acyloxy, cyano, trifluoromethyl, trifluoromethoxy, difluoromethoxy, amino, mono-C₁₋₄-alkylamino, di-C₁₋₄-alkylamino; C₁₋₄ acylamino or C₁₋₄ alkoxycarbonyl;
R^{16b} is selected from:
hydrogen;
hydroxy;
halogen (e.g. fluorine and chlorine);
cyano;
trifluoromethyl;
trifluoromethoxy;
difluoromethoxy;
amino;
C₁₋₄ alkylamino;
di-C₁₋₄ alkylamino;
C₁₋₄ alkyl; and
C₁₋₄ alkoxy; and
R^{16c} is selected from:
hydrogen;
fluorine;
chlorine; and
methyl.

135. A compound according to claim 134 wherein the compound is a compound of the formula)(IV⁰⁰): 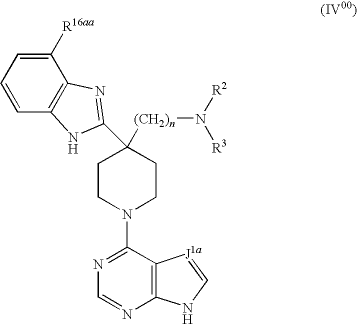
or a salt, tautomer or N-oxide thereof, wherein n, R² and R³ are as defined in any one of the preceding claims; J^{1a} is CH or N; and R^{16aa} is a monocyclic aromatic group selected from phenyl, pyridyl, pyrimidinyl, thienyl, furanyl, imidazolyl and pyrazolyl, each of which monocyclic aromatic groups is optionally substituted by methoxy, methyl, aminosulphonyl, methylsulphonyl, fluorine, chlorine, trifluoromethyl or trifluoromethoxy.

136. A compound according to claim 134 wherein the compound is a compound of the formula)(IV⁰⁰⁰): 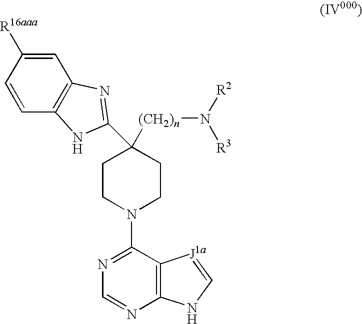
or a salt, tautomer or N-oxide thereof, wherein n, R² and R³ are as defined in any one of the preceding claims; J^{1a} is CH or N; and R^{16aaa} is selected from hydrogen, fluorine, chlorine, bromine, C₁₋₃ alkyl, hydroxy, methoxy, trifluoromethyl, di-C₁₋₂ alkylaminosulphonyl, trifluoromethoxy and trifluoromethylthio.

137. A compound according to claim 130 having the formula (V): 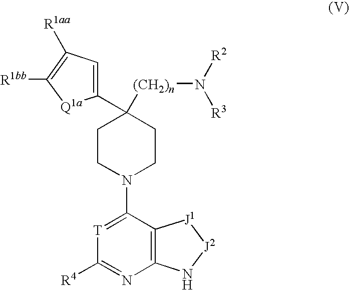
or salts, tautomers or N-oxides thereof, wherein R^{1aa} is hydrogen or a substituent R¹⁰, R^{1bb} is hydrogen or a substituent R¹⁰, Q^{1a} is NH or O; n is 0, 1 or 2; and R², R³, R⁴, R¹⁰, T, J¹ and J² are as defined in any one of the preceding claims.

138. A compound according to claim 121 in the form of a salt or N-oxide.

139. A method:
for the prophylaxis or treatment of a disease state or condition mediated by protein kinase B, which method comprises administering to a subject in need thereof a compound as defined in claim 121; or
for treating a disease or condition comprising or arising from abnormal cell growth or abnormally arrested cell death in a mammal, the method comprising administering to the mammal a compound as defined in any one of claim 121 in an amount effective to inhibit protein kinase B activity; or
of inhibiting protein kinase B, which method comprises contacting the kinase with a kinase-inhibiting compound as defined in claim 121; or
of modulating a cellular process by inhibiting the activity of a protein kinase B using a compound as defined in claim 121; or
for the prophylaxis or treatment of a disease state or condition mediated by protein kinase A, which method comprises administering to a subject in need thereof a compound as defined in claim 121; or
for treating a disease or condition comprising or arising from abnormal cell growth or abnormally arrested cell death in a mammal, the method comprising administering to the mammal a compound as defined in claim 121 in an amount effective to inhibit protein kinase A activity; or
of inhibiting protein kinase A, which method comprises contacting the kinase with a kinase-inhibiting compound as defined in claim 121; or
of modulating a cellular process by inhibiting the activity of a protein kinase A using a compound as defined in as defined in claim 121; or
for treating a disease or condition comprising or arising from abnormal cell growth or abnormally arrested cell death in a mammal, which method comprises administering to the mammal a compound as defined in claim 121 in an amount effective in inhibiting abnormal cell growth; or
for alleviating or reducing the incidence of a disease or condition comprising or arising from abnormal cell growth or abnormally arrested cell death in a mammal, which method comprises administering to the mammal a compound as defined in claim 121 in an amount effective in inhibiting abnormal cell growth; or
for the diagnosis and treatment of a disease state or condition mediated by protein kinase B, which method comprises (i) screening a patient to determine whether a disease or condition from which the patient is or may be suffering is one which would be susceptible to treatment with a compound having activity against protein kinase B; and (ii) where it is indicated that the disease or condition from which the patient is thus susceptible, thereafter administering to the patient a compound as defined in claim 121; or
for the diagnosis and treatment of a disease state or condition mediated by protein kinase A, which method comprises (i) screening a patient to determine whether a disease or condition from which the patient is or may be suffering is one which would be susceptible to treatment with a compound having activity against protein kinase A; and (ii) where it is indicated that the disease or condition from which the patient is thus susceptible, thereafter administering to the patient a compound as defined in claim 121; or
of modulating protein kinase B and/or protein kinase A; which method comprises bringing the protein kinase B and/or protein kinase A into contact with a compound as defined in claim 121.

140. A pharmaceutical composition comprising a novel compound as defined in claim 121 and a pharmaceutically acceptable carrier.


SEQUENCE LISTING (2 sequences)

SEQ ID NO: 1 - protein, 9 aa, Artificial
  Synthetic PKA specific peptide
GRTGRRNSI

SEQ ID NO: 2 - protein, 15 aa, Artificial
  Synthetic AKTide-2T
HARKRERTYSFGHHA